(19) United States
(12) Patent Application Publication
(10) Pub. No.: US 2023/0302109 A1
(43) Pub. Date: Sep. 28, 2023

(54) ANTIGEN POOL

(71) Applicants: The Francis Crick Institute Limited, London Greater London (GB); Enara Bio Limited, Oxford (GB)

(72) Inventors: George KASSIOTIS, London (GB); George YOUNG, London (GB); Jan ATTIG, London (GB); Ambrosius SNIJDERS, London (GB); David PERKINS, London (GB); Fabio MARINO, Oxford (GB); Ray JUPP, Oxford (GB); Magdalena VON ESSEN, Oxford (GB); Peter MASON, Oxford (GB); Nicola TERNETTE, Oxford (GB)

(21) Appl. No.: 18/046,670

(22) Filed: Oct. 14, 2022

Related U.S. Application Data
(63) Continuation of application No. PCT/GB21/50940, filed on Apr. 19, 2021.

(30) Foreign Application Priority Data
Apr. 17, 2020 (GB) 20170255.0

(51) Int. Cl. A61K 39/00; C07K 14/47; A61P 35/00

(52) U.S. Cl. CPC A61K 39/00119; C07K 14/4748; A61P 35/00; A61K 2039/876; A61K 2039/5158; A61K 2039/5154

(57) ABSTRACT

There are disclosed inter alia antigen pools which are useful in the treatment of cancer, particularly melanoma, especially cutaneous melanoma and uveal melanoma.

### RELATED APPLICATIONS

This application is a continuation of International Application No. PCT/GB2021/050940, filed on Apr. 19, 2021, which claims the benefit of United Kingdom Application No. 20170255.0, filed Apr. 17, 2020, the entire disclosures of which are hereby incorporated herein by reference in their entirety.

### SEQUENCE LISTING

The instant application contains a sequence listing which has been submitted electronically in ASCII ST.26 format and is hereby incorporated by reference in its entirety (said ASCII, ST.26 copy, created on Oct. 14, 2022, is named 193667_SL.xml and is 107466 bytes in size).

### FIELD OF THE INVENTION

The present invention relates to antigen pools comprising two or more different antigens. Such antigen pools are for use in the ex vivo stimulation and/or amplification of T cells derived from a human suffering from cancer (e.g. cutaneous melanoma or uveal melanoma). The present invention further relates inter alia to immunogenic pharmaceutical compositions comprising the antigen pool and a pharmaceutically acceptable carrier, processes for preparing a T cell population which is cytotoxic for cancer cells, immune cells and exosomes loaded with and/or stimulated by the antigen pool and pharmaceutical compositions thereof, their medical use and methods of treatment comprising administering the antigen pools, immunogenic pharmaceutical compositions, immune cells and exosomes.

### BACKGROUND OF THE INVENTION

As part of normal immunosurveillance for pathogenic microbes, all cells degrade intracellular proteins to produce peptides that are loaded onto Major Histocompatibility Complex (MHC) Class I molecules that are expressed on the surface of all cells. Most of these peptides, which are derived from the host cell, are recognized as self, and remain invisible to the adaptive immune system. However, peptides that are foreign (non-self), are capable of stimulating the expansion of naïve CD8+ T-cells that encode a T-cell receptor (TCR) that tightly binds the MHC I-peptide complex. This expanded T-cell population can produce effector CD8+ T-cells (including cytotoxic T-lymphocytes—CTLs) that can eliminate the foreign antigen-tagged cells, as well as memory CD8+ T-cells that can be re-amplified when the foreign antigen-tagged cells appear later in the animal's life.

MHC Class II molecules, whose expression is normally limited to professional antigen-presenting cells (APCs) such as dendritic cells (DCs), are usually loaded with peptides which have been internalised from the extracellular environment. Binding of a complementary TCR from a naïve CD4+ T-cell to the MHC II-peptide complex, in the presence of various factors, including T-cell adhesion molecules (CD54, CD48) and co-stimulatory molecules (CD40, CD80, CD86), induces the maturation of CD4+ T-cells into effector cells (e.g., T_{H}1, T_{H}2, T_{H}17, T_{FH}, T_{reg} cells). These effector CD4+ T-cells can promote B-cell differentiation to antibody-secreting plasma cells as well as facilitate the differentiation of antigen-specific CD8+ CTLs, thereby helping induce the adaptive immune response to foreign antigens, that include both short-term effector functions and longer-term immunological memory. DCs can perform the process of cross-presentation of peptide antigens by delivering exogenously-derived antigens (such as a peptide or protein released from a pathogen or a tumor cell) onto their MHC I molecules, contributing to the generation of immunological memory by providing an alternative pathway to stimulating the expansion of naïve CD8+ T-cells.

Immunological memory (specifically antigen-specific B cells/antibodies and antigen-specific CTLs) are critical players in controlling microbial infections, and immunological memory has been exploited to develop numerous vaccines that prevent the diseases caused by important pathogenic microbes. Immunological memory is also known to play a key role in controlling tumor formation, but very few efficacious cancer vaccines have been developed.

Cancer is the second leading cause of morbidity, accounting for nearly 1 in 6 of all deaths globally. Of the 8.8 million deaths caused by cancer in 2015, the cancers which claimed the most lives were from lung (1.69 million), liver (788,000), colorectal (774,000), stomach (754,000) and breast (571,000) carcinomas. The economic impact of cancer in 2010 was estimated to be USD1.16 Trillion, and the number of new cases is expected to rise by approximately 70% over the next two decades (World Health Organisation Cancer Facts 2017).

Current therapies for cutaneous melanoma are varied and are highly dependent on the location of the tumor and stage of the disease. The main treatment for a non-metastatic melanoma is surgery to remove the tumor and surrounding tissue. Later stage melanomas may require treatment comprising lymph node dissection, radiotherapy, or chemotherapy. Immune checkpoint blockade strategies, including the use of antibodies targeting negative immune regulators such PD-1/PD-L1 and CTLA4, have recently revolutionised treatments to a variety of malignancies, including melanoma (Ribas, A., & Wolchok, J. D. (2018) Science, 359:1350-1355.). The extraordinary value of checkpoint blockade therapies, and the well-recognized association of their clinical benefit with patient's adaptive immune responses (specifically T-cell based immune responses) to their own cancer antigens has re-invigorated the search for effective cancer vaccines, vaccine modalities, and cancer vaccine antigens.

Human endogenous retroviruses (HERVs) are remnants of ancestral germ line integrations of exogenous infectious retroviruses. HERVs belong to the group of endogenous retroelements that are characterised by the presence of Long Terminal Repeats (LTRs) flanking the viral genome. This group also includes the Mammalian apparent LTR Retrotransposons (MaLRs) and are therefore collectively known as LTR elements (here referred to collectively as ERV to mean all LTR elements). ERVs constitute a considerable proportion of the mammalian genome (8%), and can be grouped into approximately 100 families based on sequence homology. Many ERV sequences encode defective proviruses which share the prototypical retroviral genomic structure consisting of gag, pro, pol and env genes flanked by LTRs. Some intact ERV ORFs produce retroviral proteins which share features with proteins encoded by exogenous infectious retroviruses such as HIV-1. Such proteins may serve as antigens to induce a potent immune response (Hurst & Magiorkinis, 2015, J. Gen. Virol 96:1207-1218), suggesting that polypeptides encoded by ERVs can escape T and B-cell receptor selection processes and central and peripheral tolerance. Immune reactivity to ERV products may occur spontaneously in infection or cancer, and ERV products have been implicated as a cause of some autoimmune diseases (Kassiotis & Stoye, 2016, Nat. Rev. Immunol. 16:207-219).

Due to the accumulation of mutations and recombination events during evolution, most ERV-derived sequences have lost functional open reading frames for some or all of their genes and therefore their ability to produce infectious virus. However, these ERV elements are maintained in germline DNA like other genes and still have the potential to produce proteins from at least some of their genes. Indeed, HERV-encoded proteins have been detected in a variety of human cancers. For example, splice variants of the HERV-K env gene, Rec and Np9, are found exclusively in malignant testicular germ cells and not in healthy cells (Ruprecht et. al, 2008, Cell Mol Life Sci 65:3366-3382). Increased levels of HERV transcripts have also been observed in cancers such as those of the prostate, as compared to healthy tissue (Wang-Johanning, 2003, Cancer 98:187-197; Andersson et al., 1998, Int. J. Oncol, 12:309-313). Additionally, overexpression of HERV-E and HERV-H has been demonstrated to be immunosuppressive, which could also contribute to the development of cancer (Mangeney et al., 2001, J. Gen. Virol. 82:2515-2518). However, the exact mechanism(s) by which HERVs could contribute to the development or pathogenicity of cancer remains unknown.

In addition to deregulating the expression of surrounding neighbouring host genes, the activity and transposition of ERV regulatory elements to new genomic sites may lead to the production of novel transcripts, some of which may have oncogenic properties (Babaian & Mager, Mob. DNA, 2016, Lock et al., PNAS, 2014, 111:3534-3543).

A wide range of vaccine modalities are known. One well-described approach involves directly delivering an antigenic polypeptide to a subject with a view to raising an immune response (including B- and T-cell responses) and stimulating immunological memory. Alternatively, a polynucleotide may be administered to the subject by means of a vector such that the polynucleotide-encoded immunogenic polypeptide is expressed in vivo. The use of viral vectors, for example adenovirus vectors, has been well explored for the delivery of antigens in both prophylactic vaccination and therapeutic treatment strategies against cancer (Wold et al. Current Gene Therapy, 2013, Adenovirus Vectors for Gene Therapy, Vaccination and Cancer Gene Therapy, 13:421-433). Immunogenic peptides, polypeptides, or polynucleotides encoding them, can also be used to load patient-derived antigen presenting cells (APCs), that can then be infused into the subject as a vaccine that elicits a therapeutic or prophylactic immune response. An example of this approach is Provenge, which is presently the only FDA-approved anti-cancer vaccine.

Cancer antigens, may also be exploited in the treatment and prevention of cancer by using them to create a variety of non-vaccine therapeutic modalities. These therapies fall into two different classes: 1) antigen-binding biologics, 2) adoptive cell therapies.

Antigen-binding biologics typically consist of multivalent engineered polypeptides that recognize antigen-decorated cancer cells and facilitate their destruction. The antigen-binding components of these biologics may consist of TCR-based biologicals, including, but not limited to TCRs, high-affinity TCRs, and TCR mimetics produced by various technologies (including those based on monoclonal antibody technologies). Cytolytic moieties of these types of multivalent biologics may consist of cytotoxic chemicals, biological toxins, targeting motifs and/or immune stimulating motifs that facilitate targeting and activation of immune cells, any of which facilitate the therapeutic destruction of tumor cells.

Adoptive cell therapies may be based on a patient's own T-cells that are removed and stimulated ex vivo with vaccine antigen preparations (cultivated with T-cells in the presence or absence of other factors, including cellular and acellular components) (Yossef et al., JCI Insight. 2018 Oct. 4; 3(19). pii: 122467. doi: 10.1172/jci.insight.122467). Alternatively, adoptive cell therapies can be based on cells (including patient- or non-patient-derived cells) that have been deliberately engineered to express antigen-binding polypeptides that recognize cancer antigens. These antigen-binding polypeptides fall into the same classes as those described above for antigen-binding biologics. Thus, lymphocytes (autologous or non-autologous), that have been genetically manipulated to express cancer antigen-binding polypeptides can be administered to a patient as adoptive cell therapies to treat their cancer.

Use of ERV-derived antigens in raising an effective immune response to cancer has shown promising results in promoting tumor regression and a more favourable prognosis in murine models of cancer (Kershaw et al., 2001, Cancer Res. 61:7920-7924; Slansky et al., 2000, Immunity 13:529-538). Thus, HERV antigen-centric immunotherapy trials have been contemplated in humans (Sacha et al., 2012, J. Immunol 189:1467-1479), although progress has been restricted, in part, due to a severe limitation of identified tumor-specific ERV antigens.

WO 2005/099750 identifies anchored sequences in existing vaccines against infectious pathogens, which are common in raising cross-reactive immune responses against the HERV-K Mel tumor antigen and confers protection to melanoma.

WO 00/06598 relates to the identification of HERV-AVL3-B tumor associated genes which are preferentially expressed in melanomas, and methods and products for diagnosing and treating conditions characterised by expression of said genes.

WO 2006/119527 provides antigenic polypeptides derived from the melanoma-associated endogenous retrovirus (MERV), and their use for the detection and diagnosis of melanoma as well as prognosis of the disease. The use of antigenic polypeptides as anticancer vaccines is also disclosed.

WO 2007/137279 discloses methods and compositions for detecting, preventing and treating HERV-K+ cancers, for example with use of a HERV-K+ binding antibody to prevent or inhibit cancer cell proliferation.

WO 2006/103562 discloses a method for treating or preventing cancers in which the immunosuppressive Np9 protein from the env gene of HERV-K is expressed. The invention also relates to pharmaceutical compositions comprising nucleic acid or antibodies capable of inhibiting the activity of said protein, or immunogen or vaccinal composition capable of inducing an immune response directed against said protein.

WO 2007/109583 provides compositions and methods for preventing or treating neoplastic disease in a mammalian subject, by providing a composition comprising an enriched immune cell population reactive to a HERV-E antigen on a tumor cell.

Humer J, et al., 2006, Canc. Res., 66:1658-63 identifies a melanoma marker derived from melanoma-associated endogenous retroviruses.

There is a need to identify novel antigen pools comprising HERV-associated antigenic sequences which can be used in immunotherapy of cancer, particularly melanoma, especially cutaneous and uveal melanoma.

### SUMMARY OF THE INVENTION

The inventors have surprisingly discovered certain RNA transcripts which comprise LTR elements or are derived from genomic sequences adjacent to LTR elements which are found at high levels in cutaneous melanoma cells, but are undetectable or found at very low levels in normal, healthy tissues (see Example 1). Such transcripts are herein referred to as cancer-specific LTR-element spanning transcripts (CLTs). Further, the inventors have shown that a subset of the potential polypeptide sequences (i.e., open reading frames (ORFs)) encoded by these CLTs are translated in cancer cells, processed by components of the antigen-processing apparatus, and presented on the surface of cells found in tumor tissue in association with the class I and class II major histocompatibility complex (MHC Class I, and MHC Class II) and class I and class II human leukocyte antigen (HLA Class I, HLA Class II) molecules (see Example 2). These findings demonstrate that these polypeptides (herein referred to as CLT antigens) are, ipso facto, antigenic. Thus, cancer cell presentation of CLT antigens is expected to render these cells susceptible to elimination by T-cells that bear cognate T-cell receptors (TCRs) for the CLT antigens, and CLT antigen-based vaccination methods/regimens that amplify T-cells bearing these cognate TCRs are expected to elicit immune responses against cancer cells (and tumors containing them), particularly melanoma particularly cutaneous melanoma tumors. T-cells from melanoma subjects are indeed reactive to peptides derived from CLT antigens disclosed herein and amplify T-cells and amplify T-cell receptor sequences (see Example 3). The inventors have confirmed that T-cells specific for CLT antigens have not been deleted from normal subject's T-cell repertoire by central tolerance (see Example 4). The presence and killing activity of CLT antigen specific T-cells in ex vivo cultures of healthy donor T-cells has been determined (see Example 5). Finally, qRT-PCR studies have confirmed that CLTs are specifically expressed in RNA extracted from melanoma cell lines as compared to non-melanoma cells lines (see Example 6). The inventors have also produced fusion proteins comprising unique CLT antigens (Example 8).

The inventors have also surprisingly discovered that certain CLT antigen-encoding CLTs as well as being overexpressed in cutaneous melanoma are also overexpressed in uveal melanoma. The CLT antigen polypeptide sequences encoded by these CLTs are expected to elicit immune responses against uveal melanoma cells and tumors containing them.

The CLTs and the CLT antigens are not canonical sequences which can be readily derived from known tumor genome sequences found in the cancer genome atlas. The CLTs are transcripts resulting from complex transcription and splicing events driven by transcription control sequences of ERV origin. Since the CLTs are expressed at high level and since CLT antigen polypeptide sequences are not sequences of normal human proteins, it is expected that they will be capable of eliciting strong, specific immune responses (as indeed has been established—see Examples 3-5) and are thus suitable for therapeutic use in a cancer immunotherapy setting.

The CLT antigens discovered in the highly expressed transcripts that characterize tumor cells, which were previously not known to exist and produce protein products in man and to stimulate immune responses, can be used in several formats. For example, CLT antigen polypeptides can be directly delivered to a subject as a vaccine that elicits a therapeutic or prophylactic immune response to tumor cells. Also, nucleic acids, which may be codon optimised to enhance the expression of their encoded CLT antigens, can be directly administered or else inserted into vectors for delivery in vivo to produce the encoded protein products in a subject as a vaccine that elicits a therapeutic or prophylactic immune response to tumor cells.

Antigen pools of the present invention comprising two or more different antigens wherein each antigen is present in the form of a polypeptide and/or nucleic acid encoding the polypeptides can be used to load patient-derived antigen presenting cells (APCs), that can then be infused into the subject as a vaccine that elicits a therapeutic or prophylactic immune response to tumor cells. Further, antigen pools of the present invention comprising two or more different antigens wherein each antigen is present in the form of a polypeptide and/or nucleic acid encoding the polypeptides can be used for ex vivo stimulation of a subject's T-cells, producing a stimulated T-cell preparation that can be administered to a subject as a therapy to treat cancer. These and other applications are described in greater detail below.

Thus, the invention provides inter alia an antigen pool comprising two or more different antigens, wherein each antigen is present in the form of a polypeptide and/or a nucleic acid encoding said polypeptide, and wherein the different antigens are present in the antigen pool as separate polypeptides, nucleic acids, a fusion protein and/or a nucleic acid encoding said fusion protein, wherein the two or more different antigens have polypeptide sequences selected from:
(a) SEQ ID NO: 1 or a variant thereof, or an immunogenic fragment of SEQ ID NO: 1 or a variant thereof;
(b) SEQ ID NO: 2 or a variant thereof or an immunogenic fragment of SEQ ID NO: 2 or a variant thereof;
(c) SEQ ID NO: 3 or a variant thereof, or an immunogenic fragment of SEQ ID NO: 3 or a variant thereof;
(d) SEQ ID NO: 4 or a variant thereof, or an immunogenic fragment of SEQ ID NO: 4 or a variant thereof;
(e) SEQ ID NO: 5 or a variant thereof, or an immunogenic fragment of SEQ ID NO: 5 or a variant thereof;
(f) SEQ ID NO: 6 or a variant thereof, or an immunogenic fragment of SEQ ID NO: 6 or a variant thereof;
(g) SEQ ID NO: 7 or a variant thereof, or an immunogenic fragment of SEQ ID NO: 7 or a variant thereof; and
(h) SEQ ID NO: 8 or a variant thereof, or an immunogenic fragment of SEQ ID NO: 8 or a variant thereof.
(hereinafter referred to as “antigen pools of the invention”).

The antigen pools of the invention, as well as related aspects of the invention, are expected to be useful in a range of embodiments in cancer immunotherapy and prophylaxis, particularly immunotherapy and prophylaxis of melanoma, as discussed in more detail below.

### DESCRIPTION OF THE FIGURES

Each of FIGS. 1-38 shows an extracted MS/MS spectrum (with assigned fragment ions) of a peptide obtained from a tumor sample of a patient and either a bottom panel showing a rendering of the spectrum indicating the positions of the linear peptide sequences that have been mapped to the fragment ions or similar data shown in tabular form.

FIG. 1. Spectra for the peptide of SEQ ID NO. 9 obtained from a tumor sample of patient Mel-3.

FIG. 2. Spectra for the peptide of SEQ ID NO. 10 obtained from a tumor sample of patient Mel-3.

FIG. 3. Spectra for the peptide of SEQ ID NO. 10 obtained from a tumor sample of patient Mel-3.

FIG. 4. Spectra for the peptide of SEQ ID NO. 10 obtained from a tumor sample of patient 2MT3.

FIG. 5. Spectra for the peptide of SEQ ID NO. 11 obtained from a tumor sample of patient Mel-5.

FIG. 6. Spectra for the peptide of SEQ ID NO. 11 obtained from a tumor sample of patient Mel-16.

FIG. 7. Spectra for the peptide of SEQ ID NO. 11 obtained from a tumor sample of patient Mel-16.

FIG. 8. Spectra for the peptide of SEQ ID NO. 11 obtained from a tumor sample of patient 2MT3.

FIG. 9. Spectra for the peptide of SEQ ID NO. 11 obtained from a tumor sample of patient 2MT10.

FIG. 10 Spectra for the peptide of SEQ ID NO. 12 obtained from a tumor sample of patient Mel-5.

FIG. 11. Spectra for the peptide of SEQ ID NO. 18 obtained from a tumor sample of patient Mel-26.

FIG. 12. Spectra for the peptide of SEQ ID NO. 19 obtained from a tumor sample of patient Mel-20.

FIG. 13. Spectra for the peptide of SEQ ID NO. 19 obtained from a tumor sample of patient Mel-20.

FIG. 14. Spectra for the peptide of SEQ ID NO. 19 obtained from a tumor sample of patient 2MT4.

FIG. 15. Spectra for the peptide of SEQ ID NO. 31 obtained from a tumor sample of patient Mel-35.

FIG. 16. Spectra for the peptide of SEQ ID NO. 31 obtained from a tumor sample of patient 2MT3.

FIG. 17. Spectra for the peptide of SEQ ID NO. 32 obtained from a tumor sample of patient 1MT1.

FIG. 18. Spectra for the peptide of SEQ ID NO. 36 obtained from a tumor sample of patient Mel-3.

FIG. 19. Spectra for the peptide of SEQ ID NO. 36 obtained from a tumor sample of patient Mel-3.

FIG. 20. Spectra for the peptide of SEQ ID NO. 36 obtained from a tumor sample of patient 2MT3.

FIG. 21. Spectra for the peptide of SEQ ID NO. 36 obtained from a tumor sample of patient 2MT1.

FIG. 22. Spectra for the peptide of SEQ ID NO. 37 obtained from a tumor sample of patient Mel-40.

FIG. 23. Spectra for the peptide of SEQ ID NO. 37 obtained from a tumor sample of patient Mel-41.

FIG. 24. Spectra for the peptide of SEQ ID NO. 37 obtained from a tumor sample of patient 2MT3.

FIG. 25. Spectra for the peptide of SEQ ID NO. 38 obtained from a tumor sample of patient Mel-27.

FIG. 26. Spectra for the peptide of SEQ ID NO. 38 obtained from a tumor sample of patient Mel-39.

FIG. 27. Spectra for the peptide of SEQ ID NO. 39 obtained from a tumor sample of patient 2MT12.

FIG. 28. Spectra for the peptide of SEQ ID NO. 45 obtained from a tumor sample of patient Mel-29.

FIG. 29. Spectra for the peptide of SEQ ID NO. 48 obtained from a tumor sample of patient Mel-41.

FIG. 30. Spectra for the peptide of SEQ ID NO. 49 obtained from a tumor sample of patient Mel-41.

FIG. 31. Spectra for the peptide of SEQ ID NO. 50 obtained from a tumor sample of patient Mel-41.

FIG. 32. Spectra for the peptide of SEQ ID NO. 51 obtained from a tumor sample of patient Mel-41.

FIG. 33. Spectra for the peptide of SEQ ID NO. 52 obtained from a tumor sample of patient Mel-21.

FIG. 34. Spectra for the peptide of SEQ ID NO. 52 obtained from a tumor sample of patient 2MT3.

FIG. 35. Spectra for the peptide of SEQ ID NO. 53 obtained from a tumor sample of patient Mel-27.

FIG. 36. Spectra for the peptide of SEQ ID NO. 54 obtained from a tumor sample of patient Mel-27.

FIG. 37. Spectra for the peptide of SEQ ID NO. 54 obtained from a tumor sample of patient 2MT4.
Each of FIGS. 38-53 shows an alignment of a native MS/MS spectrum of a peptide obtained from a patient tumor sample (upper) to the native spectrum of a synthetic peptide corresponding to the same sequence (lower).

FIG. 38 shows a mass spectrometry spectrum of a peptide fragment from immunopeptidomic analysis of patient 2MT3 attributed to SEQ ID NO. 10.

FIG. 39 shows a mass spectrometry spectrum of a peptide fragment from immunopeptidomic analysis of patient 2MT3 attributed to SEQ ID NO. 11.

FIG. 40 shows a mass spectrometry spectrum of a peptide fragment from immunopeptidomic analysis of patient 2MT4 attributed to SEQ ID NO. 19.

FIG. 41 shows a mass spectrometry spectrum of a peptide fragment from immunopeptidomic analysis of patient 2MT3 attributed to SEQ ID NO. 31.

FIG. 42 shows a mass spectrometry spectrum of a peptide fragment from immunopeptidomic analysis of patient 1MT1 attributed to SEQ ID NO. 32.

FIG. 43 shows a mass spectrometry spectrum of a peptide fragment from immunopeptidomic analysis of patient 2MT3 attributed to SEQ ID NO. 36.

FIG. 44 shows a mass spectrometry spectrum of a peptide fragment from immunopeptidomic analysis of patient 2MT3 attributed to SEQ ID NO. 37.

FIG. 45 shows a mass spectrometry spectrum of a peptide fragment from immunopeptidomic analysis of patient 2MT12 attributed to SEQ ID NO. 39.

FIG. 46 shows a mass spectrometry spectrum of a peptide fragment from immunopeptidomic analysis of patient Mel-29 attributed to SEQ ID NO. 45.

FIG. 47 shows a mass spectrometry spectrum of a peptide fragment from immunopeptidomic analysis of patient Mel-41 attributed to SEQ ID NO. 48.

FIG. 48 shows a mass spectrometry spectrum of a peptide fragment from immunopeptidomic analysis of patient Mel-41 attributed to SEQ ID NO. 49.

FIG. 49 shows a mass spectrometry spectrum of a peptide fragment from immunopeptidomic analysis of patient Mel-41 attributed to SEQ ID NO. 50.

FIG. 50 shows a mass spectrometry spectrum of a peptide fragment from immunopeptidomic analysis of patient Mel-41 attributed to SEQ ID NO. 51.

FIG. 51 shows a mass spectrometry spectrum of a peptide fragment from immunopeptidomic analysis of patient Mel-21 attributed to SEQ ID NO. 52.

FIG. 52 shows a mass spectrometry spectrum of a peptide fragment from immunopeptidomic analysis of patient Mel-27 attributed to SEQ ID NO. 53.

FIG. 53 shows a mass spectrometry spectrum of a peptide fragment from immunopeptidomic analysis of patient Mel-27 attributed to SEQ ID NO. 54.

FIG. 54 panels A to C shows tumor antigen-specific T-cell amplification from patient PBMC cultures in response to cultivation with specific tumor antigen-derived peptides.

FIG. 55 panels A to D provides a summary of CLT Antigen-derived peptides (SEQ ID NO. 11, 13-15, 19-29, 33-35, 40-42) that were capable of amplifying specific TCR-bearing T-cells from melanoma patient PBMCs.

FIG. 56 shows CD8 T-cell responses from a normal blood donor to a HLA-A*02:01-restricted peptide (SEQ ID NO. 16) from CLT Antigen 1.

FIG. 57 shows CD8 T-cell responses from a normal blood donor to HLA-A*02:01-restricted peptide (SEQ ID NO. 30) from CLT Antigen 2.

FIG. 58 shows CD8 T-cell responses from a normal blood donor to HLA-A*02:01-restricted peptide (SEQ ID NO. 43) from CLT Antigen 4.

FIG. 59 shows CD8 T-cell responses from a normal blood donor to HLA-A*03:01-restricted peptide (SEQ ID NO. 47) from CLT Antigen 5.

FIG. 60 shows CD8 T-cell responses from a normal blood donor to HLA-B*07:02-restricted peptide (SEQ ID NO. 50) from CLT Antigen 6.

FIG. 61 shows CD8 T-cell responses from a normal blood donor to HLA-A*03:01-restricted peptide (SEQ ID NO. 52) from CLT Antigen 7.

FIG. 62 shows CD8 T-cell responses from a normal blood donor to HLA-A*02:01-restricted peptide (SEQ ID NO. 55) from CLT Antigen 8.

FIG. 63 panels A to D shows responsiveness to HLA-B*07:02 restricted peptides (SEQ ID NO. 17 and 44) from CLT Antigen 1 and CLT Antigen 4 respectively in memory CD45RO-positive CD8 T-cells as compared with naïve CD45RO-negative CD8 T-cells from the same donor.

FIG. 64 shows expanded, pentamer-sorted CD8 T-cells killing C1RB7-target cells pulsed with a peptide (SEQ ID NO. 44) derived from CLT Antigen 4.

FIG. 65 shows expanded, pentamer-sorted CD8 T-cells killing of CaSki cells transfected with the open reading frame of CLT Antigen 008 (SEQ ID NO. 8).

FIG. 66 panels A to G shows qRT-PCR assay results to verify the transcription of the CLT encoding CLT Antigen 1 (SEQ ID NO. 56), the CLT encoding CLT Antigen 2 (SEQ ID NO. 57), the CLT encoding CLT Antigen 3 and 4 (SEQ ID NO. 58), the CLT encoding CLT Antigen 5 (SEQ ID NO. 59), the CLT encoding CLT Antigen 6 (SEQ ID NO. 60), the CLT encoding CLT Antigen 7 (SEQ ID NO. 61) and the CLT encoding CLT Antigen 8 (SEQ ID NO. 62) in melanoma cancer cell lines or primary tissue samples.

FIG. 67 shows schematically the construction of CLT Antigen Fusion Protein 1 (SEQ ID NO. 76), the linker sequences between CLT Antigens and likely HLA binding of linker-derived epitopes. FP=fusion protein.

FIG. 68 shows schematically the construction of CLT Antigen Fusion Protein 2 (SEQ ID NO. 77), the linker sequences between CLT Antigens and likely HLA binding of linker-derived epitopes. FP=fusion protein.

FIG. 69 shows schematically the construction of CLT Antigen Fusion Protein 3 (SEQ ID NO. 78), the linker sequences between CLT Antigens and likely HLA binding of linker-derived epitopes. FP=fusion protein.

FIG. 70 shows schematically the construction of CLT Antigen Fusion Protein 4 (SEQ ID NO. 79), the linker sequences between CLT Antigens and likely HLA binding of linker-derived epitopes. FP=fusion protein.

### DESCRIPTION OF THE SEQUENCES

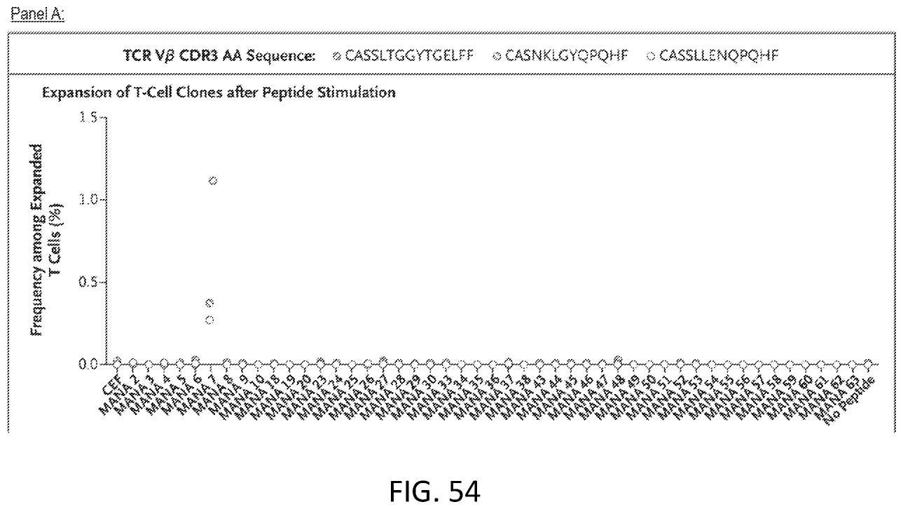

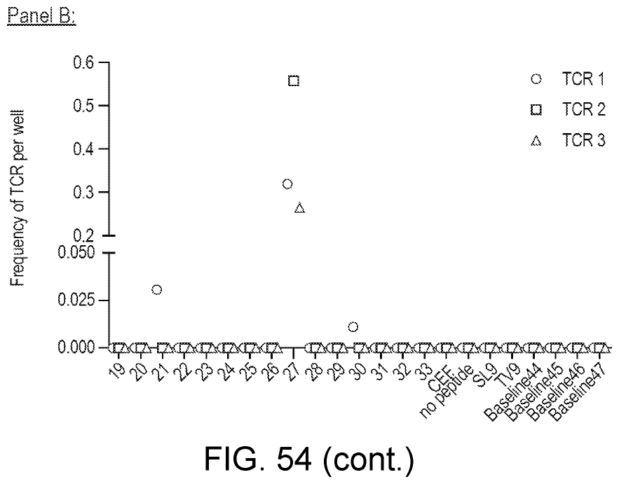

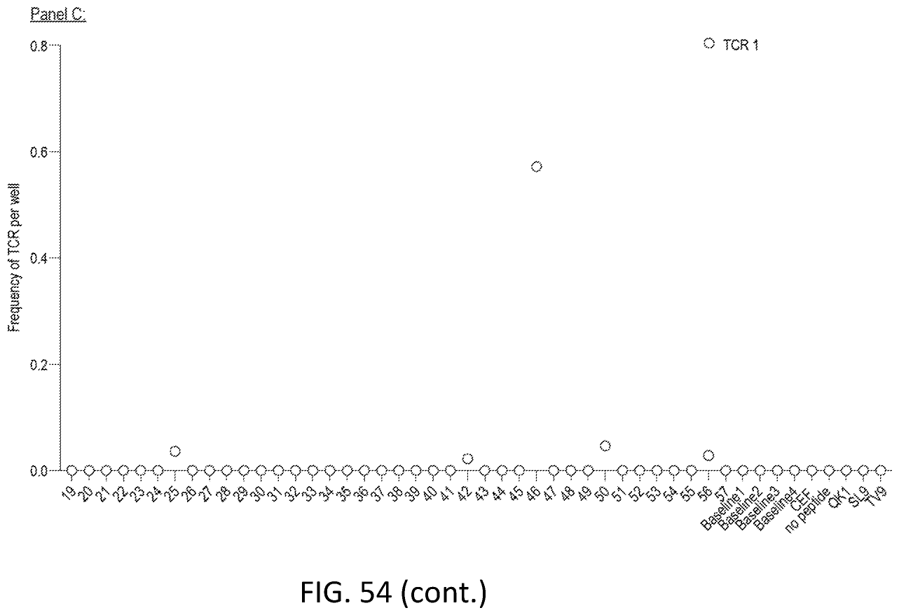

SEQ ID NO. 1 is the polypeptide sequence of CLT Antigen 1
SEQ ID NO. 2 i is the polypeptide sequence of CLT Antigen 2
SEQ ID NO. 3 is the polypeptide sequence of CLT Antigen 3
SEQ ID NO. 4 is the polypeptide sequence of CLT Antigen 4
SEQ ID NO. 5 is the polypeptide sequence of CLT Antigen 5
SEQ ID NO. 6 i is the polypeptide sequence of CLT Antigen 6
SEQ ID NO. 7 is the polypeptide sequence of CLT Antigen 7
SEQ ID NO. 8 is the polypeptide sequence of CLT Antigen 8
SEQ ID NOs. 9-17 are peptide sequences derived from CLT Antigen 1
SEQ ID NOs. 18-30 are peptide sequences derived from CLT Antigen 2
SEQ ID NOs. 31-35 are peptide sequences derived from CLT Antigen 3
SEQ ID NO. 36-44 are peptide sequences derived from CLT Antigen 4
SEQ ID NOs. 45-47 are peptide sequences derived from CLT Antigen 5
SEQ ID NO. 48-51 are peptide sequences derived from CLT Antigen 6
SEQ ID NO. 52 is a peptide sequence derived from CLT Antigen 7
SEQ ID NOs. 53-55 are peptide sequences derived from CLT Antigen 8
SEQ ID NO. 56 is the cDNA sequence of the CLT encoding CLT Antigen 1
SEQ ID NO. 57 is the cDNA sequence of the CLT encoding CLT Antigen 2
SEQ ID NO. 58 is the cDNA sequence of the CLT encoding CLT Antigens 3 and 4
SEQ ID NO. 59 is the cDNA sequence of the CLT encoding CLT Antigen 5
SEQ ID NO. 60 is the cDNA sequence of the CLT encoding CLT Antigen 6
SEQ ID NO. 61 is the cDNA sequence of the CLT encoding CLT Antigens 7
SEQ ID NO. 62 is the cDNA sequence of the CLT encoding CLT Antigen 8
SEQ ID NO. 63 is a cDNA sequence encoding CLT Antigen 1
SEQ ID NO. 64 is a cDNA sequence encoding CLT Antigen 2
SEQ ID NO. 65 is a cDNA sequence encoding CLT Antigen 3
SEQ ID NO. 66 is a cDNA sequence encoding CLT Antigen 4
SEQ ID NO. 67 is a cDNA sequence encoding CLT Antigen 5
SEQ ID NO. 68 is a cDNA sequence encoding CLT Antigen 6
SEQ ID NO. 69 is a cDNA sequence encoding CLT Antigen 7
SEQ ID NO. 70 is a cDNA sequence encoding CLT Antigen 8
SEQ ID NO. 71-75 are linker sequences used to construct CLT Antigen Fusion Proteins
SEQ ID NO. 76 is the polypeptide sequence of CLT Antigen Fusion Protein 1
SEQ ID NO. 77 is the polypeptide sequence of CLT Antigen Fusion Protein 2
SEQ ID NO. 78 is the polypeptide sequence of CLT Antigen Fusion Protein 3
SEQ ID NO. 79 is the polypeptide sequence of CLT Antigen Fusion Protein 4
SEQ ID NO. 80 is a cDNA sequence encoding CLT Antigen Fusion Protein 1
SEQ ID NO. 81 is a cDNA sequence encoding CLT Antigen Fusion Protein 2
SEQ ID NO. 82 is a cDNA sequence encoding CLT Antigen Fusion Protein 3
SEQ ID NO. 83 is a cDNA sequence encoding CLT Antigen Fusion Protein 4
SEQ ID NO. 84 is a linker sequence used to construct CLT Antigen Fusion Proteins
SEQ ID NOs: 85-87 are TCR VB CDR3 AA sequences shown in FIG. 54

### DETAILED DESCRIPTION OF THE INVENTION

### Antigen Pools

An “antigen pool” is a pool that contains two or more antigens (e.g. two, three, four, five, six, seven, eight antigens etc) where the antigens are in the form of polypeptides and/or nucleic acids. The antigen pool may be a pool of separate polypeptides or nucleic acids, or a pool of separate polypeptides and nucleic acids.

The polypeptides and/or nucleic acids in the antigen pool may be present as part of a fusion protein and/or a nucleic acid encoding the fusion protein, respectively. The term “fusion protein” refers to any protein comprising at least two polypeptides that are joined together by peptide bonds, through protein synthesis. The fusion protein may be created through the joining of two or more genes that encode for separate polypeptides that have been joined so that they are transcribed and translated as a single unit producing a single protein.

Thus, the present invention provides for an antigen pool comprising two or more different antigens, wherein each antigen is present in the form of a polypeptide and/or nucleic acid encoding said polypeptide and wherein the different antigens are present in the antigen pool as separate polypeptides or nucleic acids and/or as part of a fusion protein or nucleic acid encoding a fusion protein, wherein the two or more different antigens have polypeptide sequences selected from:
(a) SEQ ID NO: 1 or a variant thereof, or an immunogenic fragment of SEQ ID NO: 1 or a variant thereof;
(b) SEQ ID NO: 2 or a variant thereof or an immunogenic fragment of SEQ ID NO: 2 or a variant thereof;
(c) SEQ ID NO: 3 or a variant thereof, or an immunogenic fragment of SEQ ID NO: 3 or a variant thereof;
(d) SEQ ID NO: 4 or a variant thereof, or an immunogenic fragment of SEQ ID NO: 4 or a variant thereof;
(e) SEQ ID NO: 5 or a variant thereof, or an immunogenic fragment of SEQ ID NO: 5 or a variant thereof;
(f) SEQ ID NO: 6 or a variant thereof, or an immunogenic fragment of SEQ ID NO: 6 or a variant thereof;
(g) SEQ ID NO: 7 or a variant thereof, or an immunogenic fragment of SEQ ID NO: 7 or a variant thereof; and
(h) SEQ ID NO: 8 or a variant thereof, or an immunogenic fragment of SEQ ID NO: 8 or a variant thereof.

The antigen pool of the invention may comprise two or more, three or more, four or more, five or more, six or more, seven or more, or eight different antigens. The antigen pool of the invention preferably comprises two or more antigens. The antigen pool of the invention may comprise two, three, four, five, six, seven or eight different antigens. In a preferred embodiment of the invention, the antigen pool comprises six different antigens. In another preferred embodiment of the invention, the antigen pool comprises eight different antigens.

It will be understood that “two different antigens” means, for example, the combination of antigens (a) and (b) where antigen (a) is represented by a polypeptide having the sequence of SEQ ID NO: 1 or a variant thereof, or an immunogenic fragment of SEQ ID NO: 1 or a variant thereof, or a polynucleotide encoding said polypeptide, and antigen (b) is represented by a polypeptide having the sequence of SEQ ID NO: 2 or a variant thereof, or an immunogenic fragment of SEQ ID NO: 2 or a variant thereof, or a polynucleotide encoding said polypeptide. Each antigen may be represented by more than one component (e.g. more than one polypeptide or polynucleotide or a combination thereof). The same follows for any combination of two or more antigens.

In an embodiment of the invention the antigen pool comprises two different antigens.

In an embodiment of the invention the antigen pool comprises three different antigens.

In an embodiment of the invention the antigen pool comprises four different antigens.

In an embodiment of the invention the antigen pool comprises five different antigens.

In an embodiment of the invention the antigen pool comprises six different antigens.

In an embodiment of the invention the antigen pool comprises seven different antigens.

In an embodiment of the invention the antigen pool comprises eight different antigens.

In one embodiment, the antigen pool comprises as a polypeptide (optionally in the form of a fusion protein) or a polynucleotide encoding a polypeptide (optionally in the form of a fusion protein) having the polypeptide sequence: (a) SEQ ID NO: 1 or a variant thereof, or an immunogenic fragment of SEQ ID NO: 1 or a variant thereof.

In one embodiment, the antigen pool comprises as a polypeptide (optionally in the form of a fusion protein) or a polynucleotide encoding a polypeptide (optionally in the form of a fusion protein) having the polypeptide sequence: (b) SEQ ID NO: 2 or a variant thereof, or an immunogenic fragment of SEQ ID NO: 2 or a variant thereof.

In one embodiment, the antigen pool comprises as a polypeptide (optionally in the form of a fusion protein) or a polynucleotide encoding a polypeptide (optionally in the form of a fusion protein) having the polypeptide sequence: (c) SEQ ID NO: 3 or a variant thereof, or an immunogenic fragment of SEQ ID NO: 3 or a variant thereof.

In one embodiment, the antigen pool comprises as a polypeptide (optionally in the form of a fusion protein) or a polynucleotide encoding a polypeptide (optionally in the form of a fusion protein) having the polypeptide sequence: (d) SEQ ID NO: 4 or a variant thereof, or an immunogenic fragment of SEQ ID NO: 4 or a variant thereof.

In one embodiment, the antigen pool comprises as a polypeptide (optionally in the form of a fusion protein) or a polynucleotide encoding a polypeptide (optionally in the form of a fusion protein) having the polypeptide sequence: (e) SEQ ID NO: 5 or a variant thereof, or an immunogenic fragment of SEQ ID NO: 5 or a variant thereof.

In one embodiment, the antigen pool comprises as a polypeptide (optionally in the form of a fusion protein) or a polynucleotide encoding a polypeptide (optionally in the form of a fusion protein) having the polypeptide sequence: (f) SEQ ID NO: 6 or a variant thereof, or an immunogenic fragment of SEQ ID NO: 6 or a variant thereof.

In one embodiment, the antigen pool comprises as a polypeptide (optionally in the form of a fusion protein) or a polynucleotide encoding a polypeptide (optionally in the form of a fusion protein) having the polypeptide sequence: (g) SEQ ID NO: 7 or a variant thereof, or an immunogenic fragment of SEQ ID NO: 7 or a variant thereof.

In one embodiment, the antigen pool comprises as a polypeptide (optionally in the form of a fusion protein) or a polynucleotide encoding a polypeptide (optionally in the form of a fusion protein) having the polypeptide sequence: (h) SEQ ID NO: 8 or a variant thereof, or an immunogenic fragment of SEQ ID NO: 8 or a variant thereof.

In an embodiment of the invention where the antigen pool comprises six different antigens, the six different antigens suitably have the polypeptide sequences of (a), (b), (d), (f), (g) and (h). Thus, the antigen pool of the invention comprises six different antigens, wherein the antigens have the polypeptide sequences of (a), (b), (d), (f), (g) to (h).

In an embodiment of the invention where the antigen pool comprises eight different antigens, the eight different antigens suitably have the polypeptide sequences of (a) to (h). Thus, the antigen pool of the invention comprises eight different antigens, wherein the antigens have the polypeptide sequences of (a) to (h).

In an embodiment of the invention, each of the different antigens is present in the form of a separate polypeptide (i.e. not as part of a fusion protein).

In an embodiment of the invention, each of the different antigens is present as part of a fusion protein.

In an embodiment of the invention, each of the different antigens is present in the form of a separate nucleic acid (i.e. not as a polynucleotide encoding a fusion protein).

In an embodiment of the invention, each of the different antigens is present as part of a nucleic acid encoding a fusion protein.

### Polypeptides

The terms “protein”, “polypeptide” and “peptide” are used interchangeably herein and refer to any peptide-linked chain of amino acids, regardless of length, co-translational or post-translational modification.

The term “amino acid” refers to any one of the naturally occurring amino acids, as well as amino acid analogs and amino acid mimetics that function in a manner which is similar to the naturally occurring amino acids. Naturally occurring amino acids are those 20 L-amino acids encoded by the genetic code, as well as those amino acids that are later modified, e.g., hydroxyproline, γ-carboxyglutamate, and O-phosphoserine. The term “amino acid analogue” refers to a compound that has the same basic chemical structure as a naturally occurring amino acid, i.e., an a carbon that is bound to a hydrogen, a carboxyl group, an amino group, and an R group but has a modified R group or a modified peptide backbone as compared with a natural amino acid. Examples include homoserine, norleucine, methionine sulfoxide, methionine methyl sulfonium and norleucine. Amino acid mimetics refers to chemical compounds that have a structure that is different from the general chemical structure of an amino acid, but that functions in a manner similar to a naturally occurring amino acid. Suitably an amino acid is a naturally occurring amino acid or an amino acid analogue, especially a naturally occurring amino acid and in particular one of those 20 L-amino acids encoded by the genetic code.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In general, variants of antigenic polypeptide sequences present in the antigen pool of the invention include sequences having a high degree of sequence identity thereto. For example variants suitably have at least about 80% identity, more preferably at least about 85% identity and most preferably at least about 90% identity (such as at least about 95%, at least about 98% or at least about 99%) to the associated reference sequence over their whole length.

Suitably the variant is an immunogenic variant. A variant is considered to be an immunogenic variant where it elicits a response which is at least 20%, suitably at least 50% and especially at least 75% (such as at least 90%) of the activity of the reference sequence (i.e. the sequence of which the variant is a variant) e.g., in an in vitro restimulation assay of PBMC or whole blood with the polypeptide as antigen (e.g., restimulation for a period of between several hours to up to 1 year, such as up to 6 months, 1 day to 1 month or 1 to 2 weeks), that measures the activation of the cells via lymphoproliferation (e.g., T-cell proliferation), production of cytokines (e.g., IFN-gamma) in the supernatant of culture (measured by ELISA etc.) or characterisation of T-cell responses by intra and extracellular staining (e.g., using antibodies specific to immune markers, such as CD3, CD4, CD8, IL2, TNF-alpha, IFNg, Type 1 IFN, CD40L, CD69 etc.) followed by analysis with a flow cytometer.

The variant may, for example, be a conservatively modified variant. A “conservatively modified variant” is one where the alteration(s) results in the substitution of an amino acid with a functionally similar amino acid or the substitution/deletion/addition of residues which do not substantially impact the biological function of the variant. Typically, such biological function of the variants will be to induce an immune response against a melanoma e.g. a cutaneous melanoma cancer antigen.

Conservative substitution tables providing functionally similar amino acids are well known in the art. Variants can include homologues of polypeptides found in other species.

Antigens present in the antigen pool of the invention may comprise a polypeptide having a variant sequence that contains a number of substitutions, for example, conservative substitutions (for example, 1-25, such as 1-10, in particular 1-5, and especially 1 amino acid residue(s) may be altered) when compared to the reference sequence. The number of substitutions, for example, conservative substitutions, may be up to 20% e.g., up to 10% e.g., up to 5% e.g., up to 1% of the number of residues of the reference sequence. In general, conservative substitutions will fall within one of the amino-acid groupings specified below, though in some circumstances other substitutions may be possible without substantially affecting the immunogenic properties of the antigen. The following eight groups each contain amino acids that are typically conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins 1984).

Suitably such substitutions do not alter the immunological structure of an epitope (e.g., they do not occur within the epitope region as mapped in the primary sequence), and do not therefore have a significant impact on the immunogenic properties of the antigen.

Polypeptide variants also include those wherein additional amino acids are inserted compared to the reference sequence, for example, such insertions may occur at 1-10 locations (such as 1-5 locations, suitably 1 or 2 locations, in particular 1 location) and may, for example, involve the addition of 50 or fewer amino acids at each location (such as 20 or fewer, in particular 10 or fewer, especially 5 or fewer). Suitably such insertions do not occur in the region of an epitope, and do not therefore have a significant impact on the immunogenic properties of the antigen. One example of insertions includes a short stretch of histidine residues (e.g., 2-6 residues) to aid expression and/or purification of the antigen in question.

Polypeptide variants include those wherein amino acids have been deleted compared to the reference sequence, for example, such deletions may occur at 1-10 locations (such as 1-5 locations, suitably 1 or 2 locations, in particular 1 location) and may, for example, involve the deletion of 50 or fewer amino acids at each location (such as 20 or fewer, in particular 10 or fewer, especially 5 or fewer). Suitably such deletions do not occur in the region of an epitope, and do not therefore have a significant impact on the immunogenic properties of the antigen.

The skilled person will recognise that a particular protein variant may comprise substitutions, deletions and additions (or any combination thereof). For example, substitutions/deletions/additions might enhance (or have neutral effects) on binding to desired patient HLA molecules, potentially increasing immunogenicity (or leaving immunogenicity unchanged).

Immunogenic fragments of the antigenic polypeptide sequences present in the antigen pool according to the present invention will typically comprise at least 9 contiguous amino acids from the full-length polypeptide sequence (e.g., at least 9 or 10), such as at least 12 contiguous amino acids (e.g., at least 15 or at least 20 contiguous amino acids), in particular at least 50 contiguous amino acids, such as at least 100 contiguous amino acids (for example at least 200 contiguous amino acids) depending on the length of the CLT antigen. Suitably the immunogenic fragments will be at least 10%, such as at least 20%, such as at least 50%, such as at least 70% or at least 80% of the length of the full-length polypeptide sequence.

Immunogenic fragments typically comprise at least one epitope. Epitopes include B cell and T-cell epitopes and suitably immunogenic fragments comprise at least one T-cell epitope such as a CD4+ or a CD8+ T-cell epitope.

T-cell epitopes are short contiguous stretches of amino acids which are recognised by T-cells (e.g., CD4+ or CD8+ T-cells) when bound to HLA molecules. Identification of T-cell epitopes may be achieved through epitope mapping experiments which are well known to the person skilled in the art (see, for example, Paul, Fundamental Immunology, 3rd ed., 243-247 (1993); Beißbarth et al., 2005, Bioinformatics, 21(Suppl. 1): i29-i37).

As a result of the crucial involvement of the T-cell response in cancer, it is readily apparent that fragments of the full-length polypeptides of SEQ ID NOs. 1-8 which contain at least one T-cell epitope may be immunogenic and may contribute to immunoprotection.

It will be understood that in a diverse outbred population, such as humans, different HLA types mean that specific epitopes may not be recognised by all members of the population. Consequently, to maximise the level of recognition and scale of immune response to a polypeptide, it is generally desirable that an immunogenic fragment contains a plurality of the epitopes from the full-length sequence (suitably all epitopes within a CLT antigen).

Particular fragments of the polypeptides of SEQ ID NOs. 1-8 which may be of use include those containing at least one CD8+ T-cell epitope, suitably at least two CD8+ T-cell epitopes and especially all CD8+ T-cell epitopes, particularly those associated with a plurality of HLA alleles, e.g., those associated with 2, 3, 4, 5 or more alleles). Particular fragments of the polypeptides of SEQ ID NOs. 1-8 which may be of use include those containing at least one CD4+ T-cell epitope, suitably at least two CD4+ T-cell epitopes and especially all CD4+ T-cell epitopes (particularly those associated with a plurality of HLA alleles, e.g., those associated with 2, 3, 4, 5 or more alleles). However, a person skilled in design of vaccines could combine exogenous CD4+ T-cell epitopes with CD8+ T-cells epitopes and achieve desired responses to the CD8+ T-cell epitopes.

Where an individual fragment of the full-length polypeptide is used, such a fragment is considered to be immunogenic where it elicits a response which is at least 20%, suitably at least 50% and especially at least 75% (such as at least 90%) of the activity of the reference sequence (i.e., the sequence of which the fragment is a fragment) e.g., activity in an in vitro restimulation assay of PBMC or whole blood with the polypeptide as antigen (e.g., restimulation for a period of between several hours to up to 1 year, such as up to 6 months, 1 day to 1 month or 1 to 2 weeks,) that measures the activation of the cells via lymphoproliferation (e.g., T-cell proliferation), production of cytokines (e.g., IFN-gamma) in the supernatant of culture (measured by ELISA etc.) or characterisation of T-cell responses by intra and extracellular staining (e.g., using antibodies specific to immune markers, such as CD3, CD4, CD8, IL2, TNF-alpha, IFN-gamma, Type 1 IFN, CD40L, CD69 etc.) followed by analysis with a flow cytometer.

In some circumstances a plurality of fragments of the full-length polypeptide (which may or may not be overlapping and may or may not cover the entirety of the full-length sequence) may be used to obtain an equivalent biological response to the full-length sequence itself. For example, at least two immunogenic fragments (such as three, four or five) as described above, which in combination provide at least 50%, suitably at least 75% and especially at least 90% activity of the reference sequence in an in vitro restimulation assay of PBMC or whole blood (e.g., a T-cell proliferation and/or IFN-gamma production assay).

Example immunogenic fragments of antigenic polypeptides of SEQ ID NOs. 1-8, and thus example component peptides of fusion proteins of the invention, include polypeptides which comprise or consist of the sequences of SEQ ID NOs. 9-55. The sequences of SEQ ID NOs. 9-12, 18-19, 30, 31-32 and 37-39, 45, 48-54 were identified as being bound to HLA Class I molecules from immunopeptidomic analysis (see Examples 2). The sequences of SEQ ID NOs 13-17, 20-29, 33-35, 40-44 were predicted by NetMHC software as being bound to HLA Class I molecules and were used in immunological validation assays (see Examples 3, 4 and 5).

The antigenic polypeptide (a) present in the antigen pool of the invention may comprise or consist of SEQ ID NO. 1 or a variant thereof, or an immunogenic fragment of SEQ ID NO: 1 or a variant thereof. Exemplary fragments comprise or consist of any one of SEQ ID NOs. 9-12. Further exemplary fragments comprise two, three or four of SEQ ID NOs. 9-12. Further exemplary fragments comprise or consist of any one of SEQ ID NOs. 13-17. Further exemplary fragments comprise all of SEQ ID NOs. 9-17 (allowance being taken for possible sequence overlap so that any overlapping sequence does not need to be present more than once).

The antigenic polypeptide (b) present in the antigen pool of the invention may comprise or consist of SEQ ID NO. 2 or a variant thereof, or an immunogenic fragment of SEQ ID NO: 2 or a variant thereof. Exemplary fragments comprise or consist of SEQ ID NO. 18 or SEQ ID NO. 19. Further exemplary fragments comprise SEQ ID NO. 18 and SEQ ID NO. 19. Further exemplary fragments comprise or consist of any one of SEQ ID NOs. 20-30. Further exemplary fragments comprise all of SEQ ID NOs. 18-30 (allowance being taken for possible sequence overlap so that any overlapping sequence does not need to be present more than once).

The antigenic polypeptide (c) present in the antigen pool of the invention may comprise or consist of SEQ ID NO. 3 or a variant thereof, or an immunogenic fragment of SEQ ID NO: 3 or a variant thereof. Exemplary fragments comprise or consist of SEQ ID NO. 31. Further exemplary fragments comprise SEQ ID NO. 31. Further exemplary fragments comprise or consist of any one of SEQ ID NOs. 32-35. Further exemplary fragments comprise SEQ ID NO. 31 and SEQ ID NO. 32. Further exemplary fragments comprise all of SEQ ID NOs. 31-35 (allowance being taken for possible sequence overlap so that any overlapping sequence does not need to be present more than once).

The antigenic polypeptide (d) present in the antigen pool of the invention may comprise or consist of SEQ ID NO. 4 or a variant thereof, or an immunogenic fragment of SEQ ID NO: 4 or a variant thereof. Exemplary fragments comprise or consist of SEQ ID NO. 36. Further exemplary fragments comprise or consist of SEQ ID NO. 37 or SEQ ID NO. 38. Further exemplary fragments comprise or consist of SEQ ID NO. 39. Further exemplary fragments comprise or consist of any one of SEQ ID NOs. 40-44. Further exemplary fragments comprise SEQ ID NO. 36 and either SEQ ID NO. 37 or SEQ ID NO. 38. Further exemplary fragments comprise SEQ ID NO. 39 and either SEQ ID NO. 37 or SEQ ID NO. 38. Further exemplary fragments comprise all of SEQ ID NOs. 36-44 (allowance being taken for possible sequence overlap so that any overlapping sequence does not need to be present more than once).

The antigenic polypeptide (e) present in the antigen pool of the invention may comprise or consist of SEQ ID NO. 5 or a variant thereof, or an immunogenic fragment of SEQ ID NO: 5 or a variant thereof. Exemplary fragments comprise or consist of any one of SEQ ID NOs. 45-47.

The antigenic polypeptide (f) present in the antigen pool of the invention may comprise or consist of SEQ ID NO. 6 or a variant thereof, or an immunogenic fragment of SEQ ID NO: 6 or a variant thereof. Exemplary fragments comprise or consist of SEQ ID NO. 48-51.

The antigenic polypeptide (g) present in the antigen pool of the invention may comprise or consist of SEQ ID NO. 7 or a variant thereof, or an immunogenic fragment of SEQ ID NO: 7 or a variant thereof. Exemplary fragments comprise or consist of SEQ ID NO. 52.

The antigenic polypeptide (h) present in the antigen pool of the invention may comprise or consist of SEQ ID NO. 8 or a variant thereof, or an immunogenic fragment of SEQ ID NO: 8 or a variant thereof. Exemplary fragments comprise or consist of SEQ ID NO. 53-55.

### Nucleic Acids

A nucleic acid encoding an antigenic polypeptide sequence and/or a fusion protein present in the antigen pool of the invention is provided (referred to as a nucleic acid of the invention).

The terms “nucleic acid” and “polynucleotide” are used interchangeably herein and refer to a polymeric macromolecule made from nucleotide monomers particularly deoxyribonucleotide or ribonucleotide monomers. The term encompasses nucleic acids containing known nucleotide analogs or modified backbone residues or linkages, which are naturally occurring and non-naturally occurring, which have similar properties as the reference nucleic acid, and which are intended to be metabolized in a manner similar to the reference nucleotides or are intended to have extended half-life in the system. Examples of such analogs include, without limitation, phosphorothioates, phosphoramidates, methyl phosphonates, chiral-methyl phosphonates, 2-O-methyl ribonucleotides, peptide-nucleic acids (PNAs). Suitably the term “nucleic acid” refers to naturally occurring polymers of deoxyribonucleotide or ribonucleotide monomers. Suitably the nucleic acid molecules of the invention are recombinant. Recombinant means that the nucleic acid molecule is the product of at least one of cloning, restriction or ligation steps, or other procedures that result in a nucleic acid molecule that is distinct from a nucleic acid molecule found in nature (e.g., in the case of cDNA). In an embodiment the nucleic acid of the invention is an artificial nucleic acid sequence (e.g., a cDNA sequence or nucleic acid sequence with non-naturally occurring codon usage). In one embodiment, the nucleic acids of the invention are DNA. Alternatively, the nucleic acids of the invention are RNA.

DNA (deoxyribonucleic acid) and RNA (ribonucleic acid) refer to nucleic acid molecules having a backbone of sugar moieties which are deoxyribosyl and ribosyl moieties respectively. The sugar moieties may be linked to bases which are the 4 natural bases (adenine (A), guanine (G), cytosine (C) and thymine (T) in DNA and adenine (A), guanine (G), cytosine (C) and uracil (U) in RNA). As used herein, a “corresponding RNA” is an RNA having the same sequence as a reference DNA but for the substitution of thymine (T) in the DNA with uracil (U) in the RNA. The sugar moieties may also be linked to unnatural bases such as inosine, xanthosine, 7-methylguanosine, dihydrouridine and 5-methylcytidine. Natural phosphodiester linkages between sugar (deoxyribosyl/ribosyl) moieties may optionally be replaced with phosphorothioates linkages. Suitably nucleic acids of the invention consist of the natural bases attached to a deoxyribosyl or ribosyl sugar backbone with phosphodiester linkages between the sugar moieties.

The nucleic acid of the invention may be DNA. For example the nucleic acid comprises or consists of a sequence selected from SEQ ID NOs. 56-62 and 63-70. Also provided is a nucleic acid which comprises or consists of a variant of sequence selected from SEQ ID NOs. 56-62 or 63-70 which variant encodes the same amino acid sequence but has a different nucleic acid based on the degeneracy of the genetic code.

Thus, due to the degeneracy of the genetic code, a large number of different, but functionally identical nucleic acids can encode any given polypeptide. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations lead to “silent” (sometimes referred to as “degenerate” or “synonymous”) variants, which are one species of conservatively modified variations. Every nucleic acid sequence disclosed herein which encodes a polypeptide also enables every possible silent variation of the nucleic acid. One of skill will recognise that each codon in a nucleic acid (except AUG, which is ordinarily the only codon for methionine, and UGG, which is ordinarily the only codon for tryptophan) can be modified to yield a functionally identical molecule. Accordingly, each silent variation of a nucleic acid that encodes a polypeptide is implicit in each described sequence and is provided as an aspect of the invention.

Degenerate codon substitutions may also be achieved by generating sequences in which the third position of one or more selected (or all) codons is substituted with mixed-base and/or deoxyinosine residues (Batzer et al., 1991, Nucleic Acid Res. 19:5081; Ohtsuka et al., 1985, J. Biol. Chem. 260:2605-2608; Rossolini et al., 1994, Mol. Cell. Probes 8:91-98).

A nucleic acid of the invention which comprises or consists of a sequence selected from SEQ ID NOs. 56-62 and 63-70 may contain a number of silent variations (for example, 1-50, such as 1-25, in particular 1-5, and especially 1 codon(s) may be altered) when compared to the reference sequence.

In an embodiment of the invention the nucleic acid is an RNA, e.g. mRNA. RNA sequences are provided which correspond to a DNA sequence provided herein and have a ribonucleotide backbone instead of a deoxyribonucleotide backbone and have the sidechain base uracil (U) in place of thymine (T).

Thus a nucleic acid of the invention comprises or consists of the RNA equivalent of a cDNA sequence selected from SEQ ID NOs. 56-62 or 63-70 and may contain a number of silent variations (for example, 1-50, such as 1-25, in particular 1-5, and especially 1 codon(s) may be altered) when compared to the reference sequence. By “RNA equivalent” is meant an RNA sequence which contains the same genetic information as the reference cDNA sequence (i.e. contains the same codons with a ribonucleotide backbone instead of a deoxyribonucleotide backbone and having the sidechain base uracil (U) in place of thymine (T)).

The invention also comprises sequences which are complementary to the aforementioned cDNA and RNA sequences.

The nucleic acids may be codon optimised for expression in a human host cell.

Nucleic acids may be capable of being transcribed and translated into polypeptides of the invention in the case of DNA nucleic acids, and translated into polypeptides of the invention in the case of RNA nucleic acids.

### Polypeptides and Nucleic Acids

Suitably, the polypeptides and nucleic acids used in the present invention are isolated. An “isolated” polypeptide or nucleic acid is one that is removed from its original environment. For example, a naturally-occurring polypeptide or nucleic acid is isolated if it is separated from some or all of the coexisting materials in the natural system. A nucleic acid is considered to be isolated if, for example, it is cloned into a vector that is not a part of its natural environment.

“Naturally occurring” when used with reference to a polypeptide or nucleic acid sequence means a sequence found in nature and not synthetically modified.

“Artificial” when used with reference to a polypeptide or nucleic acid sequence means a sequence not found in nature which is, for example, a synthetic modification of a natural sequence, or contains an unnatural sequence.

The term “heterologous” when used with reference to the relationship of one nucleic acid or polypeptide to another nucleic acid or polypeptide indicates that the two or more sequences are not found in the same relationship to each other in nature. A “heterologous” sequence can also mean a sequence which is not isolated from, derived from, or based upon a naturally occurring nucleic acid or polypeptide sequence found in the host organism.

As noted above, variants of antigenic polypeptide sequences present in the antigen pool of the invention may preferably have at least about 80% identity, more preferably at least about 85% identity and most preferably at least about 90% identity (such as at least about 95%, at least about 98% or at least about 99%) to the associated reference sequence over their whole length.

For the purposes of comparing two closely-related polypeptide or polynucleotide sequences, the “% sequence identity” between a first sequence and a second sequence may be calculated. Polypeptide sequences are said to be the same as or identical to other polypeptide sequences, if they share 100% sequence identity over their entire length. Residues in sequences are numbered from left to right, i.e. from N- to C-terminus for polypeptides. The terms “identical” or percentage “identity”, in the context of two or more polypeptide sequences, refer to two or more sequences or sub-sequences that are the same or have a specified percentage of amino acid residues that are the same (i.e., 70% identity, optionally 75%, 80%, 85%, 90%, 95%, 98% or 99% identity over a specified region), when compared and aligned for maximum correspondence over a comparison window. Suitably, the comparison is performed over a window corresponding to the entire length of the reference sequence.

For sequence comparison, one sequence acts as the reference sequence, to which the test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percentage sequence identities for the test sequences relative to the reference sequence, based on the program parameters.

A “comparison window”, as used herein, refers to a segment in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, 1981, Adv. Appl. Math. 2:482, by the homology alignment algorithm of Needleman & Wunsch, 1970, J. Mol. Biol. 48:443, by the search for similarity method of Pearson & Lipman, 1988, Proc. Nat'l. Acad. Sci. USA 85:2444, by computerised implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Current Protocols in Molecular Biology (Ausubel et al., eds. 1995 supplement)).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, 1987, J. Mol. Evol. 35:351-360. The method used is similar to the method described by Higgins & Sharp, 1989, CABIOS 5:151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., 1984, Nuc. Acids Res. 12:387-395).

Another example of algorithm that is suitable for determining percent sequence identity and sequence similarity are the BLAST and BLAST 2.0 algorithms, which are described in Altschul et al., 1977, Nuc. Acids Res. 25:3389-3402 and Altschul et al., 1990, J. Mol. Biol. 215:403-410, respectively. Software for performing BLAST analyses is publicly available through the National Center for Biotechnology Information (website at www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighbourhood word score threshold (Altschul et al., supra). These initial neighbourhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. For amino acid sequences, the BLASTP program uses as defaults a wordlength of 3, and expectation (E) of 10, and the BLOSUM62 scoring matrix (see Henikoff & Henikoff, 1989, Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin & Altschul, 1993, Proc. Nat'l. Acad. Sci. USA 90:5873-5787). One measure of similarity provided by the BLAST algorithm is the smallest sum probability (P(N)), which provides an indication of the probability by which a match between two nucleotide or amino acid sequences would occur by chance.

A “difference” between sequences refers to an insertion, deletion or substitution of a single residue in a position of the second sequence, compared to the first sequence. Two sequences can contain one, two or more such differences. Insertions, deletions or substitutions in a second sequence which is otherwise identical (100% sequence identity) to a first sequence result in reduced % sequence identity. For example, if the identical sequences are 9 residues long, one substitution in the second sequence results in a sequence identity of 88.9%. If the identical sequences are 17 amino acid residues long, two substitutions in the second sequence results in a sequence identity of 88.2%.

Alternatively, for the purposes of comparing a first, reference sequence to a second, comparison sequence, the number of additions, substitutions and/or deletions made to the first sequence to produce the second sequence may be ascertained. An addition is the addition of one residue into the first sequence (including addition at either terminus of the first sequence). A substitution is the substitution of one residue in the first sequence with one different residue. A deletion is the deletion of one residue from the first sequence (including deletion at either terminus of the first sequence).

### Fusion Proteins (Fusion Polypeptides)

The term “fusion protein” refers to any protein comprising at least two polypeptides that are joined together by peptide bonds, through protein synthesis. The fusion protein may be created through the joining of two or more genes that encode for separate polypeptides that have been joined so that they are transcribed and translated as a single unit producing a single protein.

The invention provides an antigen pool comprising two or more different antigens, wherein the different antigens are present in the antigen pools as part of a fusion protein. Fusion proteins are expected to have the utilities described herein and may have the advantage of superior immunogenic or prophylactic or therapeutic effect (including increasing the breadth and depth of responses) as compared with the individual component polypeptides, and may be especially valuable in an outbred population.

The antigenic polypeptides present in a fusion protein of the antigen pool may be arranged in various sequential orders from the N terminus to the C terminus. The design and order of the polypeptides in the fusion proteins are described in Example 8. In particular, the order of the polypeptides in the fusion protein is important because such an order can in some cases lead to superior processing and presentation of desirable immunogenic peptide regions of a polypeptide and in other cases is necessary for optimal fusion design to reduce the likelihood of unnatural immunogenic peptides, derived from the junctions between the natural cancer-specific CLT Antigens could be presented on surface displayed Class I HLA molecules during vaccination, thus eliciting undesireable T cell responses.

The fusion proteins provide for a strong antigenic response to the component CLT Antigens, see Examples 9 & 10, and are expected to elicit minimal antigenic responses to their junction regions, see Example 8.

Any of the sequences (a) to (h) could be deployed with the initial N terminal methionine is removed. Thus, one or more of the antigenic polypeptides may comprise a polypeptide sequence lacking the N terminal methionine.

In one embodiment, when the fusion protein comprises six antigenic polypeptides (a), (b), (d), (f), (g) and (h).

In an embodiment, the fusion proteins of the invention may comprise an antigenic polypeptide having the polypeptide sequence of SEQ ID NO: 2 minus the N-terminal methionine residue. In an embodiment, the fusion proteins of the invention comprise an antigenic polypeptide having the amino acid sequence of SEQ ID NO: 6 minus the N-terminal methionine residue. In an embodiment, the fusion proteins of the invention comprise an antigenic polypeptide having the amino acid sequence of SEQ ID NO: 5 minus the N-terminal methionine residue. In an embodiment, the fusion proteins of the invention comprise an antigenic polypeptide having the amino acid sequence of SEQ ID NO: 2 minus the N-terminal methionine residue, an antigenic polypeptide having the amino acid sequence of SEQ ID NO: 6 minus the N-terminal methionine residue, and an antigenic polypeptide having the amino acid sequence of SEQ ID NO: 5 minus the N-terminal methionine residue.

When the fusion protein comprises six different antigens, wherein the antigens have the polypeptide sequences of (a), (b), (d), (f), (g) and (h), polypeptides (a), (b), (d), (f), (g) and (h) may have the polypeptide sequences:
(a) SEQ ID NO: 1 with or without the N-terminal methionine residue or a variant thereof, or an immunogenic fragment of SEQ ID NO: 1 or a variant thereof;
(b) SEQ ID NO: 2 with or without the N-terminal methionine residue or a variant thereof, or an immunogenic fragment of SEQ ID NO: 2 or a variant thereof;
(d) SEQ ID NO: 4 with or without the N-terminal methionine residue or a variant thereof, or an immunogenic fragment of SEQ ID NO: 4 or a variant thereof;
(f) SEQ ID NO: 6 with or without the N-terminal methionine residue or a variant thereof, or an immunogenic fragment of SEQ ID NO: 6 or a variant thereof;
(g) SEQ ID NO: 7 with or without the N-terminal methionine residue or a variant thereof, or an immunogenic fragment of SEQ ID NO: 7 or a variant thereof;
(h) SEQ ID NO: 8 with or without the N-terminal methionine residue or a variant thereof, or an immunogenic fragment of SEQ ID NO: 8 or a variant thereof.

In an embodiment, the six antigenic polypeptides are arranged in the order from N to C of (a), (b), (f), (g), (d) and (h).

In one suitable embodiment, the six antigenic polypeptides have the sequences of SEQ ID NOs. 1-2, 4, 6-8 and are arranged in the order from N to C of SEQ ID NO: 1, SEQ ID NO: 2, SEQ ID NO: 6, SEQ ID NO: 7, SEQ ID NO: 4 and SEQ ID NO: 8. A corresponding sequence in which the N-terminal methionine is omitted may optionally be used as explained above.

For example, the fusion protein of the invention comprises six antigenic polypeptides (a), (b), (d), (f), (g) and (h) wherein the antigenic polypeptides (a), (b), (d), (f), (g) and (h) have the amino acid sequences:
(a) SEQ ID NO: 1;
(b) SEQ ID NO: 2 minus the N-terminal methionine residue;
(d) SEQ ID NO: 4;
(f) SEQ ID NO: 6;
(g) SEQ ID NO: 7; and
(h) SEQ ID NO: 8.

Thus, suitably, SEQ ID NO: 1 is present at the N terminus and SEQ ID NO: 8 is present at the C terminus. Suitably the N-terminal methionine of SEQ ID NO: 2 is omitted. In an embodiment of the invention, the fusion protein has the sequence of SEQ ID NO: 76.

In another embodiment, when the fusion protein comprises six antigenic polypeptides (a), (b), (d), (f), (g) and (h), the six antigenic polypeptides are arranged in the order from N to C of (f), (h), (g), (b), (d) and (a).

In one suitable embodiment, the six antigenic polypeptides have the sequences of SEQ ID NOs. 1-2, 4, 6-8 and are arranged in the order from N to C of SEQ ID NO: 6, SEQ ID NO: 8, SEQ ID NO: 7, SEQ ID NO: 2, SEQ ID NO: 4 and SEQ ID NO: 1. A corresponding sequence in which the N-terminal methionine is omitted may optionally be used as explained above. Thus, suitably, SEQ ID NO: 6 is present at the N terminus and SEQ ID NO: 1 is present at the C terminus. Suitably the N-terminal methionine of SEQ ID NO: 2 is omitted. In an embodiment of the invention, the fusion protein has the sequence of SEQ ID NO: 77.

In another embodiment, the fusion protein comprises eight antigenic polypeptides (a) to (h).

When the fusion protein comprises eight different antigens, wherein the antigens have the polypeptide sequences of (a) to (h), polypeptides (a) to (h) may have the polypeptide sequences:
(a) SEQ ID NO: 1 with or without the N-terminal methionine residue or a variant thereof, or an immunogenic fragment of SEQ ID NO: 1 or a variant thereof;
(b) SEQ ID NO: 2 with or without the N-terminal methionine residue or a variant thereof, or an immunogenic fragment of SEQ ID NO: 2 or a variant thereof;
(c) SEQ ID NO: 3 with or without the N-terminal methionine residue or a variant thereof, or an immunogenic fragment of SEQ ID NO: 3 or a variant thereof;
(d) SEQ ID NO: 4 with or without the N-terminal methionine residue or a variant thereof, or an immunogenic fragment of SEQ ID NO: 4 or a variant thereof;
(e) SEQ ID NO: 5 with or without the N-terminal methionine residue or a variant thereof, or an immunogenic fragment of SEQ ID NO: 5 or a variant thereof;
(f) SEQ ID NO: 6 with or without the N-terminal methionine residue or a variant thereof, or an immunogenic fragment of SEQ ID NO: 6 or a variant thereof;
(g) SEQ ID NO: 7 with or without the N-terminal methionine residue or a variant thereof, or an immunogenic fragment of SEQ ID NO: 7 or a variant thereof;
(h) SEQ ID NO: 8 with or without the N-terminal methionine residue or a variant thereof, or an immunogenic fragment of SEQ ID NO: 8 or a variant thereof.

In an embodiment, the eight antigenic polypeptides are arranged in the order from N to C of (a), (b), (c), (g), (d), (e), (f) and (h).

In one suitable embodiment, the eight antigenic polypeptides have the sequences of SEQ ID NOs. 1-8 and are arranged in the order from N to C of SEQ ID NO: 1, SEQ ID NO: 2, SEQ ID NO: 3, SEQ ID NO: 7, SEQ ID NO: 4, SEQ ID NO: 5, SEQ ID NO: 6 and SEQ ID NO: 8. A corresponding sequence in which the N-terminal methionine is omitted may optionally be used as explained above.

For example, the fusion protein of the invention comprises eight antigenic polypeptides (a) to (h) wherein the antigenic polypeptides (a) to (h) have the amino acid sequences:
(a) SEQ ID NO: 1;
(b) SEQ ID NO: 2 minus the N-terminal methionine residue;
(c) SEQ ID NO: 3;
(d) SEQ ID NO: 4;
(e) SEQ ID NO: 5 minus the N-terminal methionine residue;
(f) SEQ ID NO: 6 minus the N-terminal methionine residue;
(g) SEQ ID NO: 7; and
(h) SEQ ID NO: 8.
   Suitably, SEQ ID NO: 1 present at the N-terminal and SEQ ID NO: 8 is present at the C terminus. Suitably the N-terminal methionine of SEQ ID NO: 2 is omitted. Suitably the N-terminal methionine of SEQ ID NO: 6 is omitted. Suitably the N-terminal methionine of SEQ ID NO: 5 is omitted. In an embodiment of the invention, the fusion protein has the sequence of SEQ ID NO: 78.

In another embodiment, when the fusion protein comprises eight antigenic polypeptides (a) to (h), the eight antigenic polypeptides are arranged in the order from N to C of (f), (c), (a), (e), (d), (h), (g) and (b).

In one suitable embodiment, the eight antigenic polypeptides have the sequences of SEQ ID NOs. 1-8 and are arranged in the order from N to C of SEQ ID NO: 6, SEQ ID NO: 3, SEQ ID NO: 1, SEQ ID NO: 5, SEQ ID NO: 4, SEQ ID NO: 8, SEQ ID NO: 7 and SEQ ID NO: 2. A corresponding sequence in which the N-terminal methionine is omitted may optionally be used as explained above.

For example, the fusion protein of the invention comprises eight antigenic polypeptides (a) to (h) wherein the antigenic polypeptides (a) to (h) have the amino acid sequences:
(a) SEQ ID NO: 1;
(b) SEQ ID NO: 2 minus the N-terminal methionine residue;
(c) SEQ ID NO: 3;
(d) SEQ ID NO: 4;
(e) SEQ ID NO: 5;
(f) SEQ ID NO: 6;
(g) SEQ ID NO: 7; and
(h) SEQ ID NO: 8.
   Suitably, SEQ ID NO: 6 is present at the N-terminal and SEQ ID NO: 2 is present at the C terminus. Suitably the N-terminal methionine of SEQ ID NO: 2 is omitted. In an embodiment of the invention, the fusion protein has the sequence of SEQ ID NO: 79.

Fusion proteins of the invention may be fused to a second or further polypeptide selected from (i) other polypeptides which are melanoma associated antigens; (ii) polypeptide sequences which are capable of enhancing an immune response (i.e. immunostimulant sequences); and (iii) polypeptide sequences, e.g. comprising universal CD4 helper epitopes, which are capable of providing strong CD4+ help to increase CD8+ T cell responses to antigen epitopes.

Exemplary fusion polypeptides comprises two or more (e.g. two, three, four, five, six, seven or eight) sequences selected from the sequences of (a) to (h).

The invention also provides nucleic acids encoding the aforementioned fusion proteins.

### Linkers

The invention provides for an antigen pool comprising two or more different antigens wherein the two or more different antigens may be present in the antigen pool as part of a fusion protein and/or a nucleic acid encoding said fusion protein. When present in a fusion protein or nucleic acid encoding said fusion protein, the two or more different antigens are joined together by one or more peptide linkers or spacers positioned between the antigenic polypeptide sequences. The antigenic polypeptide sequences present in the antigen pool of the present invention may be joined together by one or more linkers (e.g. two, three, four, five, six or seven linkers). A linker may separate each of the antigenic polypeptide sequences present in the antigen pool of the present invention. The linkers may be ‘internal’, i.e. the linkers are not present at the N terminus of the first polypeptide and the C terminus of the last polypeptide of the fusion protein. Thus, in an embodiment of the invention, when the two or more different antigens are present in a fusion protein or nucleic acid encoding the fusion protein, the two or more different antigens are joined together by one or more peptide linkers positioned between the antigenic polypeptide sequences.

The one or more linkers may be positioned between (a) and (b), (b) and (f), (f) and (g), (g) and (d), (d) and (h). In another embodiment of the invention, the one or more linkers are positioned between (f) and (h), (h) and (g), (g) and (b), (b) and (d), (d) and (a). In a further embodiment of the invention, the linkers are positioned between (a) and (b), (b) and (c), (c) and (g), (g) and (d), (d) and (e), (e) and (f), (f) and (h). In a further embodiment of the invention, the linkers are positioned between (a) and (b), (b) and (d), (d) and (f), (f) and (g), (g) and (h). In yet a further embodiment of the invention the linkers are positioned between (f) and (c), (c) and (a), (a) and (e), (e) and (d), (d) and (h), (h) and (g), (g) and (b).

The linker may refer to the cDNA encoding the linker peptide sequence, or the encoded peptide. The linkers are placed between the individual antigens of each fusion protein of the invention by creating a single construct in which the linker sequence is inserted between the C terminus of one antigenic polypeptide and the N terminus of the following antigenic polypeptide, thereby linking the antigenic polypeptides of the fusion protein together. The individual linkers used in a fusion protein may have the same sequence or they may have different sequences. In one embodiment of the invention, the linkers comprise or consist of sequences selected from SEQ ID NOs: 71-75 and 84.

In an embodiment of the invention, the fusion protein comprises or consists of a sequence selected from SEQ ID NOs: 76-79.

The linkers may be glycine based linkers, which may also include lysines, in a connector of 3 to 6 amino acids in length (see of SEQ ID NOs: 71-75 and 84). The linkers of the present invention reduce the risk of introducing unwanted immunogenic epitopes which contain the linker itself; they also prevent the unwanted epitopes created by direct fusion of the individual antigenic polypeptides.

The fusion proteins may be created through the joining of three or more genes (e.g. three, four, five, six, seven, eight, nine, ten, eleven, twelve, thirteen, fourteen, fifteen) that encode for separate antigenic polypeptide sequences and cDNAs that encode linkers that have been joined so that the resulting open reading frames are transcribed and translated as a single unit producing a single protein. Nucleic acids encoding the fusion proteins of the present invention may comprise or consist of a sequence selected from SEQ ID NOs: 80-83.

### Production of Polypeptides of the Invention

Antigenic polypeptide sequences present in the antigen pool of the invention may be obtained and manipulated using the techniques disclosed for example in Green and Sambrook 2012 Molecular Cloning: A Laboratory Manual 4th Edition Cold Spring Harbour Laboratory Press. In particular, artificial gene synthesis may be used to produce polynucleotides (Nambiar et al., 1984, Science, 223:1299-1301, Sakamar and Khorana, 1988, Nucl. Acids Res., 14:6361-6372, Wells et al., 1985, Gene, 34:315-323 and Grundstrom et al., 1985, Nucl. Acids Res., 13:3305-3316) followed by expression in a suitable organism to produce polypeptides. A gene encoding an antigenic polypeptide present in the antigen pool of the invention may be synthetically produced by, for example, solid-phase DNA synthesis. Entire genes may be synthesized de novo, without the need for precursor template DNA. To obtain the desired oligonucleotide, the building blocks are sequentially coupled to the growing oligonucleotide chain in the order required by the sequence of the product. Upon the completion of the chain assembly, the product is released from the solid phase to solution, deprotected, and collected. Products can be isolated by high-performance liquid chromatography (HPLC) to obtain the desired oligonucleotides in high purity (Verma and Eckstein, 1998, Annu. Rev. Biochem. 67:99-134). These relatively short segments are readily assembled by using a variety of gene amplification methods (Methods Mol Biol., 2012; 834:93-109) into longer DNA molecules, suitable for use in innumerable recombinant DNA-based expression systems. In the context of this invention one skilled in the art would understand that the polynucleotide sequences encoding the polypeptide antigens described in this invention could be readily used in a variety of vaccine production systems, including, for example, viral vectors.

For the purposes of production of polypeptides present in the antigen pool of the invention in a microbiological host (e.g., bacterial or fungal), nucleic acids will comprise suitable regulatory and control sequences (including promoters, termination signals etc) and sequences to promote polypeptide secretion suitable for protein production in the host. Similarly, polypeptides present in the antigen pool of the invention could be produced by transducing cultures of eukaryotic cells (e.g., Chinese hamster ovary cells or Drosophila S2 cells) with nucleic acids of the invention which have been combined with suitable regulatory and control sequences (including promoters, termination signals etc) and sequences to promote polypeptide secretion suitable for protein production in these cells.

Improved isolation of the polypeptides present in the antigen pool of the invention produced by recombinant means may optionally be facilitated through the addition of a stretch of histidine residues (commonly known as a His-tag) towards one end of the polypeptide.

Polypeptides may also be produced synthetically.

### Vectors

Genetic constructs comprising one or more of the nucleic acids of the antigen pools of the invention may be introduced into cells ex vivo such that polypeptides encoded by the nucleic acids are produced. The nucleic acid (e.g., DNA) may be present within any of a variety of delivery systems known to those of ordinary skill in the art, including nucleic acid expression systems, bacteria and some viral expression systems. Numerous gene delivery techniques are well known in the art, such as those described by Rolland, 1998, Crit. Rev. Therap. Drug Carrier Systems 15:143-198, and references cited therein. Several of these approaches are outlined below for the purpose of illustration.

A vector may comprise nucleic acid encoding regulatory elements (such as a suitable promoter and terminating signal) suitable for permitting transcription of a translationally active RNA molecule in a human host cell. A “translationally active RNA molecule” is an RNA molecule capable of being translated into a protein by a human cell's translation apparatus.

The vector may be a viral vector. The viral vector may be an adenovirus, adeno-associated virus (AAV) (e.g., AAV type 5 and type 2), alphavirus (e.g., Venezuelan equine encephalitis virus (VEEV), Sindbis virus (SIN), Semliki Forest virus (SFV)), herpes virus, arenavirus (e.g., lymphocytic choriomeningitis virus (LCMV)), measles virus, poxvirus (such as modified vaccinia Ankara (MVA)), paramyxovirus, lentivirus, or rhabdovirus (such as vesicular stomatitis virus (VSV)) vector i.e. the vector may be derived from any of the aforementioned viruses The viral vector is an adenovirus. The viral vector is a pox virus, e.g. MVA.

Adenoviruses are particularly suitable for use as a gene transfer vector because of its mid-sized genome, ease of manipulation, high titre, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1 B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP is particularly efficient during the late phase of infection, and all the mRNAs transcribed from this promoter possess a 5′-tripartite leader (TPL) sequence which makes them preferred mRNAs for translation. Replication-deficient adenovirus, which are created by from viral genomes that are deleted for one or more of the early genes are particularly useful, since they have limited replication and less possibility of pathogenic spread within a vaccinated host and to contacts of the vaccinated host.

Other Vehicles and Methods for the Introduction of Polynucleotides into Cells

The expression construct comprising one or more nucleic acid sequences may simply consist of naked recombinant DNA plasm ids. See Ulmer et al., 1993, Science 259:1745-1749 and reviewed by Cohen, 1993, Science 259:1691-1692. Transfer of the construct may be performed, for example, by any method which physically or chemically permeabilises the cell membrane. This is particularly applicable for transfer ex vivo.

Methods for the Introduction or Delivery of RNA into Cells

The expression construct comprising one or more polynucleotide sequences may consist of naked, recombinant DNA-derived RNA molecules (Ulmer et al., 2012, Vaccine 30:4414-4418). As for DNA-based expression constructs, a variety of methods can be utilized to introduce RNA molecules into cells ex vivo. The RNA-based constructs can be designed to mimic simple messenger RNA (mRNA) molecules, such that the introduced biological molecule is directly translated by the host cell's translation machinery to produce its encoded polypeptide in the cells to which it has been introduced. Alternatively, RNA molecules may be designed in a manner that allows them to self-amplify within cells they are introduced into, by incorporating into their structure genes for viral RNA-dependent RNA polymerases. Thus, these types of RNA molecules, known as self-amplifying mRNA (SAM™) molecules (Geall et al. 2012, PNAS, 109:14604-14609), share properties with some RNA-based viral vectors. Either mRNA-based or SAM™ RNAs may be further modified (e.g., by alteration of their sequences, or by use of modified nucleotides) to enhance stability and translation (Schlake et al., RNA Biology, 9: 1319-1330), and both types of RNAs may be formulated (e.g., in emulsions (Brito et al., Molecular Therapy, 2014 22:2118-2129) or lipid nanoparticles (Kranz et al., 2006, Nature, 534:396-401)) to facilitate stability and/or entry into cells ex vivo. Thus, in one embodiment of the invention, the nucleic acids are formulated in nanoparticles. Suitably the nanoparticles are lipid-based nanoparticles, e.g. cationic liposomes. Myriad formulations of modified (and non-modified) RNAs have been tested as vaccines in animal models and in man, and multiple RNA-based vaccines are being used in ongoing clinical trials.

### Pharmaceutical Compositions

The antigen pool of the invention may be formulated for delivery in pharmaceutical compositions such as immunogenic compositions (hereinafter “compositions of the invention”). Compositions of the invention suitably comprise the antigen pool of the invention together with a pharmaceutically acceptable carrier.

Thus, in an embodiment, there is provided an immunogenic pharmaceutical composition comprising an antigen pool of the invention together with a pharmaceutically acceptable carrier.

In certain preferred embodiments of the present invention, immunogenic pharmaceutical compositions of the invention are provided which comprise an antigen pool, comprising two or more (e.g. two, three, four, five, six, seven, eight) different antigens wherein each antigen is present in the form of a polypeptide and wherein the different antigens are present in the antigen pool as separate polypeptides, in combination with a pharmaceutically acceptable carrier.

In certain preferred embodiments of the present invention, immunogenic pharmaceutical compositions of the invention are provided which comprise an antigen pool comprising two or more (e.g. two, three, four, five, six, seven, eight) different antigens wherein each antigen is present in the form of a nucleic acid encoding said polypeptide and wherein the different antigens are present in the antigen pool as separate nucleic acids, in combination with a pharmaceutically acceptable carrier.

In certain preferred embodiments of the present invention, immunogenic pharmaceutical compositions of the invention are provided which comprise an antigen pool, comprising two or more (e.g. two, three, four, five, six, seven, eight) different antigens wherein each antigen is present in the form of a polypeptide and wherein the different antigens are present in the antigen pool as part of a fusion protein, in combination with a pharmaceutically acceptable carrier.

In certain preferred embodiments of the present invention, immunogenic pharmaceutical compositions of the invention are provided which comprise an antigen pool, comprising two or more (e.g. two) different antigens wherein each antigen is present in the form of a nucleic acid encoding said polypeptide and wherein the different antigens are present in the antigen pool as a nucleic acid encoding a fusion protein, in combination with a pharmaceutically acceptable carrier.

In an embodiment, the immunogenic pharmaceutical compositions of the invention may comprise an antigen pool comprising two or more (e.g. two, three, four, five, six, seven, eight) different antigens wherein each antigen is present in the form of a polypeptide or a nucleic acid encoding said polypeptide, wherein the different antigens are present in the antigen pool as separate polypeptides, nucleic acids, fusion proteins and nucleic acids encoding said fusion protein, in combination with a pharmaceutically acceptably carrier. Such compositions may provide for an enhanced immune response.

### Pharmaceutically Acceptable Salts

It will be apparent that a composition of the invention may contain pharmaceutically acceptable salts of the nucleic acids, polypeptides or fusion proteins provided herein. Such salts may be prepared from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

### Pharmaceutically Acceptable Carriers

While many pharmaceutically acceptable carriers known to those of ordinary skill in the art may be employed in the compositions of the invention, the optimal type of carrier used will vary depending on the mode of administration. Compositions of the present invention may be formulated for any appropriate manner of administration, including for example, parenteral, topical, oral, nasal, intravenous, intracranial, intraperitoneal, subcutaneous or intramuscular administration, preferably parenteral e.g., intramuscular, subcutaneous or intravenous administration. For parenteral administration, the carrier preferably comprises water and may contain buffers for pH control, stabilising agents e.g., surfactants and amino acids and tonicity modifying agents e.g., salts and sugars. If the composition is intended to be provided in lyophilised form for dilution at the point of use, the formulation may contain a lyoprotectant e.g., sugars such as trehalose. For oral administration, any of the above carriers or a solid carrier, such as mannitol, lactose, starch, magnesium stearate, sodium saccharine, talcum, cellulose, glucose, sucrose, and magnesium carbonate, may be employed.

Thus, compositions of the invention may comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, solutes that render the formulation isotonic, hypotonic or weakly hypertonic with the blood of a recipient, suspending agents, thickening agents and/or preservatives. Alternatively, compositions of the invention may be formulated as a lyophilizate.

### Immunostimulants

Compositions of the invention may also comprise one or more immunostimulants. An immunostimulant may be any substance that enhances or potentiates an immune response (antibody and/or cell-mediated) to an exogenous antigen. Examples of immunostimulants, which are often referred to as adjuvants in the context of vaccine formulations, include aluminium salts such as aluminium hydroxide gel (alum) or aluminium phosphate, saponins including QS21, immunostimulatory oligonucleotides such as CPG, oil-in-water emulsion (e.g., where the oil is squalene), aminoalkyl glucosaminide 4-phosphates, lipopolysaccharide or a derivative thereof e.g., 3-de-O-acylated monophosphoryl lipid A (3D-MPL®) and other TLR4 ligands, TLR7 ligands, TLR8 ligands, TLR9 ligands, IL-12 and interferons. Thus, suitably the one or more immunostimulants of the composition of the invention are selected from aluminium salts, saponins, immunostimulatory oligonucleotides, oil-in-water emulsions, aminoalkyl glucosaminide 4-phosphates, lipopolysaccharides and derivatives thereof and other TLR4 ligands, TLR7 ligands, TLR8 ligands and TLR9 ligands. Immunostimulants may also include monoclonal antibodies which specifically interact with other immune components, for example monoclonal antibodies that block the interaction of immune checkpoint receptors, including PD-1 and CTLA4.

In the case of recombinant-nucleic acid methods of delivery (e.g., DNA, RNA, viral vectors), the genes encoding protein-based immunostimulants may be readily delivered along with the genes encoding the polypeptides present in the antigen pools of the invention.

### Sustained Release

The compositions described herein may be administered as part of a sustained-release formulation (i.e., a formulation such as a capsule, sponge, patch or gel (composed of polysaccharides, for example)) that effects a slow/sustained release of compound following administration.

### Storage and Packaging

Compositions of the invention may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a composition of the invention may be stored in a freeze-dried condition requiring only the addition of a sterile liquid carrier (such as water or saline for injection) immediately prior to use.

### Dosage

The amount of nucleic acid, polypeptide or fusion protein in each composition of the invention may be prepared in such a way that a suitable dosage for therapeutic use will be obtained. Factors such as solubility, bioavailability, biological half-life, route of administration, product shelf life, as well as other pharmacological considerations will be contemplated by one skilled in the art of preparing such compositions, and as such, a variety of dosages and treatment regimens may be desirable.

Typically, compositions comprising a therapeutically effective amount deliver about 0.1 ug to about 1000 ug of polypeptide or fusion protein in the composition of the invention per administration, more typically about 2.5 ug to about 100 ug of polypeptide or fusion protein per administration. If delivered in the form of short, synthetic long peptides, doses could range from 1 to 200 ug/peptide/dose. In respect of polynucleotide compositions, these typically deliver about 10 ug to about 20 mg of the nucleic acid in the antigen pool of the invention per administration, more typically about 0.1 mg to about 10 mg of the nucleic acid per administration.

### Stimulated T-Cell Therapies

Autologous or non-autologous T-cells may be isolated from a subject, e.g., from peripheral blood, umbilical cord blood and/or by apheresis, and stimulated in the presence of a tumor-associated antigens which are loaded onto MHC molecules (signal 1) of APC cells, to induce proliferation of T-cells with a TCR immunospecific for this antigen.

Successful T-cell activation requires the binding of the costimulatory surface molecules B7 and CD28 on antigen-presenting cells and T-cells, respectively (signal 2). To achieve optimal T-cell activation, both signals 1 and 2 are required. Conversely, antigenic peptide stimulation (signal 1) in the absence of costimulation (signal 2) cannot induce full T-cell activation, and may result in T-cell tolerance. In addition to costimulatory molecules, there are also inhibitory molecules, such as CTLA-4 and PD-1, which induce signals to prevent T-cell activation.

Autologous or non-autologous T-cells may therefore be stimulated in the presence of an antigen pool of the invention, and expanded and transferred back to the patient at risk of or suffering from cancer whose cancer cells express a corresponding polypeptide of the antigen pool of the invention provided that the antigen-specific TCRs will recognize the antigen presented by the patient's MHC, where they will target and induce the killing of cells of said cancer which express said corresponding polypeptide.

Thus, in an embodiment of the invention, there is provided an antigen pool or composition of the invention for use in the ex vivo stimulation and/or amplification of T-cells derived from a human suffering from cancer, for subsequent reintroduction of said stimulated and/or amplified T-cells into the said human for the treatment of the said cancer in the said human.

In a further embodiment of the invention there is provided a method of treatment of cancer in a human, wherein the cells of the cancer express a sequence of a polypeptide selected from (a) to (h), which comprises taking from said human a population of white blood cells comprising at least T-cells optionally with antigen-presenting cells, stimulating and/or amplifying said T-cells in the presence of an antigen pool or composition of the invention, and reintroducing some or all of said white blood cells comprising at least stimulated and/or amplified T-cells into the human.

In any one of the above embodiments, suitably the cancer is melanoma, particularly cutaneous melanoma.

In another embodiment of the invention, there is provided a process for preparing a T-cell population which is cytotoxic for cancer cells which express a sequence of a polypeptide selected from (a) to (h) which comprises (i) obtaining T-cells optionally with antigen-presenting cells from a cancer patient and (ii) stimulating and amplifying the T-cell population ex vivo with an antigen pool or composition of the invention. The antigen pool may contain a corresponding polypeptide selected from (a) to (h) which the cancer cells express.

By “corresponding” in this context is meant that if the cancer cells express, say, SEQ ID NO. A (A being one of SEQ ID NOs. 1-8) or a variant or immunogenic fragment thereof then the T-cell population is stimulated and amplified ex vivo with SEQ ID NO. A or a variant or immunogenic fragment thereof in the form of a polypeptide, nucleic acid or fusion protein, or a composition containing one of the foregoing.

For example, in such processes, the culturing and expanding is performed in the presence of dendritic cells. The dendritic cells may be transfected with a nucleic acid molecule and express a polypeptide of the antigen pool of the invention.

In an embodiment of the invention there is provided a T-cell population obtainable by the aforementioned process (hereinafter a T-cell population of the invention).

In a further embodiment of the invention there is provided a cell which is a T-cell which has been stimulated with an antigen pool or composition of the invention (hereinafter a T-cell of the invention).

In yet a further embodiment of the invention there is provided a pharmaceutical composition comprising a T-cell population or a T-cell of the invention together with a pharmaceutically acceptable carrier. Such a composition may, for example, be a sterile composition suitable for parenteral administration.

In another embodiment of the invention, there is provided a T-cell population or T-cell of the invention for use in medicine.

There is also provided as an embodiment of the invention a method of treating a human suffering from cancer wherein the cells of the cancer express a polypeptide sequence selected from (a) to (h), wherein the cells of the cancer would express a polypeptide sequence selected from (a) to (h), which comprises administering to said human said T-cell population or T-cell of the invention or composition comprising said T-cell population or T-cell of the invention.

In a further embodiment of the invention, there is provided a T-cell population of the invention, T-cell of the invention or composition comprising said T-cell population or T-cell of the invention for use in treating cancer in a human, wherein the cells of the cancer express a polypeptide sequence selected from (a) to (h).

In any one of the above embodiments, suitably the cancer is melanoma particularly cutaneous melanoma.

The use of the T-cell population, T cell or composition in raising an immune response in a human against a cancer depends on corresponding antigenic sequences of the antigen pool (or one or more of them) being expressed by the cancer. Thus, there is a relationship between the design of the antigen pool and the antigenic sequences that the cancer expresses or is likely to express. In this context, “corresponding” means that if the cancer expresses (or is likely to express), say, SEQ ID NO. A (A being one of SEQ ID NOs. 1-8) or a variant or immunogenic fragment thereof, then the pool will include SEQ ID NO. A or a variant or immunogenic fragment thereof (optionally in the form of a fusion protein and as protein or nucleic acid). Thus, there is a relationship between the design of the antigen pool, fusion protein, nucleic acid, vector or composition and the antigenic sequences that the cancer expresses or is likely to express. The inclusion of a number of antigen sequences in the antigen pool potentially makes possible a greater immune response against a cancer or an immune response against cancer in a wider range of patients.

### Cell Therapies to Facilitate Antigen Presentation In Vivo

Any of a variety of cellular delivery vehicles may be employed within pharmaceutical compositions to facilitate production of an antigen-specific immune response. Thus the invention provides a cell which is an isolated antigen presenting cell modified by ex vivo loading with the antigen pool of the invention or composition of the invention (herein after referred to as a “APC of the invention”). Antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T-cell response and/or to be immunologically compatible with the receiver (i.e., matched HLA haplotype). APCs may generally be isolated from any of a variety of biological fluids and organs, and may be autologous, allogeneic, syngeneic or xenogeneic cells.

Certain preferred embodiments of the present invention use dendritic cells or progenitors thereof as APCs. Thus, in an embodiment, the APC of the invention is a dendritic cell. Dendritic cells are highly potent APCs (Banchereau & Steinman, 1998, Nature, 392:245-251) and have been shown to be effective as a physiological adjuvant for eliciting prophylactic or therapeutic immunity (see Timmerman & Levy, 1999, Ann. Rev. Med. 50:507-529). In general, dendritic cells may be identified based on their typical shape (stellate in situ, with marked cytoplasmic processes (dendrites) visible in vitro), their ability to take up, process and present antigens with high efficiency and their ability to activate naïve T-cell responses. Dendritic cells may, of course be engineered to express specific cell-surface receptors or ligands that are not commonly found on dendritic cells in vivo or ex vivo, and such modified dendritic cells are contemplated by the present invention. As an alternative to dendritic cells, antigen-loaded secreted vesicles (called exosomes) may be used within an immunogenic composition (see Zitvogel et al., 1998, Nature Med. 4:594-600). Thus, in an embodiment, there is provided an exosome loaded with a polypeptide, nucleic acid or fusion protein prepared from cells loaded with the antigen pool or composition of the invention.

Dendritic cells and progenitors may be obtained from peripheral blood, bone marrow, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34-positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce differentiation, maturation and proliferation of dendritic cells.

Dendritic cells are conveniently categorised as “immature” and “mature” cells, which allows a simple way to discriminate between two well-characterised phenotypes. However, this nomenclature should not be construed to exclude all possible intermediate stages of differentiation. Immature dendritic cells are characterised as APCs with a high capacity for antigen uptake and processing, which correlates with the high expression of Fcγ receptor and mannose receptor. The mature phenotype is typically characterized by a lower expression of these markers, but a high expression of cell surface molecules responsible for T-cell activation such as class I and class II MHC, adhesion molecules (e.g., CD54 and CD11) and costimulatory molecules (e.g., CD40, CD80, CD86 and 4-1BB).

APCs may also be genetically engineered e.g., transfected with a polynucleotide encoding a protein (or portion or other variant thereof) such that the polypeptide is expressed on the cell surface. Such transfection may take place ex vivo, and a pharmaceutical composition comprising such transfected cells may then be used. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., 1997, Immunology and Cell Biology 75:456-460. Antigen loading of dendritic cells may be achieved by incubating dendritic cells or progenitor cells with the polypeptide, DNA (e.g., a plasm id vector) or RNA; or with antigen-expressing recombinant bacteria or viruses (e.g., an adenovirus, adeno-associated virus (AAV) (e.g., AAV type 5 and type 2), alphavirus (e.g., Venezuelan equine encephalitis virus (VEEV), Sindbis virus (SIN), Semliki Forest virus (SFV), herpes virus, arenavirus (e.g., lymphocytic choriomeningitis virus (LCMV)), measles virus, poxvirus (such as modified vaccinia Ankara (MVA) or fowlpox), paramyxovirus, lentivirus, or rhabdovirus (such as vesicular stomatitis virus (VSV)). Prior to polypeptide loading, the polypeptides may be covalently conjugated to an immunological partner that provides T-cell help (e.g., a carrier molecule). Alternatively, a dendritic cell may be pulsed with a non-conjugated immunological partner, separately or in the presence of the antigenic polypeptide.

The invention provides for delivery of specifically designed short, chemically synthesized epitope-encoded fragments of polypeptide antigens to antigen presenting cells. Those skilled in the art will realize that these types of molecules, also known as synthetic long peptides (SLPs) provide a therapeutic platform for using the antigenic polypeptides of this invention to stimulate (or load) cells in vitro (Gornati et al., 2018, Front. Imm, 9:1484), or as a method of introducing polypeptide antigen into antigen-presenting cells in vivo (Melief & van der Burg, 2008, Nat Rev Cancer, 8:351-60).

In an embodiment, there is provided a pharmaceutical composition comprising an antigen-presenting cell of the invention, which is suitably a dendritic cell, together with a pharmaceutically acceptable carrier. Such a composition may be a sterile composition suitable for parenteral administration. See e.g., disclosure of pharmaceutical compositions supra.

In an embodiment, there is provided an antigen-presenting cell of the invention, which is suitably a dendritic cell, for use in medicine.

There is also provided a method of treating a human suffering from cancer wherein the cells of the cancer express a sequence selected from (a) to (h), wherein the cells of the cancer would express a polypeptide sequence selected from (a) to (h), which comprises administering to said human said antigen presenting cell of the invention, which is suitably a dendritic cell, or composition comprising said antigen presenting cell of the invention.

In an embodiment, there is provided an antigen presenting cell of the invention, which is suitably a dendritic cell, or composition comprising said antigen presenting cell of the invention for use in treating cancer in a human, wherein the cells of the cancer express a corresponding sequence selected from (a) to (h).

The antigen presenting cell or composition administered to the human, or the antigen presenting cell or composition for use in raising an immune response in a human depends on sequence expressed by the cancer. Thus, there is a relationship between the design of the fusion protein, nucleic acid, vector or composition and the sequence the cancer expresses.

In an embodiment, there is provided a pharmaceutical composition comprising an exosome of the invention together with a pharmaceutically acceptable carrier. Such a composition may be a sterile composition suitable for parenteral administration. See e.g., disclosure of pharmaceutical compositions supra. Compositions may optionally comprise immunostimulants—see disclosure of immunostimulants supra.

In an embodiment, there is provided an exosome of the invention for use in medicine.

There is also provided a method of treating a human suffering from cancer wherein the cells of the cancer express a polypeptide sequence selected from (a) to (h), wherein the cells of the cancer would express a sequence selected from (a) to (h), which comprises administering to said human said exosome if the invention or composition comprising said exosome of the invention.

In an embodiment, there is provided an exosome of the invention or composition comprising said exosome of the invention for use in treating cancer in a human, wherein the cells of the cancer express a corresponding sequence selected from (a) to (h).

In any one of the above embodiments, suitably the cancer is melanoma particularly cutaneous melanoma.

### Diseases to be Treated

As noted elsewhere, SEQ ID NOs. 1-8 are polypeptide sequences corresponding to CLT antigens which are over-expressed in cutaneous melanoma.

An immune response may be raised against a cancer expressing a corresponding polypeptide sequence selected from (a) to (h) or immunogenic fragments or variants thereof. In this context, “corresponding” means that if the tumor expresses (or is likely to express), say, SEQ ID NO. A (A being one of SEQ ID NOs. 1-8 or 1-10) or a variant or immunogenic fragment thereof, then the polypeptide, nucleic acid, fusion protein of the antigen pool of the invention and medicaments involving these will be based on SEQ ID NO. A or a variant or immunogenic fragment thereof.

The immune response may comprise CD8+ T-cell, a CD4+ T-cell and/or an antibody response, particularly CD8+ cytolytic T-cell response and a CD4+ helper T-cell response.

The immune response may be raised against a tumor, particularly one expressing a polypeptide sequence selected from (a) to (h).

In a preferred embodiment, the tumor is a melanoma tumor e.g. a cutaneous melanoma tumor.

The tumor may be a primary tumor or a metastatic tumor.

In one embodiment of the invention there is provided to a method of treatment of cancer in a human, wherein the cells of the cancer express the sequence of a polypeptide selected from (a) to (h) or immunogenic fragments or variants thereof, which comprises taking from said human a population of white blood cells comprising at least T-cells optionally with antigen-presenting cells, stimulating and/or amplifying said T-cells in the presence of an antigen pool or composition of the invention, and reintroducing some or all of said white blood cells at least stimulated and/or amplified T-cells into the human.

In a further embodiment of the invention there is provided a method of treating a human patient suffering from cancer wherein the cells of the cancer express a sequence selected from (a) to (h) or immunogenic fragments or variants thereof, wherein the cells of the cancer would express a sequence selected from (a) to (h) or immunogenic fragments or variants thereof, which method comprises administering to said human the T-cell population, the T-cell, antigen presenting cell, exosome or composition of the invention.

In yet a further embodiment of the invention, the invention relates to a method of treating a human suffering from cancer wherein the cells of the cancer express a sequence selected from (a) to (h) or immunogenic fragments or variants thereof, wherein the cells of the cancer would express a polypeptide sequence selected from (a) to (h) or immunogenic fragments or variants thereof, which comprises administering to said human the T-cell population, the T-cell, antigen presenting cell, exosome or composition according to the invention.

In another embodiment of the invention, there is provided a T-cell population, T-cell, antigen presenting cell, exosome or composition of the invention for use in treating cancer in a human, wherein the cells of the cancer express a corresponding sequence selected from (a) to (h) or immunogenic fragments or variants thereof.

In yet another embodiment of the invention, there is provided a method of treating a human suffering from cancer, comprising the steps of: (a) determining if the cells of said cancer express a polypeptide sequence selected from (a) to (h) or immunogenic fragments or variants thereof; and if so, (b) administering to said human a polypeptide, nucleic acid, antigen pool, composition, T-cell population, T-cell, antigen presenting cell or exosome according to the invention.

In any one of the above embodiments, suitably the cancer is melanoma particularly cutaneous melanoma.

Transcripts corresponding to SEQ ID NOs. 14 and 20 were also overexpressed in uveal melanoma. Consequently, in an alternative embodiment, the tumor is a uveal melanoma tumor and/or the tumor expresses a sequence selected from SEQ ID NOs. 1, 3 and 4. Thus, fusion proteins of the present invention may therefore be indicated in subjects having uveal cancer.

### Antigen Combinations

The antigen pools, T-cell population, T-cell, antigen presenting cell, exosome or compositions of the invention can be used in combination with other immunogenic antigens which cause an immune response to be raised against melanoma e.g. cutaneous or uveal melanoma. These other immunogenic antigens could be derived from diverse sources, they could include well-described melanoma-associated antigens, such as GPR143, PRAME, MAGE-A3 or pMel (gp100). Alternatively they could include other types of melanoma antigens, including patient-specific neoantigens (Lauss et al. (2017). Nature Communications, 8(1), 1738. http://doi.org/10.1038/s41467-017-01460-0), retained-intron neoantigens (Smart et al. (2018). Nature Biotechnology. http://doi.org/10.1038/nbt.4239), spliced variant neoantigens (Hoyos et al., Cancer Cell, 34(2), 181-183. http://doi.org/10.1016/j.ccell.2018.07.008; Kahles et al. (2018). Cancer Cell, 34(2), 211-224.e6. http://doi.org/10.1016/j.ccell.2018.07.001), melanoma antigens that fit within the category known as antigens encoding T-cell epitopes associated with impaired peptide processing (TIEPPs; Gigoux, M., & Wolchok, J. (2018). JEM, 215, 2233, Marijt et al. (2018). JEM 215, 2325), or to-be discovered neoantigens (including CLT antigens). In addition, the antigenic peptides from these various sources could also be combined with (i) non-specific immunostimulant/adjuvant species and/or (ii) an antigen, e.g. comprising universal CD4 helper epitopes, known to elicit strong CD4 helper T-cells (delivered as a polypeptides, or as polynucleotides or vectors encoding these CD4 antigens), to amplify the anti-melanoma-specific responses elicited by co-administered antigens.

Different antigens present in the form of a polypeptide and/or nucleic acid encoding the polypeptide may be formulated in the same formulation or in separate formulations. The different antigens may be provided as separate polypeptides, nucleic acids, fusion proteins in which a polypeptide is fused to a second or further polypeptide and/or nucleic acid encoding the fusion protein.

More generally, when two or more components are utilised in combination, the components could be presented, for example:
(1) as two or more individual and/or separate antigenic polypeptide components;
(2) as a fusion protein comprising both (or further) polypeptide components;
(3) as two or more polypeptide and two or more polynucleotide component;
(4) as two or more individual polynucleotide components;
(5) as a single polynucleotide encoding two or more individual polypeptide components; or
(6) as a single polynucleotide encoding a fusion protein comprising both (or further) polypeptide components.

For convenience, when a number of components are present, they are contained within a single fusion protein or a polynucleotide encoding a single fusion protein. In one embodiment of the invention all components are provided as polypeptides (e.g., within a single fusion protein). In an alternative embodiment of the invention all components are provided as polynucleotides (e.g., a single polynucleotide, such as one encoding a single fusion protein).

### Combination Therapies

Methods of treating cancer according to the invention may be performed in combination with other therapies, especially checkpoint inhibitors and interferons.

The adoptive cell therapies (APC and T-cell-based) can be used in combination with other components designed to enhance their immunogenicity, for example, to improve the magnitude and/or breadth of the elicited immune response, or provide other activities (e.g. activation of other aspects of the innate or adaptive immune response, or destruction of tumor cells).

Accordingly, a composition of the invention (i.e. an immunogenic or pharmaceutical composition) or a kit of several such compositions may comprise an antigen pool, T cell population, a T cell, antigen presenting cell or exosome of the invention together with a pharmaceutically acceptable carrier; and (i) one or more further immunogenic or immunostimulant polypeptides (e.g., interferons, IL-12, checkpoint blockade molecules or nucleic acids encoding such, or vectors comprising such nucleic acids), (ii) small molecules (e.g., HDAC inhibitors or other drugs that modify the epigenetic profile of cancer cells) or biologicals (delivered as polypeptides or nucleic acids encoding such, or vectors comprising such nucleic acids) that will enhance the translation and/or presentation of the polypeptide products that are the subject of this invention.

Checkpoint inhibitors, which block normal proteins on cancer cells, or the proteins on the T-cells that respond to them, may be a particularly important class of drugs to combine with CLT-antigen based therapies, since these inhibitors seek to overcome one of cancer's main defences against an immune system attack.

Thus, an antigen pool, immunogenic or pharmaceutical composition, T-cell, T-cell population, antigen presenting cell or exosome of the invention may be administered in combination with a checkpoint inhibitor. Example check point inhibitors are selected from PD-1 inhibitors, such as pembrolizumab, (Keytruda) and nivolumab (Opdivo), PD-L1 inhibitors, such as atezolizumab (Tecentriq), avelumab (Bavencio) and durvalumab (Imfinzi) and CTLA-4 inhibitors such as ipilimumab (Yervoy).

Interferons (e.g., alpha, beta and gamma) are a family of proteins the body makes in very small amounts. Interferons may slow down or stop the cancer cells dividing, reduce the ability of the cancer cells to protect themselves from the immune system and/or enhance multiple aspects of the adaptive immune system. Interferons are typically administered as a subcutaneous injection in, for example the thigh or abdomen.

Thus, an antigen pool, immunogenic or pharmaceutical composition T-cell, T-cell population, antigen presenting cell or exosome of the invention may be administered in combination with interferon e.g., interferon alpha.

Different modes of the invention may also be combined, for example antigen pools of the invention may be combined with an APC, a T-cell, a T-cell population or an exosome of the invention (discussed infra).

One or more modes of the invention may also be combined with conventional anti-cancer chemotherapy and/or radiation.

### EXAMPLES

### Example 1—CLT Identification

The objective was to identify cancer-specific transcripts that entirely or partially consist of LTR elements.

As a first step, we de novo assembled a comprehensive pan-cancer transcriptome. To achieve this, RNA-sequencing reads from 768 patient samples, obtained from The Cancer Genome Atlas (TCGA) consortium to represent a wide variety of cancer types (24 gender-balanced samples from each of 32 cancer types (31 primary and 1 metastatic melanoma); Table S1), were used for genome-guided assembly. The gender-balanced samples (excluding gender-specific tissues) were adapter and quality (Q20) trimmed and length filtered (both reads of the pair 35 nucleotides) using cutadapt (v1.13) (Marcel M., 2011, EMBnet J., 17:3) and kmer-normalized (k=20) using khmer (v2.0) (Crusoe et al., 2015, F1000Res., 4:900) for maximum and minimum depths of 200 and 3, respectively. Reads were mapped to GRCh38 using STAR (2.5.2b) with settings identical to those used across TCGA and passed to Trinity (v2.2.0) (Trinity, Grabherr, M. G., et al., 2011, Nat. Biotechnol., 29:644-52) for a genome-guided assembly with inbuilt in silico depth normalization disabled. The majority of assembly processes were completed within 256 GB RAM on 32-core HPC nodes, with failed processes re-run using 1.5 TB RAM nodes. Resulting contigs were poly(A)-trimmed (trimpoly within SeqClean v110222) and entropy-filtered (0.7) to remove low-quality and artefactual contigs (bbduk within BBMap v36.2). Per cancer type, the original 24 samples were quasi-mapped to the cleaned assembly using Salmon (v0.8.2 or v0.9.2) (Patro, R., et al., 2017, Nat. Methods, 14:417-419), with contigs found expressed at <0.1 transcripts per million (TPM) being removed. Those remaining were mapped to GRCh38 using GMAP (v161107) (Wu et al., 2005, Bioinf., 21:1859-1875), and contigs not aligning with ≥85% identity over ≥85% of their length were removed from the assembly. Finally, assemblies for all cancer types together were flattened and merged into the longest continuous transcripts using gffread (Cufflinks v2.2.1) (Trapnell et al., 2010, Nat. Biotech., 28:511-515). As this assembly process was specifically designed to enable assessment of repetitive elements, monoexonic transcripts were retained, but flagged. Transcript assembly completeness and quality was assessed by comparison with GENCODE v24basic and MiTranscriptome1 (Iyer et al. 2015, Nat. Genet., 47: 199-208). We compiled the list of unique splice sites represented within GENCODE and tested if the splice site was present within the transcriptome assembly within a 2-nucleotide grace window. This process resulted in the identification of 1,001,931 transcripts, 771,006 of which were spliced and 230,925 monoexonic.

Separately, the assembled contigs were overlaid with a genomic repeat sequence annotation to identify transcripts that contain an LTR element. LTR and non-LTR elements were annotated as previously described (Attig et al., 2017, Front. In Microbiol., 8:2489). Briefly, hidden Markov models (HMMs) representing known Human repeat families (Dfam 2.0 library v150923) were used to annotate GRCh38 using RepeatMasker Open-3.0 (Sm it, A., R. Hubley, and P. Green, http://www.repeatmasker.org, 1996-2010), configured with nhmmer (Wheeler et al., 2013, Bioinform., 29:2487-2489). HMM-based scanning increases the accuracy of annotation in comparison with BLAST-based methods (Hubley et al., 2016, Nuc. Acid. Res., 44:81-89). RepeatMasker annotates LTR and internal regions separately, thus tabular outputs were parsed to merge adjacent annotations for the same element. This process yielded 181,967 transcripts that contained one or more, complete or partial LTR element.

Transcripts per million (TPM) were estimated for all transcripts using Salmon and expression within each cancer type was compared with expression across 811 healthy tissue samples (healthy tissue-matched controls for all cancer types, where available, from TCGA and, separately from, GTEx (The Genotype-Tissue Expression Consortium, 2015, Science, 348:648-60). Transcripts were considered expressed in cancer if detected at more than 1 TPM in any sample and as cancer-specific if the following criteria were fulfilled: i, expressed in ≥6 of the 24 samples of each cancer type; ii, expressed at <10 TPM in ≥90% of all healthy tissue samples; iii, expressed in the cancer type of interest ≥3× the median expression in any control tissue type; and iv, expressed in the cancer type of interest ≥3× the 90th percentile of the respective healthy tissue, where available.

The list of cancer-specific transcripts was then intersected with the list of transcripts containing complete or partial LTR elements to produce a list of 5,923 transcripts that fulfilled all criteria (referred to as CLTs for Cancer-specific LTR element-spanning Transcripts).

Further curation was carried out on 403 CLTs specifically expressed in melanoma to exclude potentially misassembled contigs and those corresponding to the assembly of cellular genes. Additional manual assessment was conducted to ensure that splicing patterns were supported by the original RNA-sequencing reads from melanoma. CLTs were additionally triaged such that those where the median expression in any GTEx normal tissue exceeded 1 TPM were discarded.

Within the 403 CLTs for cutaneous melanoma, 97 CLTs passed these filters.

### Example 2—Immunopeptidomic Analysis

Mass spectrometry (MS)-based immunopeptidomics analysis is a powerful technology that allows for the direct identification of specific peptides associated with HLA molecules (pHLA) and presented on the cell surface. The technique consists of affinity purification of the pHLA from biological samples such as cells or tissues by anti-HLA antibody capture. The isolated HLA molecules and bound peptides are then separated from each other and the eluted peptides are analyzed by nano-ultra performance liquid chromatography coupled to mass spectrometry (nUPLC-MS) (Freudenmann et al., 2018, Immunology 154(3):331-345). In the mass spectrometer, specific peptides of defined charge-to-mass ratio (m/z) are selected, isolated, fragmented, and then subjected to a second round of mass spectrometry (MS/MS) to reveal the m/z of the resulting fragment ions. The fragmentation spectra (MS/MS) can then be interrogated to precisely identify the amino acid sequence of the selected peptide that gave rise to the detected fragment ions.

MS/MS spectral interpretation and subsequent peptide sequence identification relies on the match between experimental data and theoretical spectra created from peptide sequences found in a reference database. Although it is possible to search MS data by using pre-defined lists corresponding to all open reading frames (ORFs) derived from the known transcriptome or even the entire genome (Nesvizhskii et al., 2014, Nat. Methods 11: 1114-1125), interrogating these very large sequence databases leads to very high false discovery rates (FDR) that limit the identification of presented peptides. Further technical issues (e.g., mass of leucine=mass of isoleucine), and theoretical issues (e.g., peptide splicing (Liepe, et al., 2016, Science 354(6310): 354-358)) increase the limitations associated with use of very large databases, such as those produced from the known transcriptome or entire genome. Thus, in practice, it is exceptionally difficult to perform accurate immunopeptidomics analyses to identify novel antigens without reference to a well-defined set of potential polypeptide sequences (Li, et al., 2016, BMC Genomics 17 (Suppl 13):1031).

Bassani-Sternberg et al. (Bassani-Sternberg et al., 2016, Nature Commun., 7: 13404; database link: https://www.ebi.ac.uk/pride/archive/projects/PXD004894) interrogated MS/MS data collected from HLA-bound peptide samples derived from 25 cutaneous melanoma patients against the polypeptide sequences reported for the entire human proteome. These analyses revealed tens of thousands of peptides that matched to known human proteins. As expected, these peptides included peptides found within multiple tumor-associated antigens (TAA), including PRAME, MAGEA3, and TRPM1 (melastatin).

The inventors procured frozen tumor tissue from 6 patients diagnosed with melanoma. Samples between 0.05-1 g were homogenized, the lysate was centrifugate at high speed and the cleared lysate was mixed with protein A (ProA) beads covalently linked to an anti-human HLA class I monoclonal antibody (W6/32). The mixture was incubated overnight at 4° C. to improve HLA Class I molecule binding to antibody (Ternette et al., 2018 Proteomics 18, 1700465). The HLA Class I-bound peptides were eluted from the antibody by using 10% acetic acid, and the peptides were then separated from other high molecular mass components using reversed-phase column chromatography (Ternette et al., 2018). The purified, eluted peptides were subjected to nUPLC-MS, and specific peptides of defined charge-to-mass ratio (m/z) were selected within the mass spectrometer, isolated, fragmented, and subjected to a second round of mass spectrometry (MS/MS) to reveal the m/z of the resulting fragment ions (Ternette et al., 2018), producing an MS/MS dataset corresponding to the immunopeptidome for each of these tumor samples.

By applying detailed knowledge of immunopeptidomics evaluation, the inventors interrogated the spectra from the PXD004894 HLA Class I dataset for 25 melanoma patients (Bassani-Sternberg et al., 2016) and the spectra of the HLA-Class I dataset for the 6 melanoma patients prepared by the inventors with the CLT-derived ORFs (of Example 1). Three types of analyses were conducted:
Analysis A: Predicted ORFs of greater than 23 amino acid residues from a subset of approximately 1 dozen CLTs derived from those identified in Example 1 were concatenated into a single polypeptide file for each CLT, and these concatenated ORF polypeptides were interrogated against the PXD004894 HLA Class I dataset for 25 melanoma patients alongside all polypeptides found in the human proteome (UniProt database) by using the PEAKS™ software (v8.5, Bioinformatics Solutions Inc)
Analysis B: Polypeptide files consisting of each of the predicted ORFs of greater than 23 amino acid residues from a subset of approximately 1 dozen CLTs derived from those identified in Example 1 were interrogated against the PXD004894 HLA Class I dataset for 25 melanoma patients alongside all the polypeptides found in the human proteome (UniProt and masDB databases) by using the Mascot software
Analysis C: All predicted ORFs derived from the 97 CLTs identified in Example 1 of 10 or more amino acid residues in length, were interrogated against the PXD004894 HLA Class I dataset for 25 melanoma patients and the inventors' HLA Class I dataset for 6 melanoma patients alongside all the polypeptides found in the human proteome (UniProt) using PEAKS™ software (v8.5 and vX, Bioinformatics Solutions Inc)

Since the majority of Class I HLA-bound peptides found in cells are derived from constitutively expressed proteins, the simultaneous interrogation of these databases with the UniProt proteome helps to ensure that assignments of our CLT ORF sequences to MS/MS spectra are correct. The PEAKS software, like other MS/MS interrogation software, assigns a probability value (−10 lgP; see Table 1) to each spectral assignment to quantify the assignment.

The results of these studies identified >50 individual peptides that were associated with the HLA Class I molecules immunoprecipitated from tumor samples from the 25 patients examined by Bassani-Sternberg et al. and the 6 melanoma patient samples in the inventors' dataset, that corresponded to the amino acid sequence of CLT-derived ORFs, and did not correspond to polypeptide sequences present within the known human proteome (UniProt and/or masDB).

Further manual review of the peptide spectra assigned by the PEAKS software was used to confirm assignment of spectra to peptides that were mapped to 8 CLT-derived ORFs, and thus defined as CLT antigens (Table 1; SEQ ID NOs. 1-8).

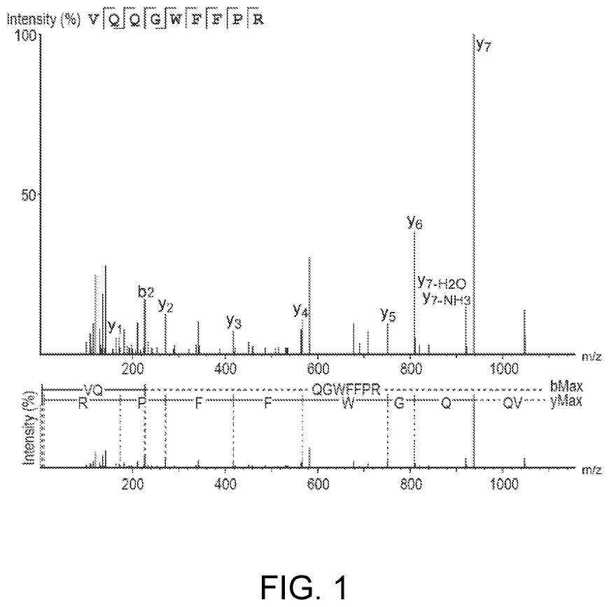

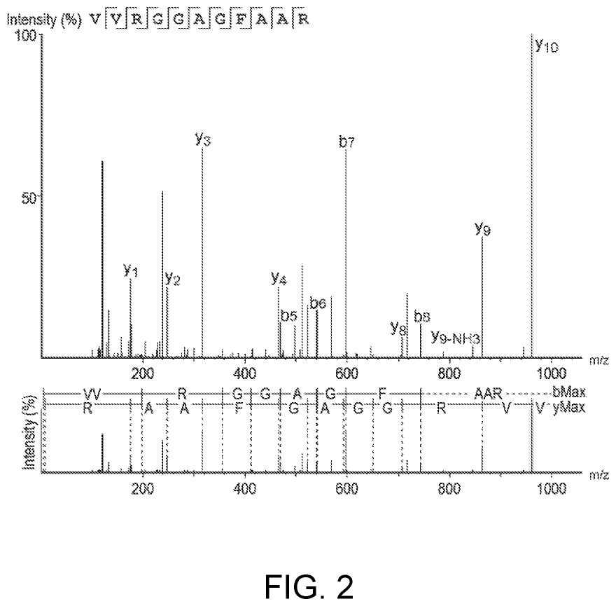

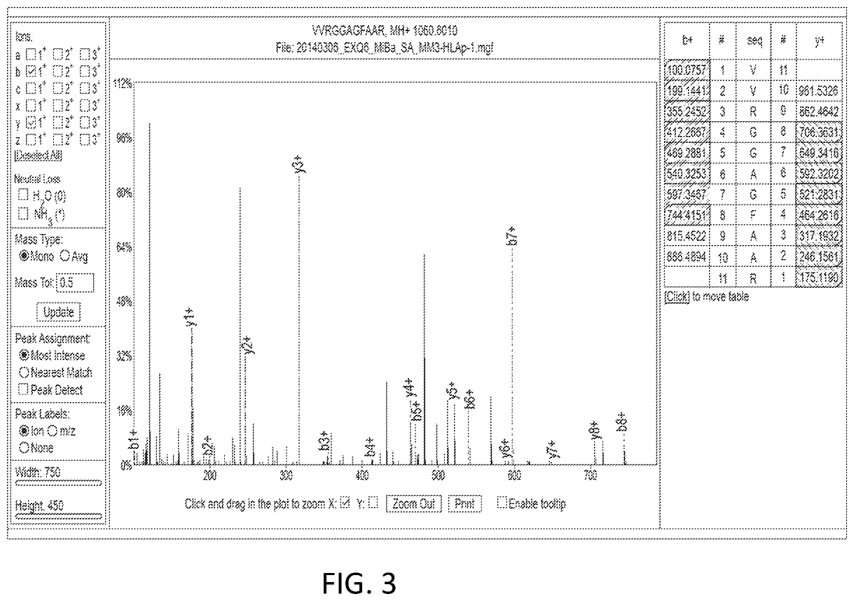

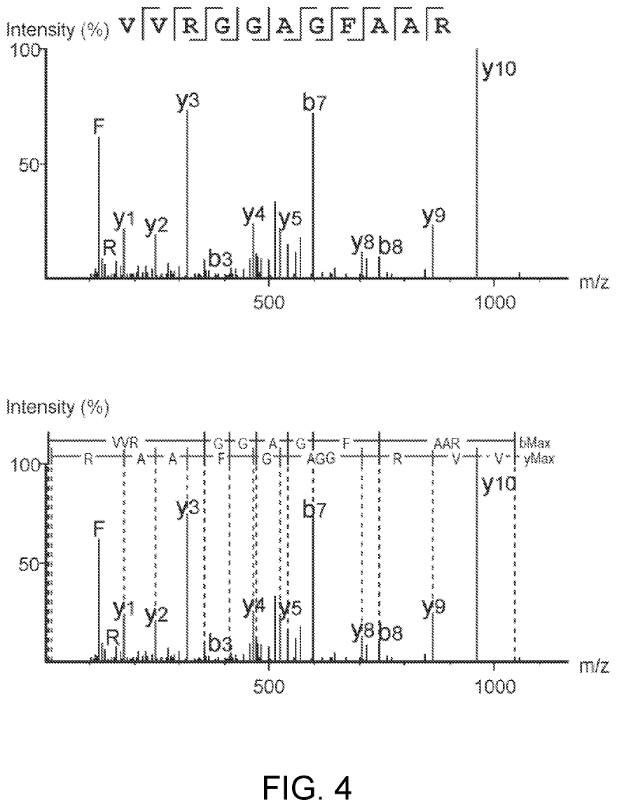

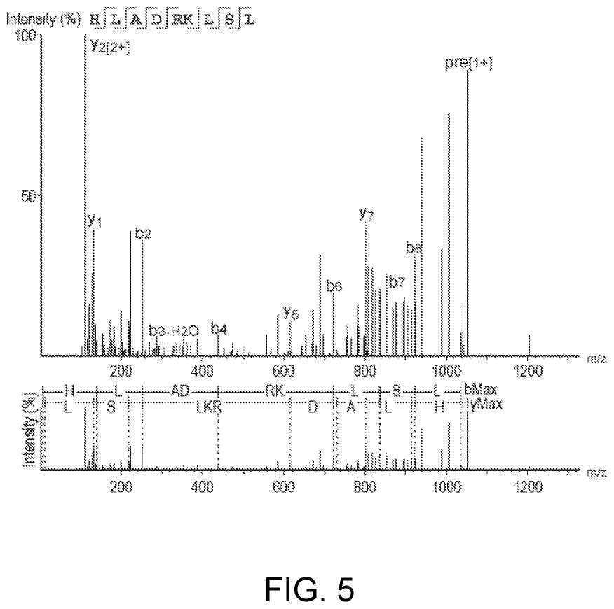

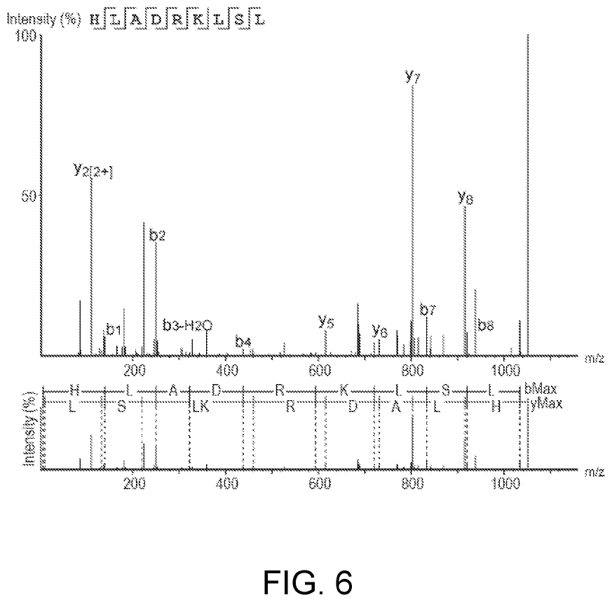

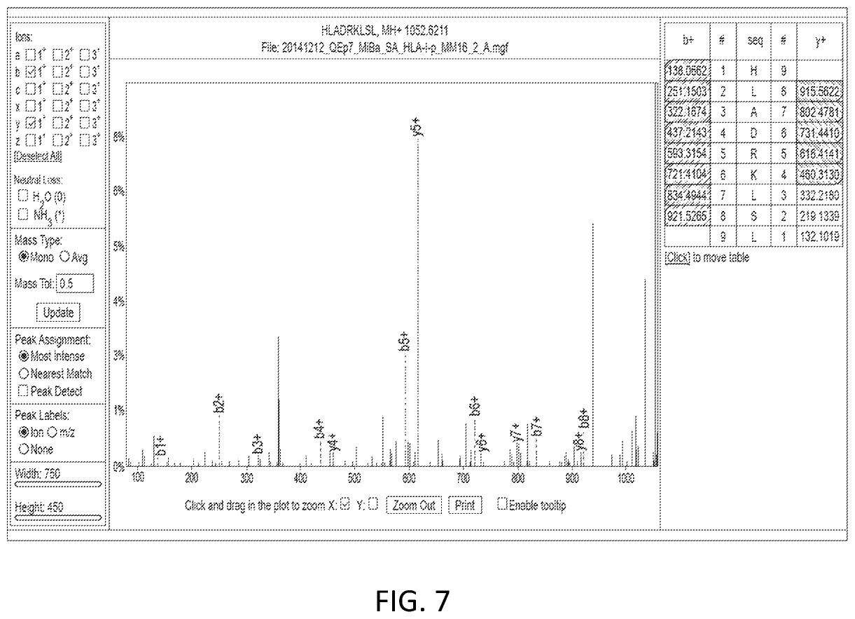

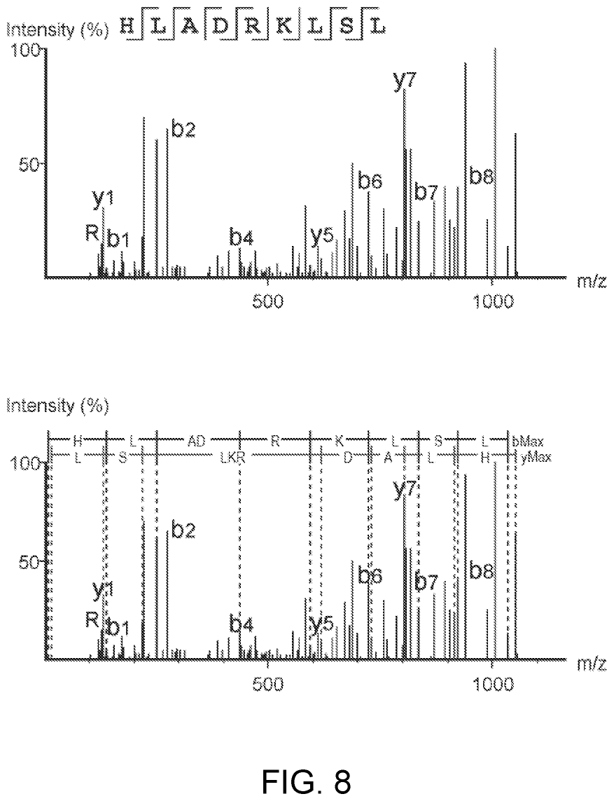

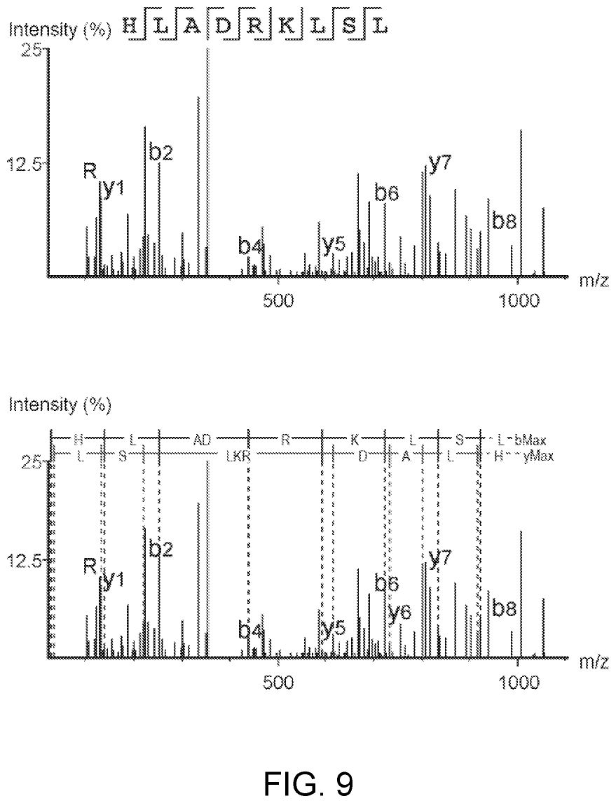

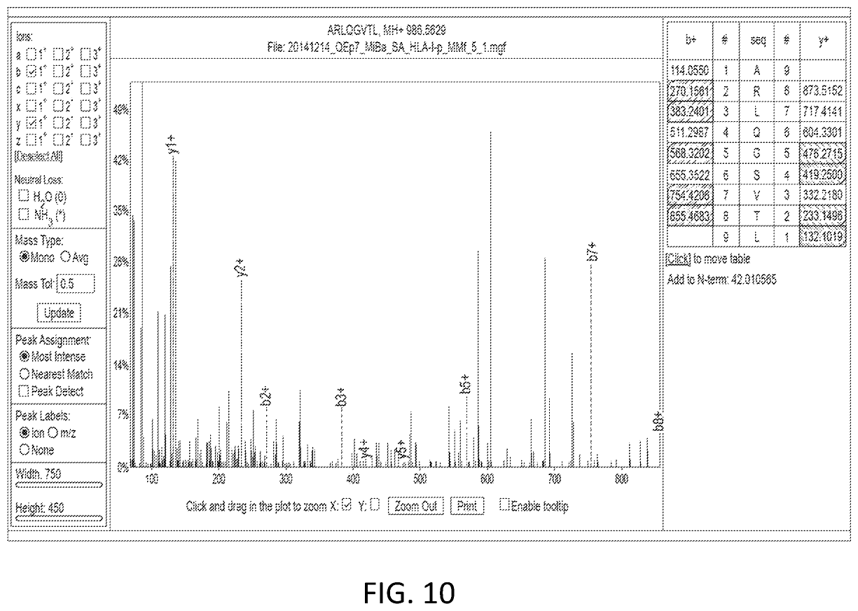

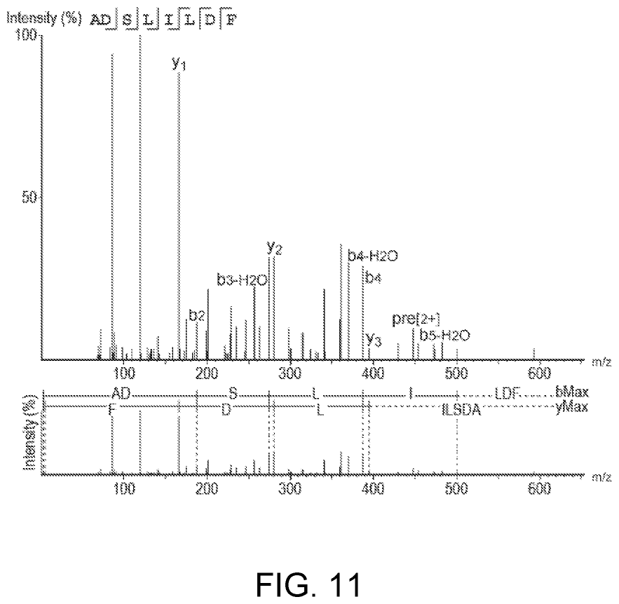

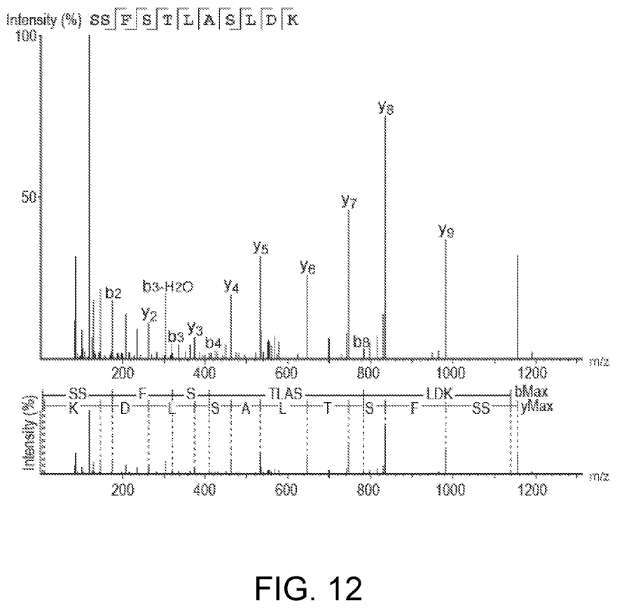

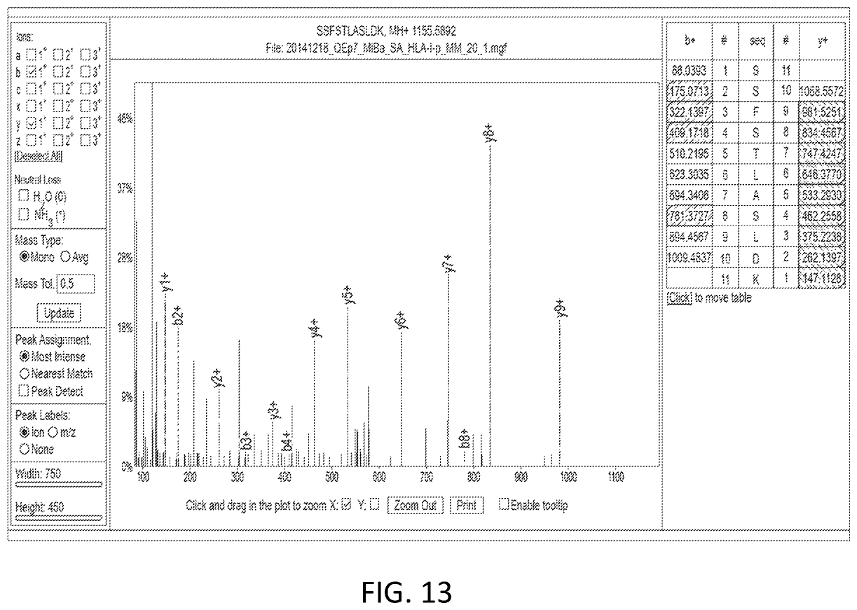

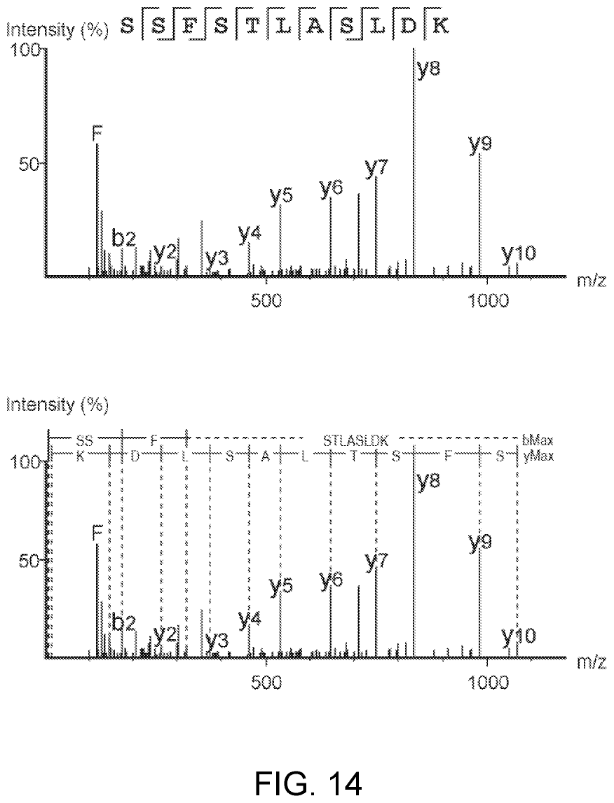

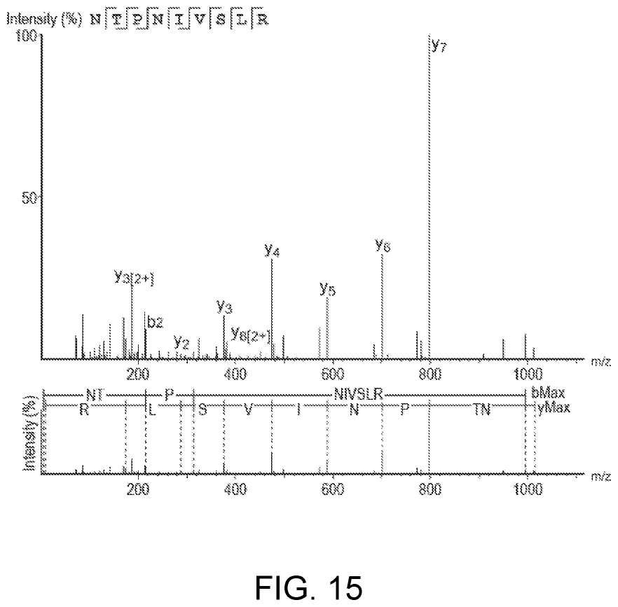

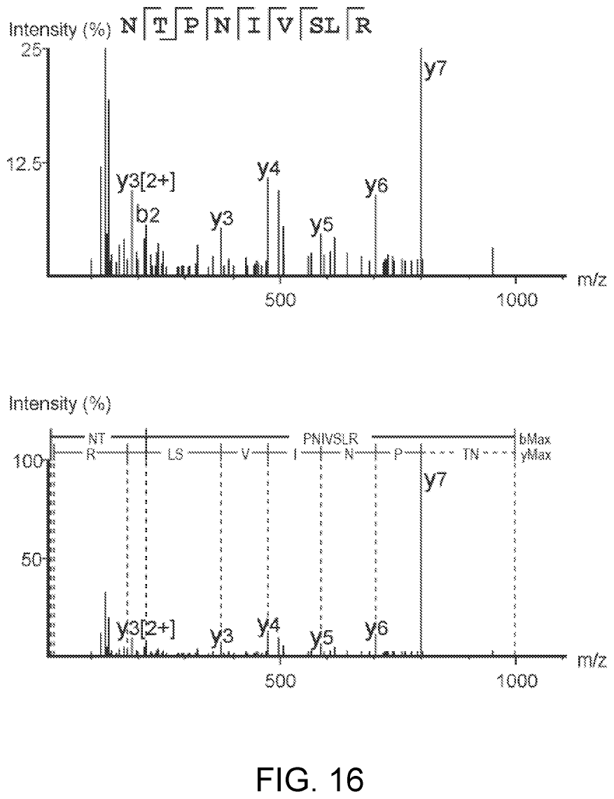

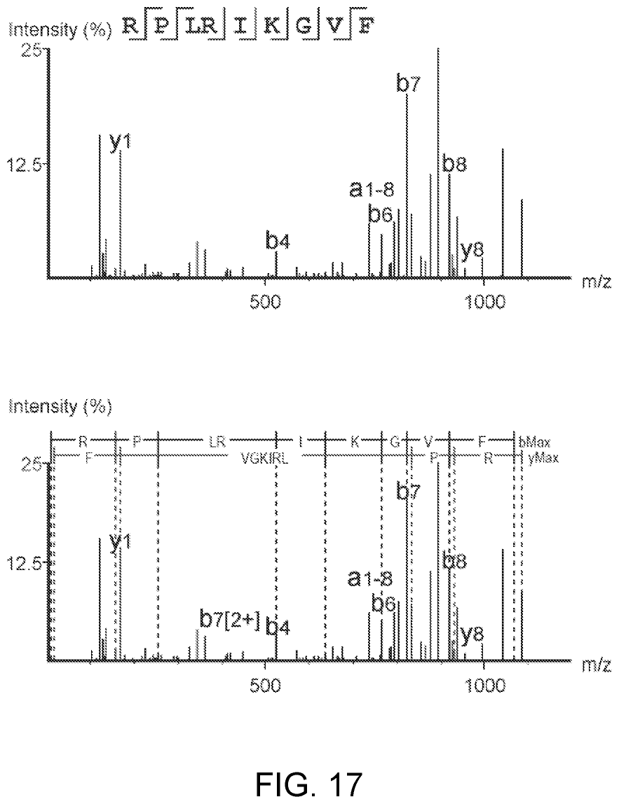

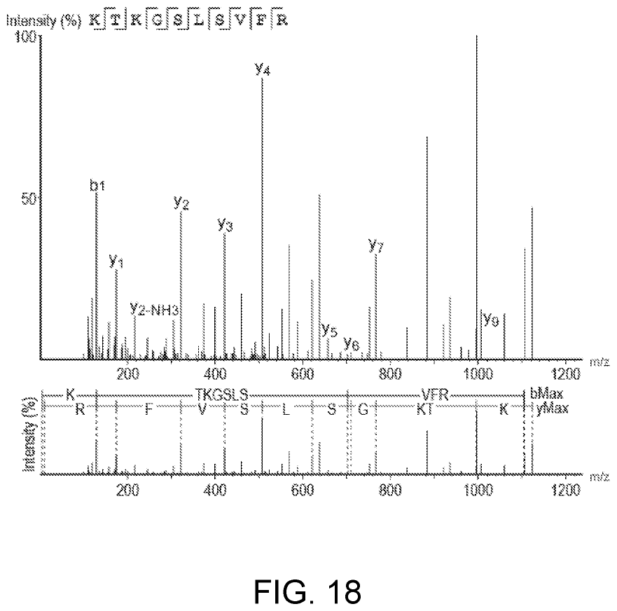

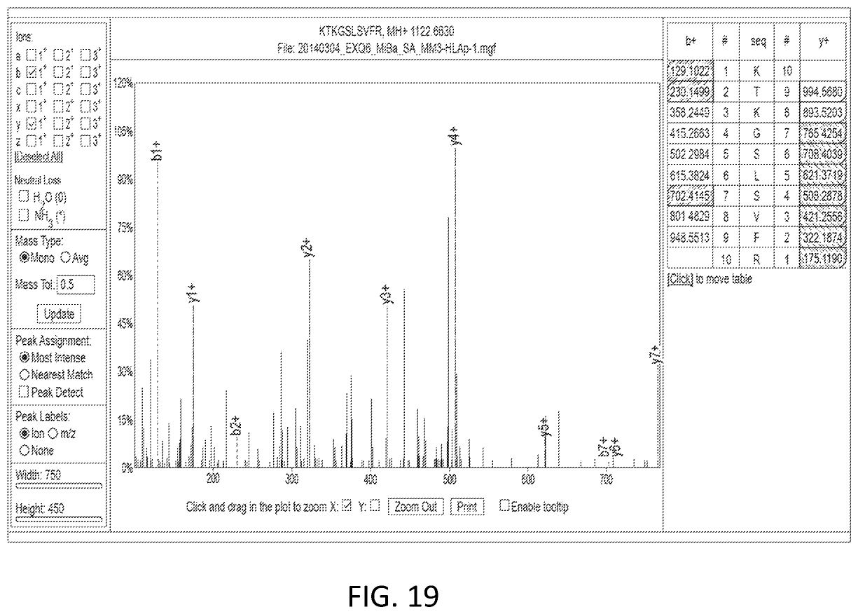

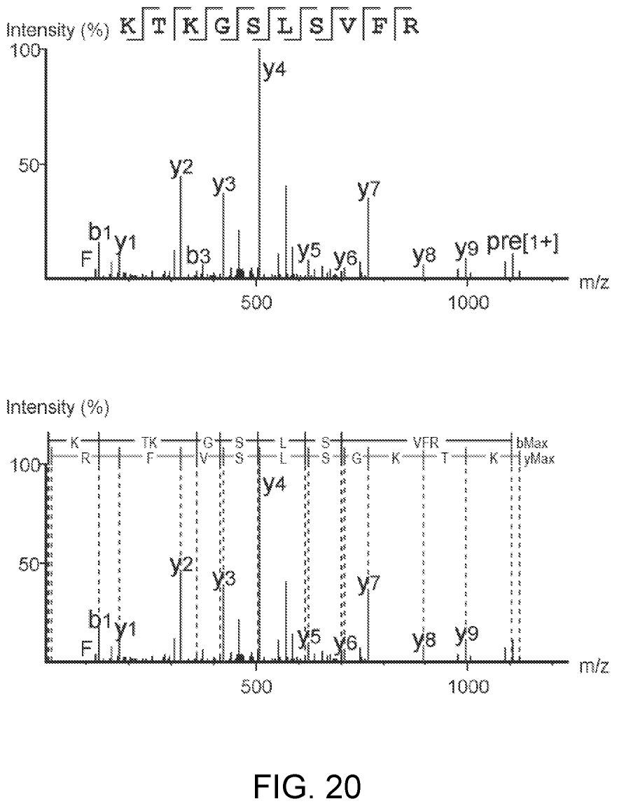

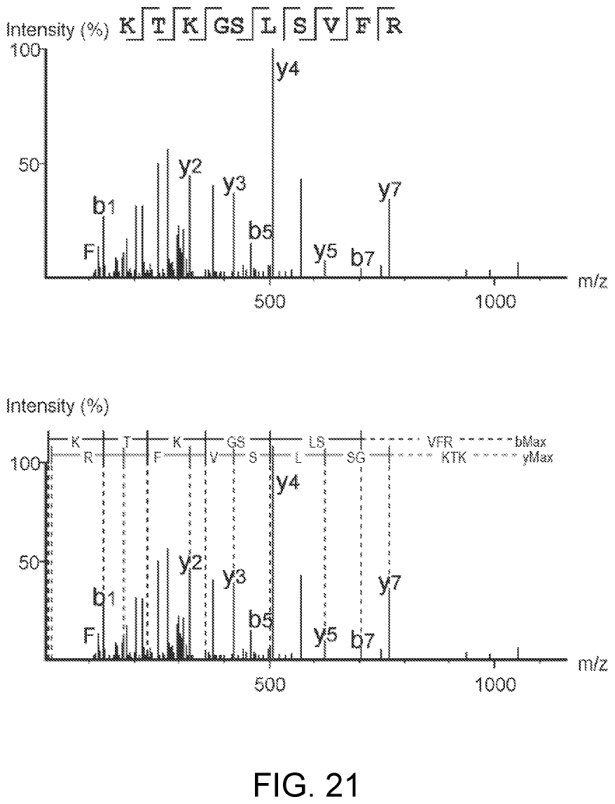

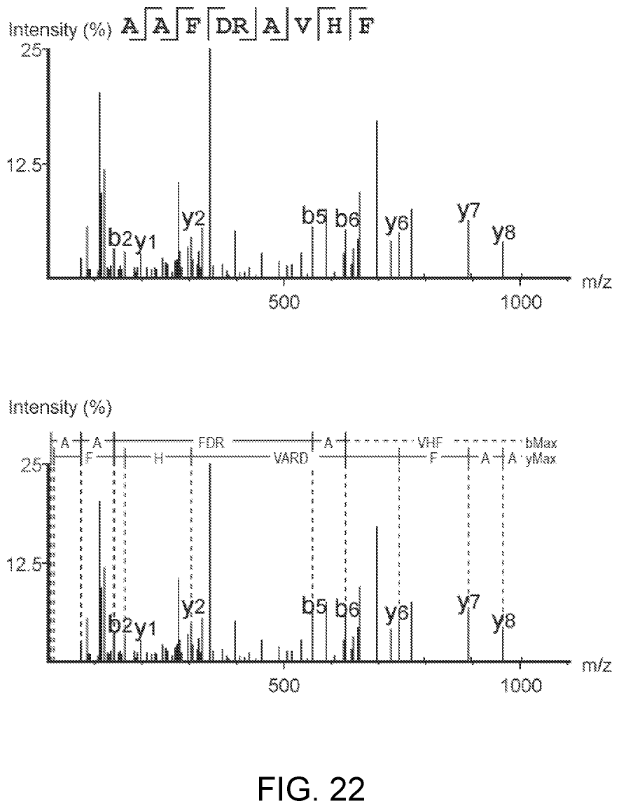

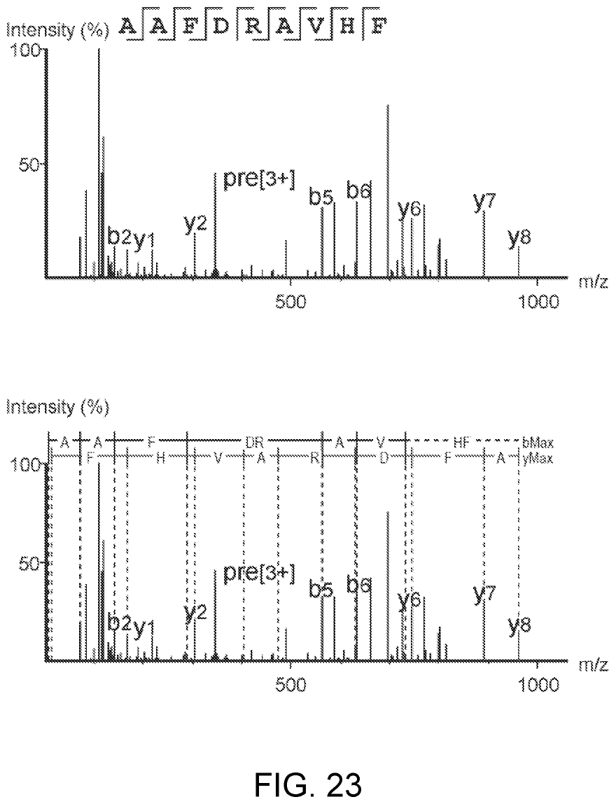

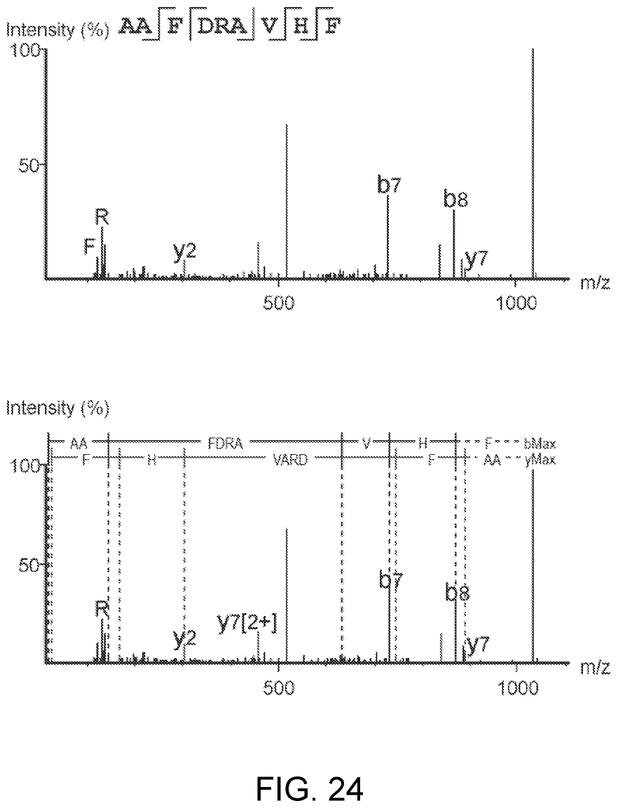

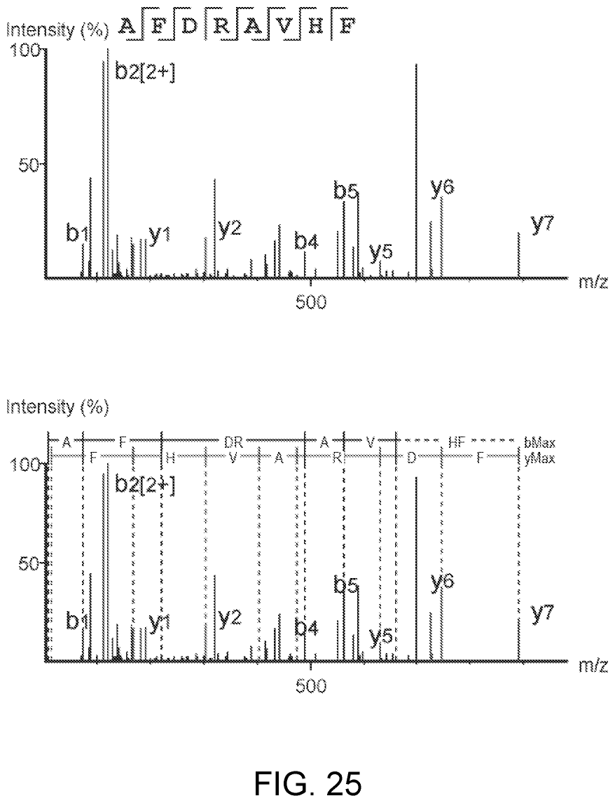

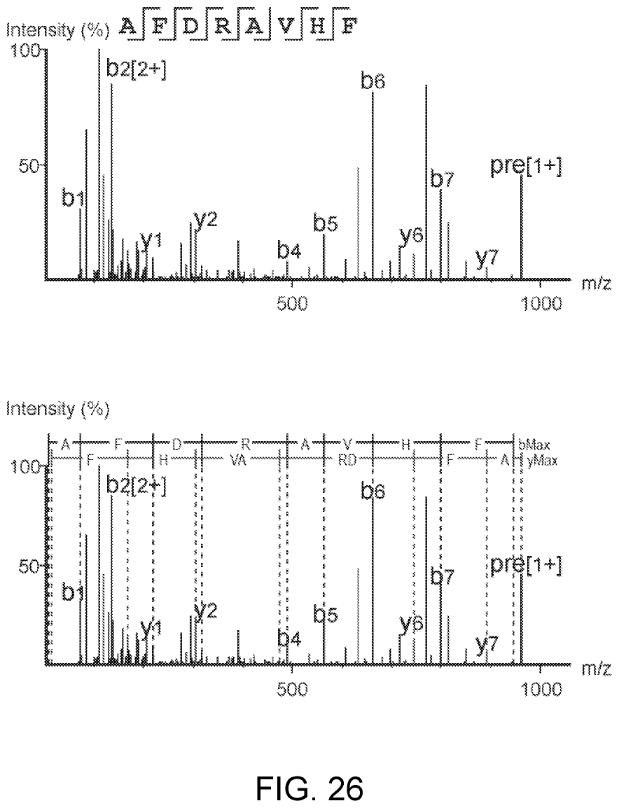

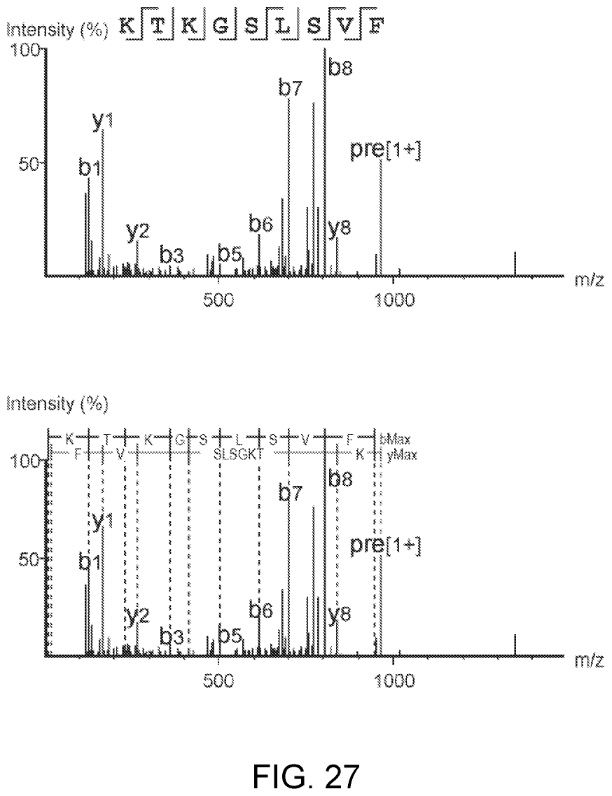

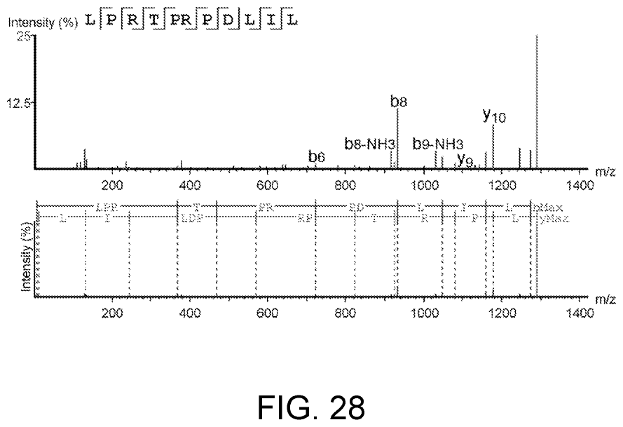

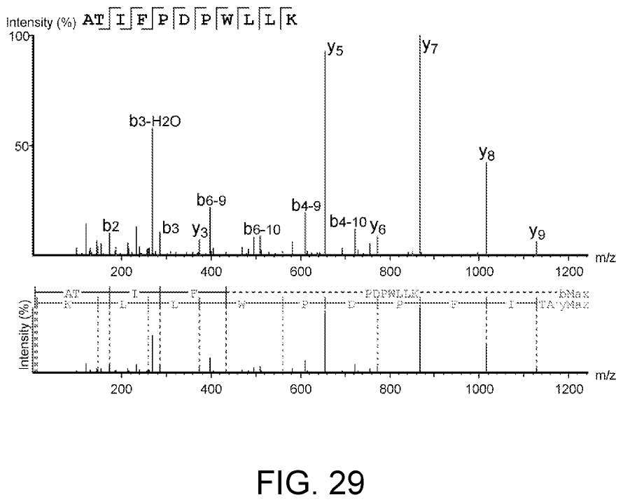

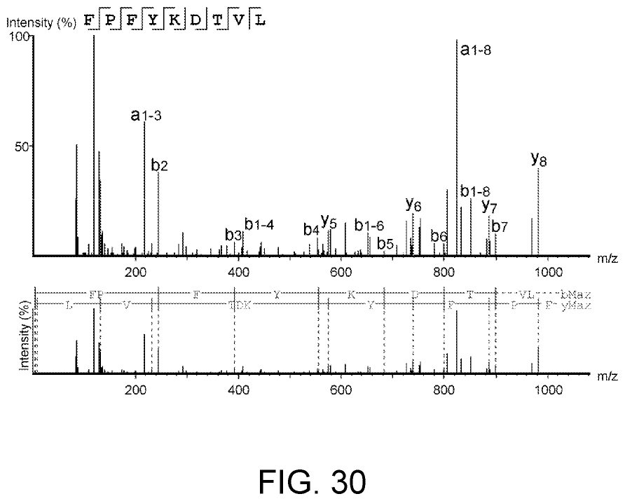

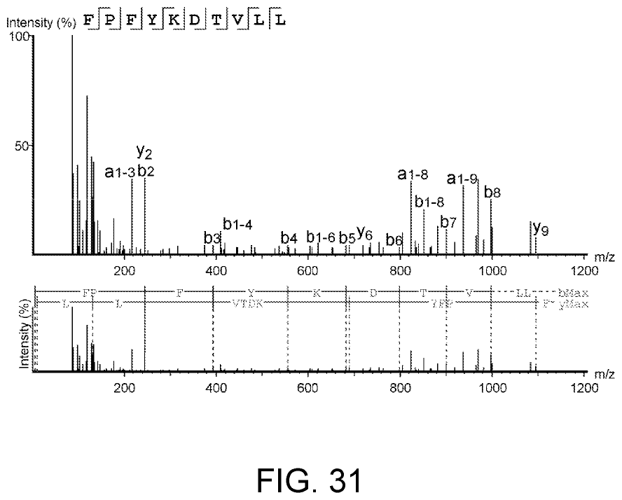

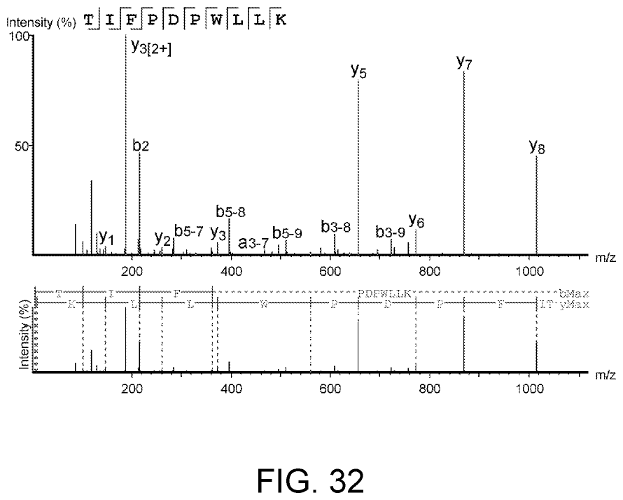

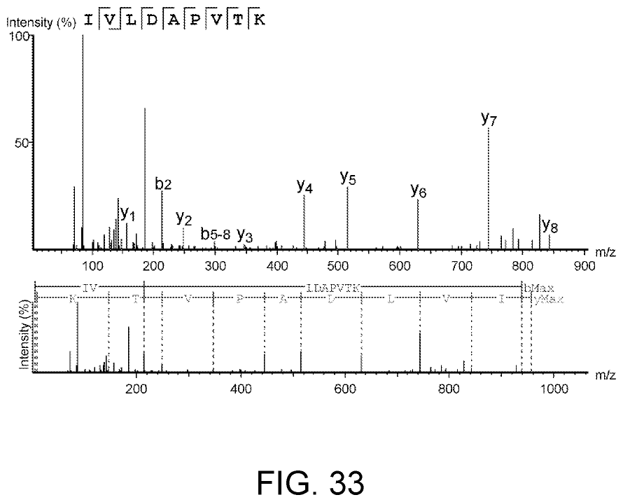

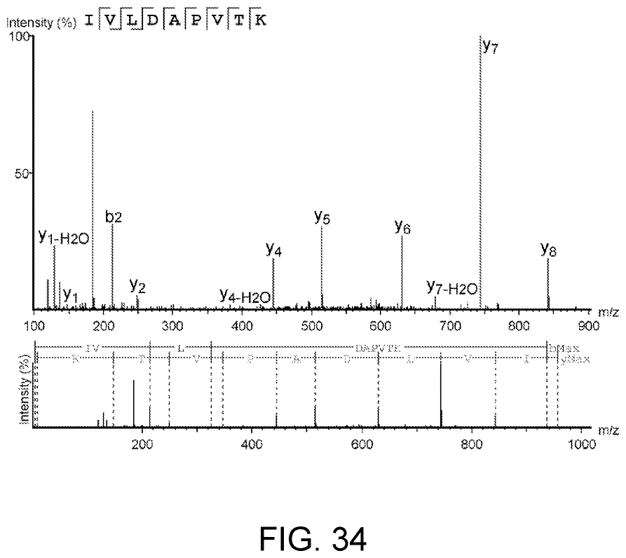

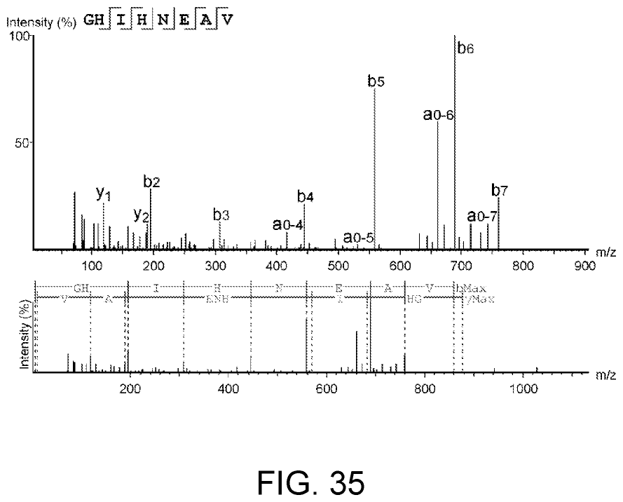

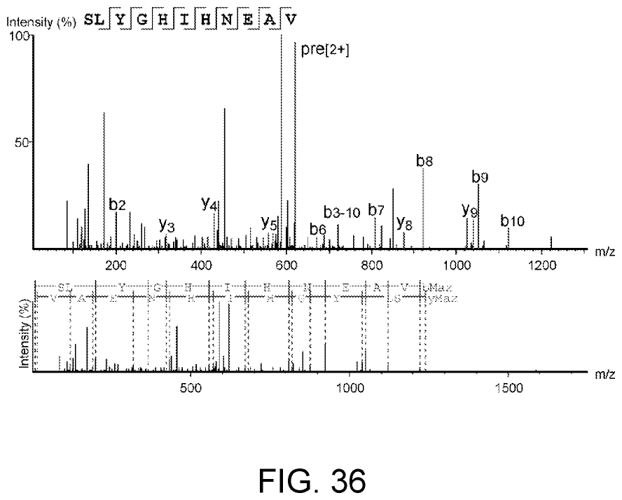

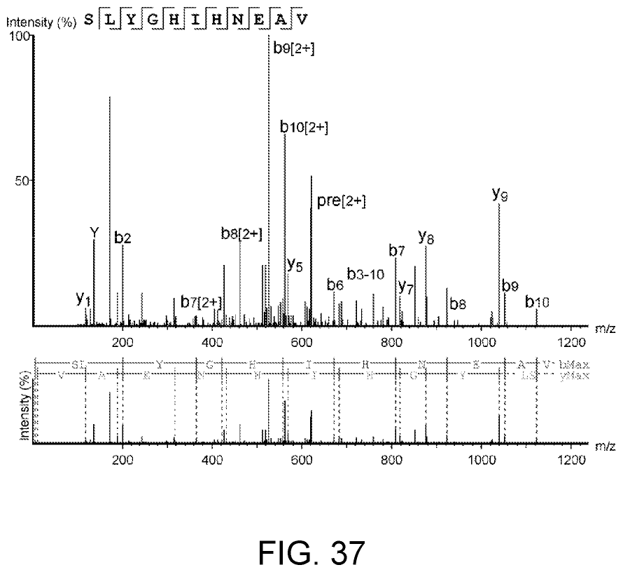

The detection of these peptides associated with the HLA Class I molecules confirms, that the 8 ORFs from which they were derived, were first translated in melanoma tissues, processed through the HLA Class I pathway and finally presented to the immune system in a complex with HLA Class I molecules. Table 1 shows the properties of the peptides found in the CLT antigens. FIGS. 1-37 show representative MS/MS spectra from each of the peptides shown in Table 1. The figures show fragment spectra for indicated peptide sequences as detected in individual patient SKCM tumors by nUPLC-MS² (images extracted by PEAKS™ software from the inventors' internal dataset or from Bassani-Sternberg et al, dataset stored in PRIDE). All fragments that have been detected are indicated in the peptide sequence above the spectrum and the most abundant fragment ions are assigned in each spectrum. In FIGS. 1-2, 4-6, 8-9, 11-12, 14-37, the lower panel of the figures illustrates the peptide sequences assigned to the MS/MS spectrum, whereas similar data are shown in tabular form on the right side of FIGS. 3, 7, 10, 13 and 19. Fragment ions are annotated as follows: b: N-terminal fragment ion; y: C-terminal fragment ion; —H₂O: water loss; —NH₃: loss of ammonia; [2+]: doubly charged peptide ion; pre: unfragmented precursor peptide ion. Consistent with the high −10 lgP scores assigned to the peptides in Table 1, these spectra contain numerous fragments that precisely match the sequences of the peptides (SEQ ID NOs. 9-12, 18-19, 31-32, 36-39, 45, 48-54) that we discovered in these analyses.

All of the peptides detected in association with HLA Class I from Table 1 that were 9 amino acid residues or more in length were assessed to determine their predicted strength of binding to HLA Class I type A and B supertypes by using the NetMHCpan 4.0 prediction software (http://www.cbs.dtu.dk/services/NetMHCpan/). The results of these prediction studies showed that all of the 17 peptides (or 9-mers contained within each full sequence) were predicted to bind to at least one of the supertypes tested (see Table 2). Amongst these, many of the sequences were predicted to bind with high confidence (low % rank scores) to specific types within the HLA Class I supertypes examined. The fact that all of the detected peptides were expected to bind to the standard set of HLA types provides additional validation around their detection. Moreover, every peptide discovered in a tumor sample from the inventors' dataset was predicted by NetMHCpan 4.0 to bind to one of the HLA types we detected in the patient sample. HLA types were not reported by Bassani-Sternberg et al. (2016, Nature Commun., 7: 13404) for every patient associated with the peptides we discovered, but where this was reported, we found matches between the known and predicted HLA types.

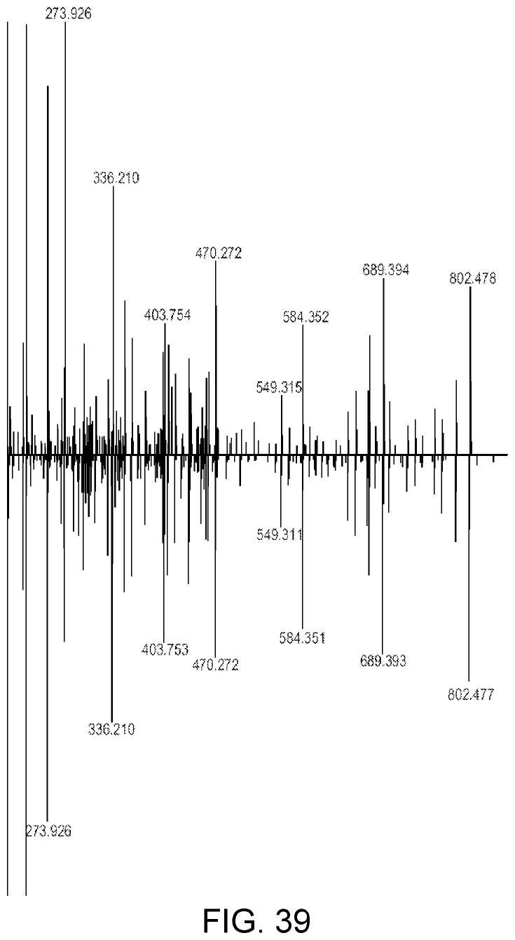

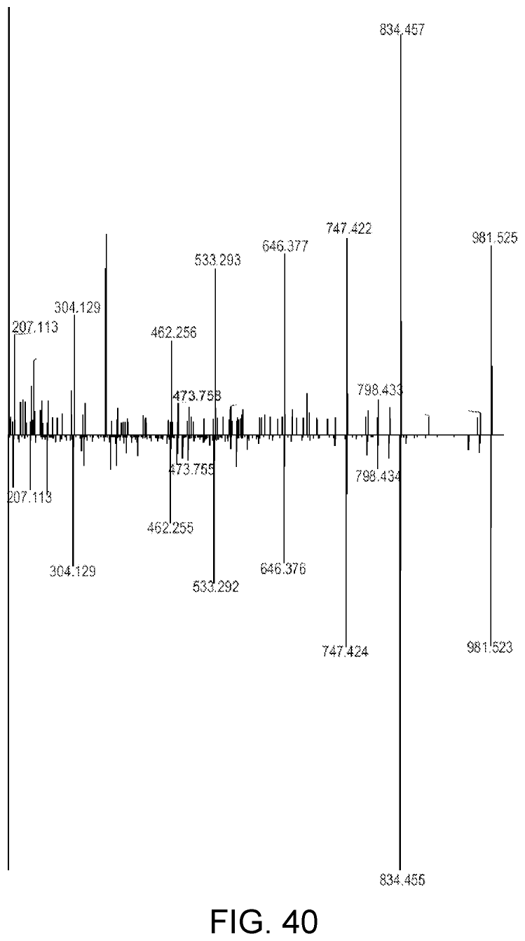

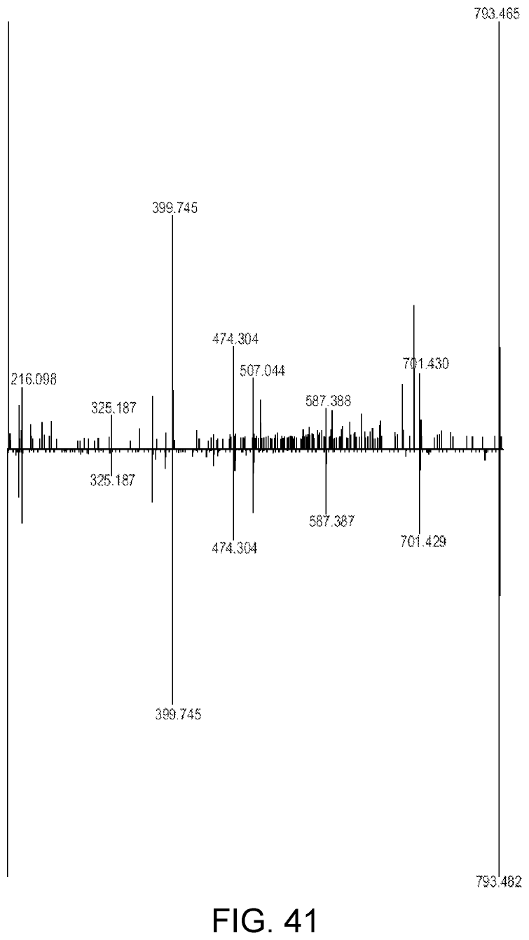

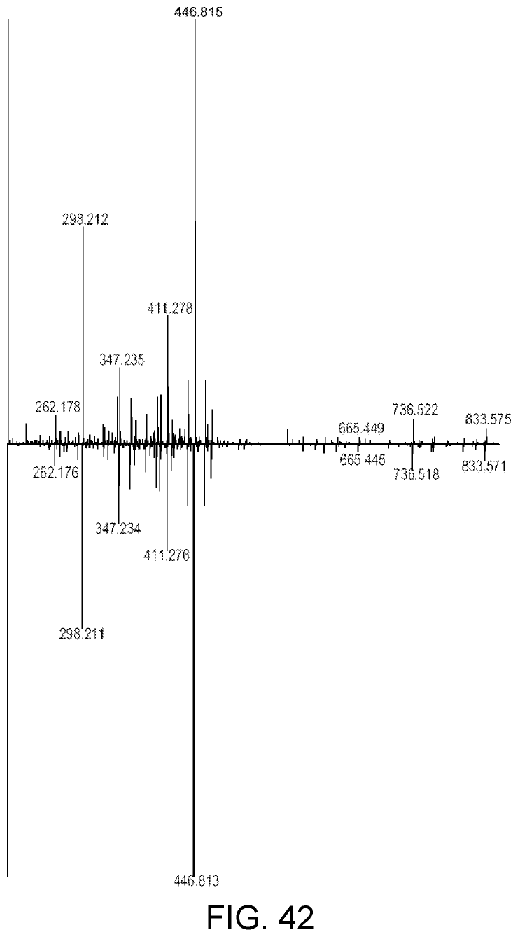

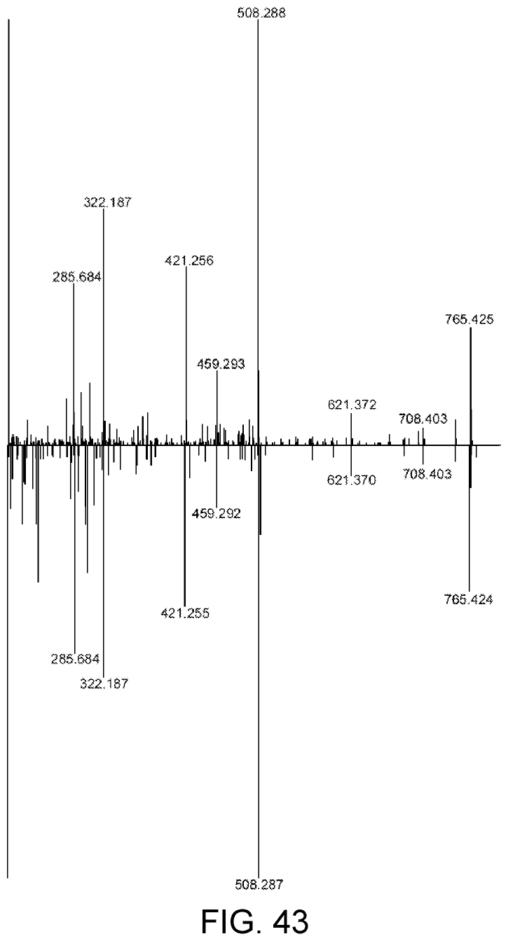

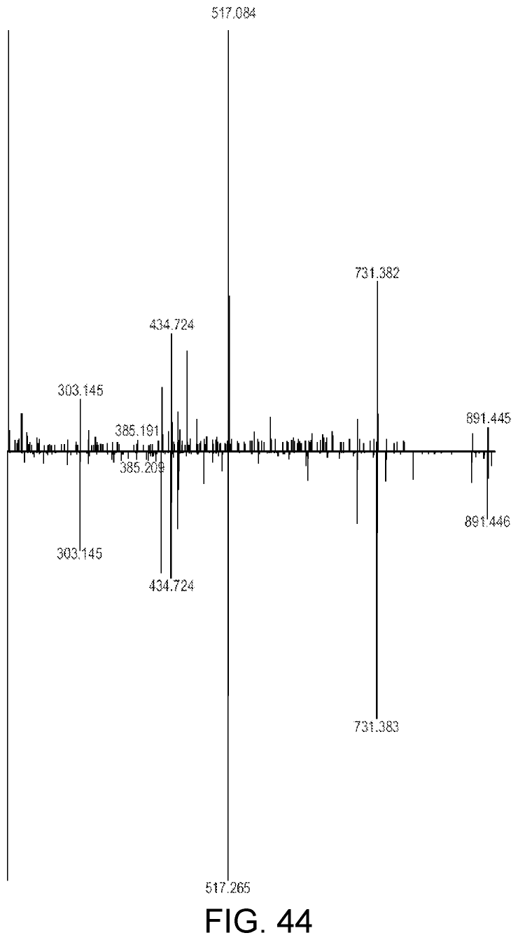

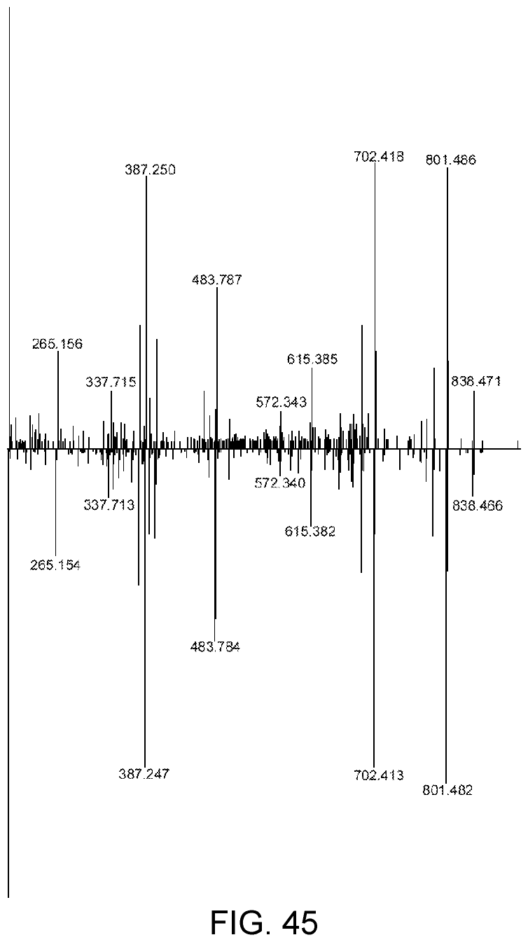

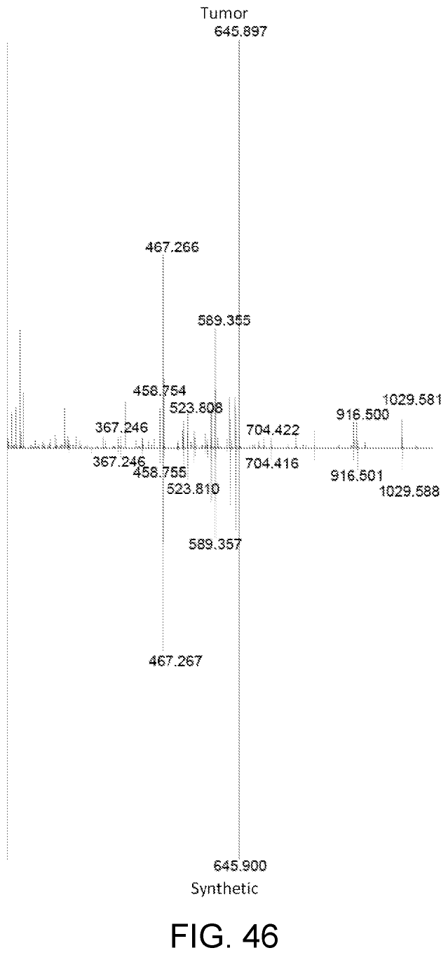

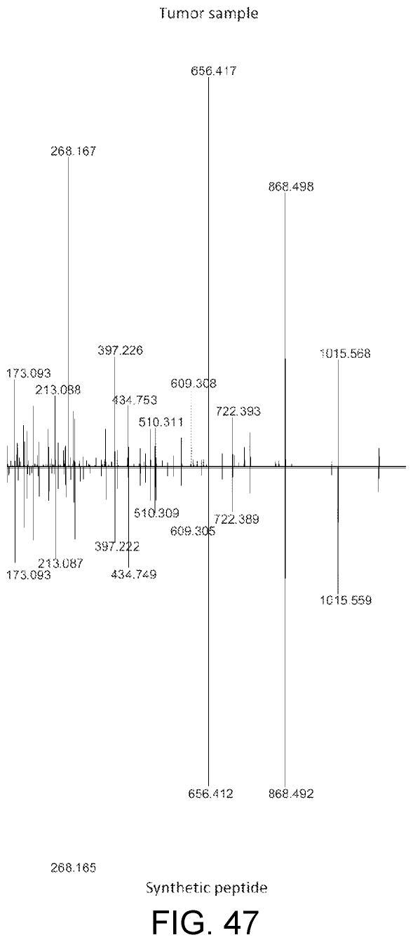

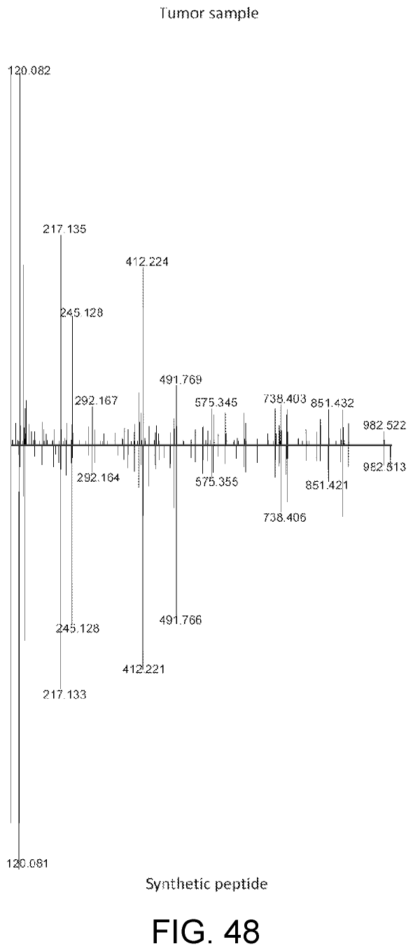

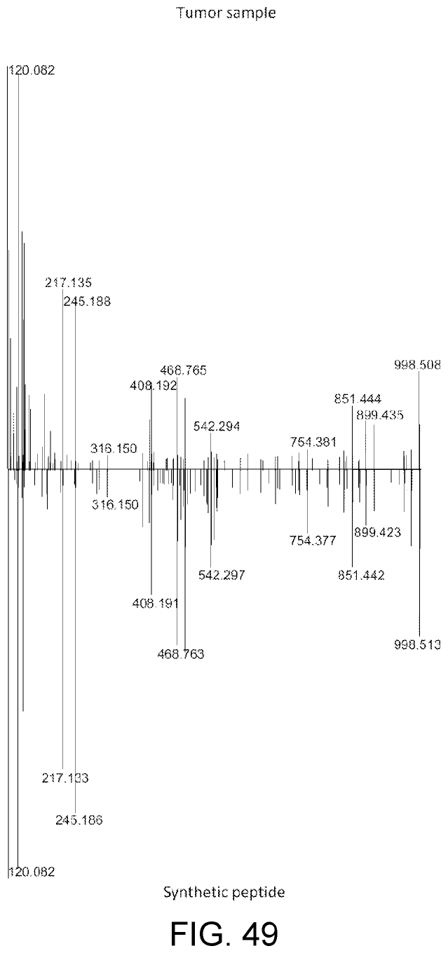

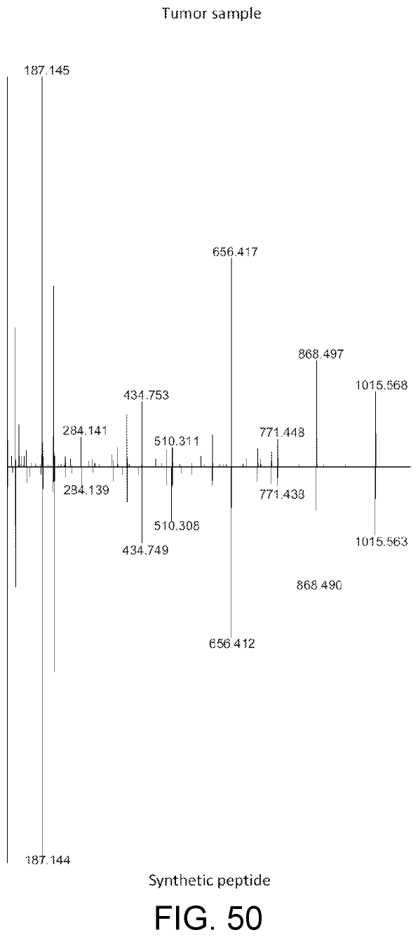

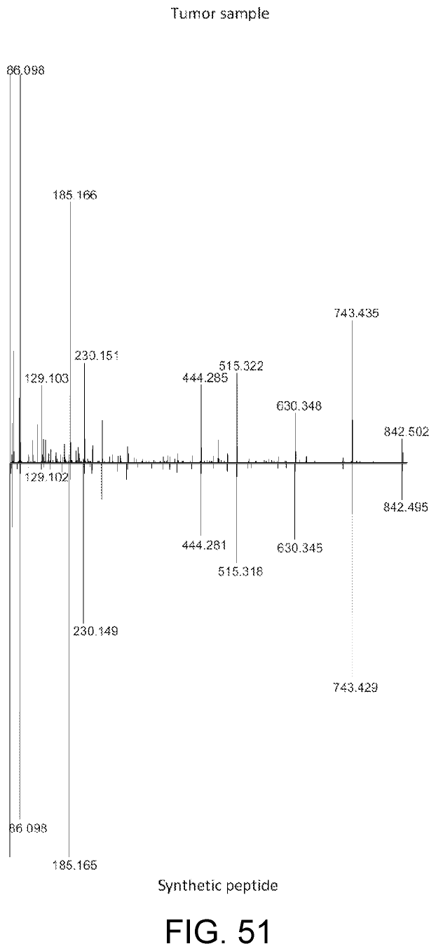

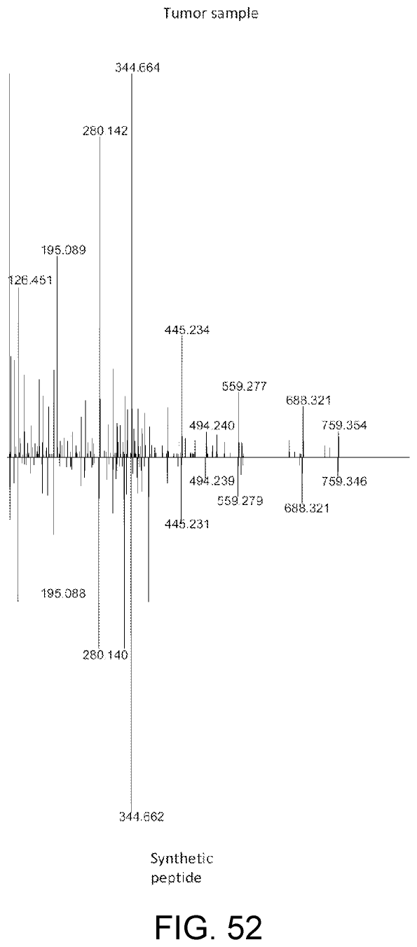

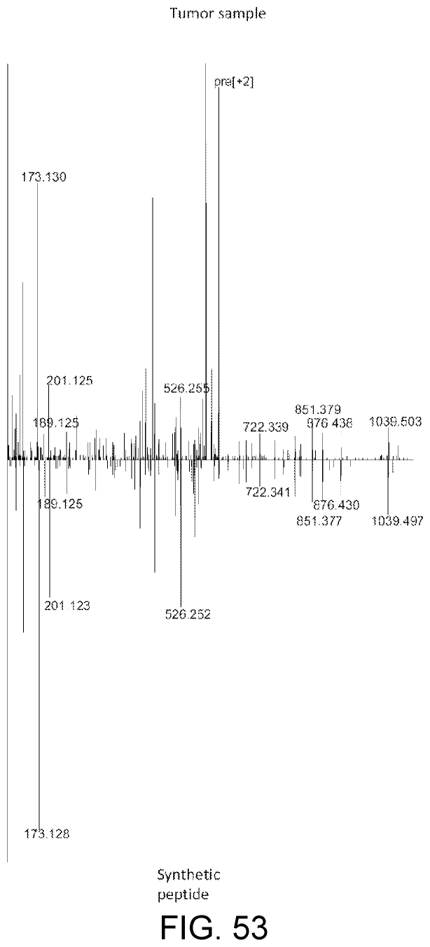

To provide further certainty of the assignment of tumor tissue-derived MS spectra to the peptide sequences that we discovered, peptides with these discovered sequences were synthesized and subjected to nUPLC-MS² using the same conditions applied to the tumor samples in the original study (Bassani-Sternberg et al., 2016, Nature Commun., 7: 13404; Inventors' data). Comparison of the spectra for selected peptides are shown in FIGS. 39-54. In each Figure the upper spectrum corresponds to the tumor sample (from the PRIDE database (Bassani-Sternberg et al., 2016, Nature Commun., 7: 13404; database link:

https://www.ebi.ac.uk/pride/archive/projects/PXD004894 or in the iventor's databse and the lower spectrum corresponds to the synthetically produced peptide of the same sequence. Selected m/z values of detected ion fragments are shown above/below each fragment peak in these MS/MS spectra. These Figures reveal a precise alignment of fragments (tiny differences in the experimentally determined m/z values between tumor- and synthetic peptide-derived fragment ions being well within the m/z tolerances of <0.05 Daltons), confirming the veracity of the assignment of each of the tumor tissue-derived spectra to the CLT-encoded peptides.

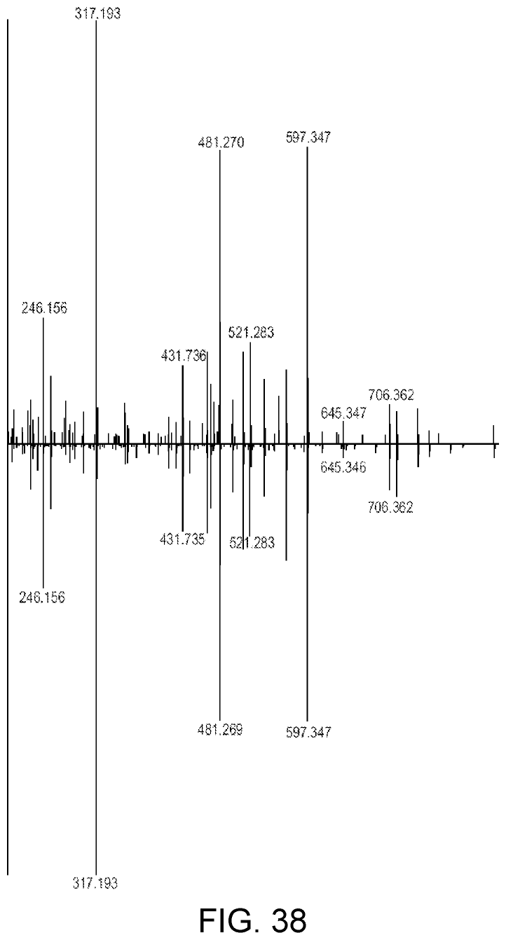

Taken together, the data shown in Tables 1 & 2 and FIGS. 1-53 supply exceptionally strong support for the translation, processing, and presentation of the corresponding CLT antigens in melanoma patients.

To further confirm the cancer-specificity of these CLTs, the inventors processed 37 normal tissue samples (10 normal skin, 9 normal lung and 18 normal breast tissue) and prepared for immunopeptidomic analysis. The inventors interrogated the spectra of the HLA-Class I dataset from these normal tissue samples, searching for all possible peptide sequences derived from the polypeptide sequences of CLT antigens 1, 2, 3, 4, 5, 6, 7 and 8, alongside all the polypeptides found in the human proteome (UniProt) using the Peaks™ software (V8.5 and X). No peptides derived from CLT antigen 1, 2, 3, 4, 5, 6, 7 or 8 were detected in the set of normal tissue samples (Table 3) providing additional evidence that the CLTs have cancer-specific expression.

In summary: the identification of immunopeptidomic peptides derived from the predicted ORFs, demonstrates that these CLTs are translated into polypeptides (SEQ ID NOs. 1-8; referred to as CLT antigens) in tumor tissue. These are then processed by the immune surveillance apparatus of the cells, and component peptides are loaded onto HLA Class I molecules, enabling the cell to be targeted for cytolysis by T cells that recognize the resulting peptide/HLA Class I complexes. Thus, these CLT antigens and their fragments are expected to be useful in a variety of therapeutic modalities for the treatment of melanoma in patients whose tumors express these antigens.

TABLE 1
List of peptides identified by immunopeptidomic analyses of melanoma tumor samples,
along with CLT antigen name and cross reference to SEQ ID NOs.

| | Peptide | CLT | CLT Ant. | | | | | | |
|---|---|---|---|---|---|---|---|---|---|
| Peptide | SEQ ID | Ant. | SEQ ID | Analysis | Patient | Peptide | - | # of | |
| sequence¹ | No. | No. | NO. | type | #² | mass³ | 10lgP⁴ | spectra⁵ | Ppm⁶ |
| VQQGWFFP | SEQ ID | 1 | SEQ ID | A | Mel-3 | 1163.59 | 15.54 | 1 | 9.4 |
| R | NO. 9 | | NO. 1 | | | | | | |
| VVRGGAGF | SEQ ID | 1 | SEQ ID | A | Mel-3 | 1059.59 | 49.12 | 8 | 5.1 |
| AAR | NO. 10 | | NO. 1 | | | | | | |
| VVRGGAGF | SEQ ID | 1 | SEQ ID | B | Mel-3 | 1059.59 | na | 1 | na |
| AAR | NO. 10 | | NO. 1 | | | | | | |
| VVRGGAGF | SEQ ID | 1 | SEQ ID | C | 2MT3 | 1059.594 | 42.49 | 6 | -0.9 |
| AAR | NO. 10 | | NO. 1 | | | | | | |
| HLADRKLS | SEQ ID | 1 | SEQ ID | A | Mel-5 | 1051.61 | 20.75 | 2 | -1.1 |
| L | NO. 11 | | NO. 1 | | | | | | |
| HLADRKLS | SEQ ID | 1 | SEQ ID | A | Mel-16 | 1051.61 | 33.22 | 30 | 5.1 |
| L | NO. 11 | | NO. 1 | | | | | | |
| HLADRKLS | SEQ ID | 1 | SEQ ID | B | Mel-16 | 1051.61 | — | 3 | |
| L | NO. 11 | | NO. 1 | | | | | | |
| HLADRKLS | SEQ ID | 1 | SEQ ID | C | 2MT3 | 1051.61 | 28.11 | 2 | −1.4 |
| L | NO. 11 | | NO. 1 | | | | | | |
| HLADRKLS | SEQ ID | 1 | SEQ ID | C | 2MT10 | 1051.61 | 21.21 | 1 | 3.4 |
| L | NO. 11 | | NO. 1 | | | | | | |
| ARLQGSVT | SEQ ID | 1 | SEQ ID | B | Mel-5 | 985.56 | na | 1 | na |
| L | NO. 12 | | NO. 1 | | | | | | |
| ADSLILDF | SEQ ID | 2 | SEQ ID | A | Mel-26 | 892.45 | 16.68 | 1 | 0.9 |
| | NO. 18 | | NO. 2 | | | | | | |
| SSFSTLAS | SEQ ID | 2 | SEQ ID | A | Mel-20 | 1154.58 | 42.38 | 2 | −6.7 |
| LDK | NO. 19 | | NO. 2 | | | | | | |
| SSFSTLAS | SEQ ID | 2 | SEQ ID | B | Mel-20 | 1154.58 | na | 2 | na |
| LDK | NO. 19 | | NO. 2 | | | | | | |
| SSFSTLAS | SEQ ID | 2 | SEQ ID | C | 2MT4 | 1154.582 | 35.88 | 1 | 0.9 |
| LDK | NO. 19 | | NO. 2 | | | | | | |
| NTPNIVSL | SEQ ID | 3 | SEQ ID | A | Mel-35 | 1012.57 | 20.91 | 2 | 3.8 |
| R | NO. 31 | | NO. 3 | | | | | | |
| NTPNIVSL | SEQ ID | 3 | SEQ ID | C | 2MT3 | 1012.567 | 18.09 | 1 | −2.8 |
| R | NO. 31 | | NO. 3 | | | | | | |
| RPLRIKGV | SEQ ID | 3 | SEQ ID | C | 1MT1 | 1084.6869 | 26.28 | 7 | 0 |
| F | NO. 32 | | NO. 3 | | | | | | |
| KTKGSLSV | SEQ ID | 4 | SEQ ID | A | Mel-3 | 1121.66 | 26.91 | 3 | 4.8 |
| FR | NO. 36 | | NO. 4 | | | | | | |
| KTKGSLSV | SEQ ID | 4 | SEQ ID | B | Mel-3 | 1121.66 | na | 2 | na |
| FR | NO. 36 | | NO. 4 | | | | | | |
| KTKGSLSV | SEQ ID | 4 | SEQ ID | C | 2MT3 | 1121.6556 | 40.46 | 6 | −0.7 |
| FR | NO. 36 | | NO. 4 | | | | | | |
| KTKGSLSV | SEQ ID | 4 | SEQ ID | C | 2MT1 | 1121.6556 | 35.73 | 2 | −0.7 |
| FR | NO. 36 | | NO. 4 | | | | | | |
| AAFDRAVH | SEQ ID | 4 | SEQ ID | A | Mel-40 | 1032.51 | 17.24 | 1 | 0.3 |
| F | NO. 37 | | NO. 4 | | | | | | |
| AAFDRAVH | SEQ ID | 4 | SEQ ID | A | Mel-41 | 1032.514 | 26.11 | 3 | 7.8 |
| F | NO. 37 | | NO. 4 | | | | | | |
| AAFDRAVH | SEQ ID | 4 | SEQ ID | C | 2MT3 | 1032.514 | 18.73 | 1 | −1.3 |
| F | NO. 37 | | NO. 4 | | | | | | |
| AFDRAVHF | SEQ ID | 4 | SEQ ID | A | Mel-27 | 961.4769 | 20.6 | 3 | 7.9 |
| | NO. 38 | | NO. 4 | | | | | | |
| AFDRAVHF | SEQ ID | 4 | SEQ ID | A | Mel-39 | 961.47 | 27.49 | 3 | 2.6 |
| | NO. 38 | | NO. 4 | | | | | | |
| KTKGSLSV | SEQ ID | 4 | SEQ ID | C | 2MT12 | 965.555 | 27.84 | 1 | 1.2 |
| F | NO. 39 | | NO. 4 | | | | | | |
| LPRTPRPD | SEQ ID. | 5 | SEQ ID | C | Mel-8 | 1289.7819 | 22.53 | 2 | 2.3 |
| LIL | NO. 45 | | NO.5 | | | | | | |
| LPRTPRPD | SEQ ID. | 5 | SEQ ID | C | Mel-16 | 1289.7819 | 28.23 | 5 | 7 |
| LIL | NO. 45 | | NO. 5 | | | | | | |
| LPRTPRPD | SEQ ID. | 5 | SEQ ID | C | Mel-29 | 1289.7819 | 28.79 | 1 | −1.4 |
| LIL | NO. 45 | | NO. 5 | | | | | | |
| ATIFPDPW | SEQ ID | 6 | SEQ ID | C | Mel-41 | 1299.7227 | 36.89 | 3 | 8.5 |
| LLK | NO. 48 | | NO. 6 | | | | | | |
| FPFYKDTV | SEQ ID | 6 | SEQ ID | C | Mel-41 | 1128.5854 | 31.32 | 5 | 8.3 |
| L | NO. 49 | | NO. 6 | | | | | | |
| FPFYKDTV | SEQ ID | 6 | SEQ ID | C | Mel-41 | 1241.6696 | 29.36 | 1 | 9.2 |
| LL | NO. 50 | | NO. 6 | | | | | | |
| TIFPDPWL | SEQ ID | 6 | SEQ ID | C | Mel-41 | 1228.6855 | 31.9 | 3 | 8.9 |
| LK | NO. 51 | | NO. 7 | | | | | | |
| IVLDAPVT | SEQ ID | 7 | SEQ ID | C | Mel-21 | 954.575 | 19.95 | 1 | 8.7 |
| K | NO. 52 | | NO. 7 | | | | | | |
| IVLDAPVT | SEQ ID | 7 | SEQ ID | C | Mel-36 | 954.575 | 22.36 | 2 | 1.6 |
| K | NO. 52 | | NO. 7 | | | | | | |
| IVLDAPVT | SEQ ID | 7 | SEQ ID | C | Mel-39 | 954.575 | 20.11 | 2 | 1.3 |
| K | NO. 52 | | NO. 7 | | | | | | |
| IVLDAPVT | SEQ ID | 7 | SEQ ID | C | 2MT3 | 954.575 | 24.77 | 7 | 0.3 |
| K | NO. 52 | | NO. 7 | | | | | | |
| GHIHNEAV | SEQ ID | 8 | SEQ ID | C | Mel-27 | 875.4249 | 21.32 | 2 | 7.9 |
| | NO. 53 | | NO. 8 | | | | | | |
| SLYGHIHN | SEQ ID | 8 | SEQ ID | C | Mel-27 | 1238.6044 | 35.11 | 1 | 8.5 |
| EAV | NO. 54 | | NO. 8 | | | | | | |
| SLYGHIHN | SEQ ID | 8 | SEQ ID | C | 2MT 4 | 1238.604 | 56.34 | 2 | 4.7 |
| EAV | NO. 54 | | NO. 8 | | | | | | |

¹HLA Class I peptides identified by mass spectrometry.
²Bassani-Sternberg et al, 2016, Nature Comm., 7: 13404 (Mel-3, Mel-5, Mel-8, Mel-16, Mel-21, Mel-27, Mel-29, Mel-30, Mel-36, Mel-39, Mel-41); Inventors' dataset (1MT1, 2MT1, 2MT3, 2MT4, 2MT10, 2MT12).
³Calculated peptide mass.
⁴PEAKS^{TM} program - 10lgP values are shown for peptides for highest match for peptide/patients for which more than one spectral detection was obtained. Values are not available (na) for peptides identified via Analysis B performed using Mascot software.
⁵Number of spectra in which peptide was detected.
⁶Deviation between observed mass and calculated mass; selected ppm values are shown for peptides for which more than one spectrum was obtained. Values are not available (na) for peptides identified via Analysis B.

TABLE 2
Predicted NetMHCpan4.0 binding of Mass Spectrometry-identified peptides (length ≥9
residues) to 18 HLA Class I Supertype Alleles (HLA-A01:01, HLA-A02:01, HLA-A03:01, HLA-
A11:01, HLA-A24:02, HLA-A25:01, HLA-A26:01, HLA-A68:01, HLA-B07:02, HLA-B08:01, HLA-
B15:01, HLA-B18:01, HLA-B27:05, HLA-B35:01, HLA-B35:03, HLA-B40:01, HLA-B40:02, HLA-
B51:01), along with CLT antigen name and cross reference to SEQ ID NOS.

| | | Number | Number | Number | | | |
|---|---|---|---|---|---|---|---|
| Predicted | | of | of | of | | | |
| to bind 18 | | alleles | alleles | alleles | | | |
| human HLA | | with a | with a | with a | | | |
| Class I | | rank | rank | rank | Peptide | CLT | |
| A & B | Peptide | score of | score | score of | SEQ ID | Antigen | Patient |
| supertypes¹ | sequence | ≤5%² | of ≤2%³ | ≤0.5%⁴ | NO | No. | #⁵ |
| YES | VQQGWFFPR | 3/18 | 0/18 | 0/18 | SEQ ID | 1 | Mel-3 |
| | | | | | NO. 9 | | |
| YES | VVRGGFAGFAAR | 4/18 | 0/18 | 0/18 | SEQ ID | 1 | Mel-3 |
| | | | | | NO. 10 | | 2MT3 |
| YES | HLADRKLSL | 10/18 | 7/18 | 2/18 | SEQ ID | 1 | Mel-5 |
| | | | | | NO. 11 | | Mel-16 |
| | | | | | | | 2MT3 |
| | | | | | | | 2MT10 |
| YES | ARLQGSVTL | 10/18 | 3/18 | 1/18 | SEQ ID | 1 | Mel-5 |
| | | | | | NO. 12 | | |
| YES | SSFSTLASLDK | 3/18 | 2/18 | 0/18 | SEQ ID | 2 | Mel-20 |
| | | | | | NO. 19 | | 2MT4 |
| YES | NTPNIVSLR | 6/18 | 3/18 | 1/18 | SEQ ID | 3 | Mel-35 |
| | | | | | NO. 31 | | 2MT3 |
| YES | RPLRIKGVF | 6/18 | 4/18 | 1/18 | SEQ ID | 3 | 2MT1 |
| | | | | | NO. 32 | | |
| YES | KTKGSLSVFR | 4/18 | 3/18 | 0/18 | SEQ ID | 4 | Mel-3 |
| | | | | | NO. 36 | | 2MT1 |
| | | | | | | | 2MT3 |
| YES | AAFDRAVHF | 18/18 | 10/18 | 5/18 | SEQ ID | 4 | Mel-40 |
| | | | | | NO. 37 | | Mel-41 |
| | | | | | | | 2MT3 |
| YES | KTKGSLSVF | 9/18 | 4/18 | 2/18 | SEQ ID | 4 | 2MT12 |
| | | | | | NO. 39 | | |
| YES | LPRTPRPDLIL | 4/18 | 1/18 | 1/18 | SEQ ID | 5 | Mel-8 |
| | | | | | NO. 45 | | Mel-16 |
| | | | | | | | Mel-29 |
| YES | ATIFPDPWLLK | 4/18 | 3/18 | 2/18 | SEQ ID | 6 | Mel-41 |
| | | | | | NO. 48 | | |
| YES | FPFYKDTVL | 10/18 | 7/18 | 5/18 | SEQ ID | 6 | Mel-41 |
| | | | | | NO. 49 | | |
| YES | FPFYKDTVLL | 8/18 | 6/18 | 4/18 | SEQ ID | 6 | Mel-41 |
| | | | | | NO. 50 | | |
| YES | TIFPDPWLLK | 6/18 | 3/18 | 3/18 | SEQ ID | 6 | Mel-41 |
| | | | | | NO. 51 | | |
| YES | IVLDAPVTK | 3/18 | 3/18 | 3/18 | SEQ ID | 7 | Mel-21, |
| | | | | | NO. 52 | | Mel-34, |
| | | | | | | | Mel-39 |
| YES | SLYGHIHNEAV | 1/18 | 1/18 | 1/18 | SEQ ID | 8 | Mel-27 |
| | | | | | NO. 54 | | |

¹Predicted binding to interrogated HLA Class I supertypes at a Rank score of ≤5.0% score.
² Number of the 18 HLA Class I supertypes that were predicted to bind with a rank score of ≤5.0%.
³ Number of the 18 HLA Class I supertypes that were predicted to bind with a rank score of ≤2.0%.
⁴ Number of the 18 HLA Class I supertypes that were predicted to bind with a rank score of ≤0.5%.
⁵ Bassani-Sternberg et al, 2016, Nature Comm., 7: 13404 (Mel-3, Mel-8, Mel-16, Mel-21, Mel-27, Mel-29, Mel-30, Mel-36, Mel-39, Mel-41); Inventors' dataset (1MT1, 2MT1, 2MT3, 2MT4, 2MT10, 2MT12).

TABLE 3
Number of peptides-derived from CLT Antigens
1 to 8 in a set of normal tissue samples.

| Antigen | Skin | Lung | Breast |
|---|---|---|---|
| CLT Antigen 1 | 0/10 | 0/9 | 0/18 |
| CLT Antigen 2 | 0/10 | 0/9 | 0/18 |
| CLT Antigen 3 | 0/10 | 0/9 | 0/18 |
| CLT Antigen 4 | 0/10 | 0/9 | 0/18 |
| CLT Antigen 5 | 0/10 | 0/9 | 0/18 |
| CLT Antigen 6 | 0/10 | 0/9 | 0/18 |
| CLT Antigen 7 | 0/10 | 0/9 | 0/18 |
| CLT Antigen 8 | 0/10 | 0/9 | 0/18 |

The results presented here in Examples 1 and 2 are in whole or part based upon data generated by the The Cancer Genome Atlas (TCGA) Research Network (http://cancergenome.nih.gov/); and the Genotype-Tissue Expression (GTEx) Project (supported by the Common Fund of the Office of the Director of the National Institutes of Health, and by NCI, NHGRI, NHLBI, NIDA, NIMH, and NINDS).

### Example 3—HERVFEST

Functional expansion of specific T-cells (FEST) technology has been used to identify therapeutically relevant tumor-derived epitopes present in the “mutation-associated neoantigen” (MANA) repertoire found in tumor cells of cancer patients based on detection of patient T-cells that react to MANA epitopes (Anagnostou et al., Cancer Discovery 2017; Le et al., Science 2017; Forde et al., NEJM 2018; Danilova et al., Cancer Immunol. Res. 2018). Application of FEST technology to CLT antigens discovered by using the methods elucidated in Example 1 & 2 (Tables 1-3, FIGS. 1-53) can be used to identify therapeutically relevant T-cell responses to CLT antigens in cancer patients.

Like other assays (e.g., ELISPOT) to identify epitope-specific T-cells in a subject who has undergone immune exposure, “FEST” technologies derive their specificity by activating/expanding the cognate T-cells in ex vivo cultures that include antigen-presenting cells and suitable antigenic peptides. The technique differs from other immunological assays in that it utilizes next-generation sequencing of the T-cell receptor (TCR) DNA sequences present in these amplified cultures (specifically: TCRseq targeting the TCR-Vβ CDR3 region) to detect the specific TCRs that are expanded in the cells cultured with individual peptides from a panel of target peptides derived from an antigen (or antigens). Application of TCRseq to tumor tissues in the same patient can also be used to demonstrate if TCRs/T-cells detected in the ex vivo, peptide-stimulated cultures are also present within the tumor-infiltrating lymphocytes found in cancer tissues in situ. Thus, MANAFEST has proven to be a powerful technology for identifying MANA epitopes that are recognized by patient T-cells, permitting identification of functionally relevant MANA peptides among the multitude of mutant peptides found by whole-exome sequencing of normal and tumor tissues from cancer patients (Le et al., Science 2017; Forde et al., NEJM 2018; Danilova et al., Cancer Immunol. Res. 2018; Smith et al., J Immunother Cancer 2019).

Application of MANAFEST methodology (Danilova et al., Cancer Immunol. Res. 2018) to CLT antigens was performed as follows. The method, which we will refer to as HERVFEST, consists of the following steps: Step 1: Peptides predicted to contain epitopes that efficiently bind selected HLA Class I alleles were identified in CLT Antigens. Step 2: PBMCs from suitable melanoma patients were matched by HLA Class I type to the peptide library selected in step 1. Step 3: PBMCs from these patients were separated into T-cell and non T-cell fractions. Non T-cells were added back to the patient's T-cells, and then divided into 20-50 wells (containing 250,000 T-cells per culture) and propagated with various T-cell growth factors and individual CLT Antigen-derived synthetic peptides (selected in step 1/2) for 10 days. Step 4: TCRseq (sequencing of the TCR-Vβ CDR3 sequences) was performed on all wells, and TCR-Vβ CDR3 sequences that were amplified in the presence of individual CLT Antigen-derived peptides (but not amplified in the presence of control peptides or in the absence of peptide stimulation) were identified. The presence of amplified TCR-Vβ CDR3 sequences in individual wells of the assay thus identifies CLT Antigen-derived peptides that elicited an immune response in the melanoma patient. Step 5: TCRseq may also be performed on tumor samples to determine whether the T-cells bearing the CLT-Antigen amplified TCRs homed to patient tumors, providing additional evidence that T-cells bearing these TCRs recognize CLT Antigen-derived peptides within a patient's tumor.

HERVFEST assays were performed with peptides derived from CLT Antigens 1-4 (SEQ ID NOs 1-4). The panel of peptides (see step 1 above) used for these studies was based on NetMHC predictions of CLT Antigen-derived peptides that were predicted to strongly bind the 8 HLA Class I types commonly found in patient tumor samples available for our analyses. CLT Antigen-derived peptides that amplified one or more TCRs in these HERVFEST assays are provided in Table 4. Table 4 also indicates the HLA Class I type(s) of the CLT antigen peptides that were tested with each patient's PBMC-derived cultures. The HLA Class I type of the patients whose PBMCs were tested in these studies and amplified one or more TCRs in the assays, are shown in Table 5.

FIG. 54 panel A shows published data demonstrating TCR amplification with NSCLC patient-specific MANA peptides (Forde et al., NEJM 2018). The vertical axis shows the prevalence of each indicated TCR Vβ CDR3 AA Sequence for wells of cells cultivated in the presence of the MANA or control peptides listed on the horizontal axis. The amplification in the well containing MANA7 indicates the patient's T-cell repertoire include T-cells that are reactive to this peptide. Panels B and C of FIG. 54 show representative TCR amplification data from PBMCs from 2 melanoma patients that were incubated in the presence of the indicated CLT Antigen peptides and control peptides. As with Panel A, the specific amplifications observed in Panels B & C demonstrate that the T-cell repertoire of these melanoma patients includes T-cells that are reactive with specific CLT Antigen-derived peptides. Panel B shows the frequency of TCRs detected in the LMSSFSTLASL-stimulated well of PBMCs from melanoma patient 222B in all wells stimulated with the panel 15 Class I HLA-A*02 peptides from CLT Antigens 1, 2 & 4. Three TCR sequences were amplified. LMSSFSTLASL (SEQ ID NO. 23) is an HLA-A*02 binding peptide derived from CLT Antigen 2. Panel C shows the frequency of TCRs detected in the MVACRIKTFR-stimulated well of PBMCs from melanoma patient 224B in all wells stimulated with the panel of 15 Class I HLA-A*02 peptides from CLT Antigens 1, 2 & 4 and 24 Class I HLA-A*03 peptides from CLT Antigens 1, 2, 3, & 4. One TCR sequence was amplified. MVACRIKTFR (SEQ ID NO. 26) is an HLA-A*03 binding peptide derived from CLT Antigen 2.

The control peptides/conditions used in these experiments were as follows: CEF=mixture of CMV, EBV, and influenza peptides; SL9, TV9 and QK1=HIV-1 control peptides; no peptide=cultivation in absence of peptide; Baseline=T-cells before culture.

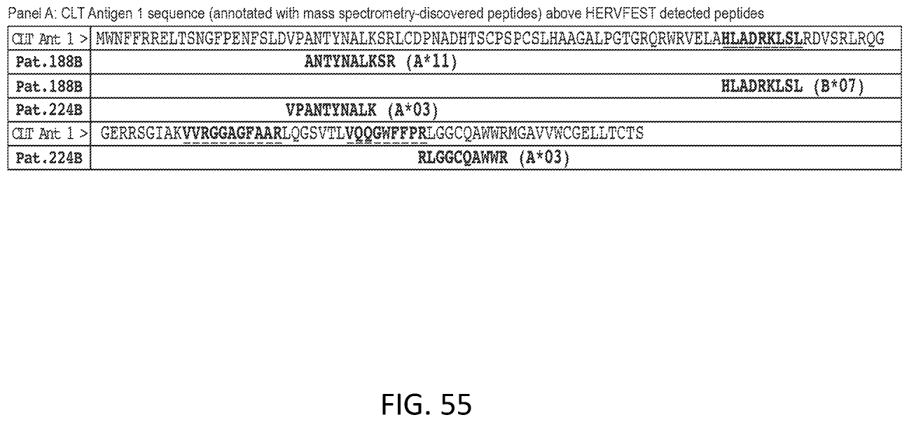

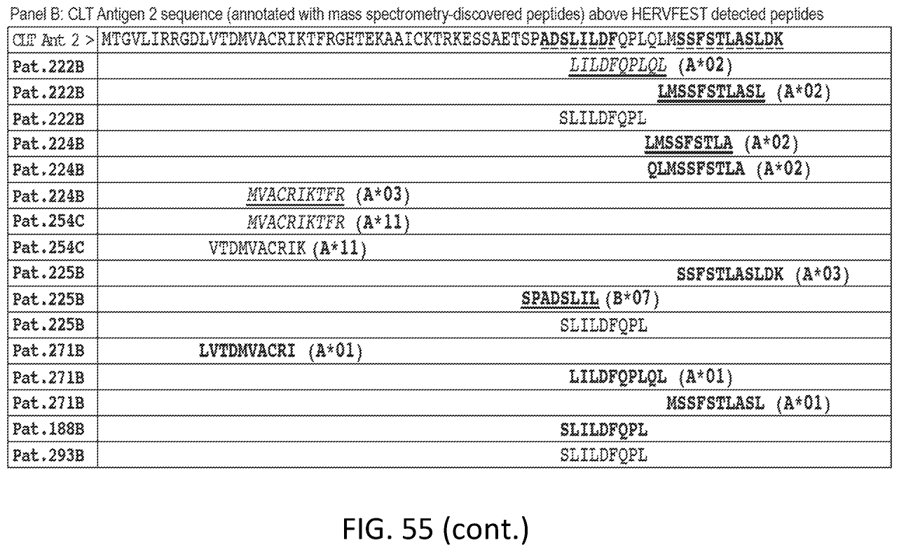

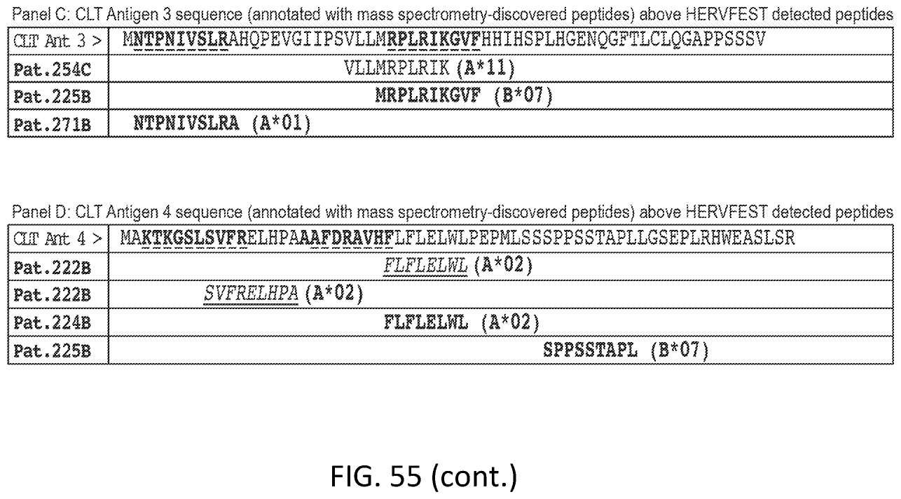

FIG. 55 shows a summary of all CLT Antigen peptides for CLT Antigens 1-4 which amplified one or more TCRs in studies completed with these patients. Each panel displays the amino acid sequences of CLT Antigens 1-4 overlaid with peptides detected by immunopeptidomic analyses (denoted by dashed underlined or bold text; see Example 2). Below these sequences, the HERVFEST-detected peptides (see FIG. 54) are displayed with the numeric identifier of the melanoma patient in which they were detected (Table 5) and the targeted HLA Class I type.

The properties of each HERVFEST detection are defined as follows:
Plain text: Significant amplification of a single TCR
Bold text: Significant amplification of multiple TCRs
Underlined italics text: Significant amplification of a single TCR which was detected in other wells
Underlined bold text: Significant amplification of multiple TCRs, at least one of which was detected in other wells

These results provide strong evidence that CLT Antigens 1-4 are present in melanoma patients and that peptides derived from these CLT antigens have elicited specific T-cell responses in these melanoma patients, confirming the value of these CLT antigens as targets for therapeutic interventions to treat melanoma.

TABLE 4
CLT Antigen-derived peptides that amplified one or more TCRs in HERVFEST assays

| | | | HLA Class I - | |
|---|---|---|---|---|
| | | | type for peptide | |
| Reference | | | (predicted by | HERVFEST |
| CLT Antigen | SEQ ID NO | Peptide sequence | NetMHC) | panel reference |
| 1 | SEQ ID NO. 13 | VPANTYNALK | HLA-A*03:01 | 37 |
| 1 | SEQ ID NO. 14 | RLGGCQAWWR | HLA-A*03:01 | 41 |
| 1 | SEQ ID NO. 15 | ANTYNALKSR | HLA-A*11:01 | 60 |
| 1 | SEQ ID NO. 11 | HLADRKLSL | HLA-B*07:02 | 109 |
| 2 | SEQ ID NO. 20 | LVTDMVACRI | HLA-A*01:01 | 8 |
| 2 | SEQ ID NO. 21 | LILDFQPLQL | HLA-A*01:01 | 13 |
| 2 | SEQ ID NO. 22 | MSSFSTLASL | HLA-A*01:01 | 15 |
| 2 | SEQ ID NO. 21 | LILDFQPLQL | HLA-A*02:01 | 25 |
| 2 | SEQ ID NO. 23 | LMSSFSTLASL | HLA-A*02:01 | 27 |
| 2 | SEQ ID NO. 24 | LMSSFSTLA | HLA-A*02:01 | 23 |
| 2 | SEQ ID NO. 25 | QLMSSFSTLA | HLA-A*02:01 | 26 |
| 2 | SEQ ID NO. 26 | MVACRIKTFR | HLA-A*03:01 | 46 |
| 2 | SEQ ID NO. 19 | SSFSTLASLDK | HLA-A*03:01 | 45 |
| 2 | SEQ ID NO. 26 | MVACRIKTFR | HLA-A*11:01 | 67 |
| 2 | SEQ ID NO. 27 | VTDMVACRIK | HLA-A*11:01 | 69 |
| 2 | SEQ ID NO. 28 | SPADSLIL | HLA-B*07:02 | 119 |
| 2 | SEQ ID NO. 29 | SLILDFQPL | HLA-A*02:01 | 20 |
| 3 | SEQ ID NO. 33 | NTPNIVSLRA | HLA-A*01:01 | 18 |
| 3 | SEQ ID NO. 34 | VLLMRPLRIK | HLA-A*11:01 | 78 |
| 3 | SEQ ID NO. 35 | MRPLRIKGVF | HLA-B*07:02 | 124 |
| 4 | SEQ ID NO. 40 | FLFLELWL | HLA-A*02:01 | 30 |
| 4 | SEQ ID NO. 41 | SVFRELHPA | HLA-A*02:01 | 29 |
| 4 | SEQ ID NO. 42 | SPPSSTAPL | HLA-B*07:02 | 132 |

TABLE 5
Characteristics of the melanoma patient PBMCs used in HERVFEST assays

| | | | | | | | HLA(s) |
|---|---|---|---|---|---|---|---|
| | | | | | | | targeted in |
| | HLA | HLA | HLA | HLA | HLA | HLA | HERVFEST |
| Patient | allele A1 | allele A2 | allele B1 | allele B2 | allele C1 | allele C2 | studies |
| 204B | A*01:01 | A*25:01 | B*03:01 | B*16:01 | C*07:01 | C*12:03 | A*01 |
| 222B | A*02:01 | A*30:02 | B*44:02 | Not | C*05:01 | B*18:01 | A*02 |
| | | | | known | | | |
| 224B | A*02:01 | A*03:01 | B*35:01 | B*40:02 | C*04:01 | C*02:02 | A*02, A*03 |
| 293B | A*02:01 | A*30:01 | B*13:02 | B*44:02 | C*06:02 | C*05:01 | A*02 |
| 254C | A*11:01 | A*32:01 | B*15:01 | B*56:01/ | C*01:02 | C*03:03 | A*11 |
| | | | | 56:55 | | | |
| 188B | A*02:01 | A*11:01 | B8*7:02 | B*14:02 | C*07:02 | C*08:02 | A*11, B*07 |
| 271B | A*01:01 | A*01:01 | B*08:01 | B*08:01 | C*07:01 | C*07:01 | A*01 |
| 225B | A*02:01 | A*03:01 | B*07:02 | B*51:01 | C*07:02 | C*14:02 | A*03, B*07 |

### Example 4—Assays to Demonstrate High-Affinity T-Cells Specific for CLT Antigens have not been Deleted from Normal Subjects' T-Cell Repertoire

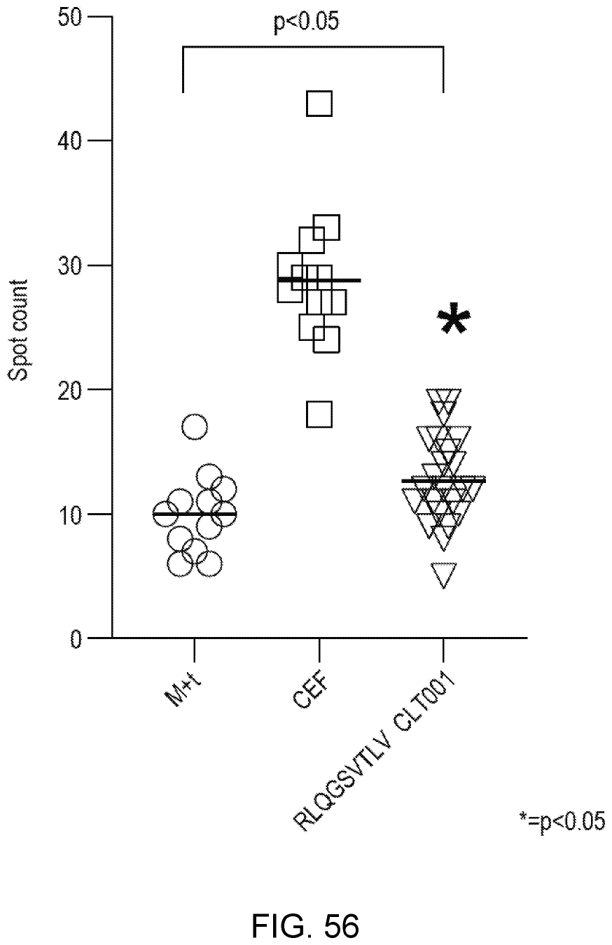

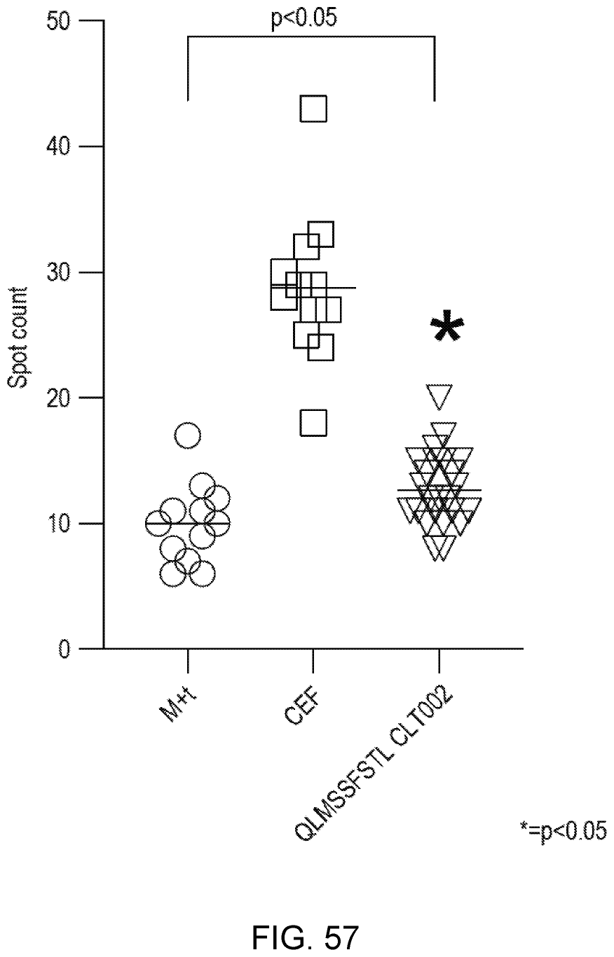

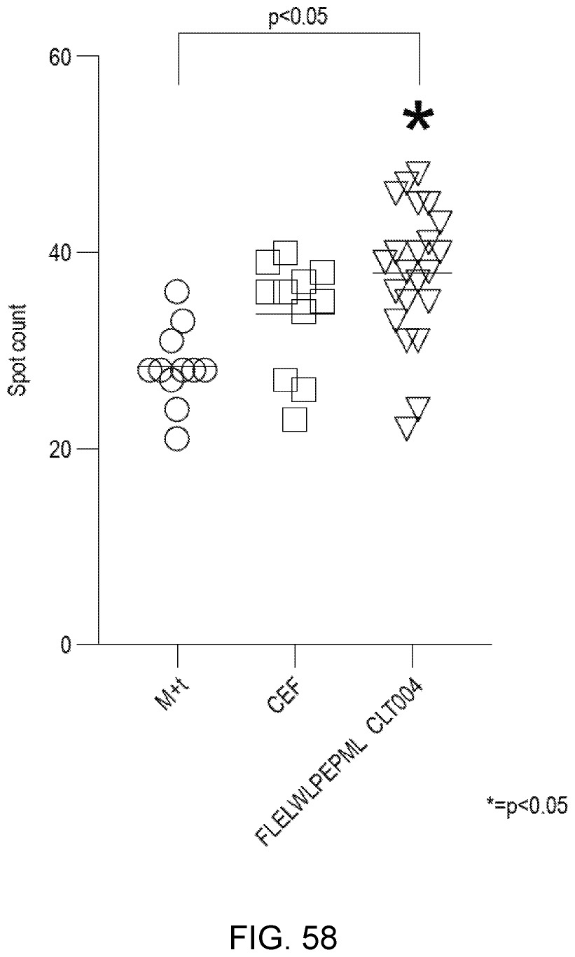

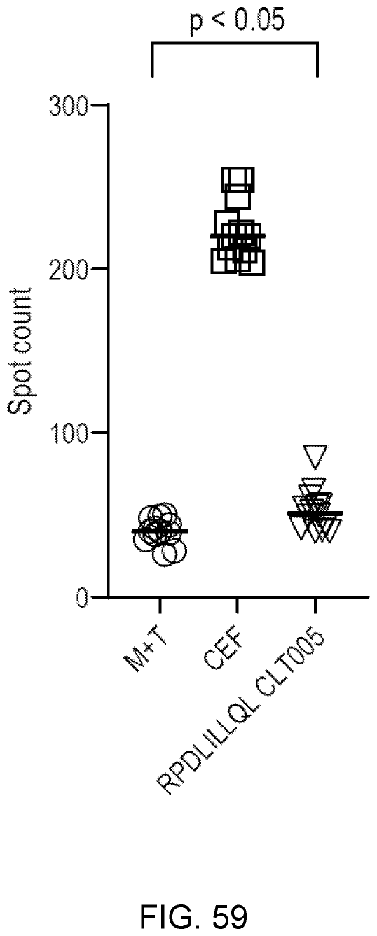

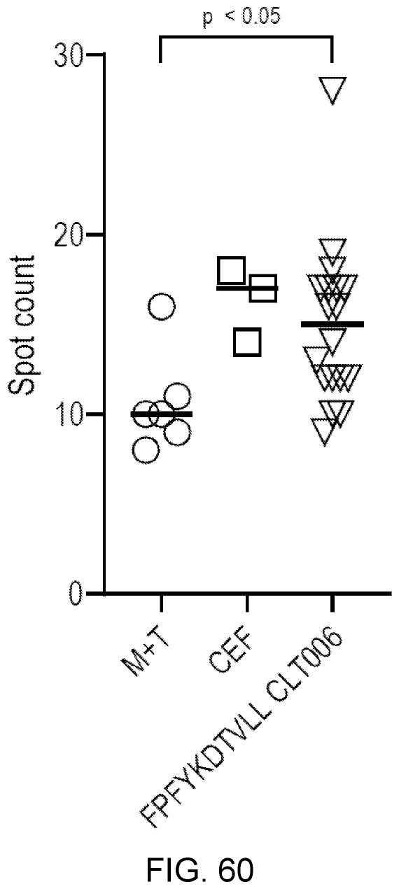

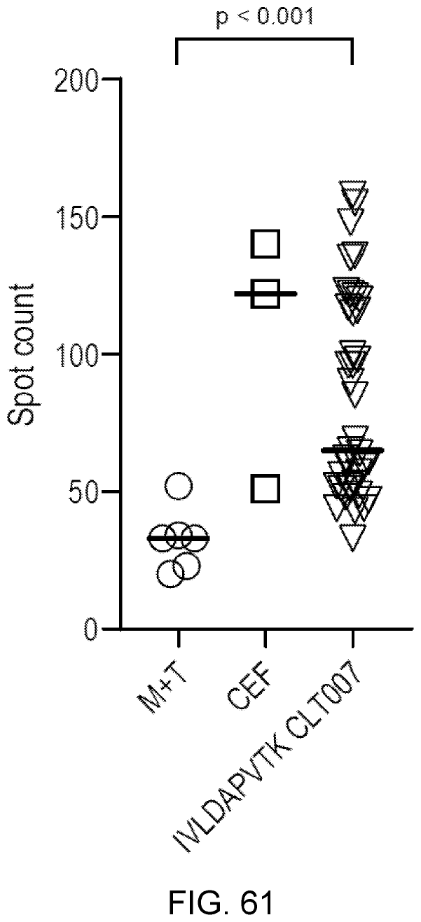

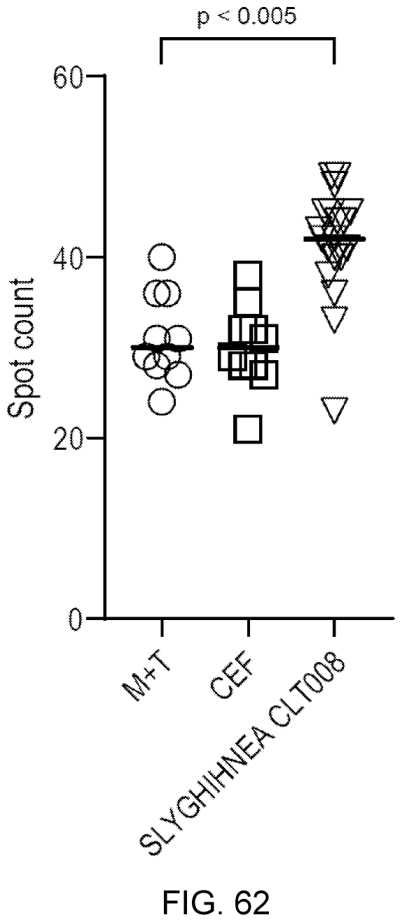

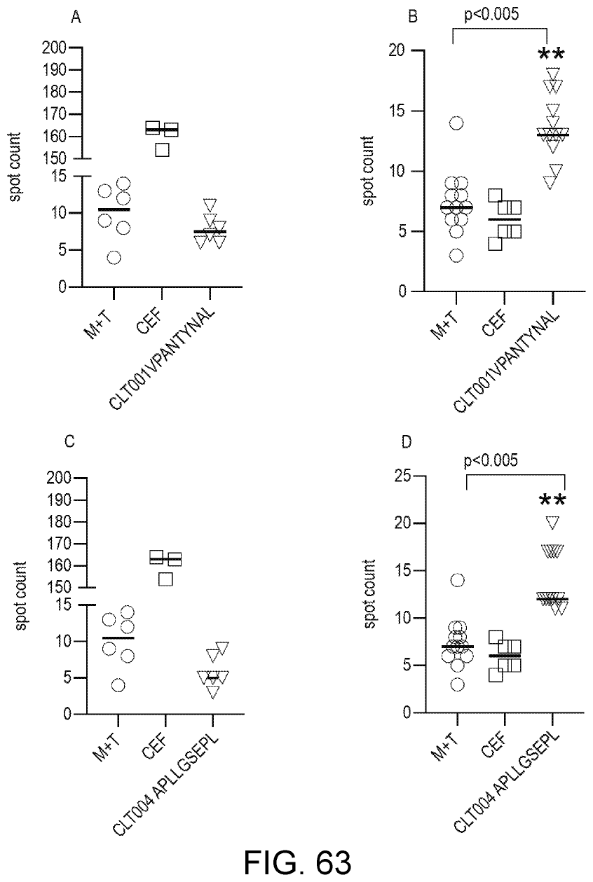

An ELISPOT assay may be used to show that CLT antigen-specific CD8 T-cells are present in the normal T-cell repertoire of healthy individuals, and thus have not been deleted by central tolerance due to the expression of cancer-specific CLT antigens in naïve and thymic tissues in these patients. This type of ELISPOT assay comprises multiple steps. Step 1: CD8 T-cells and CD14 monocytes can be isolated from the peripheral blood of normal blood donors, these cells are HLA Class I-typed to match the specific CLT antigens being tested. CD8 T-cells can be further sub-divided into naïve and memory sub-types using magnetically labelled antibodies to the memory marker CD45RO. Step 2: CD14 monocytes are pulsed with individual or pooled CLT antigen peptides for three hours prior to being co-cultured with CD8 T-cells for 14 days. Step 3: Expanded CD8 T-cells are isolated from these cultures and re-stimulated overnight with fresh monocytes pulsed with peptides. These peptides may include; individual CLT antigen peptides, irrelevant control peptides or peptides known to elicit a robust response to infectious (e.g., CMV, EBV, Flu, HCV) or self (e.g. MART-1) antigens. Re-stimulation is performed on anti-Interferon gamma (IFNγ) antibody-coated plates. The antibody captures any IFNγ secreted by the peptide-stimulated T-cells. Following overnight activation, the cells are washed from the plate and IFNγ captured on the plate is detected with further anti-IFNγ antibodies and standard colorimetric dyes. Where IFNγ-producing cells were originally on the plate, dark spots are left behind. Data derived from such assays includes spot count, median spot size and median spot intensity. These are measures of frequency of T-cells producing IFNγ and amount of IFNγ per cell. Additionally, a measure of the magnitude of the response to the CLT antigen can be derived from the stimulation index (SI) which is the specific response, measured in spot count or median spot size, divided by the background response to monocytes with no specific peptide. A metric of stimulation strength is derived by multiplying the stimulation index for spot number by the stimulation index for spot intensity. In this way, comparisons of the responses to CLT antigens and control antigens can be used to demonstrate that naïve subjects contain a robust repertoire of CLT antigen-reactive T-cells that can be expanded by vaccination with CLT antigen-based immunogenic formulations. Table 6 provides a list of CLT Antigen-derived peptides that induced significant CD8 T-cell responses from HLA-matched normal blood donors. The results are shown in FIGS. 56-63. Horizontal bars represent the mean of the data. M+t indicates the no peptide, negative control (monocytes and T cells). CEF indicates the positive control (a mixture of 23 CMV, EBV and influenza peptides). Statistical significance was measured with Kruskall Wallis test One-way Anova with correction for repeated measures with Dunns correction. FIG. 56 shows significant CD8 T-cell responses from a normal blood donor to HLA-A*02:01-restricted peptides from CLT Antigen 1 (CLT001 in the figure). The example shown in FIG. 57 demonstrates CD8 responses from a normal donor to a peptide derived from CLT Antigen 2 (CLT002 in the figure) also restricted by HLA-A*02:01. FIG. 58 shows significant CD8 T-cell responses from a normal blood donor to an HLA-A*02:01-restricted peptide from CLT Antigen 4 (CLT004 in the Figure). FIG. 59 shows significant CD8 T-cell responses from a normal blood donor to HLA-A*03:01-restricted peptide from CLT Antigen 5 (CLT005 in the Figure). FIG. 60 shows significant CD8 T-cell responses from a normal blood donor to an HLA-B*07:02-restricted peptide from CLT Antigen 6 (CLT006 in the Figure). FIG. 61 shows significant CD8 T-cell responses from a normal blood donor to an HLA-A*03:01-restricted peptide from CLT Antigen 7 (CLT007 in the Figure). FIG. 62 shows significant CD8 T-cell responses from a normal blood donor to an HLA-A*02:01-restricted peptide from CLT Antigen 8 (CLT008 in the Figure). FIG. 63 shows a lack of response to HLA-B*0702 restricted peptides from CLT Antigens 1 and 4 (CLT001 and CLT004 in the figure) in memory CD45RO-positive CD8 T-cells (panels A and C). By contrast, Naïve CD45RO-negative CD8 T-cells from the same donor respond significantly to peptides from both CLT001 and CLT004 (FIG. 63, panels B and D).

TABLE 6
CLT Antigen-derived peptides that induced signifi-
cant CD8 T-cell responses from HLA-matched normal
blood donors

| | | | HLA-type for |
|---|---|---|---|
| Reference | | | peptide |
| CLT | | Peptide | (predicted |
| Antigen | SEQ ID NO | sequence | by NetMHC) |
| 1 | SEQ ID. NO. 16 | RLQGSVTLV | HLA-A*02:01 |
| 1 | SEQ ID. NO. 17 | VPANTYNAL | HLA-B*07:02 |
| 2 | SEQ ID. NO. 30 | QLMSSFSTL | HLA-A*02:01 |
| 4 | SEQ ID. NO. 43 | FLELWLPEPML | HLA-A*02:01 |
| 4 | SEQ ID. NO. 44 | APLLGSEPL | HLA-B*07:02 |
| 5 | SEQ ID. NO. 46 | TPRPDLILL | HLA-A*03:01 |
| 5 | SEQ ID. NO. 47 | RPDLILLQL | HLA-B*07:02 |
| 6 | SEQ ID. NO. 50 | FPFYKDTVLL | HLA-B*07:02 |
| 7 | SEQ ID. NO. 52 | IVLDAPVTK | HLA-A*03:01 |
| 8 | SEQ ID. NO. 55 | SLYGHIHNEA | HLA-A*02:01 |

### Example 5—Staining Reactive T-Cells with CLT Antigen Peptide Pentamers and Demonstration of their Killing of Peptide-Pulsed or CLT-Expressing Target Cells

The presence and activity of circulating CD8 T-cells specific for CLT antigens in healthy donors and melanoma patients can be measured by using HLA Class I/peptide-pentamer (“pentamer”) staining and/or in vitro killing assays. Thus, application of these methodologies to CLT antigens discovered using the methods elucidated in Examples 1 and 2 (Table 1-3, FIGS. 1-53) can be used to demonstrate the existence of therapeutically relevant T-cell responses to the CLT antigens in cancer patients.

For these studies, CD8 T-cells isolated from healthy donor or patient blood are expanded using various cultivation methods, for example anti-CD3 and anti-CD28 coated microscopic beads plus Interleukin-2. Expanded cells can then be stained for specific CLT antigen-reactivity of their T-cell receptors using CLT peptide pentamers, which consist of pentamers of HLA Class I molecules bound to the relevant CLT Antigen peptide in the peptide-binding groove of the HLA molecule. Binding is measured by detection with phycoerythrin or allophycocyanin-conjugated antibody fragments specific for the coiled-coil multimerisation domain of the pentamer structure. In addition to the pentamer stain, further surface markers can be interrogated such as the memory marker CD45RO and the lysosomal release marker CD107a. Association of pentamer positivity with specific surface markers can be used to infer both the number and phenotype (memory versus naive/stem) of the pentamer-reactive T-cell populations

Pentamer stained cells may also be sorted and purified using a fluorescence activated cell sorter (FACS). Sorted cells may then be further tested for their ability to kill target cells in in vitro killing assays. These assays comprise a CD8 T-cell population, and a fluorescently labelled target cell population. In this case, the CD8 population is either CLT antigen-specific or CD8 T-cells pentamer-sorted and specific for a positive-control antigen known to induce a strong killing response such as Mart-1. The target cells for these studies may include peptide-pulsed T2 cells which express HLA-A*02, peptide-pulsed C1R cells transfected with HLA-A*02,03, B*07, melanoma cells lines previously shown to express the CLTs/CLT antigens, patient tumor cells or cell lines such as CaSki transfected with the CLT open reading frames. Peptides used to pulse the T2 or C1R cells include CLT antigen peptides or positive control peptides. Target cell death is indicated by take up of 7AAD. In this way, as target cells are killed, by apoptosis mediated by CD8 T-cells, they gain red fluorescence. Thus, application of such killing assays to pentamer-sorted, CLT antigen-specific CD8 T-cells can be used to enumerate the cytotoxic activity of CLT-antigen-specific T-cells in ex vivo cultures of melanoma patient or healthy donor T-cells.

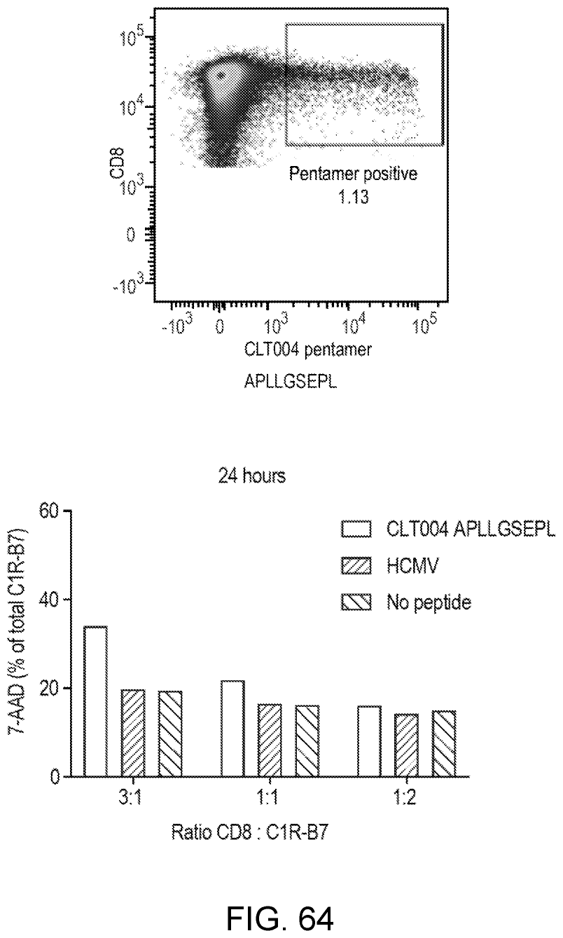

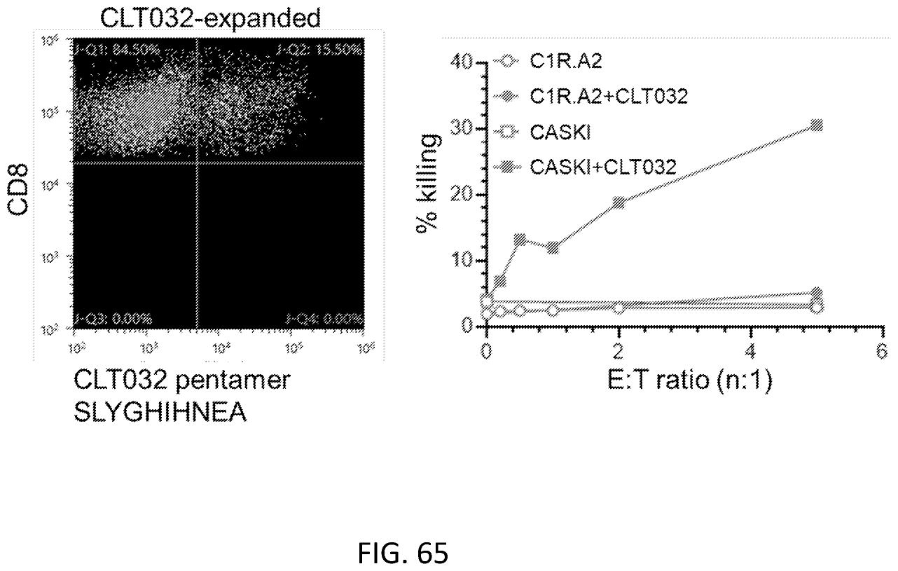

FIG. 64 shows HLA pentamer staining of healthy donor CD8 T-cells with a peptide-derived from CLT Antigen 4, peptide APPLGSEPL (top panel). The bottom panel shows antigen-specific killing of peptide pulsed C1R.B7 target cells by these CD8 T cells. The negative controls for the in vitro killing assay include an irrelevant peptide derived from human cytomegalovirus (HCMV) and no peptide. FIG. 65 shows HLA pentamer staining of healthy donor CD8 T-cells with peptides-derived from CLT Antigen 8, peptide SLYGHIHNEA following fluorescence activated cell sorting of pentamer positive cells and 14 days of expansion using anti-CD3 and anti-CD28 coated beads plus IL-2. The right-hand side panel shows very weak antigen-specific killing of peptide pulsed A2 target cells by these CD8 T cells but effective antigen specific killing of CaSki cells transfected with the open reading frame of CLT Antigen 8. The negative controls for this in vitro killing assay include an irrelevant T2 cells with no peptide and untransfected CaSki cells.

### Example 6—Assays to Validate CLT Expression in Melanoma Cells

a) qRT-PCR Validation of CLT Expression in Melanoma Cell Lines

Quantitative real-time polymerase chain reaction (qRT-PCR) is a widespread technique to determine the amount of a particular transcript present in RNA extracted from a given biological sample. Specific nucleic acid primer sequences are designed against the transcript of interest, and the region between the primers is subsequently amplified through a series of thermocyle reactions and fluorescently quantified through the use of intercalating dyes (SYBR Green). Primer pairs were designed against the CLTs and assayed against RNA extracted from melanoma cell lines or primary patient tissue. Non-melanoma cell lines were utilised as negative controls. Melanoma cell lines used included COLO 829 (ATCC reference CRL-1974), MeWo (ATCC reference HTB-65), SH-4 (ATCC reference CRL-7724) and control cell lines HepG2 (hepatocellular carcinoma, ATCC reference HB-8065), Jurkat (T-cell leukemia, ATCC reference TIB152) and MCF7 (adenocarcinoma, ATCC reference HTB-22). Patient-derived melanoma tissue was obtained from 6 primary lesions and 6 metastases, all from patients with at least stage IIC disease. RNA was extracted from each sample and reverse transcribed into cDNA following standard procedures. qRT-PCR analysis with SYBR Green detection following standard techniques was performed with primers designed against two regions of each CLT, and reference genes. Relative quantification (RQ) was calculated as:

RQ=2[Ct(REFERENCE)−Ct(TARGET)].

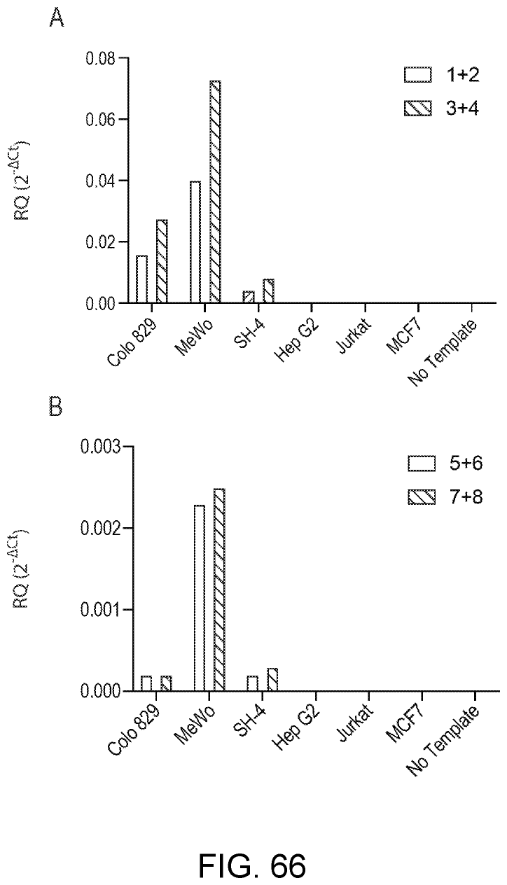

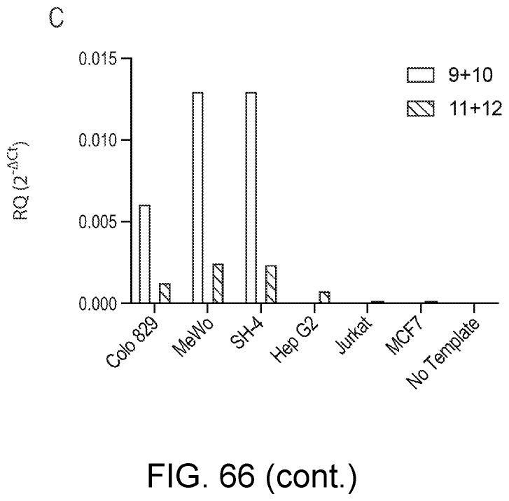

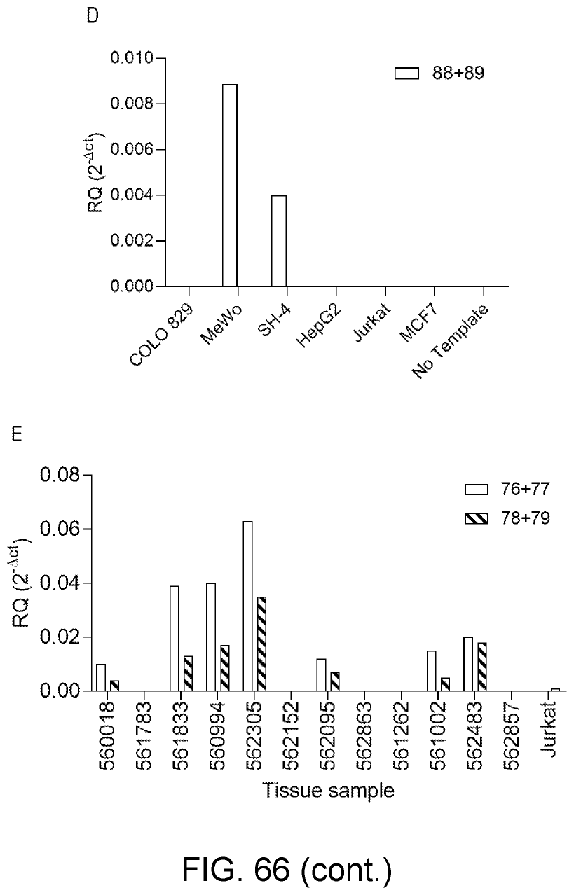

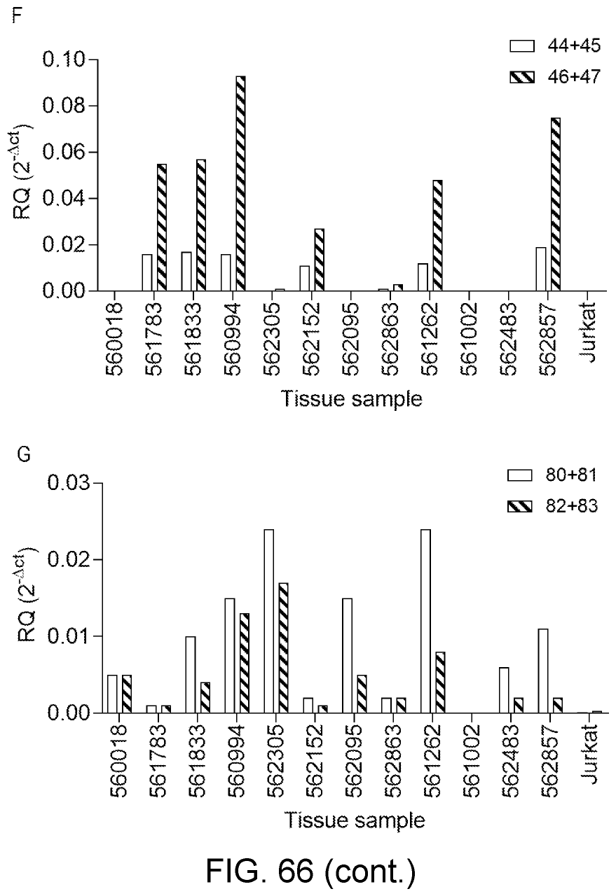

The results of these experiments are presented in FIG. 66. Panel A shows results from a qRT-PCR assay with two primer sets (1+2 and 3+4) targeting different regions of the CLT encoding CLT Antigen 1 (SEQ ID 56) on RNA extracted from three melanoma cell lines and four non-melanoma cell lines. Panel B shows results from qRT-PCR assay with two primer sets (5+6 and 7+8) targeting different regions of the CLT encoding CLT Antigen 2 (SEQ ID 57) on RNA extracted from three melanoma cell lines and four non-melanoma cell lines. Panel C shows results from qRT-PCR assay with two primer sets (9+10 AND 11+12) targeting different regions of the CLT encoding CLT Antigens 3/4 (SEQ ID 58) on RNA extracted from three melanoma cell lines and four non-melanoma cell lines. Panel D shows results from qRT-PCR assay with one primer set (88+89) targeting the CLT encoding CLT Antigen 5 (SEQ ID 59) on RNA extracted from three melanoma cell lines and four non-melanoma cell lines. Panel E shows results from qRT-PCR assay with two primer sets (76+77 AND 78+79) targeting different regions of the CLT encoding CLT Antigen 6 (SEQ ID 60) on RNA extracted from 12 melanoma tissue samples and one non-melanoma cell line. Panel F shows results from qRT-PCR assay with two primer sets (44+45 AND 46+47) targeting different regions of the CLT encoding CLT Antigen 7 (SEQ ID 61) on RNA extracted from 12 melanoma tissue samples and one non-melanoma cell line. Panel G shows results from qRT-PCR assay with two primer sets (80-81 AND 82-83) targeting different regions of the CLT encoding CLT Antigen 8 (SEQ ID 62) on RNA extracted from 12 melanoma tissue samples and one non-melanoma cell line. These results confirmed the specific expression of CLTs in RNA extracted from melanoma cell lines or tissue, compared to non-melanoma cell lines. Each CLT was detected in two or more cell lines or tissue samples analysed, with little to no expression detected in non-melanoma control cell lines.

b) RNAScope Validation of CLT Expression in Melanoma Cells In Situ

In situ hybridisation (ISH) methods of transcript expression analysis allow the presence and expression levels of a given transcript to be visualised within the histopathological context of a specimen. Traditional RNA ISH assays involve the recognition of native RNA molecules in situ with oligonucleotide probes specific to a short stretch of the desired RNA sequence, which are visualised through a signal produced by a combination of antibody or enzymatic-based colorimetric reactions. RNAScope is a recently developed in situ hybridization-based technique with more advanced probe chemistry ensuring specificity of the signal produced and allowing sensitive, single-molecule visualization of target transcripts (Wang et al 2012 J Mol Diagn. 14(1): 22-29). Positive staining for a transcript molecule appears as a small red dot in a given cell, with multiple dots indicative of multiple transcripts present.

RNAScope probes were designed against the CLTs and assayed on sections of 12 formalin-fixed, paraffin-embedded cutaneous melanoma tumor cores. Scoring of the expression signal was performed on representative images from each core as follows:
Estimated % cells with positive staining for the CLT probe, rounded up to the nearest 10
Estimated level of per cell expression across the given section as:
   0=no staining
   1=1-2 dots per cell
   2=2-6 dots per cell
   3=6-10 dots per cell
   4=>10 dots per cell

Expression of each of each CLT was detected across a number of different patient tumor cores, independently validating the discovery of CLTs from tumor-derived RNAseq data and confirming homogeneity of expression within tumor tissue across certain samples and also highlighting the presence of at least one CLT in each patient core analysed.

TABLE 10
Scoring of RNAScope in melanoma patient tissue cores

| | | CLT Antigen 1 | | CLT Antigen 2 | | CLT Antigen 3/4 | |
|---|---|---|---|---|---|---|---|
| | | (SEQ ID 56) | | (SEQ ID 57) | | (SEQ ID 58) | |
| | | % of | | % of | | % of | |
| | | (+)ve | | (+)ve | | (+)ve | |
| Tissue | Core | cells | Score | cells | Score | cells | Score |
| Melanoma | 61 | 10 | 1 | 80 | 4 | 10 | 1 |
| Melanoma | 62 | 80 | 2 | 80 | 2 | 100 | 4 |
| Melanoma | 63 | 100 | 4 | 0 | 0 | 100 | 4 |
| Melanoma | 64 | 0 | 0 | 0 | 0 | 100 | 3 |
| Melanoma | 65 | 70 | 3 | 0 | 0 | 80 | 4 |
| Melanoma | 66 | 100 | 3 | 50 | 2 | 90 | 3 |
| Melanoma | 67 | 100 | 3 | 100 | 4 | 50 | 2 |
| Melanoma | 68 | 0 | 0 | 0 | 0 | 100 | 3 |
| Melanoma | 69 | 90 | 3 | 100 | 4 | 100 | 4 |
| Melanoma | 70 | 0 | 0 | 0 | 0 | 70 | 3 |
| Melanoma | 71 | 0 | 0 | 0 | 0 | 80 | 4 |
| Melanoma | 72 | 100 | 3 | 100 | 4 | 60 | 2 |

| | | CLT Antigen 5 | | CLT Antigen 6 | | CLT Antigen 7 | | CLT Antigen 8 | |
|---|---|---|---|---|---|---|---|---|---|
| | | (SEQ ID 59) | | (SEQ ID 60) | | (SEQ ID 61) | | (SEQ ID 62) | |
| | | % of | | % of | | % of | | % of | |
| | | (+)ve | | (+)ve | | (+)ve | | (+)ve | |
| Tissue | Core | cells | Score | cells | Score | cells | Score | cells | Score |
| Melanoma | 61 | 30 | 2 | 0 | 0 | 0 | 0 | 10 | 1 |
| Melanoma | 62 | 20 | 1 | 90 | 3 | 20 | 1 | 10 | 1 |
| Melanoma | 63 | 10 | 1 | 100 | 4 | 0 | 0 | 80 | 2 |
| Melanoma | 64 | 70 | 2 | 50 | 1 | 0 | 0 | 40 | 2 |
| Melanoma | 65 | 10 | 1 | 50 | 2 | 30 | 2 | 50 | 2 |
| Melanoma | 66 | 80 | 3 | 100 | 4 | 50 | 1 | 50 | n/a |
| Melanoma | 67 | 100 | 4 | n/a | n/a | n/a | n/a | n/a | n/a |
| Melanoma | 68 | 30 | 2 | 40 | 1 | 0 | 0 | 50 | 1 |
| Melanoma | 69 | 10 | 1 | 80 | 3 | 80 | 1 | 50 | 1 |
| Melanoma | 70 | 10 | 1 | 10 | 1 | 20 | 1 | 10 | 1 |
| Melanoma | 71 | 50 | 3 | 0 | 0 | 0 | 0 | 10 | 1 |
| Melanoma | 72 | 90 | 3 | 100 | 3 | 0 | 0 | 10 | 1 |

### Example 7—Ex Vivo Stimulation of T Cells Using Pools of CLT Antigens or CLT Antigen Fusion Proteins

T cells from a healthy donor or patient with a given cancer, can be stimulated outside of the body (ex vivo) to activate T cell clones that recognise specified CLT Antigens, and subsequently rapidly expanded to generate large numbers of CLT-reactive T cells, where resultant anti-tumor activity might be anticipated. In the case of a cancer patient, such a method could be developed as an anti-cancer therapeutic. A number of steps are involved to employ this method.

A) Isolation of Relevant Patient Immune Cells

T cells from the donor (healthy or cancer patient) must be isolated but also autologous antigen presenting cells (APCs) may be required. The source of the immune cells can be obtained from peripheral blood through a blood draw or apheresis. Alternatively, T cells can be isolated from the tumor infiltrating lymphocytes (TILs) obtained from fresh biopsy or resection of a patient's tumor. APCs may be cluster of differentiation (CD)14-positive monocytes or alternatively dendritic cells (DCs) which would be derived from the monocyte fraction of the apheresis product. DCs can be generated by methods such as positive isolation via CD14 capture (for example, anti-CD14 antibodies conjugated to magnetic beads, where CD14-positive cells are labelled with the beads and captured on a magnetic column) or isolation via their adhesive properties, for example, adherence to tissue culture plastics by incubation of peripheral blood mononuclear cells (PBMCs) with cell culture dishes for a period of 4-48 hr to allow adherence of monocytes. DCs can be generated from the CD14-positive or adherent immune cell fractions by well-described methods utilising cytokines such as, but not limited to: GM-CSF, IL-4, TNFα, IL-1β, IL-6, Prostaglandin E2. Incubation with such cytokines over the course of 2-7 days allows differentiation of the CD14+ monocytes into DCs, typically that will have lost the expression of CD14 and upregulated expression of DC markers such as CD11c, high levels of MHC Class II, etc. The nature of the T cells for selection and/or stimulation could be the monocyte-depleted fraction of PBMC (in the case of apheresis origin of T cells), pan-T cell isolation using isolation techniques based on the expression of markers such as CD3, or presence or absence of markers of specific T cell subsets, for example but not limited to, CD4, CD8, CD45RO, CD45RA, CCR7, CD62L, CD27 etc.

B) Selection of CLT Antigen-Recognising T Cells

Methods can be employed to select T cells prior to stimulation with APCs. Such methods would include peptide-HLA (pHLA) multimer approaches such as tetramer, pentamer, dextramer or similar, to label T cells that express TCRs that recognise the given pHLA. Such pHLAs would be defined based on mass spectrometry (MS) experiments as described in Example 2, and/or peptides predicted to bind specific HLA allotypes based on prediction algorithms. The multimer could possess a tag, such as phycoerythrin (PE) which could be isolated using fluorescent activated sorting or via an anti-PE antibody conjugated to magnetic beads. Alternatively, an antibody to the tag could be directly conjugated to magnetic beads. To isolate different T cells that recognise different pHLAs from different CLT Antigens, multimers could be generated with the same or different tags, or different multimers could be conjugated to magnetic beads.

C) Stimulation of T Cells

In order to potentiate pre-existing (memory) or stimulate new (naïve) T cell responses from cancer patients to CLT Antigens, the patient's T cells can be exposed to APCs that are presenting peptides derived from CLT Antigens on the surface in the context of Class I and Class II HLA complexes. For example, multiple CLT Antigens (anticipated to be expressed by a patient's tumor) could be exogenously delivered to a melanoma patient's APCs to result in CLT Antigen-derived peptides being presented on HLA complexes at the surface. Introduction of multiple CLT Antigens could be through concatenated polypeptide delivery or as individual CLT Antigens, such as pooled mRNA-based methods of delivery. Alternatively, HLA molecules on APCs could be loaded with exogenous synthetic peptides derived from CLT Antigens. Methods of stabilized, mature, mRNA delivery to the APC (that is, transfection) could include classical reagents such as polyethylenimine (PEI) or calcium phosphate for nucleic acid delivery into cells. Alternatively, efficient transfection can be achieved using lipid-based reagents for transfection into APCs. These transfection reactions use synthetic, in vitro transcription reaction (IVT)-derived mRNAs formulated in lipid complexes such as lipid nanoparticles (LNP) or lipid-based lipoplexes (formed by simple mixing of mRNAs with lipid reagents). To create these mRNAs, recombinant DNA constructs containing the well-described promoter element for phage T7 DNA-dependent, RNA polymerase, followed by a cDNA encoding high-stability mRNA 5′UTR, a cDNA encoding a codon-optimized open reading frame (ORF) for a CLT Antigen, a cDNA encoding a high stability mRNA 3′UTR, a poly-A sequence of >20 nucleotides, and a unique restriction endonuclease site designed to release a functional poly-A tail, can be used as a template for IVT of suitable CLT Antigen-encoding mRNAs. To create human APC-expressing IVT mRNA-encoding antigens, lipoplex methods similar to those described by Cafri et al., Nat. Comm. 2019 could be used. Briefly, APCs (monocytes or DCs) would be plated on tissue culture flasks to achieve confluence of 70-90%. A lipid-based transfection reagent (for example, Lipotectamine™ MessengerMAX™ or FuGENE® HD or similar) would be diluted appropriately in serum-free medium such as Opti-MEM™, mixed and incubated with mRNA encoding the CLT Antigen. This could be done with multiple CLT Antigen mRNAs for transfection of a combination of CLT Antigens to the APC. Incubation times of the mRNA with the lipid reagent would be short (5-10 minutes) and at room temperature. The resultant mRNA-lipid complex would be added to APCs and incubated at 37° C./5% CO₂ for 16-72 hours, depending on optimal timepoint for presentation of translated peptides from the CLT-encoding mRNA molecules.

Delivery of CLT Antigens to APCs with such methods described should result in the expression of CLT Antigen polypeptides in the cytoplasm of the APC, which in turn will result in cellular processing of peptide fragments from the polypeptides for presentation on Class I and Class II HLA molecules. When T cells (either selected as described in (b) or unselected T cells from apheresis or TIL sources) are co-cultured with APCs expressing CLT Antigen-derived peptide-HLA complexes at the cell surface, those T cells possessing TCRs that have specificity for a given pHLA will be stimulated by engaging with the pHLA complex in addition to co-stimulatory molecules and signals from the APC. This will result in activation, differentiation and proliferation of the engaged T cell. For example, following successful transfection of APCs with IVT mRNA-encoding CLT Antigens in a method as described above, autologous CD3+ isolated T cells would be co-cultured with the APCs at a ratio of excess T cell to APC, for example 10 T cells per 1 APC (10:1), in cytokine-containing medium (such as IL-6 and IL-12 or other cytokines supplemented in the basal media used). The cells would be co-cultured for as little as overnight or up to 1 week to stimulate T cells, but typically 18-48 hours, after which the T cells could be subjected to enrichment prior to expansion, if required.

D) Enrichment of Stimulated T Cells

T cells that have been stimulated by APCs that are expressing CLT Antigens can be further enriched prior to an expansion step if required. Markers of T cell activation (such as CD137, CD107a, CD69, OX40 or other surface marker associated with an activated state) or T cell functional responses (for example, T cells secreting cytokines such as TNFα or IFNγ) could be selected for, to enrich the T cell population for those cells that might be CLT Antigen-specific. Such enrichment methods could include cell sorting by FACS or bead-based methods of capture, for example, using antibodies to CD137 or similar that are conjugated to magnetic beads. Multiple enrichment strategies could be employed, either in parallel (for example, cells double positive for CD137 and CD69) or sequentially (for example, selecting cells positive for CD137 and subsequently selecting CD137+ cells positive for CD69). Such a positive selection should remove those T cells that are likely not stimulated by the CLT Antigen-expressing APCs.

E) Rapid Expansion of Stimulated T Cells

Following stimulation of T cells with APCs that have had CLT Antigens introduced into them, bulk or enriched (see (D) above) T cells can be rapidly expanded to achieve numbers >10⁸ total cells, using methods based on those described in the literature, with potential modifications for optimisation (for example, Jin et al., J Immunother, 2012). Such methods utilise cytokines such as IL-2 and stimulatory antibodies such as anti-CD3 as well as potential irradiated autologous cells from PBMC (termed “feeder” cells). Alternatively stimulatory antibodies to CD3 and CD28 can be used to avoid the use of feeder cells. The process can be further automated or enhanced using specialized gas-permeable flasks (for example G-Rex flasks) or closed expansion system (for example WAVE bioreactor). Significant expansion of T cells (100-1000 fold) can be achieved in as little as 7-14 days, depending on the numbers of T cells at the start.

F) Testing of Expanded T Cells for Evidence of CLT Antigen Immunogenicity

To demonstrate that the ex vivo autologous stimulation process has expanded T cells that recognize CLT Antigen-expressing target cells (including tumor cells), multimers corresponding to specific CLT Antigen peptide-HLA (pHLA) complexes could be used to detect the presence of T cells with reactivity for a particular CLT Antigen pHLA. Multiple pHLAs from a combination of CLT Antigens could be used with different labels to demonstrate recognition of more than 1 CLT Antigen by the ex vivo-stimulated T cells.

Functional assays would also demonstrate the ability of the ex vivo-immunized T cells to respond to target cells presenting peptides from the CLT Antigens. This could be achieved through a variety of approaches. Firstly, cytokine release assays could be performed to test for T cell activation from co-cultivation of the ex vivo stimulated T cells with the target cells (for example, IFNγ ELISpot assays). Alternatively, T-cell mediated killing of target cells could be measured with cytotoxicity assays such as FACS-based methods to assess cell death of target cells (e.g. by 7-AAD measurement) co-cultured with the T cells, or other methods such as those that monitor markers of apoptosis of target cells or measure impedance (electrical measure of cell viability) of adherent target cells plated onto specialized surfaces.

A variety of methods could be used to create target cells for such assays. For example appropriate human cells with HLAs that match APCs used in the ex vivo stimulation could be pulsed with peptides derived from CLT Antigens that are known to be presented on Class I HLA molecules (as deconvoluted from mass spectrometry experiments—see Example 2). Further, tumor cell lines matching the HLA type of the APCs could also be assessed. Finally, primary tumor cells (in particular tumor cells from the same patient donor from which the starting T cells and APCs used for the process were derived) could be assessed.

In conclusion, these methods can be used to demonstrate that a) human T cells are able to be “immunized” with CLT Antigens using autologous APCs ex vivo, b) immunized T cells are able to be potentially enriched over non-immunized T cells, c) immunized T cells can be rapidly expanded to produce several log-fold higher number of total cells, and d) rapidly expanded, immunized T cells retain the capacity to recognize target cells that express the same HLAs and CLT Antigens they were immunized against. These data would support the likelihood that an ex vivo stimulation protocol applied to cancer patients with one or more CLT Antigen(s) would have therapeutic value in controlling cancer.

### Example 8—Methodology for CLT Antigen Fusion Protein Design

One method to facilitate delivery of a multi-polypeptide antigen mixture is by combining the ORFs for multiple component antigens into a single ORF, resulting in the synthesis of an antigenic fusion protein. Further, rather than directly linking the component polypeptides together, these can be connected by peptide linker regions to: 1) reduce the potential risk of generating novel epitopes at fusion junctions that mimicked normal human proteins (increasing safety) and 2) ensure that CLT Antigen T cell epitopes bordering the fusions/linkers are processed in a manner that mimics their presentation when expressed from individual ORFs encoded by tumor tissues (increasing effectiveness). To accomplish linkage to facilitate the design of safe and effective fusion proteins, an algorithm was developed. Simple short linkers are used in this algorithm, since these would be superior for achieving the above-mentioned goals. To this end, multiple Gly-based linkers were selected, some of which also contained Lys residues to eliminate identity to normal human proteins and facilitate processing at the ends of the component CLT antigens (GGG, GGGG, KGG, GGKGG, GGGKGG, GGK; SEQ ID NO. 71-75, 84 respectively).

For the purposes of this example, the algorithm was applied to two different sets of CLT Antigens, CLT Antigens 1, 2, 4, 6, 7 and 8 (CLT Antigen Fusion Protein 1 and 2) and CLT Antigens 1-8 (CLT Antigen Fusion Protein 3 and 4).

To accomplish the needs described above, six criteria were considered in the design of each of four individual fusion protein (CLT Antigen Fusion Protein 1/CLT Antigen Fusion Protein 2 for a six-CLT Antigen antigenic stimulus and CLT Antigen Fusion Protein 3/CLT Antigen Fusion Protein 4 for an eight-CLT Antigen antigenic stimulus). These were applied one-by-one, and then repeated, as needed, in an iterative manner to ensure that the final fusion protein candidates satisfied all criteria.

First, fusion protein sequences were designed so that no 9-mer peptides containing any portion of a linker peptide could be identical to the human proteome, as determined by a blastp search performed by Standalone Blast ver2.9.0 (AltSchul et al, J. Mol. Biol. 1990). For completeness, this blastp search was performed against three proteome sub-databases extracted from the Ensemble database (www.ensembl.org); SwissProt Human proteome, Trembl Ensembl Human up000005640 proteome, and Trembl all human proteome (created 14 Aug. 2019).

Second, fusion protein sequences were designed so that no 9-mer peptide containing any portion of a linker peptide could be a strong predicted binder (rank 0.5) for an MHC class I supertype (see below) by NetMHCpan 4.0. (Andreatta & Nielsen, Bioinformatics 2016). For the generation of a fusion protein encoding CLT Antigens suitable for use in a melanoma therapy, MHC HLA class I types important for the target population of melanoma patients was the key driver, resulting in selection of the following supertypes: HLA-A*01:01, HLA-A*02:01, HLA-A*03:01, HLA-A*11:01, HLA-A*24:02, HLA-A*25:01, HLA-A*26:01, HLA-A*68:01, HLA-B07:02, HLA-B*08:01, HLA-B*18:01, HLA-B*27:05, HLA-B*35:01, HLA-B*35:03, HLA-B*40:01, HLA-B*40:02, HLA-B51:01, HLA-C*07:01, HLA-C*07:02.

Third, fusion protein sequences were designed so that CLT Antigens for which HLA-bound peptides (see Example 2) were found precisely aligned with their C-termini were prioritized for positioning at the C-terminus of the fusion protein designs to help ensure that the C-terminal anchor residues (normally released by a stop codon when expressed in tumor tissues) would be similarly produced in the context of a fusion protein. When C-terminal placement was not possible for such CLT Antigens, linker sequences were further optimized based on proteasomal cleavage site predictions made with the NetChop 3.1 Server (Nielsen et al., Immunogenetics 2005) to select linker sequences expected to produce the C-termini found in the authentic (stop-codon-generated) CTA antigen polypeptide.

Fourth, fusion protein sequences were designed so that all 9-mer peptide sequences containing any portion of a linker peptide that were predicted to be weak binders (rank score 2.0) for a selected MHC class I supertype (see above) were altered to eliminate or reduce binding by adjusting linkers or, in some cases, by removing the N-terminal methionines from component CLT antigens. The truncation of N-terminal methionines was adopted as a strategy to eliminate 9-mers predicted to weakly bind selected MHC Class I molecules, since methionine is rarely found in the N-terminal position of MHC Class I bound peptides (Abelin et al., Immunity 2017; Alvarez et al., Molecular & Cellular Proteomics 2019). Elimination/reduction of peptide sequences predicted to be weak binders for high frequency HLA class I types (HLA-A*02:01, HLA-A*03:01, HLA-B*07:02) were prioritized for alteration. Where possible, the elimination/reduction of peptide sequences predicted to be weak binders to all other selected supertypes (see above) was achieved by using the same procedures.

Fifth, since one application of the fusion proteins is in a prime/boost antigenic stimulation regimen, the pairs of the fusion protein designs used for this purpose were designed so that there were not allowed to directly repeat CLT antigen junctions to reduce epitope repetition.

Sixth, the pairs of fusion proteins were refined to remove predicted weak binding epitopes repeated between prime and boost constructs (implemented for CLT Antigen Fusion Protein 3 and 4). This was achieved by either excluding N-terminal methionine residues (see rationale described above for N-terminal methionine removal) or by the use of alternative linkers (see above).

### Implementation of Design for CLT Antigens

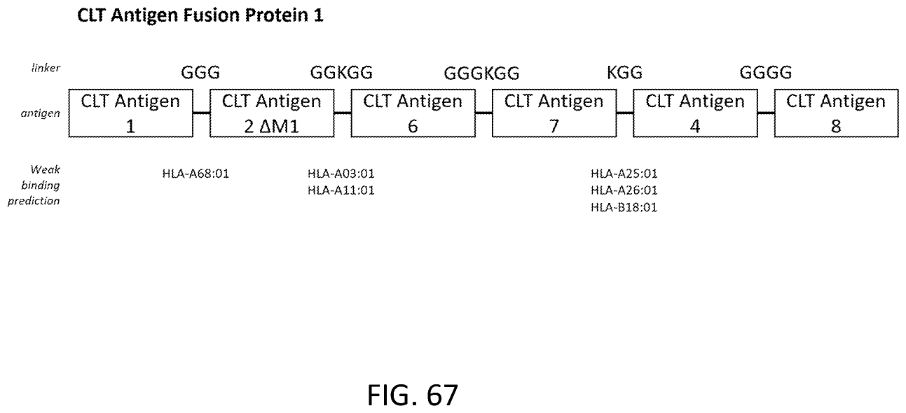

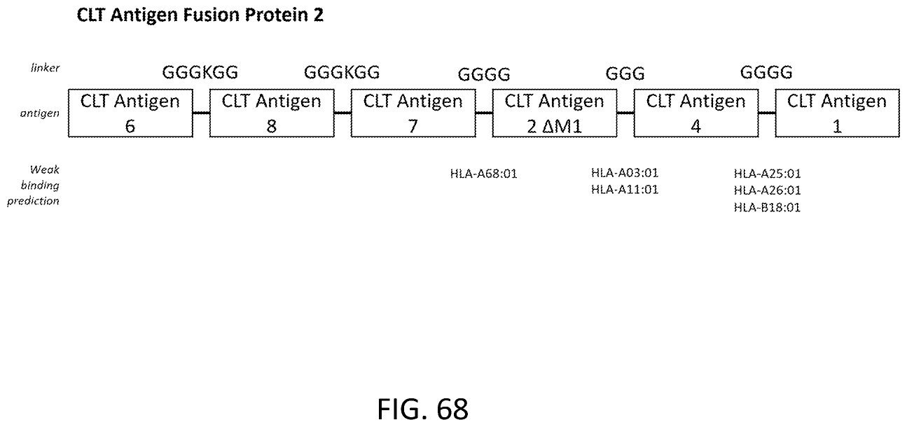

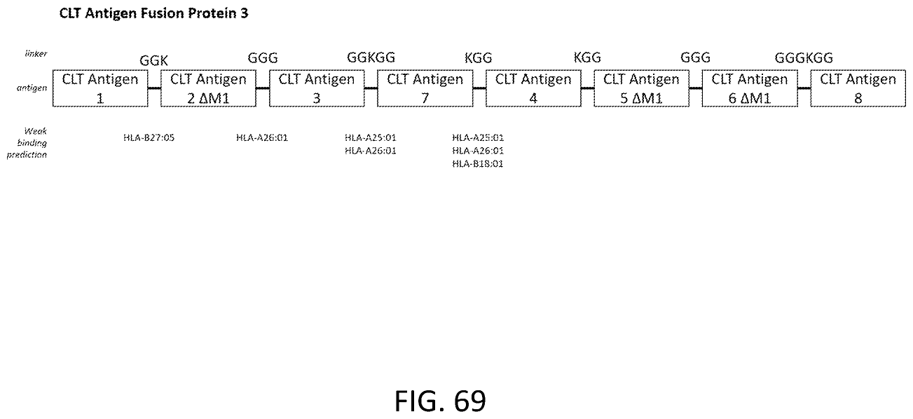

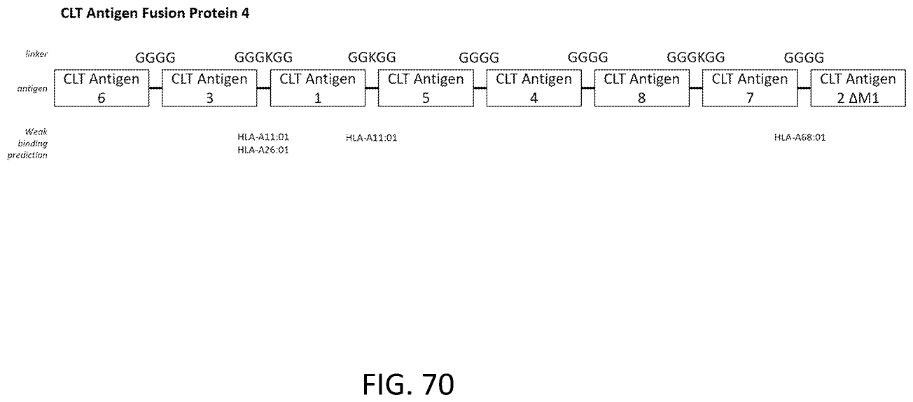

The result of the fusion protein design strategy described above was the four constructs (CLT Antigen Fusion Protein 1 (SEQ ID NO. 76), CLT Antigen Fusion Protein 2 (SEQ ID NO. 77), CLT Antigen Fusion Protein 3 (SEQ ID NO. 78), CLT Antigen Fusion Protein 4 (SEQ ID NO. 79)) shown in schematic form in FIGS. 67-70.

### Example 9—Antigenicity of Pools of CLT Antigens or CLT Antigen Fusion Proteins

Individual CLT Antigens discovered and validated as described in Examples 1-6 and the CLT Antigen Fusion Proteins designed as described in Example 8 are expected to be translated, proteolytically processed in the cytosol, and presented in association with HLA class I molecules on the cell surface. Pools (2 or more) of cDNA constructs encoding individual CLT Antigens or cDNA constructs encoding the fusion protein cassettes are transduced into human cells, and the HLA class I molecules are immunoprecipitated and subjected to MS analyses described in Example 2 for the discovery of the CLT antigens. This is done in order to demonstrate that multiple, exogenously inserted, individual CLT Antigens and/or the CLT Antigen fusion protein cassettes maintained similar antigen presentation properties of the component CLT Antigens previously identified in tumors tissues (shown Example 2).

MS-based immunopeptidomics analysis is a powerful technology that allows for the direct detection of specific peptides associated with HLA class I molecules. However, the ability to detect individual peptides is influenced by their biophysical properties, it is restricted by the proteolytical activity present in the cells and HLA alleles expressed in the cell lines used for these studies. Thus, the method will not likely discover all previously identified HLA-bound peptides in a tissue or cell sample. Nevertheless, the repertoire of HLA class I-bound peptides detected will confirm the value of combining multiple CLT Antigens, either as a pool of individual CLT Antigen-encoding constructs or as part of the fusion protein designs tested in delivering peptide epitopes from CLT Antigens.

To accomplish MS-based studies of multiple, exogenously inserted, individual CLT Antigens and/or CLT Antigen Fusion Protein designs, cultured human cells are transduced with plasmid DNAs encoding pools of individual CLT Antigens or the CLT Antigen fusion protein cassettes under control of suitable polII promoter and 5′ and 3′ UTRs. After expansion, the cultured cells are lysed and the HLA class I-peptide complexes are affinity purified by anti-HLA Class I antibody capture. The isolated HLA molecules and bound peptides are then separated from each other and the eluted peptides are analyzed by nUPLC-MS/MS. The MS/MS spectra acquired from these HLA Class I pull downs are then interrogated by using the PEAKS™ software (v8.5 and vX, Bioinformatics Solutions Inc). For MS/MS interpretation the software evaluates side-by-side all theoretical spectra of polypeptides contained in the human proteome with the polypeptide of the relevant individual CLT Antigens or CLT Antigen fusion proteins. For these studies it is essential that the repertoire of analyzed sequences contains sequences of the relevant CLT Antigens AND the human proteome since the great majority of Class I HLA-bound peptides found in cells are derived from constitutively expressed proteins.

The results of these studies identify individual CLT Antigen-derived peptides processed and presented by the HLA Class I repertoire of the transduced cells. Analyses of these data are used to demonstrate that the combination of multiple CLT Antigens, either as a mixture of individual cDNAs and/or as part of a CLT Antigen fusion protein design has resulted in effective presentation of peptides from the CLT Antigens. Moreover, the results of these studies show that epitopes derived from linker regions of the tested protein fusions are not efficiently presented in fusion protein cDNA transduced cells.

Taken together, these data can provide strong support for the translation, processing, and presentation of CLT Antigen epitopes from multiple CLT Antigens used in combination. This supports the development of therapeutic modalities designed to stimulate and expand T cells that recognize the CLT Antigen peptides/HLA Class I complexes on tumors found in patients.

### Example 10 Killing of CLT Antigen-Expressing Cell Lines

The immunogenicity of antigens derived from cells expressing multiple CLT Antigens, either derived from pools of multiple individual CLT Antigens or CLT Antigen Fusion Protein open reading frames, can be demonstrated using transfected cell lines combined with the CLT-peptide-reactive T cells described in Example 5. Killing of cell lines transfected with multiple CLT Antigens by CLT Antigen-specific CD8 T cells is used to demonstrate the existence of therapeutically relevant T-cell responses to a combination of CLT Antigens in cancer patients.

CaSki cells which have been transfected with constructs described in Examples 8 are used as targets for killing assays as described in Example 5. Relevant CD8 T cell lines isolated from healthy donors and melanoma patients using HLA-pentamers derived from CLT Antigens 1, 2, 3, 4, 5, 6, 7 and 8 are tested individually for killing ability of these transfected target cells transfected with multiple CLT Antigens or CLT Antigen fusion proteins. The negative control cells are untransfected CaSki or CaSki cells transfected with an irrelevant construct.

SEQUENCE LISTING

SEQ ID NO. 1 (Polypeptide sequence of CLT Antigen 1)
MWNFFRRELTSNGFPENFSLDVPANTYNALKSRLCDPNADHTSCPSPCSLHAAGALP
GTGRQRWRVELAHLADRKLSLRDVSRLRQGGERRSGIAVKVVRGGAGFAARLQGSV
TLVQQGWFFPRLGGCQAWWRMGAVVWCGELLTCTS
SEQ ID NO. 2 (Polypeptide sequence of CLT Antigen 2)
MTGVLIRRGDLVTDMVACRIKTFRGHTEKAAICKTRKESSAETSPADSLILDFQPLQLM
SSFSTLASLDK
SEQ ID NO. 3 (Polypeptide sequence of CLT Antigen 3)
MNTPNIVSLRAHQPEVGIIPSVLLMRPLRIKGVFHHIHSPLHGENQGFTLCLQGAPPSSS
V
SEQ ID NO. 4 (Polypeptide sequence of CLT Antigen 4)
MAKTKGSLSVFRELHPAAAFDRAVHFLFLELWLPEPMLSSSPPSSTAPLLGSEPLRHW
EASLSR
SEQ ID NO. 5 (Polypeptide sequence of CLT Antigen 5)
MLPRTPRPDLILLQLLPAGLRQLLQTSGPDNEQPIEQDLICNVC
SEQ ID NO. 6 (Polypeptide sequence of CLT Antigen 6)
MWNSLEARSPKSRCCSTGPWEAVQENLLWASLLAPGGATIFPDPWLLKASPSSLPHL
HRTSPCACLCPNFPFYKDTVLLHQGPR
SEQ ID NO. 7 (Polypeptide sequence of CLT Antigen 7)
MAMGQRTPSLAELRKSSATFLTCNLGAQAEKRSRAPGKLTYVSTIVLDAPVTKLEQGL
VMKRYKIVTQGFDYTSVES
SEQ ID NO. 8 (Polypeptide sequence of CLT Antigen 8)
MCALQGRGASPAGAGLFHWTMSPFLLGSLYGHIHNEAV
SEQ ID NO. 9 (peptide sequence derived from CLT Antigen 1)
VQQGWFFPR
SEQ ID NO. 10 (peptide sequence derived from CLT Antigen 1)
VVRGGAGFAAR
SEQ ID NO. 11 (peptide sequence derived from CLT Antigen 1)
HLADRKLSL
SEQ ID NO 12 (peptide sequence derived from CLT Antigen 1)
ARLQGSVTL
SEQ ID NO. 13 (peptide sequence derived from CLT Antigen 1)
VPANTYNALK
SEQ ID NO. 14 (peptide sequence derived from CLT Antigen 1)
RLGGCQAWWR
SEQ ID NO. 15 (peptide sequence derived from CLT Antigen 1)
ANTYNALKSR
SEQ ID NO. 16 (peptide sequence derived from CLT Antigen 1)
RLQGSVTLV
SEQ ID NO. 17 (peptide sequence derived from CLT Antigen 1)
VPANTYNAL
SEQ ID NO. 18 (peptide sequence derived from CLT Antigen 2)
ADSLILDF
SEQ ID NO. 19 (peptide sequence derived from CLT Antigen 2)
SSFSTLASLDK
SEQ ID NO. 20 (peptide sequence derived from CLT Antigen 2)
LVTDMVACRI
SEQ ID NO. 21 (peptide sequence derived from CLT Antigen 2)
LILDFQPLQL
SEQ ID NO. 22 (peptide sequence derived from CLT Antigen 2)
MSSFSTLASL
SEQ ID NO. 23 (peptide sequence derived from CLT Antigen 2)
LMSSFSTLASL
SEQ ID NO. 24 (peptide sequence derived from CLT Antigen 2)
LMSSFSTLA
SEQ ID NO. 25 (peptide sequence derived from CLT Antigen 2)
QLMSSFSTLA
SEQ ID NO. 26 (peptide sequence derived from CLT Antigen 2)
MVACRIKTFR
SEQ ID NO. 27 (peptide sequence derived from CLT Antigen 2)
VTDMVACRIK
SEQ ID NO. 28 (peptide sequence derived from CLT Antigen 2)
SPADSLIL
SEQ ID NO. 29 (peptide sequence derived from CLT Antigen 2)
SLILDFQPL
SEQ ID NO. 30 (peptide sequence derived from CLT Antigen 2)
QLMSSFSTL
SEQ ID NO. 31 (peptide sequence derived from CLT Antigen 3)
NTPNIVSLR
SEQ ID NO. 32 (peptide sequence derived from CLT Antigen 3)
RPLRIKGVF
SEQ ID NO. 33 (peptide sequence derived from CLT Antigen 3)
NTPNIVSLRA
SEQ ID NO. 34 (peptide sequence derived from CLT Antigen 3)
VLLMRPLRIK
SEQ ID NO. 35 (peptide sequence derived from CLT Antigen 3)
MRPLRIKGVF
SEQ ID NO. 36 (peptide sequence derived from CLT Antigen 4)
KTKGSLSVFR
SEQ ID NO. 37 (peptide sequence derived from CLT Antigen 4)
AAFDRAVHF
SEQ ID NO. 38 (peptide sequence derived from CLT Antigen 4)
AFDRAVHF
SEQ ID NO. 39 (peptide sequence derived from CLT Antigen 4)
KTKGSLSVF
SEQ ID NO. 40 (peptide sequence derived from CLT Antigen 4)
FLFLELWL
SEQ ID NO. 41 (peptide sequence derived from CLT Antigen 4)
SVFRELHPA
SEQ ID NO. 42 (peptide sequence derived from CLT Antigen 4)
SPPSSTAPL
SEQ ID NO. 43 (peptide sequence derived from CLT Antigen 4)
FLELWLPEPML
SEQ ID NO. 44 (peptide sequence derived from CLT Antigen 4)
APLLGSEPL
SEQ ID NO. 45 (peptide sequence derived from CLT Antigen 5)
LPRTPRPDLIL
SEQ ID NO. 46 (peptide sequence derived from CLT Antigen 5)
TPRPDLILL
SEQ ID NO. 47 (peptide sequence derived from CLT Antigen 5)
RPDLILLQL
SEQ ID NO. 48 (peptide sequence derived from CLT Antigen 6)
ATIFPDPWLLK
SEQ ID NO. 49 (peptide sequence derived from CLT Antigen 6)
FPFYKDTVL
SEQ ID NO. 50 (peptide sequence derived from CLT Antigen 6)
FPFYKDTVLL
SEQ ID NO. 51 (peptide sequence derived from CLT Antigen 6)
TIFPDPWLLK
SEQ ID NO. 52 (peptide sequence derived from CLT Antigen 7)
IVLDAPVTK
SEQ ID NO. 53 (peptide sequence derived from CLT Antigen 8)
GHIHNEAV
SEQ ID NO. 54 (peptide sequence derived from CLT Antigen 8)
SLYGHIHNEAV
SEQ ID NO. 55 (peptide sequence derived from CLT Antigen 8)
SLYGHIHNEA
SEQ ID NO. 56 (cDNA sequence of CLT encoding CLT Antigen 1)
CGGGGCCAGTCTTTCCCGTGCTATTCTCGTGATAGTGAATAAGTCTCACAAGATCT
GATGGGTTTATCAGGGGTTTTCATTTTGCTTCTTCCTCATTTTCTTTTGCTGCTGTAA
TGTAAGAAACGCCTTTTGCCTCCTGCCATAATTCTGAGGCCTCACAGCCATGTGGA
ACTTCTTCAGGAGAGAATTAACATCCAATGGATTCCCAGAAAACTTTTCCCTCGATG
TACCAGCAAACACCTACAATGCCCTGAAAAGCCGCCTCTGCGACCCCAATGCAGA
TCACACGTCCTGTCCCAGCCCCTGCAGCCTCCACGCGGCGGGTGCACTGCCAGG
CACGGGAAGGCAGCGCTGGCGAGTAGAACTGGCCCATCTCGCAGATAGGAAGCT
GAGCCTCAGGGACGTTTCACGCCTTCGTCAAGGTGGTGAGAGGAGGAGCGGGAT
TGCCGTGAAGGTGGTGAGAGGAGGAGCGGGGTTTGCTGCCCGACTTCAGGGATC
TGTCACCCTCGTCCAGCAGGGTTGGTTCTTCCCGAGGCTGGGAGGATGCCAAGCC
TGGTGGAGGATGGGGGCGGTGGTGTGGTGTGGGGAGCTTCTGACTTGCACATCC
TGAGGGAACCTTCTGCAGCTGATGTGTGAACTGGACCCCAGGCCGTGCCTCCGAG
GAATCCCCAAGGCTATGGCCCCTCAGGTCCTGCTGGGGTGTTGGCCCCCACCTCT
GCCTCAGAATGCAGGGGTTCTGCAGGGAAGCCGCAGACCAGCCTGCTGCCTTGG
GCCCTAGGGACACTGCAGCCCCAGAAAGTACTGTGGGGGACAAAAGAGTTGTTTC
TCGGGGGAGAAAACACCTGTGAGGAAATGCAGGTGCCACAGAGGGAAATCCTCCT
GGGGAGGAGGGTACCTGTTCCATCCTCGGCCGACACGGGACTGCCTGGTGCCTG
GTACCCACAGCCGCTACCTGCCGCACGCATCTCTCCATGGTTTGCTAATTACTTCC
ATTAGTTTTAAACAAACTTGACAAGAGACAGAAGGGTCCAGAGAGAAATTAAATCTA
ACTGTTTAAACATGT
SEQ ID NO. 57 (cDNA sequence of CLT encoding CLT Antigen 2)
CACCTCCATCACTGCGAATTATAATTCGACATGAGATTTGGGAGATGACACAAAAC
CAAACCATATCAGTCTTTAAAGAGTTAAGTTAAAATAAGCTCTTTAAAGTGGGCCCT
AATCCAGTATGACTGGTGTTCTTATAAGAAGAGGAGATTTGGTCACAGACATGGTT
GCATGCAGAATAAAGACTTTTCGAGGACACACTGAGAAGGCAGCCATCTGCAAAA
CAAGGAAAGAGTCCTCAGCAGAAACCAGTCCTGCAGACTCCTTGATCTTGGACTTC
CAGCCACTGCAATTGATGTCAAGCTTCAGCACCCTTGCATCTCTGGATAAATGAAA
TGTCACCCCAGCTGCCGTCCTTGTTCAGATCTGTGCAATAAAGAGCAAAGCATAAA
ACCAAGTCAAGGCTTTGAGGGAGTGACCTACAAAATGCATAATGTGAAACAATGCA
AAAGCGAAAGGTGCAAAATCCCCATCAAAGAGCTGGAGGCTGACAGATGCGCCAG
TGATAATTCCCATCTTCCAACACAGGAGCACAGCTTCCATTTTCCATAACAGAACAA
CAGCCAGAGCAGCTGGAAGGCAGGGCCGCATCCCAGACTTCCACCAACAATGGG
ATGAGACTTGACATCTGGAATCACAACCACAACAGACCTGAGAGACCCACCAGCTT
GATGACAAGCTTCTTCTTTCAAAGAAAGGTATCAGTCTGGGGGACCTAGTGCTGCA
AACCATGACAAATTAAGTGTGGCATCCCTCACTTGCATAATGGAACTCAGTGATATT
TTTTAATTAACAAGAGTTATTTTTATGTAAGCTTCTCTCATTCCTCCACTGTGCGTGC
TCGGGGGCTGGTGGTGAGGAAAAAGAAAACAGCTGTGCGGGAAGCATCAAGAAA
AGGCAAGTCATGAAGTCTTAGAGATCAGTGACATGTAAGAAAAAGAGTGAGGAGAA
AAATATTCCTACTAAAGTTTTCCATTTGTTTACCTTCCTTGTCACATAGACTTCCAAG
AGTTAGAAGTCTAGGATTTGATCTCCAAATCTTCCTGGCAGATTACTCATCTTCATT
TCATTCATATAGTCCAGGGGTTTGTACAAAGGAAGATGCCAGTTCTTCCCCAATCA
TAACTAAGATATCAAGAGATATTCTTTTGAAATGTAACAAAGGAGATCTGAAGTTCA
TCTGAAAAAAATAAATGGTTTTAGGCGGTCATGACCATGGGATGCTGGACCAGGAT
GGTAAGCTTCAGGAAACAGAATCTGGAGAATGCCCAGCTGCTCCCACAGGAAGCA
TCAGGGAAGAAGAAGAAGAGGTGTGAAGTCTGCCTTCTGCTCTGCTGGGATCCCT
TTCACATCTCCTTTGCCTCCAGGCAGTTTTGGTTCCTGGCCATTTCCAGGTGTGAC
TCACTCAGGATGGTAAGCATCTTCTCTCCTACCCAGAGTAGAGGATGAAGACCTCA
TCTCAGAGGTTGAAGGGAGCTCCAGAGAGAGGTCTCAAACTTCCAGCATTAACTG
CTAAAGAAGCTTCATGAGCTGCTGGAGAACCTGGGAAATGACCAATTATAGGGACA
GAGCTCAAATACTCTGGGACACTCTAGTAGCTGAGAAAGTTCCAACTCCAGGGTGA
TAGAGGACTGCCTGGCAAACCATCATCAAAGCAGAAGACCTGATACTAACATCACA
GGCTATGGTTTATTACTGAAGATCAGTGCTTACACCCTGCCAGAGGTTCAGAAGCA
AACTTATCATTGTTCTCCCTGGAGATGTTGGCCCACATTCTGAAAAGTGTGGTCAG
TAGTAGCAACAGAAAGCAATTGTGCTTGCCAAGCACAATGTCACTGTCCCCAGCCC
TTCCCCCAACACAACCCAGTAGGTGCTTCCTGGCTGCAAACTTGGGAAAGTCACTT
GACCTGTCTGAGGTTCCACTTCCTAATCTGGCCTGGCGAAGATAAGAAAAACAGTT
TATTTAAAGTGTCTAGCAAAGTGCTTGGACCAAAATAGGACCTCTGAAATGGTTATG
GTAGTGCTGTTAAGGTGATGTTTTAAGTGCTGATGAGCACAAAGATGGGTAAGATA
TTCCTTCTGTTAAAATCTACAGTCTAATGAGAGAGAACAAGATGAATGCACAATAAC
TGTCATTCAGAACAGGATTATGAGAAGGTGTGAATTTCTGTGAAAAATCAGAACAG
GGAGTAATATGATCCCAGGTGATTGGCAGGGGGTGGGGGTCTGGATTCAACTGGA
GAGGGAGCTGGCAGGGAAGGCTTCCTGGAGGATGAGAGTTCAACAAGGGGCAGG
TGTAGGATGTGGGTGGCCAAGTGACTGGGCAGAAGGAGCTGCAGAAGTAAGACC
CCAAATCAGGAAGACAAGGGCCTGCTGAGAAACACGAGCTACAAAGTGCAAGTGC
AGGAAGAGTTGGGATGAGATTAGAAGGGGGTCTGGGGCCAGACTGTGGAAGGCC
CAAATGCCGGGCTAAGGAGTTTGTACTTAATTCAGTGGTCAACGGGGAGTCATTG
GAGGCTGTTGAGCAGGAGAGTTGCTTTCTTTACAGCTGTGCCAGACTAAATTAAAC
CTAAACAGTACTTTATAGCTGGAAAGGGAAGGCCCAGGAATAGCTCTTGACTCAGA
AACAGGCATTGGGGAAGGTAATGAGAAACAGCCGTGACTGATCAAAGCAGAGAGG
TTAATTAAATTTGTAATTATTGTGAAAGGCCATTAAAAACCCTAGTTCACTAGAGATA
ACTGCTCTAGTGGGGCTTCAAAGACAAACGCTTCTTTTAACCTTGAATAGGGGGAT
GTTTGCTTCTCTGTGGAGGAGATATGATTAAGATACTTAATAAATGGTAGATAAACA
SEQ ID NO. 58 (cDNA sequence of CLT encoding CLT Antigens 3 and 4)
AAACACACTAAGGGCTTTGTTATGGACTGAAATGTGTCCTCTCCCCAGAGTCACAG
GATTATGAAGCCATAATCCTCAATGTGACTATATTTGGAGCAGGGGCCTTTACAGA
CATAATTAAATTAAATGAGGTCATAAGAGTGGGGCCCTAGTCTGATAGGACTGGTG
TCCTTACAAGAAGAGGGAGAGTCCTCAGAGAGTCCTCTCTCTCGGCATGGACACA
AAAGAAAAGCCATGTGAGGACACAGAGAGAAGGTGGCTGTCTACGAGCTAGGAAG
AAAGGCCTCACCGGAAACCAACCCTCACAGCAGCTCCATCTTGGACTTCCAGCCT
CCGGAACTGTGAGAAAATAAATGTTTGCAATTCAGGTCGGTTGTATTTTGTGAAGG
CCATCCTAGCAAATGAATACTCCTAACATTGTCTCTTTAAGAGCTCACCAGCCTGA
GGTAGGAATCATTCCATCTGTGTTACTAATGAGACCGCTGAGGATCAAAGGGGTTT
TCCACCACATCCACTCACCTCTACATGGCGAAAACCAAGGGTTCACTCTCTGTCTT
CAGGGAGCTCCACCCAGCAGCAGCGTTTGACAGAGCTGTTCACTTCCTCTTCCTG
GAGCTGTGGCTTCCAGAGCCCATGCTCAGCAGTTCCCCTCCTTCTTCGACTGCTC
CTCTCTTAGGCTCAGAGCCACTCAGACATTGGGAAGCAAGTTTGTCAAGATGACAG
AGAACCGAGGTAATGGATTCGAGTGATGAAACAGGAAGTTCATTCATGAGTTTTTG
GCCACACCTCCAAAGTGACGACTTAGCCAGAAATGGGATAACTGGGTTTCCCTACT
TCTCTTTTATCATCCTCAATGAGAGTGACCAAATATTAGAGCTAGATGGAACCTTAG
TGAAAATCTGGCTACTCGTCCCGTCCCACCAGCCTGCCACCCATTTCAAGTTTGAA
GAGACAAAGACACATGGACCTTATGTAATTACTGGGGATTACCCCAGGAGTCTGTG
GCAAAAGTCAGCTTCTTCCCTCCCTGCTTCCCCGCCCTGTCTCTGGTACTTTCTAC
CAACACTGGGCTGTTTCTGTGATCACACTTAAGCGTACCTAACCTGCGAATGCTGT
ATAGAAGGTGCTAATGAACATGATTTAGCTTTAACACTCAGTTTTCTAAAGGGACAC
GTGGGGGCAGCAAATGTTTAGGCAAAAACAATTCCAGTTCTAGCCTCTACTGTCTA
CATATGTGTATACATTTGGGAAACGTTTGGGAAAGGGATATTTGAGAGCTTCTTTTT
CTTTTTTGTGGTTTAGTTATTTGATGATATTGAGATTGTTTCTGAGCCATGTGCTTCA
ACATCGGATTGGGGATTTCAGAAAAAGTTTTAGTCACTGTGATTCCATTTAGCTTCC
AAATGTGTCTCTGCTAAGAGACTTAAAAGCACTCATAAATAGCACGTGTGTCTTCTT
TGCAGTGTTTGCTAATTTTGAGTCACATCTTTTTAGAAAATCATGAGATTTGGTGTC
ACAGAGACTGGAATAAATATAGTCAAACTTATTGGTGAAGATTTCCTTTAGCTGTTT
TCATAATCCATTTCCATTGTTATGATTATTGATGAATAAAACATTTTCTTTAGGTAGA
TACTTCTTTTTTCCCCCCACCTTGATTTAATGTTTCCACTCTTATTGTCAAGTTTCTT
ATTACTCCCTAATAACTCTCAATAAAATAATGATTCCTGGGAGATTATTCCTGCTTTC
CTACTATCACCTGTTGATTTGAAAAGACAGAACAATACCGTAGAAGCTTCACTAATA
CATTGAAAGATAAAATGATAATACTAAATACTAAAATATGAAAAGTGATACTAAAAGT
GGAGTCCTGGCACTAGTATTTTTTTTTTTGAGTCTTTAAATTTTATTTATTTATTTTTG
AATTTTTTAAAATTATATGTTATGTTCTGGGATACATGTGCAGAACGTGCAGGTTTG
TTACATAGGTATACAGGTCTGGCACTAGTATTTTGTTGCCACAAAATATCAAGCATG
TATCCAAACTGCTCAAGACACATTAAAGACACAGGTAATCTGTAGGCATATTCAGG
CTTGTAGTTTGCATTTTTTGGTTTTCTTGTGGCTTTCAGTGCAAGTTGAGGTAATTC
ATGGGAAACAGTCACCAAAGAAGTGCCAGTATTAGAAATCCAAGAGCCATTTCTCT
AGCTTCTTCCAGAATCAAGACTTTAGAGGTAATTTCTATCAACACTGGACATTTCCT
GTCTGCAATTAACAATGAACACATAGCATTATGTTTAATTGCAACCTGTTTAAAGCA
GATTGGATGCTAAGGTTTAAGAACACTCTTCAGTCAAAAAGGTCTTTTAATCAGGTT
TTTAATCTTGAGCACAATCTAGGACACAGCATCATAGACTAACTCATTCGAGAATAG
GTGTTGTCATCTAATCCTAACCACCCCCACCACCAACAAGCTGAATAGCTCTGGGC
TCAGTATATACATTTGTACTGGGCTCAGTACACACACCTAAGCTGGGTTCAGTATAT
GCCACTTTATAGTGAGAGGCATTTTGTAATGAGAGCTCTGGGTTCACTATATACATT
TGTACTGGGCTCA
SEQ ID NO. 59 (cDNA sequence of CLT encoding CLT Antigen 5)
CTTTCATCTTTAATTTGACCAAAATGGAAACCAGGATCATGAGAATTCCTCGGGGCT
GGTGTTGAAAGGAATTTCCCCTGCTCTTGCCAGAGTCTCGAGGGGTGGCCTCCTT
CCACGGGTGAGTAACCACAAGTCCATGTGACCGAACAAACAGCATATTCTTTTGTT
CAAAAGAGAAAAACAACATTGAAGGAAATCAGCTGAAGAAAATTGAGATGAAAGCC
AGTCCACGCCTCAGCATCCTGAGGAAATGTTCTTCCTTGATGCTCTGAGCTCTCTA
AGAAGTTACCACAAAACCAAACCCATCAGAAGTTTGCAGGACGTCCTTGTTTAGAG
CTGGGAAATAAACCACGAAACAGCGCAAAGGAGAGTCCAGGCCTGCCAATGCTTC
CGCGAACTCCTCGCCCCGACCTCATCCTTCTCCAGCTCCTACCTGCAGGCCTCAG
ACAACTTTTGCAGACCTCTGGTCCGGACAATGAACAACCCATAGAACAAGATCTGA
TTTGTAATGTTTGCTGATCTCCAAAGTGTAAATACTCCCACCATGACCAATTGGAAG
CCACTGACAAGTCCTCACTAAATGCAGAATTGAGAAGAAACACGAATAGCACATCA
TTGTATGGTATTTCCACTCTACCAATGGATGTGAATAACCTCAAGAACACATAATGT
TGAGATGTGGAAAAATCATCAGGAAGCCTCTCCCCTTCACCCTGCAGTGTCTCTGC
AATGCTGGTAGAGTGGGCTGGCACAGCCCCCGCCTCTGGCCTGGTCCGGGTCCA
ACCTGCCTGCCTCCTGGGGCAGCAGCTCCCTGAACGAAGAGCCGCGGAGACCGA
AGAACTCAGTGAATCAGCAGTTCTCCCAGATGAAACGCTGGCCTGAAAGCAGCCT
CAAGAGCTTTGGCCCGTCACCTTGCCTTGCCTTCTCTTCCTTCCTCGTCCTCTGTTT
GTTCATCTTTCCTGAAAAAATTAAGTCAGCTGTTCCCCTTAACCAATTTCCCTGGCA
TTCTGAAGGGTAGGCCACATGGCCCACCTGCCAGCTACTCCCACCTGCCAAGCCT
TCCTGACTATATTTACCCTGGTACTCCCATGTCCCGGGGCTGCTTCAGCAGAGCCA
AGGACACACCCAGGTGTTTGTTTTCTAGGTCAGATTTCCTCAGCCATGGGTGTATC
TGTGCTTGTCCCTCAAAATCCTCATAGCTCCTTTCCCCACCCCAACTTCCAGGCCA
GACGGGGTTCAGGGGTGTCTCAGCTAAGGTTCCCCTGAAGCAGACACAAATTAGT
ACAAAAGGGACTTATTAGGAGGTGATCCTTAAAAATTTGGGCAGGAGAATGGAAAG
GGTGGACACAGACAAAAAGGATGCCAAGTGAGAATGTGATGATTACTGCTGCAGT
TTTCTACAGCTGCCTGTCAAACTATCCCAACACAGTGGCATTGACCAGCAGCCATT
TTCGCTATGCCCACAGCTTCTGTGGGTCAGGCGTTTGGACAGGGCAATGGGAACA
GCTTGTTTCTGTTCCATAGTGTGTGAGGGAGGCCTCAACTGGAAAACTCAAAGACC
GGGGTGGCTTGATGACTGGAGGCTGGAACCATCCGGAAGCTTCTTGAAGCTGGGT
TGACTTACATGTGGTCTTGCATGTGAGTTGGGCTTTTCACATCATGGCTGTTGGGT
TCCAAGAGGCAGTGTCTGGGGAACAAGAGTTCCAAGACACCAAGGCAGAAGCTGC
TAGACCTTTTTTGACCTCGCTTCGGATGTCAGCAATGAAGTTCTGTGGATTCAACTG
CATTCTATTGGTTACCAGCAGTCGCTTGAATCTGATTAGCTCCAAAACTGAGTGAG
CCTCTGGCAAGAGTGCATTGCAGAAGGGCACATCCAATGAGAGGCGCTGCTGTAG
TAACAGGGACCTCTCCAATGGCCTGTTTGAGTGCTCTCAGAATATGGCACCTGGGT
TCCTCCAGAGCAAGTGATCTGAGAGAGAGCAAGGAGAAGGCCACTCTGCCTTTCA
TGACTCATCTCAGAAGTCACACACTATCACAGGTTCTGTTCTGTTCATTGGAAGTGA
GTCATGAAGTCCGGCCCACACTCAAGAGAAAGGAAATTATGCTCCTTCTTTTGAAA
TGAGTGTAAGAAAGTAAAACTTTGTCAAATGTGTATGTGTGTGTGTGTGTGTG
SEQ ID NO. 60 (cDNA sequence of CLT encoding CLT Antigen 6)
GAGGCCTCACAGCCATGTGGAACTCTCTGGAGGCCAGAAGTCCAAAATCAAGATG
TTGCAGCACTGGTCCCTGGGAGGCTGTGCAGGAGAATCTGCTCTGGGCGTCTCTC
CTGGCTCCTGGTGGTGCCACAATCTTCCCTGATCCTTGGCTTTTGAAGGCATCCCC
CAGCTCTCTGCCTCATCTTCACAGGACTTCTCCCTGTGCCTGTCTGTGCCCAAATT
TCCCCTTCTATAAGGACACAGTCCTACTGCATCAGGGCCCACGCTAATCACTTCTT
CTTCACTAATTACATCGGCAAACCCCCTATTTCCAAGTTAAGTCATGTTCTGAGGTT
AGGATTTCAATACCTGAATTTTAGGGGACATAGTTCAGCCCAAAGGACCTGGGGG
GACAGAAACACCCAGGAAAGTCCACAGGTCAGCGCCTGGACCCGCCTGGCCGTG
GGACATTGCTCGGCCGAGTTCATCCTCATAAACGCTGACGTGTGCTAGAAGATACA
ATATTAGTTTCATAAACAACAGTTTCTTTTTTTGCAAACCATACCCACAGGTCCTATT
GCCACTTTTCAGTGCTGCAGCAGCTAGTCAGATGACAACTGGGTGAGAGCTTGGT
CTCGAGTGTTTCCTGTGAGGCCAGAATATTGCTCTGATTCCTCAGGGGTGACTCAG
CCCTCCGGTGGGAAATATGTGACCTCATTTGCATTTCCTAATTGAATTGCTATCAGC
TTTCCTGCAAGACTGCACCAAAAAACAGTTCTTCCAGATTCCAGCACTGCCTGCAT
TGCCCACAGAAAGGCTGCTTTTAAATAGTTGCTAAGAGAGGGCGACAAAAGAAGG
AGAAAAGTTGCCAAATTTTTATGGTTCATCTAACTGTAAAGCTGTCAGTTTTACCCA
GCTCCCCAGAGTCGCTAAAATAATGGACAGAGGAGGCGGCACTTGAAGAAACCTC
GACATAAGAGAAATGAAATTTACAGAACCAGGAAATTAGGGGTCCCCATGCACCTG
CCCTGGCAGAATCTGCTTCCAGGCTCCTCTCAATAAACACTGCCCTGGGACTCCCA
GCGCTCTGTAAACTGATGCTTTGAAGGAGGCCATTTGTTTCTCCAGAACCTTCTGC
CAGCAGAACTCTTGCTTACCACGTGCCCCCATCAGAACTCCCACCTTCTCTAAACA
CAGTTAGGTAGCCCTCTGCATGCATTTGTTTAAAAAAATTAAATTGTGAGTAATAGA
CTATAAGTACAACAGCGTACAGAACACCAGCTAAAAAAATGATGGCAGAATGAGCA
CCTGTCTTGCCACAAGACTCCCTCCCACCCTCTGGTGTCCCCCAGCATGGCTTCCT
GCTGCCCCCTGAAAGGAACCCTCAACCGACTGTAACTCTGCACCTCAGTGCCACC
CGAATTTGGACTTCATGGGAGTGCGGTCACTCGGCTGACACGTGTTCCTGCTGCC
AGGCACCTGCCTGCTCTCCTTCTTTGGTCTGTTTGTGAGATTCGCTGGCTTTGTTG
CTTCTAGCTGCAGTTTGTTCACTTTTACTGCCGTATAATATTCCACTTTAGGAGCCC
ACTGCAATTTATTGACTCATTGCAATGGTGAGGGCATGTGTGTTGTTTCCAGCTTTT
GGTGATTATGCGTAAGATTTCTCTTAACGTCCCGTACAGAGTTCTTGGTATTACCTG
CCTATACTGCTATGGGCATACACCAAAAACGAGGCATATGTATATTTGACTTTAATA
GTAATTCTGAATTGCTGTCTAAAGTGTTTGTACCATTGTACACTCCCATCAAGAGTA
TACGAGTGCTGCAAATGTCCTTTCCCAGACTCTGCCTTATTGTCTCATTTTTAAGAT
ATATGTTTTGATGATCAGAAGTTCTTTTTTTTTTTTTTTTTTTTTGACAGGCTTTTGCT
CTGTTGCCCAGGCTGGAGTGCAGTGGCATGATCTCAGCTCACTGCAACCTCTACC
TCTCGGGCTCAAGTGATTCTCCTGTTTGAGACTCCCTAGTAGCTGGGATTACAGGT
GTGCACCACCATGCCTAGCTAATTTTTGTATTTTTAGTGGAGACAGGGTTTCACCAT
GTTAGCCAGGCTGGTCTCGAACTCCTGACCTTGGGTGATCTGCCCACCTTGGCCT
CCCAAAGTGCTGGGATTACAGGCGTGAGCCACCGCACCTGGCAGAAGTTCTTATT
TTTAATAAAGTCCAGTGATCATTCTTTCCCGTGTTTCACGCTTCACGTGCCATGTGC
GAGGAAGCTTTCCACACCCGGGGGTCAGTTCACTCCCAGCTGCATTGCTTTGCCT
TTCCTGTTTAGGTTTTCAGTCCACCTCAAATGGATTTTGGTTGTGGTGTGAGGTAG
GGGTCATTTTACTTTTTTCTTAAAGATGCCCCATTGGCCCAGCATCATTTATTGAAA
AGACAGTCTTTCCTCTGTTGTTTCTGTAGAAATGGGCAAACTTTCCCCCAAATGGC
CAGATGGTAAATATTTTAGGCATCTCAGGTTAAGGGGCAAAATTGAGGACATCGTA
TAGGTACTTACATAACCCTTTAAAATGGAACCATTTAAAAATGTAAAAACCAGTCTTA
GCTTGCTGGCCATAAAAGGCCTGTGGGCTACAGCATGCTGGCACCTGCTCTACAG
CACTGCTCTTATAATAAAAAATCAAGCGTCTGCACCCTGGGCATTTGACAAGGCTC
ATCAGATTGTAAAGATGAACTGGGTGGATTTTGTAGTATGAGAGTTATACGTCCAT
GAAGCTGATTTTTAAACACATTACATGTCTTTTTCTGGGCTCATTATTTTTGTTCCAA
GTGTCTATCTAATCATCTTTGCTTCAAAATGACACCGTTTTAATGGCTGTAGTTCAG
TGGTTCTCAAAAGGGGGCAGTTTTGCCCCCCAGGGAACATTTGGCAGTGGCTGGA
AAGAGCTTTGGTTGCATATGGAGGTTATGGGGCTTTACCTGGCGTTTAGTGGGTAG
AGGCCAGGAGTCTGGCTTACTATCCTGTAGCACACAGGACAGCCCCAACAACTTG
TCTGGAAGGATGTGTCCTCCCATCTTTTTCTTGTTCTAGGCTACCTTCGCCCTTTCC
ATTTCCATTAAAAAAT
SEQ ID NO. 61 (cDNA sequence of CLT encoding CLT Antigen 7)
GTGTGCCCTTTGCAGCAAAGAGCGTGGTTCCTCCTCTTCCTGGGTGTAAACTGCG
ATTCAAAAAGCCAGGTGGGAAGCCCTGTAGTAGGGACTCTGGCTTTGTCCCTGTTT
CCCCCTTTTCTTCCTCTTCACCCACTAAAACCCTGTTTTACTCACGGTTCAAATTGT
TTGGCAGCCTGAATTTTCATGGCCATGGGACAAAGAACCCCGTCTTTAGCTGAATT
AAGGAAAAGTTCTGCAACATTTTTGACGTGCAACTTGGGGGCTCAAGCGGAGAAG
CGAAGCAGAGCCCCTGGAAAACTCACATATGTGTCAACAATAGTCCTTGATGCCCC
TGTAACAAAACTTGAGCAAGGCCTGGTGATGAAGAGATACAAGATTGTCACCCAAG
GATTTGATTATACCTCTGTTGAAAGTTAATGCACACTCAACCGAATATGGCAATTGG
TACTCTCAATTCTATATTCTCAACAATTACAGTGAAGATACAAGTAGAATACAATCAC
TGCCACATTTTTTGAAATTCTGTGTAAACTGTAATATTCTGTCAACATTAATGACATT
TGAAGTGTCCTGTCAAAACAATTGCAGCTACTTTATGTATAAAACATATTAAATAGG
CTCATCCAACTTGCATTCTTTATTAAGCTTATTTTCATATTTTTTCCTATGGATGAACT
TAAAAATAATTTTGTTTCTTAATTAAGATTCTATGCATGAAGATGCTGAATAATTTAA
GACAATTGTTCATTCAAATAGTTGCTAATTACACCCTCCTGGCATAGTTATTGTATTA
TTACTACATTTAGGAATAATATGCTGTACTACTTGGACTTGAAAATGTTTCTGACATT
TTAATGAACACACTACTTAGTTATATTTTACAAGGGTTTTCAGTGAACCACAGAGGA
TTAAAAAATGTCATTCAAGGGTTGTAGATAATTAAACTGACTGAATATAAGAAGCCT
CATATGGAAGTGAAAATTATGTATGAATTTTGTTGAGCTGGAAATGTGTTTTACTAA
ATGACTTCAGATTTGTTACTTTTAAATACAAGATGAAGGGAATCAAGGGGATACTTT
CTTCTCACCTCAATTTGTTCCATTTGCATAAGCTATTTTATCTTTCAATATCCCTTTT
GATATTTCATTTTGGCCCTTGAATACACTAAAGATTATTTAAAATAAATAAATGTTGA
CTTTGAAATACTGCTATATATATTCATTGCAATACATCAGGTGGGAAATTGGTGCAA
TTCAGTCACACACACAACCTCATAGATCAGATTTCCAGTTAAGTTTTAATAGAAAGA
GATTTTAGATAGACATGACTTTCAATAAAATGGAAGAATTTTTATTAGTTTCAAAATA
ATGATTTTACTTGCTTATCAACACAGTTGACCTTTCCCCACAGTACTATGATCTTCTT
AGTACCTCTGCTATAATTATATTAATGTCTTCCTATATCTAGAAATTTGATTATTATTC
TATTTTTATGGATCATTAAATTTTTCTATTCAAGAATGATCCCTATAGTCAACTTCCT
GAGGGATTTCTGCTTGCTATTTTCTGTTTCAATTGTGTGCATTTCATATTTACTATTA
AAGATTTTAAATAATGACACATTGTTTAAGCGGTTGTAAGTCTGATTGTTATAAAGCT
CTCTGGAGAATTCTGTAAGCTTTCACATCTGATGGAGAACTTCAATGAGAACATCTT
TAATGTGGAATTCTTGGACAAGAAACTACAGATTATCCATGGTTCAGAAGATATGAT
GAATTAGTAACTATTTTTCCGTACATAAAAAGTCAATCTTCCTAACCAGTTTGTTGTT
TTAGCTAAATGAATCGGTCAAATATTTTAGTTATATTAGCAATATTTTGTCTCTAAAA
TATCTTGACATAAAAC
SEQ ID NO 62 (cDNA sequence of CLT encoding CLT Antigen 8)
GGGTCCCTGGCTCGGCTCTACCCCCATGGATCTAGGTGAGGACAGGCGCTCCTG
CTTTCCCGCCCAAATGTTGTATTTTCCAAGCCTACCCTTGCCGGCCACGCCCCCAA
CCTGGGCCTATAAAAACCGCCCCCCGCAGGCCCTAGCGGGCAGACACACTGAAG
TCGCTAGACATTTGAGGAACACATCCGGGGAAGAAGACACAGGTGGCTGGTCATG
GAGAGCCCGCTGGGGGAAGAGCACACAGACAGGCACCGGCAGGCCATTGACCAG
CGGGACAAGGTGGAGTTTGGCTGGGGCAGTGGGAGGAGAGCTGGGGCCGCCGA
GCTGCCCTACTCCAGGGGAAAACCACCTCCCTTCTCGCTCCCCCATCACCGGAGA
GCTACTTCCACTCAATACAACTTTGCACTCATTCTCCAAGCCACGTGTGACCAATTT
TTCCGGTACACCAAGGCGAGAAATCCGGGGATACAGAAAGCCCTCTGTCCTTGCG
ATAAGGTAGAGGGTCCAATTGAGCTAACACAAGCTGCCTATAGACGGCAAACTAAG
AGAGCACCCAGTAACACACGCCTGCTGGGGCTTCAGGAGCACACACGTCCACTG
GGGCTTCGGGAGCTGTAAACAGTCAGCCCTAGACACTGTCGTGGGATCGGAGCC
CCACAGCCTGCCTGTCTGTATGCTCCTCTAGAGGTCTGAGCAGCGGGGCGCTGAA
GAAACGAGCCACACTCCCATCACACGCCCTGAGAGGAGGACAAGGGAACCCGTA
CCGTTTCACTTGTGATAGAGATAAAGTTATTATTGTTGTAATTTTAACTTATAGAACT
ATAATATAATGGTACAATGATATATATTGACAATATTCCTCTTTCACCCACATTTTGT
ATCTGTGTTCAAGATTAAATGAAAAAGGATATTCTCAAAAAGCTAGCAAAACCAAAA
GCAAGACGTTATGCCAAATGTAAACATATTTCTGTTTAGCAAATAGCAGGAGTGTAT
AAAACATTTCTCTTTTCACATAACAGAATGTTCTATGCTTACTGTATTAGTTAACAAA
TTCAAGTCTGTTTATTTTGTTTGAAATTCCACTTCATCCATAAATTACAGCATTACAC
AATAACACCAGAAGGACAATATCACCATTGTTTGCTTTTACAGTCATCTCAGCCCAC
AAAATGCTTCCCATTGTCAGCTTGCTATCATCAAGCTATTGTTGTTTTCCTCTTTCTG
GGGTCTTGTATTAATATCATTCAAATTATTTAGACACTCAACAGTGTTTTTGCTATCA
GTGCAAACCTCTAAAGAGCTGGAGCCCCAGCGTGATGACCAAATAACCCTTGACT
ATTTATCTTGCCGTAAGTCATTATTTCCTGAGGCCCTGGAGAAGCTGTGTTGCCAT
GTGTGCACTGCAGGGACGGGGAGCTTCTCCTGCCGGAGCTGGTTTGTTCCACTGG
ACAATGAGCCCTTTTCTGCTTGGCTCTCTGTATGGGCACATACACAATGAGGCGGT
TTAGGGAAGAGGGGTGATGTGGGTCATTGATAACAACAATCCCCGAACTTCTTAAA
GAATTGTTGAGCCCCCTAAAAATATGTTGTCTTTATGAATTATCCTTAACCCTTTTTA
ATTGCATAAAAACTTCAGGCACTTGAAAAAAATTAAAAAACGAAAAGTAAGTCTGTC
TCTAGTATCCCTCTTCTCCTTTGTAGAATTACATCCTTTATTCACTCTGCCACTATTT
ATTATGTGCCTCCTGTGTGTAAGACATACTGTTAGTCATTGGAAATACAGAAATGAA
TGGGACAGACACACTTCTTGCCCTCATGGAACTTAGGGCCTAATGGGAGATACAAT
GTTAAGGTTGTTTCTAGTGTTTTTTTTTTTTTTTAAGAATATGTTCTACATGTATACAT
GTAATATGTATTCTCTCCCATTTTTAAAAAACATAAATGGTGGTTTACTATGTGTACG
CTTTTTGGTTTTGCATTATTCTTTTAACAGTATGTAGAGGATGCATTTTCTACTGTAT
AGTGTTCTGTCCCCCTTAAATAGCACTCTGATTTTATTTTGGGGGGGAATCACGACT
TTCTAATTTTGCATACTCCTTGTGGGATTGTAAATCAGGTCCCCTGTCCTCAAGTAG
CCAATGGTTAGGTATGCAACCCAGCTTATCTCTCTTTAGACTCAATCTCAAGCAGAT
CCAGAGTTACTCAGGATCAGAACAATATTTGAAAGGCATTACCAGAATCCAGACAA
GATGATGGAGCAATACCTGATGCCCAGTGGTCTAGGGTAGCCGATTCCTGTTCTG
CTCTCCAGGCTCCTGTCCATTCTGTGGATCAACTCATATTGCTCCAATTCATTTTGT
TTTGCTTGCATAAGCCAGAATTAACTTCTGTTGCTTGTAAACAAAGAGAGTTAACCA
AAGACATACAGTATATCAGAGTATAGAGACCTACTTTACTCTCGGTCATTGCCTGCA
TGAGTCTTCTTTATATGGATGTATCACAGTTTATTTAACCACTCCCTTGTAAGTGGA
CATTTAAGTCTAGTGTTCTGTAAATAAAAGGTCAGAATATACGTCTGTATACAGTAT
ATATTCTTGCATATCCACCTTTGGACAGATGTGTGAATGTGTTTTGTAGAAATACAT
TTGTAGAAATGCAACTGCTGGGTCAAAGAATTAGTAGATTTTTAATAACATCAAACA
GCGTTGAAGGCCCCCATATAAGAATAACAACTACTGACTGAAGACATAACTAATTA
AAAAAATTAATTACAGCTTATTTGTAATAACTTCTTATTGTCACTGAGTGAAAAGGTA
ATTCTCGTTGAATTTACGAAAAGTGACTAATAGGAAATTTAGAAAACTCAGAGAATA
TATAAAAACACAAAGAAAAGCCAGCCACCAAGTCGCTTATAATTCTCTCACCAACA
GTGGCAGAATTACATTTAGTCATCATCATTATTCTTACATCCAGTTTTATAGTTATTT
TTGAAGAGGATTATTATCAACTATCAACTCTGTCATAGCTGGAAGTAGAGGCCACT
AAAACAGATTTCTTAAACTCCAAGTACTGTATATGGATTTTCTAC
SEQ ID NO. 63 (cDNA sequence encoding CLT Antigen 1)
ATGTGGAACTTCTTCAGGAGAGAATTAACATCCAATGGATTCCCAGAAAACTTTTCC
CTCGATGTACCAGCAAACACCTACAATGCCCTGAAAAGCCGCCTCTGCGACCCCA
ATGCAGATCACACGTCCTGTCCCAGCCCCTGCAGCCTCCACGCGGCGGGTGCACT
GCCAGGCACGGGAAGGCAGCGCTGGCGAGTAGAACTGGCCCATCTCGCAGATAG
GAAGCTGAGCCTCAGGGACGTTTCACGCCTTCGTCAAGGTGGTGAGAGGAGGAG
CGGGATTGCCGTGAAGGTGGTGAGAGGAGGAGCGGGGTTTGCTGCCCGACTTCA
GGGATCTGTCACCCTCGTCCAGCAGGGTTGGTTCTTCCCGAGGCTGGGAGGATGC
CAAGCCTGGTGGAGGATGGGGGCGGTGGTGTGGTGTGGGGAGCTTCTGACTTGC
ACATCC
SEQ ID NO. 64 (cDNA sequence encoding CLT Antigen 2)
ATGACTGGTGTTCTTATAAGAAGAGGAGATTTGGTCACAGACATGGTTGCATGCAG
AATAAAGACTTTTCGAGGACACACTGAGAAGGCAGCCATCTGCAAAACAAGGAAAG
AGTCCTCAGCAGAAACCAGTCCTGCAGACTCCTTGATCTTGGACTTCCAGCCACTG
CAATTGATGTCAAGCTTCAGCACCCTTGCATCTCTGGATAAA
SEQ ID NO. 65 (cDNA sequence encoding CLT Antigen 3)
ATGAATACTCCTAACATTGTCTCTTTAAGAGCTCACCAGCCTGAGGTAGGAATCATT
CCATCTGTGTTACTAATGAGACCGCTGAGGATCAAAGGGGTTTTCCACCACATCCA
CTCACCTCTACATGGCGAAAACCAAGGGTTCACTCTCTGTCTTCAGGGAGCTCCAC
CCAGCAGCAGCGTT
SEQ ID NO. 66 (cDNA sequence encoding CLT Antigen 4)
ATGGCGAAAACCAAGGGTTCACTCTCTGTCTTCAGGGAGCTCCACCCAGCAGCAG
CGTTTGACAGAGCTGTTCACTTCCTCTTCCTGGAGCTGTGGCTTCCAGAGCCCATG
CTCAGCAGTTCCCCTCCTTCTTCGACTGCTCCTCTCTTAGGCTCAGAGCCACTCAG
ACATTGGGAAGCAAGTTTGTCAAGA
SEQ ID NO. 67 (cDNA sequence encoding CLT Antigen 5)
ATGCTTCCGCGAACTCCTCGCCCCGACCTCATCCTTCTCCAGCTCCTACCTGCAG
GCCTCAGACAACTTTTGCAGACCTCTGGTCCGGACAATGAACAACCCATAGAACAA
GATCTGATTTGTAATGTTTGCTGA
SEQ ID NO. 68 (cDNA sequence encoding CLT Antigen 6)
ATGTGGAACTCTCTGGAGGCCAGAAGTCCAAAATCAAGATGTTGCAGCACTGGTC
CCTGGGAGGCTGTGCAGGAGAATCTGCTCTGGGCGTCTCTCCTGGCTCCTGGTG
GTGCCACAATCTTCCCTGATCCTTGGCTTTTGAAGGCATCCCCCAGCTCTCTGCCT
CATCTTCACAGGACTTCTCCCTGTGCCTGTCTGTGCCCAAATTTCCCCTTCTATAAG
GACACAGTCCTACTGCATCAGGGCCCACGCTAA
SEQ ID NO. 69 (cDNA sequence encoding CLT Antigen 7)
ATGGCCATGGGACAAAGAACCCCGTCTTTAGCTGAATTAAGGAAAAGTTCTGCAA
CATTTTTGACGTGCAACTTGGGGGCTCAAGCGGAGAAGCGAAGCAGAGCCCCTGG
AAAACTCACATATGTGTCAACAATAGTCCTTGATGCCCCTGTAACAAAACTTGAGCA
AGGCCTGGTGATGAAGAGATACAAGATTGTCACCCAAGGATTTGATTATACCTCTG
TTGAAAGTTAA
SEQ ID NO. 70 (cDNA sequence encoding CLT Antigen 8)
ATGTGTGCACTGCAGGGACGGGGAGCTTCTCCTGCCGGAGCTGGTTTGTTCCACT
GGACAATGAGCCCTTTTCTGCTTGGCTCTCTGTATGGGCACATACACAATGAGGCG
GTTTAG
SEQ ID NO. 71 (linker sequence used in CLT Antigen Fusion Proteins 1, 2, 3 and 4)
GGG
SEQ ID NO. 72 (linker sequence used in CLT Antigen Fusion Proteins 1, 2 and 4)
GGGG
SEQ ID NO. 73 (linker sequence used in CLT Antigen Fusion Proteins 1 and 3)
KGG
SEQ ID NO. 74 (linker sequence used in CLT Antigen Fusion Proteins 1, 3 and 4)
GGKGG
SEQ ID NO. 75 (linker sequence used in CLT Antigen Fusion Proteins 1, 2, 3 and 4)
GGGKGG
SEQ ID NO. 76 (polypeptide sequence of CLT Antigen Fusion Protein 1)
MWNFFRRELTSNGFPENFSLDVPANTYNALKSRLCDPNADHTSCPSPCSLHAAGALP
GTGRQRWRVELAHLADRKLSLRDVSRLRQGGERRSGIAVKVVRGGAGFAARLQGSV
TLVQQGWFFPRLGGCQAWWRMGAVVWCGELLTCTSGGGTGVLIRRGDLVTDMVAC
RIKTFRGHTEKAAICKTRKESSAETSPADSLILDFQPLQLMSSFSTLASLDKGGKGGMW
NSLEARSPKSRCCSTGPWEAVQENLLWASLLAPGGATIFPDPWLLKASPSSLPHLHRT
SPCACLCPNFPFYKDTVLLHQGPRGGGKGGMAMGQRTPSLAELRKSSATFLTCNLGA
QAEKRSRAPGKLTYVSTIVLDAPVTKLEQGLVMKRYKIVTQGFDYTSVESKGGMAKTK
GSLSVFRELHPAAAFDRAVHFLFLELWLPEPMLSSSPPSSTAPLLGSEPLRHWEASLS
RGGGGMCALQGRGASPAGAGLFHWTMSPFLLGSLYGHIHNEAV
SEQ ID NO. 77 (polypeptide sequence of CLT Antigen Fusion Protein 2)
MWNSLEARSPKSRCCSTGPWEAVQENLLWASLLAPGGATIFPDPWLLKASPSSLPHL
HRTSPCACLCPNFPFYKDTVLLHQGPRGGGKGGMCALQGRGASPAGAGLFHWTMS
PFLLGSLYGHIHNEAVGGGKGGMAMGQRTPSLAELRKSSATFLTCNLGAQAEKRSRA
PGKLTYVSTIVLDAPVTKLEQGLVMKRYKIVTQGFDYTSVESGGGGTGVLIRRGDLVTD
MVACRIKTFRGHTEKAAICKTRKESSAETSPADSLILDFQPLQLMSSFSTLASLDKGGG
MAKTKGSLSVFRELHPAAAFDRAVHFLFLELWLPEPMLSSSPPSSTAPLLGSEPLRHW
EASLSRGGGGMWNFFRRELTSNGFPENFSLDVPANTYNALKSRLCDPNADHTSCPSP
CSLHAAGALPGTGRQRWRVELAHLADRKLSLRDVSRLRQGGERRSGIAVKVVRGGA
GFAARLQGSVTLVQQGWFFPRLGGCQAWWRMGAVVWCGELLTCTS
SEQ ID NO. 78 (polypeptide sequence of CLT Antigen Fusion Protein 3)
MWNFFRRELTSNGFPENFSLDVPANTYNALKSRLCDPNADHTSCPSPCSLHAAGALP
GTGRQRWRVELAHLADRKLSLRDVSRLRQGGERRSGIAVKVVRGGAGFAARLQGSV
TLVQQGWFFPRLGGCQAWWRMGAVVWCGELLTCTSGGKTGVLIRRGDLVTDMVAC
RIKTFRGHTEKAAICKTRKESSAETSPADSLILDFQPLQLMSSFSTLASLDKGGGMNTP
NIVSLRAHQPEVGIIPSVLLMRPLRIKGVFHHIHSPLHGENQGFTLCLQGAPPSSSVGG
KGGMAMGQRTPSLAELRKSSATFLTCNLGAQAEKRSRAPGKLTYVSTIVLDAPVTKLE
QGLVMKRYKIVTQGFDYTSVESKGGMAKTKGSLSVFRELHPAAAFDRAVHFLFLELWL
PEPMLSSSPPSSTAPLLGSEPLRHWEASLSRKGGLPRTPRPDLILLQLLPAGLRQLLQT
SGPDNEQPIEQDLICNVCGGGWNSLEARSPKSRCCSTGPWEAVQENLLWASLLAPG
GATIFPDPWLLKASPSSLPHLHRTSPCACLCPNFPFYKDTVLLHQGPRGGGKGGMCA
LQGRGASPAGAGLFHWTMSPFLLGSLYGHIHNEAV
SEQ ID NO. 79 (polypeptide sequence of CLT Antigen Fusion Protein 4)
MWNSLEARSPKSRCCSTGPWEAVQENLLWASLLAPGGATIFPDPWLLKASPSSLPHL
HRTSPCACLCPNFPFYKDTVLLHQGPRGGGGMNTPNIVSLRAHQPEVGIIPSVLLMRP
LRIKGVFHHIHSPLHGENQGFTLCLQGAPPSSSVGGGKGGMWNFFRRELTSNGFPEN
FSLDVPANTYNALKSRLCDPNADHTSCPSPCSLHAAGALPGTGRQRWRVELAHLADR
KLSLRDVSRLRQGGERRSGIAVKVVRGGAGFAARLQGSVTLVQQGWFFPRLGGCQA
WWRMGAVVWCGELLTCTSGGGGMLPRTPRPDLILLQLLPAGLRQLLQTSGPDNEQPI
EQDLICNVCGGGGMAKTKGSLSVFRELHPAAAFDRAVHFLFLELWLPEPMLSSSPPSS
TAPLLGSEPLRHWEASLSRGGGGMCALQGRGASPAGAGLFHWTMSPFLLGSLYGHI
HNEAVGGGKGGMAMGQRTPSLAELRKSSATFLTCNLGAQAEKRSRAPGKLTYVSTIV
LDAPVTKLEQGLVMKRYKIVTQGFDYTSVESGGGGTGVLIRRGDLVTDMVACRIKTFR
GHTEKAAICKTRKESSAETSPADSLILDFQPLQLMSSFSTLASLDK
SEQ ID NO. 80 (codon-optimised cDNA sequence encoding CLT Antigen Fusion
Protein 1)
ATGTGGAATTTCTTCCGGCGCGAGCTGACCAGCAACGGCTTCCCTGAGAACTTCA
GCCTGGACGTGCCCGCCAACACCTACAATGCCCTGAAGTCCAGACTGTGCGACCC
CAACGCCGATCACACCAGCTGTCCATCTCCATGTTCTCTGCATGCCGCTGGCGCT
CTGCCTGGAACAGGCAGACAAAGATGGCGCGTGGAACTGGCCCATCTGGCCGAT
AGAAAGCTGAGCCTGAGGGATGTGTCCCGGCTGAGACAAGGCGGCGAGAGAAGA
TCTGGAATCGCCGTGAAGGTCGTCAGAGGCGGAGCTGGATTTGCCGCTAGACTGC
AGGGAAGCGTGACCCTGGTTCAGCAAGGCTGGTTCTTCCCTAGACTCGGCGGTTG
TCAAGCCTGGTGGCGAATGGGAGCTGTTGTTTGGTGCGGCGAGCTGCTGACCTGT
ACATCTGGCGGAGGAACAGGCGTGCTGATCAGAAGAGGCGACCTGGTCACAGAC
ATGGTGGCCTGCAGAATCAAGACCTTCAGAGGCCACACAGAGAAGGCCGCCATCT
GCAAGACCCGGAAAGAGTCTAGCGCCGAGACAAGCCCTGCCGACTCTCTGATCCT
GGACTTCCAGCCTCTGCAGCTGATGAGCAGCTTTAGCACACTGGCTAGCCTGGAC
AAAGGCGGAAAAGGCGGCATGTGGAACAGCCTGGAAGCCAGATCTCCCAAGAGC
CGGTGTTGTAGCACAGGCCCTTGGGAAGCTGTGCAAGAGAATCTGCTGTGGGCCT
CTCTGCTTGCTCCTGGCGGAGCCACCATCTTTCCAGATCCTTGGCTGCTGAAGGC
TAGCCCCAGCTCTCTGCCTCATCTGCACAGAACAAGCCCCTGCGCCTGCCTGTGT
CCTAACTTCCCATTCTACAAGGACACCGTGCTGCTGCATCAGGGCCCTAGAGGTG
GTGGAAAAGGTGGAATGGCCATGGGCCAGAGAACACCTTCTCTGGCTGAGCTGAG
AAAGAGCAGCGCCACCTTCCTGACCTGCAATCTGGGAGCCCAGGCCGAGAAGAG
ATCTAGAGCCCCTGGCAAGCTGACCTACGTGTCCACCATTGTGCTGGACGCCCCT
GTGACCAAGCTCGAACAGGGACTCGTGATGAAGCGGTACAAGATCGTGACCCAGG
GCTTCGACTACACCAGCGTGGAATCTAAAGGCGGAATGGCTAAGACCAAGGGCAG
CCTGAGCGTGTTCAGAGAACTGCATCCTGCCGCCGCTTTCGACAGAGCCGTGCAC
TTCCTGTTTCTGGAACTGTGGCTGCCCGAGCCTATGCTGTCTAGCAGCCCTCCTAG
CTCTACAGCCCCTCTGCTGGGATCTGAGCCTCTGAGACACTGGGAAGCCAGCCTG
TCTAGAGGCGGTGGCGGAATGTGTGCTCTGCAAGGCAGAGGCGCTTCTCCTGCTG
GTGCCGGACTGTTCCACTGGACAATGAGCCCATTTCTGCTGGGGAGCCTGTACGG
CCACATCCACAATGAGGCCGTCTGA
SEQ ID NO. 81 (codon-optimised cDNA sequence encoding CLT Antigen Fusion
Protein 2)
ATGTGGAACTCCCTAGAAGCGAGATCCCCGAAATCTAGATGTTGTTCTACTGGACC
GTGGGAAGCCGTACAAGAAAATCTACTATGGGCCTCTCTACTAGCACCAGGTGGT
GCAACAATTTTTCCAGATCCGTGGCTATTGAAGGCCTCGCCATCTTCTTTACCGCA
TCTACACAGAACATCCCCGTGTGCTTGTCTATGTCCGAACTTTCCGTTCTACAAGG
ACACCGTACTACTACATCAAGGACCAAGAGGTGGTGGAAAAGGTGGAATGTGTGC
ACTACAAGGAAGAGGTGCTAGTCCAGCTGGTGCTGGATTATTCCATTGGACAATGT
CCCCGTTCCTACTAGGATCTCTATACGGACACATCCACAACGAAGCTGTCGGAGG
TGGAAAAGGTGGAATGGCTATGGGACAAAGAACACCATCTCTAGCGGAGCTAAGA
AAGTCCTCTGCGACATTCCTAACCTGTAACTTGGGAGCGCAAGCGGAGAAAAGAT
CTAGAGCACCTGGAAAGCTAACCTATGTCTCCACCATAGTACTAGATGCGCCGGTC
ACAAAGCTAGAACAGGGACTAGTAATGAAGAGATACAAGATCGTCACCCAGGGATT
CGACTACACCTCTGTAGAATCTGGTGGTGGTGGAACCGGTGTCCTAATTAGAAGA
GGAGATCTAGTCACCGATATGGTCGCGTGTAGAATCAAGACATTCAGAGGACATAC
AGAGAAGGCGGCGATCTGCAAGACAAGAAAAGAATCTTCTGCGGAAACCTCTCCG
GCGGACTCTCTAATCTTAGATTTTCAGCCGCTACAGCTAATGTCCTCCTTCTCTACA
TTGGCCTCGCTAGACAAAGGTGGTGGAATGGCGAAAACGAAGGGATCCTTGTCCG
TATTCAGAGAACTACATCCAGCTGCGGCTTTCGATAGAGCGGTACATTTCCTATTC
CTAGAGCTATGGCTACCGGAACCGATGCTATCTTCTAGTCCACCATCTTCTACAGC
GCCGCTATTAGGATCTGAACCGCTAAGACATTGGGAAGCGAGTCTATCTAGAGGT
GGTGGTGGAATGTGGAACTTCTTCAGAAGAGAGCTAACCTCCAACGGATTCCCCG
AGAACTTCTCTTTAGATGTACCGGCGAACACCTACAACGCGCTAAAGTCTAGATTG
TGTGATCCGAACGCGGACCATACTTCTTGTCCATCTCCGTGTTCTTTACATGCTGC
TGGTGCTTTACCTGGAACGGGTAGACAAAGATGGAGAGTAGAACTAGCGCACCTA
GCGGACAGAAAGCTATCCCTAAGAGATGTCTCCAGACTAAGACAAGGTGGAGAGA
GAAGATCTGGAATCGCGGTCAAAGTAGTCAGAGGTGGTGCAGGATTTGCTGCGAG
ATTACAGGGATCTGTAACCTTGGTACAGCAAGGATGGTTCTTCCCAAGACTTGGAG
GATGTCAAGCTTGGTGGAGAATGGGAGCTGTAGTTTGGTGCGGAGAACTATTGAC
ATGTACATCTTGA
SEQ ID NO. 82 (codon-optimised cDNA sequence encoding CLT Antigen Fusion
Protein 3)
ATGTGGAATTTCTTCCGGCGCGAGCTGACCAGCAACGGCTTCCCTGAGAACTTCA
GCCTGGACGTGCCCGCCAACACCTACAATGCCCTGAAGTCCAGACTGTGCGACCC
CAACGCCGATCACACCAGCTGTCCATCTCCATGTTCTCTGCATGCCGCTGGCGCT
CTGCCTGGAACAGGCAGACAAAGATGGCGCGTGGAACTGGCCCATCTGGCCGAT
AGAAAGCTGAGCCTGAGGGATGTGTCCCGGCTGAGACAAGGCGGCGAGAGAAGA
TCTGGAATCGCCGTGAAGGTCGTCAGAGGCGGAGCTGGATTTGCCGCTAGACTGC
AGGGAAGCGTGACCCTGGTTCAGCAAGGCTGGTTCTTCCCTAGACTCGGCGGTTG
TCAAGCCTGGTGGCGAATGGGAGCTGTTGTTTGGTGCGGCGAGCTGCTGACATGT
ACAAGCGGCGGAAAAACCGGCGTGCTGATCAGAAGAGGCGACCTGGTCACAGAC
ATGGTGGCCTGCAGAATCAAGACCTTCAGAGGCCACACAGAGAAGGCCGCCATCT
GCAAGACCCGGAAAGAGTCTAGCGCCGAGACAAGCCCTGCCGACTCTCTGATCCT
GGACTTCCAGCCTCTGCAGCTGATGAGCAGCTTTAGCACACTGGCTAGCCTGGAC
AAAGGCGGCGGAATGAACACCCCTAACATCGTGTCCCTGAGAGCCCACCAGCCTG
AAGTGGGAATCATCCCTAGCGTGCTGCTGATGCGGCCCCTGAGAATCAAAGGCGT
GTTCCACCACATTCACAGCCCTCTGCACGGCGAGAATCAGGGCTTCACCTTGTGT
CTGCAAGGCGCCCCTCCTAGCAGTTCTGTTGGTGGCAAAGGCGGAATGGCCATG
GGCCAGAGAACACCATCTCTGGCCGAGCTGAGAAAGAGCAGCGCCACCTTCCTGA
CCTGTAACCTGGGAGCCCAGGCCGAGAAGAGATCTAGAGCCCCTGGCAAGCTGA
CCTACGTGTCCACCATTGTGCTGGACGCCCCTGTGACCAAGCTCGAACAGGGACT
CGTGATGAAGCGGTACAAGATCGTGACCCAGGGCTTCGACTACACCAGCGTGGAA
TCTAAAGGTGGCATGGCCAAGACCAAGGGCAGCCTGTCCGTGTTCAGAGAGCTGC
ATCCTGCCGCCGCTTTCGATAGAGCCGTGCACTTCCTGTTTCTGGAACTGTGGCTG
CCCGAGCCTATGCTGTCTAGCAGCCCTCCATCTAGCACAGCCCCACTGCTGGGAT
CTGAGCCTCTGAGACACTGGGAAGCCAGCCTGAGCAGAAAAGGCGGCCTGCCTA
GAACACCCAGACCTGACCTGATTCTGCTGCAGCTGCTGCCTGCTGGACTGAGACA
ACTGCTGCAGACAAGCGGCCCTGACAACGAGCAGCCTATCGAGCAGGACCTGATC
TGCAATGTGTGCGGAGGCGGCTGGAACAGCCTGGAAGCCAGATCTCCTAAGAGC
CGGTGCTGTAGCACAGGCCCTTGGGAAGCTGTGCAAGAGAATCTGCTGTGGGCCT
CTCTGCTTGCTCCTGGCGGAGCCACCATCTTTCCAGATCCTTGGCTGCTGAAGGC
TAGCCCCTCTAGCCTGCCTCATCTGCACAGAACAAGCCCCTGCGCCTGCCTGTGT
CCTAACTTCCCATTCTACAAGGACACAGTGCTGCTGCATCAGGGCCCTCGAGGTG
GCGGAAAAGGTGGAATGTGTGCCCTTCAAGGCAGAGGCGCTTCTCCTGCTGGTGC
CGGACTGTTCCACTGGACAATGAGCCCATTTCTGCTGGGGAGCCTGTACGGCCAC
ATCCACAATGAAGCCGTCTGA
SEQ ID NO. 83 (codon-optimised cDNA sequence encoding CLT Antigen Fusion
Protein 4)
ATGTGGAACTCCCTAGAAGCGAGATCCCCGAAATCTAGATGTTGTTCTACTGGACC
GTGGGAAGCCGTACAAGAAAATCTACTATGGGCCTCTCTACTAGCACCAGGTGGT
GCAACAATTTTTCCAGATCCGTGGCTATTGAAGGCCTCGCCATCTTCTTTACCGCA
TCTACACAGAACATCCCCGTGTGCTTGTCTATGTCCGAACTTTCCGTTCTACAAGG
ACACCGTACTACTACATCAAGGACCAAGAGGTGGTGGTGGAATGAATACTCCGAA
CATCGTATCTCTAAGAGCGCATCAACCGGAAGTTGGAATTATCCCGTCCGTCCTAC
TAATGAGACCGCTAAGAATCAAGGGAGTGTTCCACCATATCCACTCTCCACTACAC
GGAGAAAACCAGGGATTCACCCTATGTTTACAAGGTGCTCCACCGTCCTCTAGTGT
TGGAGGTGGAAAAGGTGGAATGTGGAATTTCTTCAGAAGAGAGCTAACCTCCAAC
GGATTCCCCGAGAACTTCTCTTTAGATGTACCGGCGAACACCTACAACGCGCTAAA
GTCTAGATTGTGTGATCCGAACGCGGACCATACTTCTTGTCCATCTCCATGTTCTCT
ACATGCTGCTGGTGCTTTACCTGGAACTGGTAGACAAAGATGGAGAGTAGAACTA
GCGCACCTAGCGGACAGAAAGCTATCCCTAAGAGATGTCTCCAGACTAAGACAAG
GTGGAGAGAGAAGATCTGGAATCGCGGTCAAAGTAGTTAGAGGTGGTGCAGGATT
TGCGGCGAGATTACAAGGATCTGTAACCCTAGTACAGCAAGGATGGTTCTTCCCAA
GACTTGGAGGATGTCAAGCTTGGTGGAGAATGGGAGCTGTAGTTTGGTGCGGAGA
ACTACTAACATGTACCTCTGGTGGTGGTGGAATGCTACCAAGAACACCAAGACCG
GACCTAATCCTACTACAACTACTACCAGCGGGATTGAGACAGCTACTACAAACATC
TGGACCGGATAACGAACAGCCGATCGAACAGGATCTAATCTGTAACGTATGCGGA
GGTGGTGGAATGGCGAAAACAAAGGGATCCTTGTCCGTGTTCAGAGAACTACATC
CAGCTGCGGCTTTTGATAGAGCGGTACACTTCCTATTCCTAGAGCTATGGCTACCG
GAACCGATGTTATCTTCTTCCCCACCATCTTCTACAGCGCCGTTATTAGGATCTGAA
CCGTTGAGACATTGGGAAGCGAGTCTATCTAGAGGTGGTGGTGGAATGTGTGCGT
TACAAGGAAGAGGTGCTAGTCCAGCTGGTGCCGGATTATTTCATTGGACAATGTCC
CCGTTCCTACTAGGATCTCTATACGGACACATCCACAACGAAGCTGTCGGTGGTG
GAAAAGGTGGAATGGCTATGGGACAAAGAACACCATCTCTAGCGGAGCTAAGAAA
GTCCTCTGCGACATTCCTAACCTGTAACTTGGGAGCGCAAGCGGAGAAAAGATCT
AGAGCACCTGGAAAGCTAACCTATGTCTCCACCATAGTACTAGATGCGCCGGTCA
CAAAGCTAGAACAGGGACTAGTAATGAAGAGATACAAGATCGTCACGCAGGGATT
CGACTACACCTCTGTAGAATCCGGTGGTGGTGGTACAGGTGTCCTAATTAGAAGA
GGAGATCTAGTCACCGATATGGTCGCGTGTAGAATCAAGACATTCAGAGGACATAC
AGAGAAGGCGGCGATCTGCAAGACAAGAAAAGAATCTTCTGCGGAAACCTCTCCG
GCGGACTCTCTAATCTTAGATTTTCAGCCGCTACAGCTAATGTCCTCCTTCTCTACT
TTGGCCTCCTTGGATAAGTGA
SEQ ID NO: 84 (linker sequence used in CLT Antigen Fusion Protein 3)
GGK
SEQ ID NO: 85 (TCR VB CDR3 AA sequence)
CASSLTGGYTGELFF
SEQ ID NO: 86 (TCR VB CDR3 AA sequence)
CASNKLGYQPQHF
SEQ ID NO: 87 (TCR VB CDR3 AA sequence)
CASSLLENQPQHF

1. An antigen pool comprising two or more different antigens, wherein each antigen is present in the form of a polypeptide and/or a nucleic acid encoding said polypeptide, and wherein the different antigens are present in the antigen pool as separate polypeptides or nucleic acids and/or as part of a fusion protein or a nucleic acid encoding a fusion protein, wherein the two or more different antigens have polypeptide sequences selected from:
(a) SEQ ID NO: 1 or a variant thereof, or an immunogenic fragment of SEQ ID NO: 1 or a variant thereof;
(b) SEQ ID NO: 2 or a variant thereof or an immunogenic fragment of SEQ ID NO: 2 or a variant thereof;
(c) SEQ ID NO: 3 or a variant thereof, or an immunogenic fragment of SEQ ID NO: 3 or a variant thereof;
(d) SEQ ID NO: 4 or a variant thereof, or an immunogenic fragment of SEQ ID NO: 4 or a variant thereof;
(e) SEQ ID NO: 5 or a variant thereof, or an immunogenic fragment of SEQ ID NO: 5 or a variant thereof;
(f) SEQ ID NO: 6 or a variant thereof, or an immunogenic fragment of SEQ ID NO: 6 or a variant thereof;
(g) SEQ ID NO: 7 or a variant thereof, or an immunogenic fragment of SEQ ID NO: 7 or a variant thereof; and
(h) SEQ ID NO: 8 or a variant thereof, or an immunogenic fragment of SEQ ID NO: 8 or a variant thereof.

2. The antigen pool according to claim 1, wherein the two or more different antigens when present in a fusion protein or nucleic acid encoding a fusion protein are joined together by one or more peptide linkers positioned between the antigen polypeptide sequences.

3. (canceled)

4. The antigen pool according to claim 1, wherein the antigen pool comprises six different antigens, wherein the antigens have the polypeptide sequences of (a), (b), (d), (f), (g) and (h).

5. (canceled)

6. The antigen pool according to claim 1 which is an antigen pool in the form of nucleic acids, wherein the nucleic acid is RNA, e.g. messenger RNA.

7. The antigen pool according to claim 6, wherein the nucleic acids are formulated in nanoparticles.

8. (canceled)

9. An immunogenic pharmaceutical composition comprising the antigen pool according to claim 1 and a pharmaceutically acceptable carrier.

10. The composition according to claim 9, wherein the composition further comprises one or more immunostimulants.

11. (canceled)

12. (canceled)

13. A method of treatment of cancer in a human, wherein the cells of the cancer express the sequence of a polypeptide selected from (a) to (h), which comprises taking from said human a population of white blood cells comprising at least T-cells optionally with antigen-presenting cells, stimulating and/or amplifying said T-cells in the presence of an antigen pool according to claim 1, and reintroducing some or all of said white blood cells at least stimulated and/or amplified T-cells into the human.

14. The method according to claim 13, wherein the cancer is melanoma e.g. cutaneous melanoma or uveal melanoma.

15. A process for preparing a T-cell population which is cytotoxic for cancer cells which express a sequence selected from (a) to (h), which comprises (i) obtaining T-cells optionally with antigen-presenting cells from a cancer patient and (ii) stimulating and amplifying the T-cell population ex vivo with an antigen pool according to claim 1.

16. A T-cell population obtainable by the process of claim 15.

17. A T-cell which has been stimulated with an antigen pool according to claim 1.

18. An antigen presenting cell modified by ex vivo loading with the antigen pool according to claim 1.

19. The antigen presenting cell of claim 18 which is a monocyte or a cell derived from a monocyte, e.g. a dendritic cell.

20. An exosome loaded with a polypeptide or nucleic acid prepared from cells loaded with the antigen pool according to claim 1.

21. A pharmaceutical composition comprising the T-cell population according to claim 16 together with a pharmaceutically acceptable carrier.

22. (canceled)

23. A method of treating a human suffering from cancer wherein the cells of the cancer express a sequence selected from (a) to (h), wherein the cells of the cancer would express a polypeptide sequence selected from (a) to (h), which comprises administering to said human the T-cell population according to claim 16.

24. (canceled)

25. The method according to claim 23, wherein the cancer is melanoma e.g. cutaneous melanoma or uveal melanoma.

26. A method of treating a human suffering from cancer, comprising the steps of:
(a) determining if the cells of said cancer express a polypeptide sequence selected from (a) to (h); and if so
(b) administering to said human a T-cell population according to claim 16.

27. The method according to claim 26, wherein the cancer is melanoma e.g. cutaneous melanoma or uveal melanoma.


SEQUENCE LISTING (87 sequences)

SEQ ID NO: 1 - protein, 148 aa, Homo sapiens
MWNFFRRELTSNGFPENFSLDVPANTYNALKSRLCDPNADHTSCPSPCSLHAAGALPGTGRQRWRVELAHLADRKLSLRDVSRLRQGGERRSGIAVKVVRGGAGFAARLQGSVTLVQQGWFFPRLGGCQAWWRMGAVVWCGELLTCTS

SEQ ID NO: 2 - protein, 70 aa, Homo sapiens
MTGVLIRRGDLVTDMVACRIKTFRGHTEKAAICKTRKESSAETSPADSLILDFQPLQLMSSFSTLASLDK

SEQ ID NO: 3 - protein, 61 aa, Homo sapiens
MNTPNIVSLRAHQPEVGIIPSVLLMRPLRIKGVFHHIHSPLHGENQGFTLCLQGAPPSSSV

SEQ ID NO: 4 - protein, 64 aa, Homo sapiens
MAKTKGSLSVFRELHPAAAFDRAVHFLFLELWLPEPMLSSSPPSSTAPLLGSEPLRHWEASLSR

SEQ ID NO: 5 - protein, 44 aa, Homo sapiens
MLPRTPRPDLILLQLLPAGLRQLLQTSGPDNEQPIEQDLICNVC

SEQ ID NO: 6 - protein, 84 aa, Homo sapiens
MWNSLEARSPKSRCCSTGPWEAVQENLLWASLLAPGGATIFPDPWLLKASPSSLPHLHRTSPCACLCPNFPFYKDTVLLHQGPR

SEQ ID NO: 7 - protein, 77 aa, Homo sapiens [sequence omitted]

SEQ ID NO: 8 - protein, 38 aa, Homo sapiens
MCALQGRGASPAGAGLFHWTMSPFLLGSLYGHIHNEAV

SEQ ID NO: 9 - protein, 9 aa, Homo sapiens
VQQGWFFPR

SEQ ID NO: 10 - protein, 11 aa, Homo sapiens
VVRGGAGFAAR

SEQ ID NO: 11 - protein, 9 aa, Homo sapiens
HLADRKLSL

SEQ ID NO: 12 - protein, 9 aa, Homo sapiens
ARLQGSVTL

SEQ ID NO: 13 - protein, 10 aa, Homo sapiens
VPANTYNALK

SEQ ID NO: 14 - protein, 10 aa, Homo sapiens
RLGGCQAWWR

SEQ ID NO: 15 - protein, 10 aa, Homo sapiens
ANTYNALKSR

SEQ ID NO: 16 - protein, 9 aa, Homo sapiens
RLQGSVTLV

SEQ ID NO: 17 - protein, 9 aa, Homo sapiens
VPANTYNAL

SEQ ID NO: 18 - protein, 8 aa, Homo sapiens
ADSLILDF

SEQ ID NO: 19 - protein, 11 aa, Homo sapiens
SSFSTLASLDK

SEQ ID NO: 20 - protein, 10 aa, Homo sapiens
LVTDMVACRI

SEQ ID NO: 21 - protein, 10 aa, Homo sapiens
LILDFQPLQL

SEQ ID NO: 22 - protein, 10 aa, Homo sapiens
MSSFSTLASL

SEQ ID NO: 23 - protein, 11 aa, Homo sapiens
LMSSFSTLASL

SEQ ID NO: 24 - protein, 9 aa, Homo sapiens
LMSSFSTLA

SEQ ID NO: 25 - protein, 10 aa, Homo sapiens
QLMSSFSTLA

SEQ ID NO: 26 - protein, 10 aa, Homo sapiens
MVACRIKTFR

SEQ ID NO: 27 - protein, 10 aa, Homo sapiens
VTDMVACRIK

SEQ ID NO: 28 - protein, 8 aa, Homo sapiens
SPADSLIL

SEQ ID NO: 29 - protein, 9 aa, Homo sapiens
SLILDFQPL

SEQ ID NO: 30 - protein, 9 aa, Homo sapiens
QLMSSFSTL

SEQ ID NO: 31 - protein, 9 aa, Homo sapiens
NTPNIVSLR

SEQ ID NO: 32 - protein, 9 aa, Homo sapiens
RPLRIKGVF

SEQ ID NO: 33 - protein, 10 aa, Homo sapiens
NTPNIVSLRA

SEQ ID NO: 34 - protein, 10 aa, Homo sapiens
VLLMRPLRIK

SEQ ID NO: 35 - protein, 10 aa, Homo sapiens
MRPLRIKGVF

SEQ ID NO: 36 - protein, 10 aa, Homo sapiens
KTKGSLSVFR

SEQ ID NO: 37 - protein, 9 aa, Homo sapiens
AAFDRAVHF

SEQ ID NO: 38 - protein, 8 aa, Homo sapiens
AFDRAVHF

SEQ ID NO: 39 - protein, 9 aa, Homo sapiens
KTKGSLSVF

SEQ ID NO: 40 - protein, 8 aa, Homo sapiens
FLFLELWL

SEQ ID NO: 41 - protein, 9 aa, Homo sapiens
SVFRELHPA

SEQ ID NO: 42 - protein, 9 aa, Homo sapiens
SPPSSTAPL

SEQ ID NO: 43 - protein, 11 aa, Homo sapiens
FLELWLPEPML

SEQ ID NO: 44 - protein, 9 aa, Homo sapiens
APLLGSEPL

SEQ ID NO: 45 - protein, 11 aa, Homo sapiens
LPRTPRPDLIL

SEQ ID NO: 46 - protein, 9 aa, Homo sapiens
TPRPDLILL

SEQ ID NO: 47 - protein, 9 aa, Homo sapiens
RPDLILLQL

SEQ ID NO: 48 - protein, 11 aa, Homo sapiens
ATIFPDPWLLK

SEQ ID NO: 49 - protein, 9 aa, Homo sapiens
FPFYKDTVL

SEQ ID NO: 50 - protein, 10 aa, Homo sapiens
FPFYKDTVLL

SEQ ID NO: 51 - protein, 10 aa, Homo sapiens
TIFPDPWLLK

SEQ ID NO: 52 - protein, 9 aa, Homo sapiens
IVLDAPVTK

SEQ ID NO: 53 - protein, 8 aa, Homo sapiens
GHIHNEAV

SEQ ID NO: 54 - protein, 11 aa, Homo sapiens
SLYGHIHNEAV

SEQ ID NO: 55 - protein, 10 aa, Homo sapiens
SLYGHIHNEA

SEQ ID NO: 56 - DNA, 1063 bp, Homo sapiens
  source 1..1063: mol_type = other DNA
cggggccagtctttcccgtgctattctcgtgatagtgaataagtctcacaagatctgatgggtttatcaggggttttcattttgcttcttcctcattttcttttgctgctgtaatgtaagaaacgccttttgcctcctgccataattctgaggcctcacagccatgtggaacttcttcaggagagaattaacatccaatggattcccagaaaacttttccctcgatgtaccagcaaacacctacaatgccctgaaaagccgcctctgcgaccccaatgcagatcacacgtcctgtcccagcccctgcagcctccacgcggcgggtgcactgccaggcacgggaaggcagcgctggcgagtagaactggcccatctcgcagataggaagctgagcctcagggacgtttcacgccttcgtcaaggtggtgagaggaggagcgggattgccgtgaaggtggtgagaggaggagcggggtttgctgcccgacttcagggatctgtcaccctcgtccagcagggttggttcttcccgaggctgggaggatgccaagcctggtggaggatgggggcggtggtgtggtgtggggagcttctgacttgcacatcctgagggaaccttctgcagctgatgtgtgaactggaccccaggccgtgcctccgaggaatccccaaggctatggcccctcaggtcctgctggggtgttggcccccacctctgcctcagaatgcaggggttctgcagggaagccgcagaccagcctgctgccttgggccctagggacactgcagccccagaaagtactgtgggggacaaaagagttgtttctcgggggagaaaacacctgtgaggaaatgcaggtgccacagagggaaatcctcctggggaggagggtacctgttccatcctcggccgacacgggactgcctggtgcctggtacccacagccgctacctgccgcacgcatctctccatggtttgctaattacttccattagttttaaacaaacttgacaagagacagaagggtccagagagaaattaaatctaactgtttaaacatgt

SEQ ID NO: 57 - DNA, 2851 bp, Homo sapiens
  source 1..2851: mol_type = other DNA
cacctccatcactgcgaattataattcgacatgagatttgggagatgacacaaaaccaaaccatatcagtctttaaagagttaagttaaaataagctctttaaagtgggccctaatccagtatgactggtgttcttataagaagaggagatttggtcacagacatggttgcatgcagaataaagacttttcgaggacacactgagaaggcagccatctgcaaaacaaggaaagagtcctcagcagaaaccagtcctgcagactccttgatcttggacttccagccactgcaattgatgtcaagcttcagcacccttgcatctctggataaatgaaatgtcaccccagctgccgtccttgttcagatctgtgcaataaagagcaaagcataaaaccaagtcaaggctttgagggagtgacctacaaaatgcataatgtgaaacaatgcaaaagcgaaaggtgcaaaatccccatcaaagagctggaggctgacagatgcgccagtgataattcccatcttccaacacaggagcacagcttccattttccataacagaacaacagccagagcagctggaaggcagggccgcatcccagacttccaccaacaatgggatgagacttgacatctggaatcacaaccacaacagacctgagagacccaccagcttgatgacaagcttcttctttcaaagaaaggtatcagtctgggggacctagtgctgcaaaccatgacaaattaagtgtggcatccctcacttgcataatggaactcagtgatattttttaattaacaagagttatttttatgtaagcttctctcattcctccactgtgcgtgctcgggggctggtggtgaggaaaaagaaaacagctgtgcgggaagcatcaagaaaaggcaagtcatgaagtcttagagatcagtgacatgtaagaaaaagagtgaggagaaaaatattcctactaaagttttccatttgtttaccttccttgtcacatagacttccaagagttagaagtctaggatttgatctccaaatcttcctggcagattactcatcttcatttcattcatatagtccaggggtttgtacaaaggaagatgccagttcttccccaatcataactaagatatcaagagatattcttttgaaatgtaacaaaggagatctgaagttcatctgaaaaaaataaatggttttaggcggtcatgaccatgggatgctggaccaggatggtaagcttcaggaaacagaatctggagaatgcccagctgctcccacaggaagcatcagggaagaagaagaagaggtgtgaagtctgccttctgctctgctgggatccctttcacatctcctttgcctccaggcagttttggttcctggccatttccaggtgtgactcactcaggatggtaagcatcttctctcctacccagagtagaggatgaagacctcatctcagaggttgaagggagctccagagagaggtctcaaacttccagcattaactgctaaagaagcttcatgagctgctggagaacctgggaaatgaccaattatagggacagagctcaaatactctgggacactctagtagctgagaaagttccaactccagggtgatagaggactgcctggcaaaccatcatcaaagcagaagacctgatactaacatcacaggctatggtttattactgaagatcagtgcttacaccctgccagaggttcagaagcaaacttatcattgttctccctggagatgttggcccacattctgaaaagtgtggtcagtagtagcaacagaaagcaattgtgcttgccaagcacaatgtcactgtccccagcccttcccccaacacaacccagtaggtgcttcctggctgcaaacttgggaaagtcacttgacctgtctgaggttccacttcctaatctggcctggcgaagataagaaaaacagtttatttaaagtgtctagcaaagtgcttggaccaaaataggacctctgaaatggttatggtagtgctgttaaggtgatgttttaagtgctgatgagcacaaagatgggtaagatattccttctgttaaaatctacagtctaatgagagagaacaagatgaatgcacaataactgtcattcagaacaggattatgagaaggtgtgaatttctgtgaaaaatcagaacagggagtaatatgatcccaggtgattggcagggggtgggggtctggattcaactggagagggagctggcagggaaggcttcctggaggatgagagttcaacaaggggcaggtgtaggatgtgggtggccaagtgactgggcagaaggagctgcagaagtaagaccccaaatcaggaagacaagggcctgctgagaaacacgagctacaaagtgcaagtgcaggaagagttgggatgagattagaagggggtctggggccagactgtggaaggcccaaatgccgggctaaggagtttgtacttaattcagtggtcaacggggagtcattggaggctgttgagcaggagagttgctttctttacagctgtgccagactaaattaaacctaaacagtactttatagctggaaagggaaggcccaggaatagctcttgactcagaaacaggcattggggaaggtaatgagaaacagccgtgactgatcaaagcagagaggttaattaaatttgtaattattgtgaaaggccattaaaaaccctagttcactagagataactgctctagtggggcttcaaagacaaacgcttcttttaaccttgaatagggggatgtttgcttctctgtggaggagatatgattaagatacttaataaatggtagataaaca

SEQ ID NO: 58 - DNA, 2614 bp, Homo sapiens
  source 1..2614: mol_type = other DNA
aaacacactaagggctttgttatggactgaaatgtgtcctctccccagagtcacaggattatgaagccataatcctcaatgtgactatatttggagcaggggcctttacagacataattaaattaaatgaggtcataagagtggggccctagtctgataggactggtgtccttacaagaagagggagagtcctcagagagtcctctctctcggcatggacacaaaagaaaagccatgtgaggacacagagagaaggtggctgtctacgagctaggaagaaaggcctcaccggaaaccaaccctcacagcagctccatcttggacttccagcctccggaactgtgagaaaataaatgtttgcaattcaggtcggttgtattttgtgaaggccatcctagcaaatgaatactcctaacattgtctctttaagagctcaccagcctgaggtaggaatcattccatctgtgttactaatgagaccgctgaggatcaaaggggttttccaccacatccactcacctctacatggcgaaaaccaagggttcactctctgtcttcagggagctccacccagcagcagcgtttgacagagctgttcacttcctcttcctggagctgtggcttccagagcccatgctcagcagttcccctccttcttcgactgctcctctcttaggctcagagccactcagacattgggaagcaagtttgtcaagatgacagagaaccgaggtaatggattcgagtgatgaaacaggaagttcattcatgagtttttggccacacctccaaagtgacgacttagccagaaatgggataactgggtttccctacttctcttttatcatcctcaatgagagtgaccaaatattagagctagatggaaccttagtgaaaatctggctactcgtcccgtcccaccagcctgccacccatttcaagtttgaagagacaaagacacatggaccttatgtaattactggggattaccccaggagtctgtggcaaaagtcagcttcttccctccctgcttccccgccctgtctctggtactttctaccaacactgggctgtttctgtgatcacacttaagcgtacctaacctgcgaatgctgtatagaaggtgctaatgaacatgatttagctttaacactcagttttctaaagggacacgtgggggcagcaaatgtttaggcaaaaacaattccagttctagcctctactgtctacatatgtgtatacatttgggaaacgtttgggaaagggatatttgagagcttctttttcttttttgtggtttagttatttgatgatattgagattgtttctgagccatgtgcttcaacatcggattggggatttcagaaaaagttttagtcactgtgattccatttagcttccaaatgtgtctctgctaagagacttaaaagcactcataaatagcacgtgtgtcttctttgcagtgtttgctaattttgagtcacatctttttagaaaatcatgagatttggtgtcacagagactggaataaatatagtcaaacttattggtgaagatttcctttagctgttttcataatccatttccattgttatgattattgatgaataaaacattttctttaggtagatacttcttttttccccccaccttgatttaatgtttccactcttattgtcaagtttcttattactccctaataactctcaataaaataatgattcctgggagattattcctgctttcctactatcacctgttgatttgaaaagacagaacaataccgtagaagcttcactaatacattgaaagataaaatgataatactaaatactaaaatatgaaaagtgatactaaaagtggagtcctggcactagtatttttttttttgagtctttaaattttatttatttatttttgaattttttaaaattatatgttatgttctgggatacatgtgcagaacgtgcaggtttgttacataggtatacaggtctggcactagtattttgttgccacaaaatatcaagcatgtatccaaactgctcaagacacattaaagacacaggtaatctgtaggcatattcaggcttgtagtttgcattttttggttttcttgtggctttcagtgcaagttgaggtaattcatgggaaacagtcaccaaagaagtgccagtattagaaatccaagagccatttctctagcttcttccagaatcaagactttagaggtaatttctatcaacactggacatttcctgtctgcaattaacaatgaacacatagcattatgtttaattgcaacctgtttaaagcagattggatgctaaggtttaagaacactcttcagtcaaaaaggtcttttaatcaggtttttaatcttgagcacaatctaggacacagcatcatagactaactcattcgagaataggtgttgtcatctaatcctaaccacccccaccaccaacaagctgaatagctctgggctcagtatatacatttgtactgggctcagtacacacacctaagctgggttcagtatatgccactttatagtgagaggcattttgtaatgagagctctgggttcactatatacatttgtactgggctca

SEQ ID NO: 59 - DNA, 2172 bp, Homo sapiens
  source 1..2172: mol_type = other DNA
ctttcatctttaatttgaccaaaatggaaaccaggatcatgagaattcctcggggctggtgttgaaaggaatttcccctgctcttgccagagtctcgaggggtggcctccttccacgggtgagtaaccacaagtccatgtgaccgaacaaacagcatattcttttgttcaaaagagaaaaacaacattgaaggaaatcagctgaagaaaattgagatgaaagccagtccacgcctcagcatcctgaggaaatgttcttccttgatgctctgagctctctaagaagttaccacaaaaccaaacccatcagaagtttgcaggacgtccttgtttagagctgggaaataaaccacgaaacagcgcaaaggagagtccaggcctgccaatgcttccgcgaactcctcgccccgacctcatccttctccagctcctacctgcaggcctcagacaacttttgcagacctctggtccggacaatgaacaacccatagaacaagatctgatttgtaatgtttgctgatctccaaagtgtaaatactcccaccatgaccaattggaagccactgacaagtcctcactaaatgcagaattgagaagaaacacgaatagcacatcattgtatggtatttccactctaccaatggatgtgaataacctcaagaacacataatgttgagatgtggaaaaatcatcaggaagcctctccccttcaccctgcagtgtctctgcaatgctggtagagtgggctggcacagcccccgcctctggcctggtccgggtccaacctgcctgcctcctggggcagcagctccctgaacgaagagccgcggagaccgaagaactcagtgaatcagcagttctcccagatgaaacgctggcctgaaagcagcctcaagagctttggcccgtcaccttgccttgccttctcttccttcctcgtcctctgtttgttcatctttcctgaaaaaattaagtcagctgttccccttaaccaatttccctggcattctgaagggtaggccacatggcccacctgccagctactcccacctgccaagccttcctgactatatttaccctggtactcccatgtcccggggctgcttcagcagagccaaggacacacccaggtgtttgttttctaggtcagatttcctcagccatgggtgtatctgtgcttgtccctcaaaatcctcatagctcctttccccaccccaacttccaggccagacggggttcaggggtgtctcagctaaggttcccctgaagcagacacaaattagtacaaaagggacttattaggaggtgatccttaaaaatttgggcaggagaatggaaagggtggacacagacaaaaaggatgccaagtgagaatgtgatgattactgctgcagttttctacagctgcctgtcaaactatcccaacacagtggcattgaccagcagccattttcgctatgcccacagcttctgtgggtcaggcgtttggacagggcaatgggaacagcttgtttctgttccatagtgtgtgagggaggcctcaactggaaaactcaaagaccggggtggcttgatgactggaggctggaaccatccggaagcttcttgaagctgggttgacttacatgtggtcttgcatgtgagttgggcttttcacatcatggctgttgggttccaagaggcagtgtctggggaacaagagttccaagacaccaaggcagaagctgctagaccttttttgacctcgcttcggatgtcagcaatgaagttctgtggattcaactgcattctattggttaccagcagtcgcttgaatctgattagctccaaaactgagtgagcctctggcaagagtgcattgcagaagggcacatccaatgagaggcgctgctgtagtaacagggacctctccaatggcctgtttgagtgctctcagaatatggcacctgggttcctccagagcaagtgatctgagagagagcaaggagaaggccactctgcctttcatgactcatctcagaagtcacacactatcacaggttctgttctgttcattggaagtgagtcatgaagtccggcccacactcaagagaaaggaaattatgctccttcttttgaaatgagtgtaagaaagtaaaactttgtcaaatgtgtatgtgtgtgtgtgtgtgtg

SEQ ID NO: 60 - DNA, 3103 bp, Homo sapiens
  source 1..3103: mol_type = other DNA
gaggcctcacagccatgtggaactctctggaggccagaagtccaaaatcaagatgttgcagcactggtccctgggaggctgtgcaggagaatctgctctgggcgtctctcctggctcctggtggtgccacaatcttccctgatccttggcttttgaaggcatcccccagctctctgcctcatcttcacaggacttctccctgtgcctgtctgtgcccaaatttccccttctataaggacacagtcctactgcatcagggcccacgctaatcacttcttcttcactaattacatcggcaaaccccctatttccaagttaagtcatgttctgaggttaggatttcaatacctgaattttaggggacatagttcagcccaaaggacctggggggacagaaacacccaggaaagtccacaggtcagcgcctggacccgcctggccgtgggacattgctcggccgagttcatcctcataaacgctgacgtgtgctagaagatacaatattagtttcataaacaacagtttctttttttgcaaaccatacccacaggtcctattgccacttttcagtgctgcagcagctagtcagatgacaactgggtgagagcttggtctcgagtgtttcctgtgaggccagaatattgctctgattcctcaggggtgactcagccctccggtgggaaatatgtgacctcatttgcatttcctaattgaattgctatcagctttcctgcaagactgcaccaaaaaacagttcttccagattccagcactgcctgcattgcccacagaaaggctgcttttaaatagttgctaagagagggcgacaaaagaaggagaaaagttgccaaatttttatggttcatctaactgtaaagctgtcagttttacccagctccccagagtcgctaaaataatggacagaggaggcggcacttgaagaaacctcgacataagagaaatgaaatttacagaaccaggaaattaggggtccccatgcacctgccctggcagaatctgcttccaggctcctctcaataaacactgccctgggactcccagcgctctgtaaactgatgctttgaaggaggccatttgtttctccagaaccttctgccagcagaactcttgcttaccacgtgcccccatcagaactcccaccttctctaaacacagttaggtagccctctgcatgcatttgtttaaaaaaattaaattgtgagtaatagactataagtacaacagcgtacagaacaccagctaaaaaaatgatggcagaatgagcacctgtcttgccacaagactccctcccaccctctggtgtcccccagcatggcttcctgctgccccctgaaaggaaccctcaaccgactgtaactctgcacctcagtgccacccgaatttggacttcatgggagtgcggtcactcggctgacacgtgttcctgctgccaggcacctgcctgctctccttctttggtctgtttgtgagattcgctggctttgttgcttctagctgcagtttgttcacttttactgccgtataatattccactttaggagcccactgcaatttattgactcattgcaatggtgagggcatgtgtgttgtttccagcttttggtgattatgcgtaagatttctcttaacgtcccgtacagagttcttggtattacctgcctatactgctatgggcatacaccaaaaacgaggcatatgtatatttgactttaatagtaattctgaattgctgtctaaagtgtttgtaccattgtacactcccatcaagagtatacgagtgctgcaaatgtcctttcccagactctgccttattgtctcatttttaagatatatgttttgatgatcagaagttctttttttttttttttttttttgacaggcttttgctctgttgcccaggctggagtgcagtggcatgatctcagctcactgcaacctctacctctcgggctcaagtgattctcctgtttgagactccctagtagctgggattacaggtgtgcaccaccatgcctagctaatttttgtatttttagtggagacagggtttcaccatgttagccaggctggtctcgaactcctgaccttgggtgatctgcccaccttggcctcccaaagtgctgggattacaggcgtgagccaccgcacctggcagaagttcttatttttaataaagtccagtgatcattctttcccgtgtttcacgcttcacgtgccatgtgcgaggaagctttccacacccgggggtcagttcactcccagctgcattgctttgcctttcctgtttaggttttcagtccacctcaaatggattttggttgtggtgtgaggtaggggtcattttacttttttcttaaagatgccccattggcccagcatcatttattgaaaagacagtctttcctctgttgtttctgtagaaatgggcaaactttcccccaaatggccagatggtaaatattttaggcatctcaggttaaggggcaaaattgaggacatcgtataggtacttacataaccctttaaaatggaaccatttaaaaatgtaaaaaccagtcttagcttgctggccataaaaggcctgtgggctacagcatgctggcacctgctctacagcactgctcttataataaaaaatcaagcgtctgcaccctgggcatttgacaaggctcatcagattgtaaagatgaactgggtggattttgtagtatgagagttatacgtccatgaagctgatttttaaacacattacatgtctttttctgggctcattatttttgttccaagtgtctatctaatcatctttgcttcaaaatgacaccgttttaatggctgtagttcagtggttctcaaaagggggcagttttgccccccagggaacatttggcagtggctggaaagagctttggttgcatatggaggttatggggctttacctggcgtttagtgggtagaggccaggagtctggcttactatcctgtagcacacaggacagccccaacaacttgtctggaaggatgtgtcctcccatctttttcttgttctaggctaccttcgccctttccatttccattaaaaaat

SEQ ID NO: 61 - protein, 1907 aa, Homo sapiens
GTGTGCCCTTTGCAGCAAAGAGCGTGGTTCCTCCTCTTCCTGGGTGTAAACTGCGATTCAAAAAGCCAGGTGGGAAGCCCTGTAGTAGGGACTCTGGCTTTGTCCCTGTTTCCCCCTTTTCTTCCTCTTCACCCACTAAAACCCTGTTTTACTCACGGTTCAAATTGTTTGGCAGCCTGAATTTTCATGGCCATGGGACAAAGAACCCCGTCTTTAGCTGAATTAAGGAAAAGTTCTGCAACATTTTTGACGTGCAACTTGGGGGCTCAAGCGGAGAAGCGAAGCAGAGCCCCTGGAAAACTCACATATGTGTCAACAATAGTCCTTGATGCCCCTGTAACAAAACTTGAGCAAGGCCTGGTGATGAAGAGATACAAGATTGTCACCCAAGGATTTGATTATACCTCTGTTGAAAGTTAATGCACACTCAACCGAATATGGCAATTGGTACTCTCAATTCTATATTCTCAACAATTACAGTGAAGATACAAGTAGAATACAATCACTGCCACATTTTTTGAAATTCTGTGTAAACTGTAATATTCTGTCAACATTAATGACATTTGAAGTGTCCTGTCAAAACAATTGCAGCTACTTTATGTATAAAACATATTAAATAGGCTCATCCAACTTGCATTCTTTATTAAGCTTATTTTCATATTTTTTCCTATGGATGAACTTAAAAATAATTTTGTTTCTTAATTAAGATTCTATGCATGAAGATGCTGAATAATTTAAGACAATTGTTCATTCAAATAGTTGCTAATTACACCCTCCTGGCATAGTTATTGTATTATTACTACATTTAGGAATAATATGCTGTACTACTTGGACTTGAAAATGTTTCTGACATTTTAATGAACACACTACTTAGTTATATTTTACAAGGGTTTTCAGTGAACCACAGAGGATTAAAAAATGTCATTCAAGGGTTGTAGATAATTAAACTGACTGAATATAAGAAGCCTCATATGGAAGTGAAAATTATGTATGAATTTTGTTGAGCTGGAAATGTGTTTTACTAAATGACTTCAGATTTGTTACTTTTAAATACAAGATGAAGGGAATCAAGGGGATACTTTCTTCTCACCTCAATTTGTTCCATTTGCATAAGCTATTTTATCTTTCAATATCCCTTTTGATATTTCATTTTGGCCCTTGAATACACTAAAGATTATTTAAAATAAATAAATGTTGACTTTGAAATACTGCTATATATATTCATTGCAATACATCAGGTGGGAAATTGGTGCAATTCAGTCACACACACAACCTCATAGATCAGATTTCCAGTTAAGTTTTAATAGAAAGAGATTTTAGATAGACATGACTTTCAATAAAATGGAAGAATTTTTATTAGTTTCAAAATAATGATTTTACTTGCTTATCAACACAGTTGACCTTTCCCCACAGTACTATGATCTTCTTAGTACCTCTGCTATAATTATATTAATGTCTTCCTATATCTAGAAATTTGATTATTATTCTATTTTTATGGATCATTAAATTTTTCTATTCAAGAATGATCCCTATAGTCAACTTCCTGAGGGATTTCTGCTTGCTATTTTCTGTTTCAATTGTGTGCATTTCATATTTACTATTAAAGATTTTAAATAATGACACATTGTTTAAGCGGTTGTAAGTCTGATTGTTATAAAGCTCTCTGGAGAATTCTGTAAGCTTTCACATCTGATGGAGAACTTCAATGAGAACATCTTTAATGTGGAATTCTTGGACAAGAAACTACAGATTATCCATGGTTCAGAAGATATGATGAATTAGTAACTATTTTTCCGTACATAAAAAGTCAATCTTCCTAACCAGTTTGTTGTTTTAGCTAAATGAATCGGTCAAATATTTTAGTTATATTAGCAATATTTTGTCTCTAAAATATCTTGACATAAAAC

SEQ ID NO: 62 - DNA, 3091 bp, Homo sapiens
  source 1..3091: mol_type = other DNA
gggtccctggctcggctctacccccatggatctaggtgaggacaggcgctcctgctttcccgcccaaatgttgtattttccaagcctacccttgccggccacgcccccaacctgggcctataaaaaccgccccccgcaggccctagcgggcagacacactgaagtcgctagacatttgaggaacacatccggggaagaagacacaggtggctggtcatggagagcccgctgggggaagagcacacagacaggcaccggcaggccattgaccagcgggacaaggtggagtttggctggggcagtgggaggagagctggggccgccgagctgccctactccaggggaaaaccacctcccttctcgctcccccatcaccggagagctacttccactcaatacaactttgcactcattctccaagccacgtgtgaccaatttttccggtacaccaaggcgagaaatccggggatacagaaagccctctgtccttgcgataaggtagagggtccaattgagctaacacaagctgcctatagacggcaaactaagagagcacccagtaacacacgcctgctggggcttcaggagcacacacgtccactggggcttcgggagctgtaaacagtcagccctagacactgtcgtgggatcggagccccacagcctgcctgtctgtatgctcctctagaggtctgagcagcggggcgctgaagaaacgagccacactcccatcacacgccctgagaggaggacaagggaacccgtaccgtttcacttgtgatagagataaagttattattgttgtaattttaacttatagaactataatataatggtacaatgatatatattgacaatattcctctttcacccacattttgtatctgtgttcaagattaaatgaaaaaggatattctcaaaaagctagcaaaaccaaaagcaagacgttatgccaaatgtaaacatatttctgtttagcaaatagcaggagtgtataaaacatttctcttttcacataacagaatgttctatgcttactgtattagttaacaaattcaagtctgtttattttgtttgaaattccacttcatccataaattacagcattacacaataacaccagaaggacaatatcaccattgtttgcttttacagtcatctcagcccacaaaatgcttcccattgtcagcttgctatcatcaagctattgttgttttcctctttctggggtcttgtattaatatcattcaaattatttagacactcaacagtgtttttgctatcagtgcaaacctctaaagagctggagccccagcgtgatgaccaaataacccttgactatttatcttgccgtaagtcattatttcctgaggccctggagaagctgtgttgccatgtgtgcactgcagggacggggagcttctcctgccggagctggtttgttccactggacaatgagcccttttctgcttggctctctgtatgggcacatacacaatgaggcggtttagggaagaggggtgatgtgggtcattgataacaacaatccccgaacttcttaaagaattgttgagccccctaaaaatatgttgtctttatgaattatccttaaccctttttaattgcataaaaacttcaggcacttgaaaaaaattaaaaaacgaaaagtaagtctgtctctagtatccctcttctcctttgtagaattacatcctttattcactctgccactatttattatgtgcctcctgtgtgtaagacatactgttagtcattggaaatacagaaatgaatgggacagacacacttcttgccctcatggaacttagggcctaatgggagatacaatgttaaggttgtttctagtgtttttttttttttttaagaatatgttctacatgtatacatgtaatatgtattctctcccatttttaaaaaacataaatggtggtttactatgtgtacgctttttggttttgcattattcttttaacagtatgtagaggatgcattttctactgtatagtgttctgtcccccttaaatagcactctgattttattttgggggggaatcacgactttctaattttgcatactccttgtgggattgtaaatcaggtcccctgtcctcaagtagccaatggttaggtatgcaacccagcttatctctctttagactcaatctcaagcagatccagagttactcaggatcagaacaatatttgaaaggcattaccagaatccagacaagatgatggagcaatacctgatgcccagtggtctagggtagccgattcctgttctgctctccaggctcctgtccattctgtggatcaactcatattgctccaattcattttgttttgcttgcataagccagaattaacttctgttgcttgtaaacaaagagagttaaccaaagacatacagtatatcagagtatagagacctactttactctcggtcattgcctgcatgagtcttctttatatggatgtatcacagtttatttaaccactcccttgtaagtggacatttaagtctagtgttctgtaaataaaaggtcagaatatacgtctgtatacagtatatattcttgcatatccacctttggacagatgtgtgaatgtgttttgtagaaatacatttgtagaaatgcaactgctgggtcaaagaattagtagatttttaataacatcaaacagcgttgaaggcccccatataagaataacaactactgactgaagacataactaattaaaaaaattaattacagcttatttgtaataacttcttattgtcactgagtgaaaaggtaattctcgttgaatttacgaaaagtgactaataggaaatttagaaaactcagagaatatataaaaacacaaagaaaagccagccaccaagtcgcttataattctctcaccaacagtggcagaattacatttagtcatcatcattattcttacatccagttttatagttatttttgaagaggattattatcaactatcaactctgtcatagctggaagtagaggccactaaaacagatttcttaaactccaagtactgtatatggattttctac

SEQ ID NO: 63 - DNA, 444 bp, Homo sapiens
  source 1..444: mol_type = other DNA
atgtggaacttcttcaggagagaattaacatccaatggattcccagaaaacttttccctcgatgtaccagcaaacacctacaatgccctgaaaagccgcctctgcgaccccaatgcagatcacacgtcctgtcccagcccctgcagcctccacgcggcgggtgcactgccaggcacgggaaggcagcgctggcgagtagaactggcccatctcgcagataggaagctgagcctcagggacgtttcacgccttcgtcaaggtggtgagaggaggagcgggattgccgtgaaggtggtgagaggaggagcggggtttgctgcccgacttcagggatctgtcaccctcgtccagcagggttggttcttcccgaggctgggaggatgccaagcctggtggaggatgggggcggtggtgtggtgtggggagcttctgacttgcacatcc

SEQ ID NO: 64 - DNA, 210 bp, Homo sapiens
  source 1..210: mol_type = other DNA
atgactggtgttcttataagaagaggagatttggtcacagacatggttgcatgcagaataaagacttttcgaggacacactgagaaggcagccatctgcaaaacaaggaaagagtcctcagcagaaaccagtcctgcagactccttgatcttggacttccagccactgcaattgatgtcaagcttcagcacccttgcatctctggataaa

SEQ ID NO: 65 - DNA, 183 bp, Homo sapiens
  source 1..183: mol_type = other DNA
atgaatactcctaacattgtctctttaagagctcaccagcctgaggtaggaatcattccatctgtgttactaatgagaccgctgaggatcaaaggggttttccaccacatccactcacctctacatggcgaaaaccaagggttcactctctgtcttcagggagctccacccagcagcagcgtt

SEQ ID NO: 66 - DNA, 192 bp, Homo sapiens
  source 1..192: mol_type = other DNA
atggcgaaaaccaagggttcactctctgtcttcagggagctccacccagcagcagcgtttgacagagctgttcacttcctcttcctggagctgtggcttccagagcccatgctcagcagttcccctccttcttcgactgctcctctcttaggctcagagccactcagacattgggaagcaagtttgtcaaga

SEQ ID NO: 67 - DNA, 135 bp, Homo sapiens
  source 1..135: mol_type = other DNA
atgcttccgcgaactcctcgccccgacctcatccttctccagctcctacctgcaggcctcagacaacttttgcagacctctggtccggacaatgaacaacccatagaacaagatctgatttgtaatgtttgctga

SEQ ID NO: 68 - DNA, 255 bp, Homo sapiens
  source 1..255: mol_type = other DNA
atgtggaactctctggaggccagaagtccaaaatcaagatgttgcagcactggtccctgggaggctgtgcaggagaatctgctctgggcgtctctcctggctcctggtggtgccacaatcttccctgatccttggcttttgaaggcatcccccagctctctgcctcatcttcacaggacttctccctgtgcctgtctgtgcccaaatttccccttctataaggacacagtcctactgcatcagggcccacgctaa

SEQ ID NO: 69 - DNA, 234 bp, Homo sapiens
  source 1..234: mol_type = other DNA
atggccatgggacaaagaaccccgtctttagctgaattaaggaaaagttctgcaacatttttgacgtgcaacttgggggctcaagcggagaagcgaagcagagcccctggaaaactcacatatgtgtcaacaatagtccttgatgcccctgtaacaaaacttgagcaaggcctggtgatgaagagatacaagattgtcacccaaggatttgattatacctctgttgaaagttaa

SEQ ID NO: 70 - DNA, 117 bp, Homo sapiens
  source 1..117: mol_type = other DNA
atgtgtgcactgcagggacggggagcttctcctgccggagctggtttgttccactggacaatgagcccttttctgcttggctctctgtatgggcacatacacaatgaggcggtttag

SEQ ID NO: 71 - skipped

SEQ ID NO: 72 - protein, 4 aa, synthetic construct
  REGION 1..4: note = Linker sequence used in CLT Antigen Fusion Proteins 1, 2 and 4
GGGG

SEQ ID NO: 73 - skipped

SEQ ID NO: 74 - protein, 5 aa, synthetic construct
  REGION 1..5: note = Linker sequence used in CLT Antigen Fusion Proteins 1, 3 and 4
GGKGG

SEQ ID NO: 75 - protein, 6 aa, synthetic construct
  REGION 1..6: note = Linker sequence used in CLT Antigen Fusion Proteins 1, 2, 3 and 4
GGGKGG

SEQ ID NO: 76 - protein, 501 aa, synthetic construct
  REGION 1..501: note = Polypeptide sequence of CLT Antigen Fusion Protein 1
MWNFFRRELTSNGFPENFSLDVPANTYNALKSRLCDPNADHTSCPSPCSLHAAGALPGTGRQRWRVELAHLADRKLSLRDVSRLRQGGERRSGIAVKVVRGGAGFAARLQGSVTLVQQGWFFPRLGGCQAWWRMGAVVWCGELLTCTSGGGTGVLIRRGDLVTDMVACRIKTFRGHTEKAAICKTRKESSAETSPADSLILDFQPLQLMSSFSTLASLDKGGKGGMWNSLEARSPKSRCCSTGPWEAVQENLLWASLLAPGGATIFPDPWLLKASPSSLPHLHRTSPCACLCPNFPFYKDTVLLHQGPRGGGKGGMAMGQRTPSLAELRKSSATFLTCNLGAQAEKRSRAPGKLTYVSTIVLDAPVTKLEQGLVMKRYKIVTQGFDYTSVESKGGMAKTKGSLSVFRELHPAAAFDRAVHFLFLELWLPEPMLSSSPPSSTAPLLGSEPLRHWEASLSRGGGGMCALQGRGASPAGAGLFHWTMSPFLLGSLYGHIHNEAV

SEQ ID NO: 77 - protein, 503 aa, synthetic construct
  REGION 1..503: note = Polypeptide sequence of CLT Antigen Fusion Protein 2
MWNSLEARSPKSRCCSTGPWEAVQENLLWASLLAPGGATIFPDPWLLKASPSSLPHLHRTSPCACLCPNFPFYKDTVLLHQGPRGGGKGGMCALQGRGASPAGAGLFHWTMSPFLLGSLYGHIHNEAVGGGKGGMAMGQRTPSLAELRKSSATFLTCNLGAQAEKRSRAPGKLTYVSTIVLDAPVTKLEQGLVMKRYKIVTQGFDYTSVESGGGGTGVLIRRGDLVTDMVACRIKTFRGHTEKAAICKTRKESSAETSPADSLILDFQPLQLMSSFSTLASLDKGGGMAKTKGSLSVFRELHPAAAFDRAVHFLFLELWLPEPMLSSSPPSSTAPLLGSEPLRHWEASLSRGGGGMWNFFRRELTSNGFPENFSLDVPANTYNALKSRLCDPNADHTSCPSPCSLHAAGALPGTGRQRWRVELAHLADRKLSLRDVSRLRQGGERRSGIAVKVVRGGAGFAARLQGSVTLVQQGWFFPRLGGCQAWWRMGAVVWCGELLTCTS

SEQ ID NO: 78 - protein, 609 aa, synthetic construct
  REGION 1..609: note = Polypeptide sequence of CLT Antigen Fusion Protein 3
MWNFFRRELTSNGFPENFSLDVPANTYNALKSRLCDPNADHTSCPSPCSLHAAGALPGTGRQRWRVELAHLADRKLSLRDVSRLRQGGERRSGIAVKVVRGGAGFAARLQGSVTLVQQGWFFPRLGGCQAWWRMGAVVWCGELLTCTSGGKTGVLIRRGDLVTDMVACRIKTFRGHTEKAAICKTRKESSAETSPADSLILDFQPLQLMSSFSTLASLDKGGGMNTPNIVSLRAHQPEVGIIPSVLLMRPLRIKGVFHHIHSPLHGENQGFTLCLQGAPPSSSVGGKGGMAMGQRTPSLAELRKSSATFLTCNLGAQAEKRSRAPGKLTYVSTIVLDAPVTKLEQGLVMKRYKIVTQGFDYTSVESKGGMAKTKGSLSVFRELHPAAAFDRAVHFLFLELWLPEPMLSSSPPSSTAPLLGSEPLRHWEASLSRKGGLPRTPRPDLILLQLLPAGLRQLLQTSGPDNEQPIEQDLICNVCGGGWNSLEARSPKSRCCSTGPWEAVQENLLWASLLAPGGATIFPDPWLLKASPSSLPHLHRTSPCACLCPNFPFYKDTVLLHQGPRGGGKGGMCALQGRGASPAGAGLFHWTMSPFLLGSLYGHIHNEAV

SEQ ID NO: 79 - protein, 617 aa, synthetic construct
  REGION 1..617: note = Polypeptide sequence of CLT Antigen Fusion Protein 4
MWNSLEARSPKSRCCSTGPWEAVQENLLWASLLAPGGATIFPDPWLLKASPSSLPHLHRTSPCACLCPNFPFYKDTVLLHQGPRGGGGMNTPNIVSLRAHQPEVGIIPSVLLMRPLRIKGVFHHIHSPLHGENQGFTLCLQGAPPSSSVGGGKGGMWNFFRRELTSNGFPENFSLDVPANTYNALKSRLCDPNADHTSCPSPCSLHAAGALPGTGRQRWRVELAHLADRKLSLRDVSRLRQGGERRSGIAVKVVRGGAGFAARLQGSVTLVQQGWFFPRLGGCQAWWRMGAVVWCGELLTCTSGGGGMLPRTPRPDLILLQLLPAGLRQLLQTSGPDNEQPIEQDLICNVCGGGGMAKTKGSLSVFRELHPAAAFDRAVHFLFLELWLPEPMLSSSPPSSTAPLLGSEPLRHWEASLSRGGGGMCALQGRGASPAGAGLFHWTMSPFLLGSLYGHIHNEAVGGGKGGMAMGQRTPSLAELRKSSATFLTCNLGAQAEKRSRAPGKLTYVSTIVLDAPVTKLEQGLVMKRYKIVTQGFDYTSVESGGGGTGVLIRRGDLVTDMVACRIKTFRGHTEKAAICKTRKESSAETSPADSLILDFQPLQLMSSFSTLASLDK

SEQ ID NO: 80 - DNA, 1506 bp, synthetic construct
  misc_feature 1..1506: note = Codon-optimised cDNA sequence encoding CLT Antigen Fusion Protein1
  source 1..1506: mol_type = other DNA
atgtggaatttcttccggcgcgagctgaccagcaacggcttccctgagaacttcagcctggacgtgcccgccaacacctacaatgccctgaagtccagactgtgcgaccccaacgccgatcacaccagctgtccatctccatgttctctgcatgccgctggcgctctgcctggaacaggcagacaaagatggcgcgtggaactggcccatctggccgatagaaagctgagcctgagggatgtgtcccggctgagacaaggcggcgagagaagatctggaatcgccgtgaaggtcgtcagaggcggagctggatttgccgctagactgcagggaagcgtgaccctggttcagcaaggctggttcttccctagactcggcggttgtcaagcctggtggcgaatgggagctgttgtttggtgcggcgagctgctgacctgtacatctggcggaggaacaggcgtgctgatcagaagaggcgacctggtcacagacatggtggcctgcagaatcaagaccttcagaggccacacagagaaggccgccatctgcaagacccggaaagagtctagcgccgagacaagccctgccgactctctgatcctggacttccagcctctgcagctgatgagcagctttagcacactggctagcctggacaaaggcggaaaaggcggcatgtggaacagcctggaagccagatctcccaagagccggtgttgtagcacaggcccttgggaagctgtgcaagagaatctgctgtgggcctctctgcttgctcctggcggagccaccatctttccagatccttggctgctgaaggctagccccagctctctgcctcatctgcacagaacaagcccctgcgcctgcctgtgtcctaacttcccattctacaaggacaccgtgctgctgcatcagggccctagaggtggtggaaaaggtggaatggccatgggccagagaacaccttctctggctgagctgagaaagagcagcgccaccttcctgacctgcaatctgggagcccaggccgagaagagatctagagcccctggcaagctgacctacgtgtccaccattgtgctggacgcccctgtgaccaagctcgaacagggactcgtgatgaagcggtacaagatcgtgacccagggcttcgactacaccagcgtggaatctaaaggcggaatggctaagaccaagggcagcctgagcgtgttcagagaactgcatcctgccgccgctttcgacagagccgtgcacttcctgtttctggaactgtggctgcccgagcctatgctgtctagcagccctcctagctctacagcccctctgctgggatctgagcctctgagacactgggaagccagcctgtctagaggcggtggcggaatgtgtgctctgcaaggcagaggcgcttctcctgctggtgccggactgttccactggacaatgagcccatttctgctggggagcctgtacggccacatccacaatgaggccgtctga

SEQ ID NO: 81 - DNA, 1512 bp, synthetic construct
  misc_feature 1..1512: note = Codon-optimised cDNA sequence encoding CLT Antigen Fusion Protein2
  source 1..1512: mol_type = other DNA
atgtggaactccctagaagcgagatccccgaaatctagatgttgttctactggaccgtgggaagccgtacaagaaaatctactatgggcctctctactagcaccaggtggtgcaacaatttttccagatccgtggctattgaaggcctcgccatcttctttaccgcatctacacagaacatccccgtgtgcttgtctatgtccgaactttccgttctacaaggacaccgtactactacatcaaggaccaagaggtggtggaaaaggtggaatgtgtgcactacaaggaagaggtgctagtccagctggtgctggattattccattggacaatgtccccgttcctactaggatctctatacggacacatccacaacgaagctgtcggaggtggaaaaggtggaatggctatgggacaaagaacaccatctctagcggagctaagaaagtcctctgcgacattcctaacctgtaacttgggagcgcaagcggagaaaagatctagagcacctggaaagctaacctatgtctccaccatagtactagatgcgccggtcacaaagctagaacagggactagtaatgaagagatacaagatcgtcacccagggattcgactacacctctgtagaatctggtggtggtggaaccggtgtcctaattagaagaggagatctagtcaccgatatggtcgcgtgtagaatcaagacattcagaggacatacagagaaggcggcgatctgcaagacaagaaaagaatcttctgcggaaacctctccggcggactctctaatcttagattttcagccgctacagctaatgtcctccttctctacattggcctcgctagacaaaggtggtggaatggcgaaaacgaagggatccttgtccgtattcagagaactacatccagctgcggctttcgatagagcggtacatttcctattcctagagctatggctaccggaaccgatgctatcttctagtccaccatcttctacagcgccgctattaggatctgaaccgctaagacattgggaagcgagtctatctagaggtggtggtggaatgtggaacttcttcagaagagagctaacctccaacggattccccgagaacttctctttagatgtaccggcgaacacctacaacgcgctaaagtctagattgtgtgatccgaacgcggaccatacttcttgtccatctccgtgttctttacatgctgctggtgctttacctggaacgggtagacaaagatggagagtagaactagcgcacctagcggacagaaagctatccctaagagatgtctccagactaagacaaggtggagagagaagatctggaatcgcggtcaaagtagtcagaggtggtgcaggatttgctgcgagattacagggatctgtaaccttggtacagcaaggatggttcttcccaagacttggaggatgtcaagcttggtggagaatgggagctgtagtttggtgcggagaactattgacatgtacatcttga

SEQ ID NO: 82 - DNA, 1830 bp, synthetic construct
  misc_feature 1..1830: note = Codon-optimised cDNA sequence encoding CLT Antigen Fusion Protein3
  source 1..1830: mol_type = other DNA
atgtggaatttcttccggcgcgagctgaccagcaacggcttccctgagaacttcagcctggacgtgcccgccaacacctacaatgccctgaagtccagactgtgcgaccccaacgccgatcacaccagctgtccatctccatgttctctgcatgccgctggcgctctgcctggaacaggcagacaaagatggcgcgtggaactggcccatctggccgatagaaagctgagcctgagggatgtgtcccggctgagacaaggcggcgagagaagatctggaatcgccgtgaaggtcgtcagaggcggagctggatttgccgctagactgcagggaagcgtgaccctggttcagcaaggctggttcttccctagactcggcggttgtcaagcctggtggcgaatgggagctgttgtttggtgcggcgagctgctgacatgtacaagcggcggaaaaaccggcgtgctgatcagaagaggcgacctggtcacagacatggtggcctgcagaatcaagaccttcagaggccacacagagaaggccgccatctgcaagacccggaaagagtctagcgccgagacaagccctgccgactctctgatcctggacttccagcctctgcagctgatgagcagctttagcacactggctagcctggacaaaggcggcggaatgaacacccctaacatcgtgtccctgagagcccaccagcctgaagtgggaatcatccctagcgtgctgctgatgcggcccctgagaatcaaaggcgtgttccaccacattcacagccctctgcacggcgagaatcagggcttcaccttgtgtctgcaaggcgcccctcctagcagttctgttggtggcaaaggcggaatggccatgggccagagaacaccatctctggccgagctgagaaagagcagcgccaccttcctgacctgtaacctgggagcccaggccgagaagagatctagagcccctggcaagctgacctacgtgtccaccattgtgctggacgcccctgtgaccaagctcgaacagggactcgtgatgaagcggtacaagatcgtgacccagggcttcgactacaccagcgtggaatctaaaggtggcatggccaagaccaagggcagcctgtccgtgttcagagagctgcatcctgccgccgctttcgatagagccgtgcacttcctgtttctggaactgtggctgcccgagcctatgctgtctagcagccctccatctagcacagccccactgctgggatctgagcctctgagacactgggaagccagcctgagcagaaaaggcggcctgcctagaacacccagacctgacctgattctgctgcagctgctgcctgctggactgagacaactgctgcagacaagcggccctgacaacgagcagcctatcgagcaggacctgatctgcaatgtgtgcggaggcggctggaacagcctggaagccagatctcctaagagccggtgctgtagcacaggcccttgggaagctgtgcaagagaatctgctgtgggcctctctgcttgctcctggcggagccaccatctttccagatccttggctgctgaaggctagcccctctagcctgcctcatctgcacagaacaagcccctgcgcctgcctgtgtcctaacttcccattctacaaggacacagtgctgctgcatcagggccctcgaggtggcggaaaaggtggaatgtgtgcccttcaaggcagaggcgcttctcctgctggtgccggactgttccactggacaatgagcccatttctgctggggagcctgtacggccacatccacaatgaagccgtctga

SEQ ID NO: 83 - DNA, 1854 bp, synthetic construct
  misc_feature 1..1854: note = Codon-optimised cDNA sequence encoding CLT Antigen Fusion Protein4
  source 1..1854: mol_type = other DNA
atgtggaactccctagaagcgagatccccgaaatctagatgttgttctactggaccgtgggaagccgtacaagaaaatctactatgggcctctctactagcaccaggtggtgcaacaatttttccagatccgtggctattgaaggcctcgccatcttctttaccgcatctacacagaacatccccgtgtgcttgtctatgtccgaactttccgttctacaaggacaccgtactactacatcaaggaccaagaggtggtggtggaatgaatactccgaacatcgtatctctaagagcgcatcaaccggaagttggaattatcccgtccgtcctactaatgagaccgctaagaatcaagggagtgttccaccatatccactctccactacacggagaaaaccagggattcaccctatgtttacaaggtgctccaccgtcctctagtgttggaggtggaaaaggtggaatgtggaatttcttcagaagagagctaacctccaacggattccccgagaacttctctttagatgtaccggcgaacacctacaacgcgctaaagtctagattgtgtgatccgaacgcggaccatacttcttgtccatctccatgttctctacatgctgctggtgctttacctggaactggtagacaaagatggagagtagaactagcgcacctagcggacagaaagctatccctaagagatgtctccagactaagacaaggtggagagagaagatctggaatcgcggtcaaagtagttagaggtggtgcaggatttgcggcgagattacaaggatctgtaaccctagtacagcaaggatggttcttcccaagacttggaggatgtcaagcttggtggagaatgggagctgtagtttggtgcggagaactactaacatgtacctctggtggtggtggaatgctaccaagaacaccaagaccggacctaatcctactacaactactaccagcgggattgagacagctactacaaacatctggaccggataacgaacagccgatcgaacaggatctaatctgtaacgtatgcggaggtggtggaatggcgaaaacaaagggatccttgtccgtgttcagagaactacatccagctgcggcttttgatagagcggtacacttcctattcctagagctatggctaccggaaccgatgttatcttcttccccaccatcttctacagcgccgttattaggatctgaaccgttgagacattgggaagcgagtctatctagaggtggtggtggaatgtgtgcgttacaaggaagaggtgctagtccagctggtgccggattatttcattggacaatgtccccgttcctactaggatctctatacggacacatccacaacgaagctgtcggtggtggaaaaggtggaatggctatgggacaaagaacaccatctctagcggagctaagaaagtcctctgcgacattcctaacctgtaacttgggagcgcaagcggagaaaagatctagagcacctggaaagctaacctatgtctccaccatagtactagatgcgccggtcacaaagctagaacagggactagtaatgaagagatacaagatcgtcacgcagggattcgactacacctctgtagaatccggtggtggtggtacaggtgtcctaattagaagaggagatctagtcaccgatatggtcgcgtgtagaatcaagacattcagaggacatacagagaaggcggcgatctgcaagacaagaaaagaatcttctgcggaaacctctccggcggactctctaatcttagattttcagccgctacagctaatgtcctccttctctactttggcctccttggataagtga

SEQ ID NO: 84 - skipped

SEQ ID NO: 85 - protein, 15 aa, synthetic construct
  REGION 1..15: note = TCR VB CDR3 AA sequence
CASSLTGGYTGELFF

SEQ ID NO: 86 - protein, 13 aa, synthetic construct
  REGION 1..13: note = TCR VB CDR3 AA sequence
CASNKLGYQPQHF

SEQ ID NO: 87 - protein, 13 aa, synthetic construct
  REGION 1..13: note = TCR VB CDR3 AA sequence
CASSLLENQPQHF